SEQUENCE LISTING (513 sequences)

SEQ ID NO: 1 - protein, 42 aa, Artificial Sequence
  source: /note=“Description of Artificial Sequence Synthetic polypeptide”
  MOD_RES (5)..(5): Any amino acid, amino acid analogue or not present
  MOD_RES (7)..(8): Any amino acid, amino acid analogue or not present
  MOD_RES (11)..(11): Any amino acid, amino acid analogue or not present
  MOD_RES (17)..(17): Any amino acid, amino acid analogue or not present
  MOD_RES (22)..(24): Any amino acid, amino acid analogue or not present
  MOD_RES (26)..(26): Any amino acid, amino acid analogue or not present
  MOD_RES (28)..(28): Any amino acid, amino acid analogue or not present
  MOD_RES (32)..(32): Any amino acid, amino acid analogue or not present
  MOD_RES (39)..(42): Any amino acid, amino acid analogue or not present
GSGVXIXXKCXGSKQCXDPCKXXXGXRXGKCXNKKCKCXXXX

SEQ ID NO: 2 - protein, 42 aa, Artificial Sequence
  source: /note=“Description of Artificial Sequence Synthetic polypeptide”
  VARIANT (5)..(5): /replace=“Arg”
  VARIANT (7)..(7): /replace=“Asn”
  VARIANT (8)..(8): /replace=“Ile”
  VARIANT (11)..(11): /replace=“Thr” or “Arg” or “Lys”
  VARIANT (17)..(17): /replace=“Leu”
  VARIANT (22)..(22): /replace=“Arg” or “Lys”
  VARIANT (23)..(23): /replace=“Lys” or “Arg”
  VARIANT (24)..(24): /replace=“Ala”
  VARIANT (26)..(26): /replace=“Met”
  VARIANT (28)..(28): /replace=“Asn”
  VARIANT (32)..(32): /replace=“Ile”
  VARIANT (39)..(39): /replace=“Thr”
  VARIANT (40)..(40): /replace=“Pro”
  VARIANT (41)..(41): /replace=“ ”
  VARIANT (42)..(42): /replace=“ ”
  SITE (1)..(42): /note=“Variant residues given in the sequence have no preference with respect to those in the annotations for variant positions”
GSGVPIPVKCSGSKQCYDPCKQATGCRFGKCMNKKCKCYGCG

SEQ ID NO: 3 - protein, 41 aa, Artificial Sequence
  source: /note=“Description of Artificial Sequence Synthetic polypeptide”
  MOD_RES (3)..(6): Any amino acid, amino acid analogue or not present
  MOD_RES (8)..(8): Any amino acid, amino acid analogue or not present
  MOD_RES (10)..(10): Any amino acid, amino acid analogue or not present
  MOD_RES (17)..(17): Any amino acid, amino acid analogue or not present
  MOD_RES (21)..(22): Any amino acid, amino acid analogue or not present
  MOD_RES (26)..(28): Any amino acid, amino acid analogue or not present
  MOD_RES (31)..(32): Any amino acid, amino acid analogue or not present
  MOD_RES (34)..(34): Any amino acid, amino acid analogue or not present
GSXXXXIXCXGSKQCYXPCKXXTGCXXXKCXXKXCKCYGCG

SEQ ID NO: 4 - protein, 41 aa, Artificial Sequence
  source: /note=“Description of Artificial Sequence Synthetic polypeptide”
  VARIANT (3)..(3): /replace=“ ”
  VARIANT (4)..(4): /replace=“ ”
  VARIANT (5)..(5): /replace=“Gly” or “ ”
  VARIANT (6)..(6): /replace=“Ser” or “ ”
  VARIANT (8)..(8): /replace=“Ser”
  VARIANT (10)..(10): /replace=“Thr”
  VARIANT (17)..(17): /replace=“Asp”
  VARIANT (21)..(21): /replace=“Arg”
  VARIANT (22)..(22): /replace=“Lys”
  VARIANT (26)..(26): /replace=“Pro”
  VARIANT (27)..(27): /replace=“Gln”
  VARIANT (28)..(28): /replace=“Ala”
  VARIANT (31)..(31): /replace=“Leu”
  VARIANT (32)..(32): /replace=“Gln”
  VARIANT (34)..(34): /replace=“Ser”
  SITE (1)..(41): /note=“Variant residues given in the sequence have no preference with respect to those in the annotations for variant positions”
GSGSEVIRCSGSKQCYGPCKQQTGCTNSKCMNKVCKCYGCG

SEQ ID NO: 5 - protein, 42 aa, Artificial Sequence
  source: /note=“Description of Artificial Sequence Synthetic polypeptide”
  MOD_RES (3)..(5): Any amino acid, amino acid analogue or not present
  MOD_RES (7)..(7): Any amino acid, amino acid analogue or not present
  MOD_RES (9)..(9): Any amino acid, amino acid analogue or not present
  MOD_RES (11)..(11): Any amino acid, amino acid analogue or not present
  MOD_RES (13)..(14): Any amino acid, amino acid analogue or not present
  MOD_RES (16)..(17): Any amino acid, amino acid analogue or not present
  MOD_RES (20)..(20): Any amino acid, amino acid analogue or not present
  MOD_RES (24)..(24): Any amino acid, amino acid analogue or not present
  MOD_RES (33)..(33): Any amino acid, amino acid analogue or not present
  MOD_RES (35)..(35): Any amino acid, amino acid analogue or not present
  MOD_RES (38)..(38): Any amino acid, amino acid analogue or not present
  MOD_RES (42)..(42): Any amino acid, amino acid analogue or not present
GSXXXVXIXVXCXXSXXCLXPCKXAGMRFGKCXNXKCXCTPX

SEQ ID NO: 6 - protein, 42 aa, Artificial Sequence
  source: /note=“Description of Artificial Sequence Synthetic polypeptide”
  VARIANT (3)..(3): /replace=“ ”
  VARIANT (4)..(4): /replace=“Ser” or “ ”
  VARIANT (5)..(5): /replace=“ Ser” or “ ”
  VARIANT (7)..(7): /replace=“Arg”
  VARIANT (9)..(9): /replace=“Pro”
  VARIANT (11)..(11): /replace=“Ser”
  VARIANT (13)..(13): /replace=“Lys”
  VARIANT (14)..(14): /replace=“His”
  VARIANT (16)..(16): /replace=“Gly”
  VARIANT (17)..(17): /replace=“Gln”
  VARIANT (20)..(20): /replace=“Lys”
  VARIANT (24)..(24): /replace=“Asp”
  VARIANT (33)..(33): /replace=“Met”
  VARIANT (35)..(35): /replace=“Gly”
  VARIANT (38)..(38): /replace=“Asp”
  VARIANT (42)..(42): /replace=“ ”
  SITE (1)..(42): /note=“Variant residues given in the sequence have no preference with respect to those in the annotations for variant positions”
GSGGGVPINVKCRGSRDCLDPCKKAGMRFGKCINSKCHCTPK

SEQ ID NO: 7 - protein, 37 aa, Artificial Sequence
  source: /note=“Description of Artificial Sequence Synthetic polypeptide”
  MOD_RES (3)..(3): Any amino acid
  MOD_RES (5)..(5): Any amino acid
  MOD_RES (9)..(9): Any amino acid
  MOD_RES (15)..(15): Any amino acid
  MOD_RES (20)..(20): Any amino acid
  MOD_RES (22)..(22): Any amino acid
  MOD_RES (24)..(24): Any amino acid
  MOD_RES (32)..(32): Any amino acid
  MOD_RES (37)..(37): Any amino acid
  source: /note=“See specification as filed for detailed description of substitutions and preferred embodiments”
GSXVXVKCXGSKQCXPCKRXGXRXGKCINKKXCKCYX

SEQ ID NO: 8 - protein, 34 aa, Artificial Sequence
  source: /note=“Description of Artificial Sequence Synthetic polypeptide”
  MOD_RES (3)..(3): Any amino acid
  MOD_RES (7)..(7): Any amino acid
  MOD_RES (13)..(13): Any amino acid
  MOD_RES (15)..(15): Any amino acid
  MOD_RES (18)..(18): Any amino acid
  MOD_RES (20)..(20): Any amino acid
  MOD_RES (29)..(29): Any amino acid
  MOD_RES (34)..(34): Any amino acid
  source: /note=“See specification as filed for detailed description of substitutions and preferred embodiments”
GSXGCVXKCRPGXKXCCXPXKRCSRRFGXKKCKX

SEQ ID NO: 9 - protein, 43 aa, Artificial Sequence
  source: /note=“Description of Artificial Sequence Synthetic polypeptide”
  MOD_RES (3)..(3): Any amino acid
  MOD_RES (5)..(7): Any amino acid
  MOD_RES (11)..(11): Any amino acid
  MOD_RES (17)..(18): Any amino acid
  MOD_RES (23)..(24): Any amino acid
  MOD_RES (26)..(26): Any amino acid
  MOD_RES (28)..(28): Any amino acid
  MOD_RES (36)..(36): Any amino acid
  MOD_RES (41)..(43): Any amino acid
  source: /note=“See specification as filed for detailed description of substitutions and preferred embodiments”
GSXVXXXVKCXGSKQCXXPCKRXXGXRXGKCINKKXCKCYXXX

SEQ ID NO: 10 - protein, 47 aa, Artificial Sequence
  source: /note=“Description of Artificial Sequence Synthetic polypeptide”
  MOD_RES (3)..(5): Any amino acid
  MOD_RES (9)..(12): Any amino acid
  MOD_RES (18)..(18): Any amino acid
  MOD_RES (20)..(20): Any amino acid
  MOD_RES (23)..(23): Any amino acid
  MOD_RES (25)..(25): Any amino acid
  MOD_RES (34)..(37): Any amino acid
  MOD_RES (42)..(47): Any amino acid
  source: /note=“See specification as filed for detailed description of substitutions and preferred embodiments”
GSXXXGCVXXXXKCRPGXKXCCXPXKRCSRRFGXXXXKKCKXXXXXX

SEQ ID NO: 11 - protein, 42 aa, Artificial Sequence
  source: /note=“Description of Artificial Sequence Synthetic polypeptide”
  MOD_RES (5)..(5): Any amino acid, amino acid analogue or not present
  MOD_RES (7)..(8): Any amino acid, amino acid analogue or not present
  MOD_RES (11)..(11): Any amino acid, amino acid analogue or not present
  MOD_RES (17)..(17): Any amino acid, amino acid analogue or not present
  MOD_RES (22)..(24): Any amino acid, amino acid analogue or not present
  MOD_RES (26)..(26): Any amino acid, amino acid analogue or not present
  MOD_RES (28)..(28): Any amino acid, amino acid analogue or not present
  MOD_RES (32)..(32): Any amino acid, amino acid analogue or not present
  MOD_RES (39)..(42): Any amino acid, amino acid analogue or not present
GSGVXIXXRCXGSRQCXDPCRXXXGXRXGRCXNRRCRCXXXX

SEQ ID NO: 12 - protein, 42 aa, Artificial Sequence
  source: /note=“Description of Artificial Sequence Synthetic polypeptide”
  VARIANT (5)..(5): /replace=“Arg”
  VARIANT (7)..(7): /replace=“Asn”
  VARIANT (8)..(8): /replace=“Ile”
  VARIANT (11)..(11): /replace=“Thr” or “Arg” or “Lys”
  VARIANT (17)..(17): /replace=“Leu”
  VARIANT (22)..(22): /replace=“Arg” or “Lys”
  VARIANT (23)..(23): /replace=“Lys” or “Arg”
  VARIANT (24)..(24): /replace=“Ala”
  VARIANT (26)..(26): /replace=“Met”
  VARIANT (28)..(28): /replace=“Asn”
  VARIANT (32)..(32): /replace=“Ile”
  VARIANT (39)..(39): /replace=“Thr”
  VARIANT (40)..(40): /replace=“Pro”
  VARIANT (41)..(41): /replace=“ ”
  VARIANT (42)..(42): /replace=“ ”
  SITE (1)..(42): /note=“Variant residues given in the sequence have no preference with respect to those in the annotations for variant positions”
GSGVPIPVRCSGSRQCYDPCRQATGCRFGRCMNRRCRCYGCG

SEQ ID NO: 13 - protein, 41 aa, Artificial Sequence
  source: /note=“Description of Artificial Sequence Synthetic polypeptide”
  MOD_RES (3)..(6): Any amino acid, amino acid analogue or not present
  MOD_RES (8)..(8): Any amino acid, amino acid analogue or not present
  MOD_RES (10)..(10): Any amino acid, amino acid analogue or not present
  MOD_RES (17)..(17): Any amino acid, amino acid analogue or not present
  MOD_RES (21)..(22): Any amino acid, amino acid analogue or not present
  MOD_RES (26)..(28): Any amino acid, amino acid analogue or not present
  MOD_RES (31)..(32): Any amino acid, amino acid analogue or not present
  MOD_RES (34)..(34): Any amino acid, amino acid analogue or not present
GSXXXXIXCXGSRQCYXPCRXXTGCXXXRCXXRXCRCYGCG

SEQ ID NO: 14 - protein, 41 aa, Artificial Sequence
  source: /note=“Description of Artificial Sequence Synthetic polypeptide”
  VARIANT (3)..(3): /replace=“ ”
  VARIANT (4)..(4): /replace=“ ”
  VARIANT (5)..(5): /replace=“Gly” or “ ”
  VARIANT (6)..(6): /replace=“Ser” or “ ”
  VARIANT (8)..(8): /replace=“Ser”
  VARIANT (10)..(10): /replace=“Thr”
  VARIANT (17)..(17): /replace=“Asp”
  VARIANT (21)..(21): /replace=“Arg”
  VARIANT (22)..(22): /replace=“Arg” or “Lys”
  VARIANT (26)..(26): /replace=“Pro”
  VARIANT (27)..(27): /replace=“Gln”
  VARIANT (28)..(28): /replace=“Ala”
  VARIANT (31)..(31): /replace=“Leu”
  VARIANT (32)..(32): /replace=“Gln”
  VARIANT (34)..(34): /replace=“Ser”
  SITE (1)..(41): /note=“Variant residues given in the sequence have no preference with respect to those in the annotations for variant positions”
GSGSEVIRCSGSRQCYGPCRQQTGCTNSRCMNRVCRCYGCG

SEQ ID NO: 15 - protein, 42 aa, Artificial Sequence
  source: /note=“Description of Artificial Sequence Synthetic polypeptide”
  MOD_RES (3)..(5): Any amino acid, amino acid analogue or not present
  MOD_RES (7)..(7): Any amino acid, amino acid analogue or not present
  MOD_RES (9)..(9): Any amino acid, amino acid analogue or not present
  MOD_RES (11)..(11): Any amino acid, amino acid analogue or not present
  MOD_RES (13)..(14): Any amino acid, amino acid analogue or not present
  MOD_RES (16)..(17): Any amino acid, amino acid analogue or not present
  MOD_RES (20)..(20): Any amino acid, amino acid analogue or not present
  MOD_RES (24)..(24): Any amino acid, amino acid analogue or not present
  MOD_RES (33)..(33): Any amino acid, amino acid analogue or not present
  MOD_RES (35)..(35): Any amino acid, amino acid analogue or not present
  MOD_RES (38)..(38): Any amino acid, amino acid analogue or not present
  MOD_RES (42)..(42): Any amino acid, amino acid analogue or not present
GSXXXVXIXVXCXXSXXCLXPCRXAGMRFGRCXNXRCXCTPX

SEQ ID NO: 16 - protein, 42 aa, Artificial Sequence
  source: /note=“Description of Artificial Sequence Synthetic polypeptide”
  VARIANT (3)..(3): /replace=“ ”
  VARIANT (4)..(4): /replace=“Ser” or “ ”
  VARIANT (5)..(5): /replace=“Ser” or “ ”
  VARIANT (7)..(7): /replace=“Arg”
  VARIANT (9)..(9): /replace=“Pro”
  VARIANT (11)..(11): /replace=“Lys” or “Ser”
  VARIANT (13)..(13): /replace=“Lys”
  VARIANT (14)..(14): /replace=“His”
  VARIANT (16)..(16): /replace=“Gly”
  VARIANT (17)..(17): /replace=“Gln”
  VARIANT (20)..(20): /replace=“Arg” or “Lys”
  VARIANT (24)..(24): /replace=“Arg” or “Asp”
  VARIANT (33)..(33): /replace=“Met”
  VARIANT (35)..(35): /replace=“Gly”
  VARIANT (38)..(38): /replace=“Asp”
  VARIANT (42)..(42): /replace=“Arg” or “ ”
  SITE (1)..(42): /note=“Variant residues given in the sequence have no preference with respect to those in the annotations for variant positions”
GSGGGVPINVRCRGSRDCLDPCRKAGMRFGRCINSRCHCTPK

SEQ ID NO: 17 - protein, 37 aa, Artificial Sequence
  source: /note=“Description of Artificial Sequence Synthetic polypeptide”
  MOD_RES (3)..(3): Any amino acid
  MOD_RES (5)..(5): Any amino acid
  MOD_RES (9)..(9): Any amino acid
  MOD_RES (15)..(15): Any amino acid
  MOD_RES (20)..(20): Any amino acid
  MOD_RES (22)..(22): Any amino acid
  MOD_RES (24)..(24): Any amino acid
  MOD_RES (32)..(32): Any amino acid
  MOD_RES (37)..(37): Any amino acid
  source: /note=“See specification as filed for detailed description of substitutions and preferred embodiments”
GSXVXVRCXGSRQCXPCRRXGXRXGRCINRRXCRCYX

SEQ ID NO: 18 - protein, 34 aa, Artificial Sequence
  source: /note=“Description of Artificial Sequence Synthetic polypeptide”
  MOD_RES (3)..(3): Any amino acid
  MOD_RES (7)..(7): Any amino acid
  MOD_RES (13)..(13): Any amino acid
  MOD_RES (15)..(15): Any amino acid
  MOD_RES (18)..(18): Any amino acid
  MOD_RES (20)..(20): Any amino acid
  MOD_RES (29)..(29): Any amino acid
  MOD_RES (34)..(34): Any amino acid
  source: /note=“See specification as filed for detailed description of substitutions and preferred embodiments”
GSXGCVXRCRPGXRXCCXPXRRCSRRFGXRRCRX

SEQ ID NO: 19 - protein, 43 aa, Artificial Sequence
  source: /note=“Description of Artificial Sequence Synthetic polypeptide”
  MOD_RES (3)..(3): Any amino acid, amino acid analogue or not present
  MOD_RES (5)..(7): Any amino acid, amino acid analogue or not present
  MOD_RES (11)..(11): Any amino acid, amino acid analogue or not present
  MOD_RES (17)..(18): Any amino acid, amino acid analogue or not present
  MOD_RES (23)..(24): Any amino acid, amino acid analogue or not present
  MOD_RES (26)..(26): Any amino acid, amino acid analogue or not present
  MOD_RES (28)..(28): Any amino acid, amino acid analogue or not present
  MOD_RES (36)..(36): Any amino acid, amino acid analogue or not present
  MOD_RES (41)..(43): Any amino acid, amino acid analogue or not present
GSXVXXXVRCXGSRQCXXPCRRXXGXRXGRCINRRXCRCYXXX

SEQ ID NO: 20 - protein, 47 aa, Artificial Sequence
  source: /note=“Description of Artificial Sequence Synthetic polypeptide”
  MOD_RES (3)..(5): Any amino acid, amino acid analogue or not present
  MOD_RES (9)..(12): Any amino acid, amino acid analogue or not present
  MOD_RES (18)..(18): Any amino acid, amino acid analogue or not present
  MOD_RES (20)..(20): Any amino acid, amino acid analogue or not present
  MOD_RES (23)..(23): Any amino acid, amino acid analogue or not present
  MOD_RES (25)..(25): Any amino acid, amino acid analogue or not present
  MOD_RES (34)..(37): Any amino acid, amino acid analogue or not present
  MOD_RES (42)..(47): Any amino acid, amino acid analogue or not present
GSXXXGCVXXXXRCRPGXRXCCXPXRRCSRRFGXXXXRRCRXXXXXX

SEQ ID NO: 21 - protein, 34 aa, Artificial Sequence
  source: /note=“Description of Artificial Sequence Synthetic polypeptide”
  source: /note=“See specification as filed for detailed description of substitutions and preferred embodiments”
GSGIVCKVCKIICGMQGKKVNICKAPIKCKCKKG

SEQ ID NO: 22 - protein, 37 aa, Artificial Sequence
  source: /note=“Description of Artificial Sequence Synthetic polypeptide”
  source: /note=“See specification as filed for detailed description of substitutions and preferred embodiments”
GSSEKDCIKHLQRCRENKDCCSKKCSRRGTNPEKRCR

SEQ ID NO: 23 - protein, 39 aa, Artificial Sequence
  source: /note=“Description of Artificial Sequence Synthetic polypeptide”
  source: /note=“See specification as filed for detailed description of substitutions and preferred embodiments”
GSVRIPVSCKHSGQCLKPCKDAGMRFGKCMNGKCDCTPK

SEQ ID NO: 24 - protein, 39 aa, Artificial Sequence
  source: /note=“Description of Artificial Sequence Synthetic polypeptide”
  source: /note=“See specification as filed for detailed description of substitutions and preferred embodiments”
GSGVPINVKCRGSRDCLDPCKKAGMRFGKCINSKCHCTP

SEQ ID NO: 25 - protein, 35 aa, Artificial Sequence
  source: /note=“Description of Artificial Sequence Synthetic polypeptide”
  source: /note=“See specification as filed for detailed description of substitutions and preferred embodiments”
GSAVCVYRTCDKDCKRRGYRSGKCINNACKCYPYG

SEQ ID NO: 26 - protein, 37 aa, Artificial Sequence
  source: /note=“Description of Artificial Sequence Synthetic polypeptide”
  source: /note=“See specification as filed for detailed description of substitutions and preferred embodiments”
GSISCTGSKQCYDPCKRKTGCPNAKCMNKSCKCYGCG

SEQ ID NO: 27 - protein, 40 aa, Artificial Sequence
  source: /note=“Description of Artificial Sequence Synthetic polypeptide”
  source: /note=“See specification as filed for detailed description of substitutions and preferred embodiments”
GSQVQTNVKCQGGSCASVCRREIGVAAGKCINGKCVCYRN

SEQ ID NO: 28 - protein, 39 aa, Artificial Sequence
  source: /note=“Description of Artificial Sequence Synthetic polypeptide”
  source: /note=“See specification as filed for detailed description of substitutions and preferred embodiments”
GSEVIRCSGSKQCYGPCKQQTGCTNSKCMNKVCKCYGCG

SEQ ID NO: 29 - protein, 35 aa, Artificial Sequence
  source: /note=“Description of Artificial Sequence Synthetic polypeptide”
  source: /note=“See specification as filed for detailed description of substitutions and preferred embodiments”
GSACKGVFDACTPGKNECCPNRVCSDKHKWCKWKL

SEQ ID NO: 30 - protein, 40 aa, Artificial Sequence
  source: /note=“Description of Artificial Sequence Synthetic polypeptide”
  source: /note=“See specification as filed for detailed description of substitutions and preferred embodiments”
GSQIYTSKECNGSSECYSHCEGITGKRSGKCINKKCYCYR

SEQ ID NO: 31 - protein, 37 aa, Artificial Sequence
  source: /note=“Description of Artificial Sequence Synthetic polypeptide”
  source: /note=“See specification as filed for detailed description of substitutions and preferred embodiments”
GSGCLEFWWKCNPNDDKCCRPKLKCSKLFKLCNFSFG

SEQ ID NO: 32 - protein, 39 aa, Artificial Sequence
  source: /note=“Description of Artificial Sequence Synthetic polypeptide”
GSDCVRFWGKCSQTSDCCPHLACKSKWPRNICVWDGSVG

SEQ ID NO: 33 - protein, 35 aa, Artificial Sequence
  source: /note=“Description of Artificial Sequence Synthetic polypeptide”
  source: /note=“See specification as filed for detailed description of substitutions and preferred embodiments”
GSGCFGYKCDYYKGCCSGYVCSPTWKWCVRPGPGR

SEQ ID NO: 34 - protein, 62 aa, Artificial Sequence
  source: /note=“Description of Artificial Sequence Synthetic polypeptide”
GSMNAKFILLLVLTTMMLLPDTKGAEVIRCSGSKQCYGPCKQQTGCTNSKCMNKVCKCYGCG

SEQ ID NO: 35 - protein, 63 aa, Artificial Sequence
  source: /note=“Description of Artificial Sequence Synthetic polypeptide”
GSMNAKLIYLLLVVTTMTLMFDTAQAVDIMCSGPKQCYGPCKKETGCPNAKCMNRRCKCYGCV

SEQ ID NO: 36 - protein, 64 aa, Artificial Sequence
  source: /note=“Description of Artificial Sequence Synthetic polypeptide”
GSMNAKLIYLLLVVTTMMLTFDTTQAGDIKCSGTRQCWGPCKKQTTCTNSKCMNGKCKCYGCVG

SEQ ID NO: 37 - protein, 63 aa, Artificial Sequence
  source: /note=“Description of Artificial Sequence Synthetic polypeptide”
GSMNTKFIFLLLVVTNTMMLFDTKPVEGISCTGSKQCYDPCKRKTGCPNAKCMNKSCKCYGCG

SEQ ID NO: 38 - protein, 40 aa, Artificial Sequence
  source: /note=“Description of Artificial Sequence Synthetic polypeptide”
GSGVPINVKCSGSRDCLEPCKKAGMRFGKCINRKCHCTPK

SEQ ID NO: 39 - protein, 40 aa, Artificial Sequence
  source: /note=“Description of Artificial Sequence Synthetic polypeptide”
GSGVPINVKCTGSPQCLKPCKDAGMRFGKCINGKCHCTPK

SEQ ID NO: 40 - protein, 40 aa, Artificial Sequence
  source: /note=“Description of Artificial Sequence Synthetic polypeptide”
GSGVIINVKCKISRQCLEPCKKAGMRFGKCMNGKCHCTPK

SEQ ID NO: 41 - protein, 40 aa, Artificial Sequence
  source: /note=“Description of Artificial Sequence Synthetic polypeptide”
GSGVPINVKCRGSPQCIQPCRDAGMRFGKCMNGKCHCTPQ

SEQ ID NO: 42 - protein, 40 aa, Artificial Sequence
  source: /note=“Description of Artificial Sequence Synthetic polypeptide”
GSGVEINVKCTGSHQCIKPCKDAGMRFGKCINRKCHCTPK

SEQ ID NO: 43 - protein, 40 aa, Artificial Sequence
  source: /note=“Description of Artificial Sequence Synthetic polypeptide”
GSGVEINVKCSGSPQCLKPCKDAGMRFGKCMNRKCHCTPK

SEQ ID NO: 44 - protein, 40 aa, Artificial Sequence
  source: /note=“Description of Artificial Sequence Synthetic polypeptide”
GSGVPTDVKCRGSPQCIQPCKDAGMRFGKCMNGKCHCTPK

SEQ ID NO: 45 - protein, 40 aa, Artificial Sequence
  source: /note=“Description of Artificial Sequence Synthetic polypeptide”
GSGVPINVSCTGSPQCIKPCKDAGMRFGKCMNRKCHCTPK

SEQ ID NO: 46 - protein, 40 aa, Artificial Sequence
  source: /note=“Description of Artificial Sequence Synthetic polypeptide”
GSGVPINVPCTGSPQCIKPCKDAGMRFGKCMNRKCHCTPK

SEQ ID NO: 47 - protein, 39 aa, Artificial Sequence
  source: /note=“Description of Artificial Sequence Synthetic polypeptide”
GSVGINVKCKHSGQCLKPCKDAGMRFGKCINGKCDCTPK

SEQ ID NO: 48 - protein, 39 aa, Artificial Sequence
  source: /note=“Description of Artificial Sequence Synthetic polypeptide”
GSVGINVKCKHSGQCLKPCKDAGMRFGKCMNGKCDCTPK

SEQ ID NO: 49 - protein, 39 aa, Artificial Sequence
  source: /note=“Description of Artificial Sequence Synthetic polypeptide”
GSVGIPVSCKHSGQCIKPCKDAGMRFGKCMNRKCDCTPK

SEQ ID NO: 50 - protein, 38 aa, Artificial Sequence
  source: /note=“Description of Artificial Sequence Synthetic polypeptide”
GSRKGCFKEGHSCPKTAPCCRPLVCKGPSPNTKKCTRP

SEQ ID NO: 51 - protein, 34 aa, Artificial Sequence
  source: /note=“Description of Artificial Sequence Synthetic polypeptide”
GSSFCIPFKPCKSDENCCKKFKCKTTGIVKLCRW

SEQ ID NO: 52 - protein, 38 aa, Artificial Sequence
  source: /note=“Description of Artificial Sequence Synthetic polypeptide”
GSLKGCLPRNRFCNALSGPRCCSGLRCKELSIWASKCL

SEQ ID NO: 53 - protein, 36 aa, Artificial Sequence
  source: /note=“Description of Artificial Sequence Synthetic polypeptide”
GSGNYCLRGRCLPGGRKCCNGRPCECFAKICSCKPK

SEQ ID NO: 54 - protein, 31 aa, Artificial Sequence
  source: /note=“Description of Artificial Sequence Synthetic polypeptide”
GSTVKCGGCNRKCCPGGCRSGKCINGKCQCY

SEQ ID NO: 55 - protein, 31 aa, Artificial Sequence
  source: /note=“Description of Artificial Sequence Synthetic polypeptide”
GSGCMKEYCAGQCRGKVSQDYCLKHCKCIPR

SEQ ID NO: 56 - protein, 36 aa, Artificial Sequence
  source: /note=“Description of Artificial Sequence Synthetic polypeptide”
GSACLGFGEKCNPSNDKCCKSSSLVCSQKHKWCKYG

SEQ ID NO: 57 - protein, 39 aa, Artificial Sequence
  source: /note=“Description of Artificial Sequence Synthetic polypeptide”
GSRGGCLPHNRFCNALSGPRCCSGLRCKELSIRDSRCLG

SEQ ID NO: 58 - protein, 38 aa, Artificial Sequence
  source: /note=“Description of Artificial Sequence Synthetic polypeptide”
GSRGGCLPRNKFCNPSSGPRCCSGLTCKELNIWASKCL

SEQ ID NO: 59 - protein, 35 aa, Artificial Sequence
  source: /note=“Description of Artificial Sequence Synthetic polypeptide”
GSQRSCAKPGDMCMGIKCCDGQCGCNRGTGRCFCK

SEQ ID NO: 60 - protein, 42 aa, Artificial Sequence
  source: /note=“Description of Artificial Sequence Synthetic polypeptide”
GSARGCADAYKSCNHPRTCCDGYNGYKRACICSGSNCKCKKS

SEQ ID NO: 61 - protein, 39 aa, Artificial Sequence
  source: /note=“Description of Artificial Sequence Synthetic polypeptide”
GSRGGCLPHNRFCNALSGPRCCSGLRCKELSIWDSRCLG

SEQ ID NO: 62 - protein, 39 aa, Artificial Sequence
  source: /note=“Description of Artificial Sequence Synthetic polypeptide”
GSRGGCLPHNRFCNALSGPRCCSGLKCKELSIYDSRCLG

SEQ ID NO: 63 - protein, 39 aa, Artificial Sequence
  source: /note=“Description of Artificial Sequence Synthetic polypeptide”
GSRGGCLPHNRFCNALSGPRCCSRLKCKELSIWDSRCLG

SEQ ID NO: 64 - protein, 39 aa, Artificial Sequence
  source: /note=“Description of Artificial Sequence Synthetic polypeptide”
GSRGGCLPHNRFCNALTGPRCCSRLRCKELSIWDSICLG

SEQ ID NO: 65 - protein, 36 aa, Artificial Sequence
  source: /note=“Description of Artificial Sequence Synthetic polypeptide”
GSSCADAYKSCDSLKCCNNRTCMCSMIGTNCTCRKK

SEQ ID NO: 66 - protein, 32 aa, Artificial Sequence
  source: /note=“Description of Artificial Sequence Synthetic polypeptide”
GSERRCLPAGKTCVRGPMRVPCCGSCSQNKCT

SEQ ID NO: 67 - protein, 33 aa, Artificial Sequence
  source: /note=“Description of Artificial Sequence Synthetic polypeptide”
GSLCSREGEFCYKLRKCCAGFYCKAFVLHCYRN

SEQ ID NO: 68 - protein, 25 aa, Artificial Sequence
  source: /note=“Description of Artificial Sequence Synthetic peptide”
GSACGSCRKKCKGSGKCINGRCKCY

SEQ ID NO: 69 - protein, 25 aa, Artificial Sequence
  source: /note=“Description of Artificial Sequence Synthetic peptide”
GSACGSCRKKCKGPGKCINGRCKCY

SEQ ID NO: 70 - protein, 34 aa, Artificial Sequence
  source: /note=“Description of Artificial Sequence Synthetic polypeptide”
GSACQGYMRKCGRDKPPCCKKLECSKTWRWCVWN

SEQ ID NO: 71 - protein, 32 aa, Artificial Sequence
  source: /note=“Description of Artificial Sequence Synthetic polypeptide”
GSGRYCQKWMWTCDSKRACCEGLRCKLWCRKI

SEQ ID NO: 72 - protein, 36 aa, Artificial Sequence
  source: /note=“Description of Artificial Sequence Synthetic polypeptide”
GSNAKCRGSPECLPKCKEAIGKAAGKCMNGKCKCYP

SEQ ID NO: 73 - protein, 36 aa, Artificial Sequence
  source: /note=“Description of Artificial Sequence Synthetic polypeptide”
GSNVKCRGSKECLPACKAAVGKAAGKCMNGKCKCYP

SEQ ID NO: 74 - protein, 36 aa, Artificial Sequence
  source: /note=“Description of Artificial Sequence Synthetic polypeptide”
GSNVKCRGSPECLPKCKEAIGKSAGKCMNGKCKCYP

SEQ ID NO: 75 - protein, 36 aa, Artificial Sequence
  source: /note=“Description of Artificial Sequence Synthetic polypeptide”
GSNAKCRGSPECLPKCKQAIGKAAGKCMNGKCKCYP

SEQ ID NO: 76 - protein, 35 aa, Artificial Sequence
  source: /note=“Description of Artificial Sequence Synthetic polypeptide”
GSRGYCAEKGIKCHNIHCCSGLTCKCKGSSCVCRK

SEQ ID NO: 77 - protein, 33 aa, Artificial Sequence
  source: /note=“Description of Artificial Sequence Synthetic polypeptide”
GSERGCKLTFWKCKNKKECCGWNACALGICMPR

SEQ ID NO: 78 - protein, 41 aa, Artificial Sequence
  source: /note=“Description of Artificial Sequence Synthetic polypeptide”
GSKKKCIAKDYGRCKWGGTPCCRGRGCICSIMGTNCECKPR

SEQ ID NO: 79 - protein, 31 aa, Artificial Sequence
  source: /note=“Description of Artificial Sequence Synthetic polypeptide”
GSGCKLTFWKCKNKKECCGWNACALGICMPR

SEQ ID NO: 80 - protein, 34 aa, Artificial Sequence
  source: /note=“Description of Artificial Sequence Synthetic polypeptide”
GSACKGLFVTCTPGKDECCPNHVCSSKHKWCKYK

SEQ ID NO: 81 - protein, 38 aa, Artificial Sequence
  source: /note=“Description of Artificial Sequence Synthetic polypeptide”
GSIACAPRGLLCFRDKECCKGLTCKGRFVNTWPTFCLV

SEQ ID NO: 82 - protein, 38 aa, Artificial Sequence
  source: /note=“Description of Artificial Sequence Synthetic polypeptide”
GSACAGLYKKCGKGVNTCCENRPCKCDLAMGNCICKKK

SEQ ID NO: 83 - protein, 40 aa, Artificial Sequence
  source: /note=“Description of Artificial Sequence Synthetic polypeptide”
GSFTCAISCDIKVNGKPCKGSGEKKCSGGWSCKFNVCVKV

SEQ ID NO: 84 - protein, 35 aa, Artificial Sequence
  source: /note=“Description of Artificial Sequence Synthetic polypeptide”
GSGFCAQKGIKCHDIHCCTNLKCVREGSNRVCRKA

SEQ ID NO: 85 - protein, 44 aa, Artificial Sequence
  source: /note=“Description of Artificial Sequence Synthetic polypeptide”
GSCAKKRNWCGKNEDCCCPMKCIYAWYNQQGSCQSTITGLFKKC

SEQ ID NO: 86 - protein, 30 aa, Artificial Sequence
  source: /note=“Description of Artificial Sequence Synthetic polypeptide”
GSYCQKWMWTCDSARKCCEGLVCRLWCKKI

SEQ ID NO: 87 - protein, 39 aa, Artificial Sequence
  source: /note=“Description of Artificial Sequence Synthetic polypeptide”
GSRGGCLPHNKFCNALSGPRCCSGLKCKELTIWNTKCLE

SEQ ID NO: 88 - protein, 36 aa, Artificial Sequence
  source: /note=“Description of Artificial Sequence Synthetic polypeptide”
GSNVKCTGSKQCLPACKAAVGKAAGKCMNGKCKCYT

SEQ ID NO: 89 - protein, 35 aa, Artificial Sequence
  source: /note=“Description of Artificial Sequence Synthetic polypeptide”
GSQRSCAKPGEMCMRIKCCDGQCGCNRGTGRCFCK

SEQ ID NO: 90 - protein, 38 aa, Artificial Sequence
  source: /note=“Description of Artificial Sequence Synthetic polypeptide”
GSGCIPKHKRCTWSGPKCCNNISCHCNISGTLCKCRPG

SEQ ID NO: 91 - protein, 35 aa, Artificial Sequence
  source: /note=“Description of Artificial Sequence Synthetic polypeptide”
GSNYCVAKRCRPGGRQCCSGKPCACVGKVCKCPRD

SEQ ID NO: 92 - protein, 39 aa, Artificial Sequence
  source: /note=“Description of Artificial Sequence Synthetic polypeptide”
GSERGCSGAYKRCSSSQRCCEGRPCVCSAINSNCKCRKT

SEQ ID NO: 93 - protein, 38 aa, Artificial Sequence
  source: /note=“Description of Artificial Sequence Synthetic polypeptide”
GSRYCPRNPEACYNYCLRTGRPGGYCGGRSRITCFCFR

SEQ ID NO: 94 - protein, 35 aa, Artificial Sequence
  source: /note=“Description of Artificial Sequence Synthetic polypeptide”
GSQRSCAKPGEMCMGIKCCDGQCGCNRGTGRCFCK

SEQ ID NO: 95 - protein, 38 aa, Artificial Sequence
  source: /note=“Description of Artificial Sequence Synthetic polypeptide”
GSRRGCFKEGKWCPKSAPCCAPLKCKGPSIKQQKCVRE

SEQ ID NO: 96 - protein, 33 aa, Artificial Sequence
  source: /note=“Description of Artificial Sequence Synthetic polypeptide”
GSTVKCGGCNRKCCAGGCRSGKCINGKCQCYGR

SEQ ID NO: 97 - protein, 31 aa, Artificial Sequence
  source: /note=“Description of Artificial Sequence Synthetic polypeptide”
GSERRCEPSGKPCRPLMRIPCCGSCVRGKCA

SEQ ID NO: 98 - protein, 38 aa, Artificial Sequence
  source: /note=“Description of Artificial Sequence Synthetic polypeptide”
GSRGGCLPRNKFCNPSSGPRCCSGLTCKELNIWANKCL

SEQ ID NO: 99 - protein, 44 aa, Artificial Sequence
  source: /note=“Description of Artificial Sequence Synthetic polypeptide”
GSCAKKRNWCGKNEDCCCPMKCIYAWYNQQGSCQTTITGLFKKC

SEQ ID NO: 100 - protein, 39 aa, Artificial Sequence
  source: /note=“Description of Artificial Sequence Synthetic polypeptide”
GSVRIPVSCKHSGQCLKPCKDAGMRTGKCMNGKCDCTPK

SEQ ID NO: 101 - protein, 22 aa, Artificial Sequence
  source: /note=“Description of Artificial Sequence Synthetic peptide”
GSVKCTTSKDCWPPCKKVTGRA

SEQ ID NO: 102 - protein, 34 aa, Artificial Sequence
  source: /note=“Description of Artificial Sequence Synthetic polypeptide”
GSGIVCRVCRIICGMQGRRVNICRAPIRCRCRRG

SEQ ID NO: 103 - protein, 37 aa, Artificial Sequence
  source: /note=“Description of Artificial Sequence Synthetic polypeptide”
GSSERDCIRHLQRCRENRDCCSRRCSRRGTNPERRCR

SEQ ID NO: 104 - protein, 39 aa, Artificial Sequence
  source: /note=“Description of Artificial Sequence Synthetic polypeptide”
GSVRIPVSCRHSGQCLRPCRDAGMRFGRCMNGRCDCTPR

SEQ ID NO: 105 - protein, 39 aa, Artificial Sequence
  source: /note=“Description of Artificial Sequence Synthetic polypeptide”
GSGVPINVRCRGSRDCLDPCRRAGMRFGRCINSRCHCTP

SEQ ID NO: 106 - protein, 35 aa, Artificial Sequence
  source: /note=“Description of Artificial Sequence Synthetic polypeptide”
GSAVCVYRTCDRDCRRRGYRSGRCINNACRCYPYG

SEQ ID NO: 107 - protein, 37 aa, Artificial Sequence
  source: /note=“Description of Artificial Sequence Synthetic polypeptide”
GSISCTGSRQCYDPCRRRTGCPNARCMNRSCRCYGCG

SEQ ID NO: 108 - protein, 40 aa, Artificial Sequence
  source: /note=“Description of Artificial Sequence Synthetic polypeptide”
GSQVQTNVRCQGGSCASVCRREIGVAAGRCINGRCVCYRN

SEQ ID NO: 109 - protein, 39 aa, Artificial Sequence
  source: /note=“Description of Artificial Sequence Synthetic polypeptide”
GSEVIRCSGSRQCYGPCRQQTGCTNSRCMNRVCRCYGCG

SEQ ID NO: 110 - protein, 35 aa, Artificial Sequence
  source: /note=“Description of Artificial Sequence Synthetic polypeptide”
GSACRGVFDACTPGRNECCPNRVCSDRHRWCRWRL

SEQ ID NO: 111 - protein, 40 aa, Artificial Sequence
  source: /note=“Description of Artificial Sequence Synthetic polypeptide”
GSQIYTSRECNGSSECYSHCEGITGRRSGRCINRRCYCYR

SEQ ID NO: 112 - protein, 37 aa, Artificial Sequence
  source: /note=“Description of Artificial Sequence Synthetic polypeptide”
GSGCLEFWWRCNPNDDRCCRPRLRCSRLFRLCNFSFG

SEQ ID NO: 113 - protein, 39 aa, Artificial Sequence
  source: /note=“Description of Artificial Sequence Synthetic polypeptide”
GSDCVRFWGRCSQTSDCCPHLACRSRWPRNICVWDGSVG

SEQ ID NO: 114 - protein, 35 aa, Artificial Sequence
  source: /note=“Description of Artificial Sequence Synthetic polypeptide”
GSGCFGYRCDYYRGCCSGYVCSPTWRWCVRPGPGR

SEQ ID NO: 115 - protein, 62 aa, Artificial Sequence
  source: /note=“Description of Artificial Sequence Synthetic polypeptide”
GSMNARFILLLVLTTMMLLPDTRGAEVIRCSGSRQCYGPCRQQTGCTNSRCMNRVCRCYGCG

SEQ ID NO: 116 - protein, 63 aa, Artificial Sequence
  source: /note=“Description of Artificial Sequence Synthetic polypeptide”
GSMNARLIYLLLVVTTMTLMFDTAQAVDIMCSGPRQCYGPCRRETGCPNARCMNRRCRCYGCV

SEQ ID NO: 117 - protein, 64 aa, Artificial Sequence
  source: /note=“Description of Artificial Sequence Synthetic polypeptide”
GSMNARLIYLLLVVTTMMLTFDTTQAGDIRCSGTRQCWGPCRRQTTCTNSRCMNGRCRCYGCVG

SEQ ID NO: 118 - protein, 63 aa, Artificial Sequence
  source: /note=“Description of Artificial Sequence Synthetic polypeptide”
GSMNTRFIFLLLVVTNTMMLFDTRPVEGISCTGSRQCYDPCRRRTGCPNARCMNRSCRCYGCG

SEQ ID NO: 119 - protein, 40 aa, Artificial Sequence
  source: /note=“Description of Artificial Sequence Synthetic polypeptide”
GSGVPINVRCSGSRDCLEPCRRAGMRFGRCINRRCHCTPR

SEQ ID NO: 120 - protein, 40 aa, Artificial Sequence
  source: /note=“Description of Artificial Sequence Synthetic polypeptide”
GSGVPINVRCTGSPQCLRPCRDAGMRFGRCINGRCHCTPR

SEQ ID NO: 121 - protein, 40 aa, Artificial Sequence
  source: /note=“Description of Artificial Sequence Synthetic polypeptide”
GSGVIINVRCRISRQCLEPCRRAGMRFGRCMNGRCHCTPR

SEQ ID NO: 122 - protein, 40 aa, Artificial Sequence
  source: /note=“Description of Artificial Sequence Synthetic polypeptide”
GSGVPINVRCRGSPQCIQPCRDAGMRFGRCMNGRCHCTPQ

SEQ ID NO: 123 - protein, 40 aa, Artificial Sequence
  source: /note=“Description of Artificial Sequence Synthetic polypeptide”
GSGVEINVRCTGSHQCIRPCRDAGMRFGRCINRRCHCTPR

SEQ ID NO: 124 - protein, 40 aa, Artificial Sequence
  source: /note=“Description of Artificial Sequence Synthetic polypeptide”
GSGVEINVRCSGSPQCLRPCRDAGMRFGRCMNRRCHCTPR

SEQ ID NO: 125 - protein, 40 aa, Artificial Sequence
  source: /note=“Description of Artificial Sequence Synthetic polypeptide”
GSGVPTDVRCRGSPQCIQPCRDAGMRFGRCMNGRCHCTPR

SEQ ID NO: 126 - protein, 40 aa, Artificial Sequence
  source: /note=“Description of Artificial Sequence Synthetic polypeptide”
GSGVPINVSCTGSPQCIRPCRDAGMRFGRCMNRRCHCTPR

SEQ ID NO: 127 - protein, 40 aa, Artificial Sequence
  source: /note=“Description of Artificial Sequence Synthetic polypeptide”
GSGVPINVPCTGSPQCIRPCRDAGMRFGRCMNRRCHCTPR

SEQ ID NO: 128 - protein, 39 aa, Artificial Sequence
  source: /note=“Description of Artificial Sequence Synthetic polypeptide”
GSVGINVRCRHSGQCLRPCRDAGMRFGRCINGRCDCTPR

SEQ ID NO: 129 - protein, 39 aa, Artificial Sequence
  source: /note=“Description of Artificial Sequence Synthetic polypeptide”
GSVGINVRCRHSGQCLRPCRDAGMRFGRCMNGRCDCTPR

SEQ ID NO: 130 - protein, 39 aa, Artificial Sequence
  source: /note=“Description of Artificial Sequence Synthetic polypeptide”
GSVGIPVSCRHSGQCIRPCRDAGMRFGRCMNRRCDCTPR

SEQ ID NO: 131 - protein, 38 aa, Artificial Sequence
  source: /note=“Description of Artificial Sequence Synthetic polypeptide”
GSRRGCFREGHSCPRTAPCCRPLVCRGPSPNTRRCTRP

SEQ ID NO: 132 - protein, 34 aa, Artificial Sequence
  source: /note=“Description of Artificial Sequence Synthetic polypeptide”
GSSFCIPFRPCRSDENCCRRFRCRTTGIVRLCRW

SEQ ID NO: 133 - protein, 38 aa, Artificial Sequence
  source: /note=“Description of Artificial Sequence Synthetic polypeptide”
GSLRGCLPRNRFCNALSGPRCCSGLRCRELSIWASRCL

SEQ ID NO: 134 - protein, 36 aa, Artificial Sequence
  source: /note=“Description of Artificial Sequence Synthetic polypeptide”
GSGNYCLRGRCLPGGRRCCNGRPCECFARICSCRPR

SEQ ID NO: 135 - protein, 31 aa, Artificial Sequence
  source: /note=“Description of Artificial Sequence Synthetic polypeptide”
GSTVRCGGCNRRCCPGGCRSGRCINGRCQCY

SEQ ID NO: 136 - protein, 31 aa, Artificial Sequence
  source: /note=“Description of Artificial Sequence Synthetic polypeptide”
GSGCMREYCAGQCRGRVSQDYCLRHCRCIPR

SEQ ID NO: 137 - protein, 36 aa, Artificial Sequence
  source: /note=“Description of Artificial Sequence Synthetic polypeptide”
GSACLGFGERCNPSNDRCCRSSSLVCSQRHRWCRYG

SEQ ID NO: 138 - protein, 39 aa, Artificial Sequence
  source: /note=“Description of Artificial Sequence Synthetic polypeptide”
GSRGGCLPHNRFCNALSGPRCCSGLRCRELSIRDSRCLG

SEQ ID NO: 139 - protein, 38 aa, Artificial Sequence
  source: /note=“Description of Artificial Sequence Synthetic polypeptide”
GSRGGCLPRNRFCNPSSGPRCCSGLTCRELNIWASRCL

SEQ ID NO: 140 - protein, 35 aa, Artificial Sequence
  source: /note=“Description of Artificial Sequence Synthetic polypeptide”
GSQRSCARPGDMCMGIRCCDGQCGCNRGTGRCFCR

SEQ ID NO: 141 - protein, 42 aa, Artificial Sequence
  source: /note=“Description of Artificial Sequence Synthetic polypeptide”
GSARGCADAYRSCNHPRTCCDGYNGYRRACICSGSNCRCRRS

SEQ ID NO: 142 - protein, 39 aa, Artificial Sequence
  source: /note=“Description of Artificial Sequence Synthetic polypeptide”
GSRGGCLPHNRFCNALSGPRCCSGLRCRELSIWDSRCLG

SEQ ID NO: 143 - protein, 39 aa, Artificial Sequence
  source: /note=“Description of Artificial Sequence Synthetic polypeptide”
GSRGGCLPHNRFCNALSGPRCCSGLRCRELSIYDSRCLG

SEQ ID NO: 144 - protein, 39 aa, Artificial Sequence
  source: /note=“Description of Artificial Sequence Synthetic polypeptide”
GSRGGCLPHNRFCNALSGPRCCSRLRCRELSIWDSRCLG

SEQ ID NO: 145 - protein, 39 aa, Artificial Sequence
  source: /note=“Description of Artificial Sequence Synthetic polypeptide”
GSRGGCLPHNRFCNALTGPRCCSRLRCRELSIWDSICLG

SEQ ID NO: 146 - protein, 36 aa, Artificial Sequence
  source: /note=“Description of Artificial Sequence Synthetic polypeptide”
GSSCADAYKSCDSLRCCNNRTCMCSMIGTNCTCRRR

SEQ ID NO: 147 - protein, 32 aa, Artificial Sequence
  source: /note=“Description of Artificial Sequence Synthetic polypeptide”
GSERRCLPAGRTCVRGPMRVPCCGSCSQNRCT

SEQ ID NO: 148 - protein, 33 aa, Artificial Sequence
  source: /note=“Description of Artificial Sequence Synthetic polypeptide”
GSLCSREGEFCYRLRRCCAGFYCRAFVLHCYRN

SEQ ID NO: 149 - protein, 25 aa, Artificial Sequence
  source: /note=“Description of Artificial Sequence Synthetic peptide”
GSACGSCRRRCRGSGRCINGRCRCY

SEQ ID NO: 150 - protein, 25 aa, Artificial Sequence
  source: /note=“Description of Artificial Sequence Synthetic peptide”
GSACGSCRRRCRGPGRCINGRCRCY

SEQ ID NO: 151 - protein, 34 aa, Artificial Sequence
  source: /note=“Description of Artificial Sequence Synthetic polypeptide”
GSACQGYMRRCGRDRPPCCRRLECSRTWRWCVWN

SEQ ID NO: 152 - protein, 32 aa, Artificial Sequence
  source: /note=“Description of Artificial Sequence Synthetic polypeptide”
GSGRYCQRWMWTCDSRRACCEGLRCRLWCRRI

SEQ ID NO: 153 - protein, 36 aa, Artificial Sequence
  source: /note=“Description of Artificial Sequence Synthetic polypeptide”
GSNARCRGSPECLPRCREAIGRAAGRCMNGRCRCYP

SEQ ID NO: 154 - protein, 36 aa, Artificial Sequence
  source: /note=“Description of Artificial Sequence Synthetic polypeptide”
GSNVRCRGSRECLPACRAAVGRAAGRCMNGRCRCYP

SEQ ID NO: 155 - protein, 36 aa, Artificial Sequence
  source: /note=“Description of Artificial Sequence Synthetic polypeptide”
GSNVRCRGSPECLPRCREAIGRSAGRCMNGRCRCYP

SEQ ID NO: 156 - protein, 36 aa, Artificial Sequence
  source: /note=“Description of Artificial Sequence Synthetic polypeptide”
GSNARCRGSPECLPRCRQAIGRAAGRCMNGRCRCYP

SEQ ID NO: 157 - protein, 35 aa, Artificial Sequence
  source: /note=“Description of Artificial Sequence Synthetic polypeptide”
GSRGYCAERGIRCHNIHCCSGLTCRCRGSSCVCRR

SEQ ID NO: 158 - protein, 33 aa, Artificial Sequence
  source: /note=“Description of Artificial Sequence Synthetic polypeptide”
GSERGCRLTFWRCRNRRECCGWNACALGICMPR

SEQ ID NO: 159 - protein, 41 aa, Artificial Sequence
  source: /note=“Description of Artificial Sequence Synthetic polypeptide”
GSRRRCIARDYGRCRWGGTPCCRGRGCICSIMGTNCECRPR

SEQ ID NO: 160 - protein, 31 aa, Artificial Sequence
  source: /note=“Description of Artificial Sequence Synthetic polypeptide”
GSGCRLTFWRCRNRRECCGWNACALGICMPR

SEQ ID NO: 161 - protein, 34 aa, Artificial Sequence
  source: /note=“Description of Artificial Sequence Synthetic polypeptide”
GSACRGLFVTCTPGRDECCPNHVCSSRHRWCRYR

SEQ ID NO: 162 - protein, 38 aa, Artificial Sequence
  source: /note=“Description of Artificial Sequence Synthetic polypeptide”
GSIACAPRGLLCFRDRECCRGLTCRGRFVNTWPTFCLV

SEQ ID NO: 163 - protein, 38 aa, Artificial Sequence
  source: /note=“Description of Artificial Sequence Synthetic polypeptide”
GSACAGLYRRCGRGVNTCCENRPCRCDLAMGNCICRRR

SEQ ID NO: 164 - protein, 40 aa, Artificial Sequence
  source: /note=“Description of Artificial Sequence Synthetic polypeptide”
GSFTCAISCDIRVNGRPCRGSGERRCSGGWSCRFNVCVRV

SEQ ID NO: 165 - protein, 35 aa, Artificial Sequence
  source: /note=“Description of Artificial Sequence Synthetic polypeptide”
GSGFCAQRGIRCHDIHCCTNLRCVREGSNRVCRRA

SEQ ID NO: 166 - protein, 44 aa, Artificial Sequence
  source: /note=“Description of Artificial Sequence Synthetic polypeptide”
GSCARRRNWCGRNEDCCCPMRCIYAWYNQQGSCQSTITGLFRRC

SEQ ID NO: 167 - protein, 30 aa, Artificial Sequence
  source: /note=“Description of Artificial Sequence Synthetic polypeptide”
GSYCQRWMWTCDSARRCCEGLVCRLWCRRI

SEQ ID NO: 168 - protein, 39 aa, Artificial Sequence
  source: /note=“Description of Artificial Sequence Synthetic polypeptide”
GSRGGCLPHNRFCNALSGPRCCSGLRCRELTIWNTRCLE

SEQ ID NO: 169 - protein, 36 aa, Artificial Sequence
  source: /note=“Description of Artificial Sequence Synthetic polypeptide”
GSNVRCTGSRQCLPACRAAVGRAAGRCMNGRCRCYT

SEQ ID NO: 170 - protein, 35 aa, Artificial Sequence
  source: /note=“Description of Artificial Sequence Synthetic polypeptide”
GSQRSCARPGEMCMRIRCCDGQCGCNRGTGRCFCR

SEQ ID NO: 171 - protein, 38 aa, Artificial Sequence
  source: /note=“Description of Artificial Sequence Synthetic polypeptide”
GSGCIPRHRRCTWSGPRCCNNISCHCNISGTLCRCRPG

SEQ ID NO: 172 - protein, 35 aa, Artificial Sequence
  source: /note=“Description of Artificial Sequence Synthetic polypeptide”
GSNYCVARRCRPGGRQCCSGRPCACVGRVCRCPRD

SEQ ID NO: 173 - protein, 39 aa, Artificial Sequence
  source: /note=“Description of Artificial Sequence Synthetic polypeptide”
GSERGCSGAYRRCSSSQRCCEGRPCVCSAINSNCRCRRT

SEQ ID NO: 174 - protein, 35 aa, Artificial Sequence
  source: /note=“Description of Artificial Sequence Synthetic polypeptide”
GSQRSCARPGEMCMGIRCCDGQCGCNRGTGRCFCR

SEQ ID NO: 175 - protein, 38 aa, Artificial Sequence
  source: /note=“Description of Artificial Sequence Synthetic polypeptide”
GSRRGCFREGRWCPRSAPCCAPLRCRGPSIRQQRCVRE

SEQ ID NO: 176 - protein, 33 aa, Artificial Sequence
  source: /note=“Description of Artificial Sequence Synthetic polypeptide”
GSTVRCGGCNRRCCAGGCRSGRCINGRCQCYGR

SEQ ID NO: 177 - protein, 31 aa, Artificial Sequence
  source: /note=“Description of Artificial Sequence Synthetic polypeptide”
GSERRCEPSGRPCRPLMRIPCCGSCVRGRCA

SEQ ID NO: 178 - protein, 38 aa, Artificial Sequence
  source: /note=“Description of Artificial Sequence Synthetic polypeptide”
GSRGGCLPRNRFCNPSSGPRCCSGLTCRELNIWANRCL

SEQ ID NO: 179 - protein, 44 aa, Artificial Sequence
  source: /note=“Description of Artificial Sequence Synthetic polypeptide”
GSCARRRNWCGRNEDCCCPMRCIYAWYNQQGSCQTTITGLFRRC

SEQ ID NO: 180 - protein, 39 aa, Artificial Sequence
  source: /note=“Description of Artificial Sequence Synthetic polypeptide”
GSVRIPVSCRHSGQCLRPCRDAGMRTGRCMNGRCDCTPR

SEQ ID NO: 181 - protein, 40 aa, Artificial Sequence
  source: /note=“Description of Artificial Sequence Synthetic polypeptide”
GSQKILSNRCNNSSECIPHCIRIFGTRAAKCINRKCYCYP

SEQ ID NO: 182 - protein, 34 aa, Artificial Sequence
  source: /note=“Description of Artificial Sequence Synthetic polypeptide”
GSAVCNLKRCQLSCRSLGLLGKCIGDKCECVKHG

SEQ ID NO: 183 - protein, 39 aa, Artificial Sequence
  source: /note=“Description of Artificial Sequence Synthetic polypeptide”
GSISIGIRCSPSIDLCEGQCRIRRYFTGYCSGDTCHCSG

SEQ ID NO: 184 - protein, 35 aa, Artificial Sequence
  source: /note=“Description of Artificial Sequence Synthetic polypeptide”
GSGDCLPHLRRCRENNDCCSRRCRRRGANPERRCR

SEQ ID NO: 185 - protein, 36 aa, Artificial Sequence
  source: /note=“Description of Artificial Sequence Synthetic polypeptide”
GSSCEPGRTFRDRCNTCKCGADGRSAACTLRACPNQ

SEQ ID NO: 186 - protein, 35 aa, Artificial Sequence
  source: /note=“Description of Artificial Sequence Synthetic polypeptide”
GSGDCLPHLKRCKADNDCCGKKCKRRGTNAEKRCR

SEQ ID NO: 187 - protein, 35 aa, Artificial Sequence
  source: /note=“Description of Artificial Sequence Synthetic polypeptide”
GSGDCLPHLKRCKENNDCCSKKCKRRGTNPEKRCR

SEQ ID NO: 188 - protein, 35 aa, Artificial Sequence
  source: /note=“Description of Artificial Sequence Synthetic polypeptide”
GSKDCLKKLKLCKENKDCCSKSCKRRGTNIEKRCR

SEQ ID NO: 189 - protein, 35 aa, Artificial Sequence
  source: /note=“Description of Artificial Sequence Synthetic polypeptide”
GSGDCLPHLKRCKENNDCCSKKCKRRGANPEKRCR

SEQ ID NO: 190 - protein, 39 aa, Artificial Sequence
  source: /note=“Description of Artificial Sequence Synthetic polypeptide”
GSVFINVKCRGSPECLPKCKEAIGKSAGKCMNGKCKCYP

SEQ ID NO: 191 - protein, 39 aa, Artificial Sequence
  source: /note=“Description of Artificial Sequence Synthetic polypeptide”
GSVFINAKCRGSPECLPKCKEAIGKAAGKCMNGKCKCYP

SEQ ID NO: 192 - protein, 38 aa, Artificial Sequence
  source: /note=“Description of Artificial Sequence Synthetic polypeptide”
GSVIINVKCKISRQCLEPCKKAGMRFGKCMNGKCHCTP

SEQ ID NO: 193 - protein, 38 aa, Artificial Sequence
  source: /note=“Description of Artificial Sequence Synthetic polypeptide”
GSVPTDVKCRGSPQCIQPCKDAGMRFGKCMNGKCHCTP

SEQ ID NO: 194 - protein, 38 aa, Artificial Sequence
  source: /note=“Description of Artificial Sequence Synthetic polypeptide”
GSVRIPVSCKHSGQCLKPCKDAGMRFGKCMNGKCDCTP

SEQ ID NO: 195 - protein, 38 aa, Artificial Sequence
  source: /note=“Description of Artificial Sequence Synthetic polypeptide”
GSVRIPVSCRHSGQCLRPCRDAGMRFGRCMNGRCDCTP

SEQ ID NO: 196 - protein, 35 aa, Artificial Sequence
  source: /note=“Description of Artificial Sequence Synthetic polypeptide”
GSTNVSCTTSKECWSVCQRLHNTSRGKCMNKKCRC

SEQ ID NO: 197 - protein, 34 aa, Artificial Sequence
  source: /note=“Description of Artificial Sequence Synthetic polypeptide”
GSNVKCTGSKQCLPACKAAVGKAAGKCMNGKCKC

SEQ ID NO: 198 - protein, 39 aa, Artificial Sequence
  source: /note=“Description of Artificial Sequence Synthetic polypeptide”
GSGVPINVRCRGSRDCLDPCRGAGERHGRCGNSRCHCTP

SEQ ID NO: 199 - protein, 39 aa, Artificial Sequence
  source: /note=“Description of Artificial Sequence Synthetic polypeptide”
GSVRIPVSCRHSGQCLRPCRDAGERHGRCGGGRCDCTPR

SEQ ID NO: 200 - protein, 40 aa, Artificial Sequence
  source: /note=“Description of Artificial Sequence Synthetic polypeptide”
GSQVQTNVRCQGGSCGSVCRREGGGAGGGCGNGRCGCYRN

SEQ ID NO: 201 - protein, 34 aa, Artificial Sequence
  source: /note=“Description of Artificial Sequence Synthetic polypeptide”
GSIKCSESYQCFPVCKSRFGKTNGRCVNGFCDCF

SEQ ID NO: 202 - protein, 35 aa, Artificial Sequence
  source: /note=“Description of Artificial Sequence Synthetic polypeptide”
GSVKCSSPQQCLKPCKAAFGISAGGKCINGKCKCY

SEQ ID NO: 203 - protein, 34 aa, Artificial Sequence
  source: /note=“Description of Artificial Sequence Synthetic polypeptide”
GSVSCSASSQCWPVCKKLFGTYRGKCMNSKCRCY

SEQ ID NO: 204 - protein, 34 aa, Artificial Sequence
  source: /note=“Description of Artificial Sequence Synthetic polypeptide”
GSESCTASNQCWSICKRLHNTNRGKCMNKKCRCY

SEQ ID NO: 205 - protein, 34 aa, Artificial Sequence
  source: /note=“Description of Artificial Sequence Synthetic polypeptide”
GSVSCTTSKECWSVCEKLYNTSRGKCMNKKCRCY

SEQ ID NO: 206 - protein, 34 aa, Artificial Sequence
  source: /note=“Description of Artificial Sequence Synthetic polypeptide”
GSMRCKSSKECLVKCKQATGRPNGKCMNRKCKCY

SEQ ID NO: 207 - protein, 34 aa, Artificial Sequence
  source: /note=“Description of Artificial Sequence Synthetic polypeptide”
GSIKCTLSKDCYSPCKKETGCPRAKCINRNCKCY

SEQ ID NO: 208 - protein, 34 aa, Artificial Sequence
  source: /note=“Description of Artificial Sequence Synthetic polypeptide”
GSIRCSGSRDCYSPCMKQTGCPNAKCINKSCKCY

SEQ ID NO: 209 - protein, 34 aa, Artificial Sequence
  source: /note=“Description of Artificial Sequence Synthetic polypeptide”
GSIRCSGTRECYAPCQKLTGCLNAKCMNKACKCY

SEQ ID NO: 210 - protein, 34 aa, Artificial Sequence
  source: /note=“Description of Artificial Sequence Synthetic polypeptide”
GSISCTNPKQCYPHCKKETGYPNAKCMNRKCKCF

SEQ ID NO: 211 - protein, 34 aa, Artificial Sequence
  source: /note=“Description of Artificial Sequence Synthetic polypeptide”
GSASCRTPKDCADPCRKETGCPYGKCMNRKCKCN

SEQ ID NO: 212 - protein, 33 aa, Artificial Sequence
  source: /note=“Description of Artificial Sequence Synthetic polypeptide”
GSTSCISPKQCTEPCRAKGCKHGKCMNRKCHCM

SEQ ID NO: 213 - protein, 33 aa, Artificial Sequence
  source: /note=“Description of Artificial Sequence Synthetic polypeptide”
GSKECTGPQHCTNFCRKNKCTHGKCMNRKCKCF

SEQ ID NO: 214 - protein, 34 aa, Artificial Sequence
  source: /note=“Description of Artificial Sequence Synthetic polypeptide”
GSIKCRTPKDCADPCRKQTGCPHAKCMNKTCRCH

SEQ ID NO: 215 - protein, 34 aa, Artificial Sequence
  source: /note=“Description of Artificial Sequence Synthetic polypeptide”
GSVKCTTSKECWPPCKAATGKAAGKCMNKKCKCQ

SEQ ID NO: 216 - protein, 34 aa, Artificial Sequence
  source: /note=“Description of Artificial Sequence Synthetic polypeptide”
GSLECGASRECYDPCFKAFGRAHGKCMNNKCRCY

SEQ ID NO: 217 - protein, 34 aa, Artificial Sequence
  source: /note=“Description of Artificial Sequence Synthetic polypeptide”
GSEKCFATSQCWTPCKKAIGSLQSKCMNGKCKCY

SEQ ID NO: 218 - protein, 34 aa, Artificial Sequence
  source: /note=“Description of Artificial Sequence Synthetic polypeptide”
GSVRCYASRECWEPCRRVTGSAQAKCQNNQCRCY

SEQ ID NO: 219 - protein, 34 aa, Artificial Sequence
  source: /note=“Description of Artificial Sequence Synthetic polypeptide”
GSVKCSASRECWVACKKVTGSGQGKCQNNQCRCY

SEQ ID NO: 220 - protein, 34 aa, Artificial Sequence
  source: /note=“Description of Artificial Sequence Synthetic polypeptide”
GSVKCISSQECWIACKKVTGRFEGKCQNRQCRCY

SEQ ID NO: 221 - protein, 34 aa, Artificial Sequence
  source: /note=“Description of Artificial Sequence Synthetic polypeptide”
GSVRCYDSRQCWIACKKVTGSTQGKCQNKQCRCY

SEQ ID NO: 222 - protein, 34 aa, Artificial Sequence
  source: /note=“Description of Artificial Sequence Synthetic polypeptide”
GSVDCTVSKECWAPCKAAFGVDRGKCMGKKCKCY

SEQ ID NO: 223 - protein, 34 aa, Artificial Sequence
  source: /note=“Description of Artificial Sequence Synthetic polypeptide”
GSAKCRGSPECLPKCKEAIGKAAGKCMNGKCKCY

SEQ ID NO: 224 - protein, 33 aa, Artificial Sequence
  source: /note=“Description of Artificial Sequence Synthetic polypeptide”
GSKKCQGGSCASVCRRVIGVAAGKCINGRCVCY

SEQ ID NO: 225 - protein, 34 aa, Artificial Sequence
  source: /note=“Description of Artificial Sequence Synthetic polypeptide”
GSKKCSNTSQCYKTCEKVVGVAAGKCMNGKCICY

SEQ ID NO: 226 - protein, 34 aa, Artificial Sequence
  source: /note=“Description of Artificial Sequence Synthetic polypeptide”
GSVKCSGSSKCVKICIDRYNTRGAKCINGRCTCY

SEQ ID NO: 227 - protein, 34 aa, Artificial Sequence
  source: /note=“Description of Artificial Sequence Synthetic polypeptide”
GSNRCNNSSECIPHCIRIFGTRAAKCINRKCYCY

SEQ ID NO: 228 - protein, 34 aa, Artificial Sequence
  source: /note=“Description of Artificial Sequence Synthetic polypeptide”
GSKECNGSSECYSHCEGITGKRSGKCINKKCYCY

SEQ ID NO: 229 - protein, 31 aa, Artificial Sequence
  source: /note=“Description of Artificial Sequence Synthetic polypeptide”
GSAFCNLRRCELSCRSLGLLGKCIGEECKCV

SEQ ID NO: 230 - protein, 31 aa, Artificial Sequence
  source: /note=“Description of Artificial Sequence Synthetic polypeptide”
GSAVCNLKRCQLSCRSLGLLGKCIGDKCECV

SEQ ID NO: 231 - protein, 32 aa, Artificial Sequence
  source: /note=“Description of Artificial Sequence Synthetic polypeptide”
GSAACYSSDCRVKCVAMGFSSGKCINSKCKCY

SEQ ID NO: 232 - protein, 29 aa, Artificial Sequence
  source: /note=“Description of Artificial Sequence Synthetic peptide”
GSAICATDADCSRKCPGNPPCRNGFCACT

SEQ ID NO: 233 - protein, 29 aa, Artificial Sequence
  source: /note=“Description of Artificial Sequence Synthetic peptide”
GSTECQIKNDCQRYCQSVKECKYGKCYCN

SEQ ID NO: 234 - protein, 29 aa, Artificial Sequence
  source: /note=“Description of Artificial Sequence Synthetic peptide”
GSTQCQSVRDCQQYCLTPDRCSYGTCYCK

SEQ ID NO: 235 - protein, 34 aa, Artificial Sequence
  source: /note=“Description of Artificial Sequence Synthetic polypeptide”
GSVSCRYGSDCAEPCKRLKCLLPSKCINGKCTCY

SEQ ID NO: 236 - protein, 34 aa, Artificial Sequence
  source: /note=“Description of Artificial Sequence Synthetic polypeptide”
GSIKCRYPADCHIMCRKVTGRAEGKCMNGKCTCY

SEQ ID NO: 237 - protein, 34 aa, Artificial Sequence
  source: /note=“Description of Artificial Sequence Synthetic polypeptide”
GSIKCSSSSSCYEPCRGVTGRAHGKCMNGRCTCY

SEQ ID NO: 238 - protein, 34 aa, Artificial Sequence
  source: /note=“Description of Artificial Sequence Synthetic polypeptide”
GSVKCTGSKQCLPACKAAVGKAAGKCMNGKCKCY

SEQ ID NO: 239 - protein, 33 aa, Artificial Sequence
  source: /note=“Description of Artificial Sequence Synthetic polypeptide”
GSVSCKHSGQCIKPCKDAGMRFGKCMNRKCDCT

SEQ ID NO: 240 - protein, 33 aa, Artificial Sequence
  source: /note=“Description of Artificial Sequence Synthetic polypeptide”
GSVKCRGSPQCIQPCRDAGMRFGKCMNGKCHCT

SEQ ID NO: 241 - protein, 35 aa, Artificial Sequence
  source: /note=“Description of Artificial Sequence Synthetic polypeptide”
GSVKCTSPKQCLPPCKAQFGIRAGAKCMNGKCKCY

SEQ ID NO: 242 - protein, 35 aa, Artificial Sequence
  source: /note=“Description of Artificial Sequence Synthetic polypeptide”
GSVKCTSPKQCSKPCKELYGSSAGAKCMNGKCKCY

SEQ ID NO: 243 - protein, 35 aa, Artificial Sequence
  source: /note=“Description of Artificial Sequence Synthetic polypeptide”
GSVKCTSPKQCLPPCKEIYGRHAGAKCMNGKCHCS

SEQ ID NO: 244 - protein, 34 aa, Artificial Sequence
  source: /note=“Description of Artificial Sequence Synthetic polypeptide”
GSVKCTGSKQCWPVCKQMFGKPNGKCMNGKCRCY

SEQ ID NO: 245 - protein, 33 aa, Artificial Sequence
  source: /note=“Description of Artificial Sequence Synthetic polypeptide”
GSVKCRGSRDCLDPCKKAGMRFGKCINSKCHCT

SEQ ID NO: 246 - protein, 29 aa, Artificial Sequence
  source: /note=“Description of Artificial Sequence Synthetic peptide”
GSVRCVTDDDCFRKCPGNPSCKRGFCACK

SEQ ID NO: 247 - protein, 34 aa, Artificial Sequence
  source: /note=“Description of Artificial Sequence Synthetic polypeptide”
GSVPCNNSRPCVPVCIREVNNKNGKCSNGKCLCY

SEQ ID NO: 248 - protein, 40 aa, Artificial Sequence
  source: /note=“Description of Artificial Sequence Synthetic polypeptide”
  MOD_RES (3)..(3): Any amino acid, amino acid analogue or not present
  MOD_RES (5)..(6): Any amino acid, amino acid analogue or not present
  MOD_RES (9)..(9): Any amino acid, amino acid analogue or not present
  MOD_RES (15)..(15): Any amino acid, amino acid analogue or not present
  MOD_RES (20)..(22): Any amino acid, amino acid analogue or not present
  MOD_RES (24)..(24): Any amino acid, amino acid analogue or not present
  MOD_RES (26)..(26): Any amino acid, amino acid analogue or not present
  MOD_RES (30)..(30): Any amino acid, amino acid analogue or not present
  MOD_RES (37)..(40): Any amino acid, amino acid analogue or not present
GVXIXXKCXGSKQCXDPCKXXXGXRXGKCXNKKCKCXXXX

SEQ ID NO: 249 - protein, 40 aa, Artificial Sequence
  source: /note=“Description of Artificial Sequence Synthetic polypeptide”
  VARIANT (3)..(3): /replace=“Arg”
  VARIANT (5)..(5): /replace=“Asn”
  VARIANT (6)..(6): /replace=“Ile”
  VARIANT (9)..(9): /replace=“Thr” or “Arg” or “Lys”
  VARIANT (15)..(15): /replace=“Leu”
  VARIANT (20)..(20): /replace=“Arg” or “Lys”
  VARIANT (21)..(21): /replace=“Lys” or “Arg”
  VARIANT (22)..(22): /replace=“Ala”
  VARIANT (24)..(24): /replace=“Met”
  VARIANT (26)..(26): /replace=“Asn”
  VARIANT (30)..(30): /replace=“Ile”
  VARIANT (37)..(37): /replace=“Thr”
  VARIANT (38)..(38): /replace=“Pro”
  VARIANT (39)..(39): /replace=“ ”
  VARIANT (40)..(40): /replace=“ ”
  SITE (1)..(40): /note=“Variant residues given in the sequence have no preference with respect to those in the annotations for variant positions”
GVPIPVKCSGSKQCYDPCKQATGCRFGKCMNKKCKCYGCG

SEQ ID NO: 250 - protein, 39 aa, Artificial Sequence
  source: /note=“Description of Artificial Sequence Synthetic polypeptide”
  MOD_RES (1)..(4): Any amino acid, amino acid analogue or not present
  MOD_RES (6)..(6): Any amino acid, amino acid analogue or not present
  MOD_RES (8)..(8): Any amino acid, amino acid analogue or not present
  MOD_RES (15)..(15): Any amino acid, amino acid analogue or not present
  MOD_RES (19)..(20): Any amino acid, amino acid analogue or not present
  MOD_RES (24)..(26): Any amino acid, amino acid analogue or not present
  MOD_RES (29)..(30): Any amino acid, amino acid analogue or not present
  MOD_RES (32)..(32): Any amino acid, amino acid analogue or not present
XXXXIXCXGSKQCYXPCKXXTGCXXXKCXXKXCKCYGCG

SEQ ID NO: 251 - protein, 39 aa, Artificial Sequence; identical to SEQ ID NO: 28
  source: /note=“Description of Artificial Sequence Synthetic polypeptide”
  VARIANT (1)..(1): /replace=“ ”
  VARIANT (2)..(2): /replace=“ ”
  VARIANT (3)..(3): /replace=“Gly” or “ ”
  VARIANT (4)..(4): /replace=“Ser” or “ ”
  VARIANT (6)..(6): /replace=“Ser”
  VARIANT (8)..(8): /replace=“Thr”
  VARIANT (15)..(15): /replace=“Asp”
  VARIANT (19)..(19): /replace=“Arg”
  VARIANT (20)..(20): /replace=“Lys”
  VARIANT (24)..(24): /replace=“Pro”
  VARIANT (25)..(25): /replace=“Gln”
  VARIANT (26)..(26): /replace=“Ala”
  VARIANT (29)..(29): /replace=“Leu”
  VARIANT (30)..(30): /replace=“Gln”
  VARIANT (32)..(32): /replace=“Ser”
  SITE (1)..(39): /note=“Variant residues given in the sequence have no preference with respect to those in the annotations for variant positions”

SEQ ID NO: 252 - protein, 40 aa, Artificial Sequence
  source: /note=“Description of Artificial Sequence Synthetic polypeptide”
  MOD_RES (1)..(3): Any amino acid, amino acid analogue or not present
  MOD_RES (5)..(5): Any amino acid, amino acid analogue or not present
  MOD_RES (7)..(7): Any amino acid, amino acid analogue or not present
  MOD_RES (9)..(9): Any amino acid, amino acid analogue or not present
  MOD_RES (11)..(12): Any amino acid, amino acid analogue or not present
  MOD_RES (14)..(15): Any amino acid, amino acid analogue or not present
  MOD_RES (18)..(18): Any amino acid, amino acid analogue or not present
  MOD_RES (22)..(22): Any amino acid, amino acid analogue or not present
  MOD_RES (31)..(31): Any amino acid, amino acid analogue or not present
  MOD_RES (33)..(33): Any amino acid, amino acid analogue or not present
  MOD_RES (36)..(36): Any amino acid, amino acid analogue or not present
  MOD_RES (40)..(40): Any amino acid, amino acid analogue or not present
XXXVXIXVXCXXSXXCLXPCKXAGMRFGKCXNXKCXCTPX

SEQ ID NO: 253 - protein, 40 aa, Artificial Sequence
  source: /note=“Description of Artificial Sequence Synthetic polypeptide”
  VARIANT (1)..(1): /replace=“ ”
  VARIANT (2)..(2): /replace=“Ser” or “ ”
  VARIANT (3)..(3): /replace=“Ser” or “ ”
  VARIANT (5)..(5): /replace=“Arg”
  VARIANT (7)..(7): /replace=“Pro”
  VARIANT (9)..(9): /replace=“Ser”
  VARIANT (11)..(11): /replace=“Lys”
  VARIANT (12)..(12): /replace=“His”
  VARIANT (14)..(14): /replace=“Gly”
  VARIANT (15)..(15): /replace=“Gln”
  VARIANT (18)..(18): /replace=“Lys”
  VARIANT (22)..(22): /replace=“Asp”
  VARIANT (31)..(31): /replace=“Met”
  VARIANT (33)..(33): /replace=“Gly”
  VARIANT (36)..(36): /replace=“Asp”
  VARIANT (40)..(40): /replace=“ ”
  SITE (1)..(40): /note=“Variant residues given in the sequence have no preference with respect to those in the annotations for variant positions”
GGGVPINVKCRGSRDCLDPCKKAGMRFGKCINSKCHCTPK

SEQ ID NO: 254 - protein, 35 aa, Artificial Sequence
  source: /note=“Description of Artificial Sequence Synthetic polypeptide”
  MOD_RES (1)..(1): Any amino acid
  MOD_RES (3)..(3): Any amino acid
  MOD_RES (7)..(7): Any amino acid
  MOD_RES (13)..(13): Any amino acid
  MOD_RES (18)..(18): Any amino acid
  MOD_RES (20)..(20): Any amino acid
  MOD_RES (22)..(22): Any amino acid
  MOD_RES (30)..(30): Any amino acid
  MOD_RES (35)..(35): Any amino acid
  source: /note=“See specification as filed for detailed description of substitutions and preferred embodiments”
XVXVKCXGSKQCXPCKRXGXRXGKCINKKXCKCYX

SEQ ID NO: 255 - protein, 32 aa, Artificial Sequence
  source: /note=“Description of Artificial Sequence Synthetic polypeptide”
  MOD_RES (1)..(1): Any amino acid
  MOD_RES (5)..(5): Any amino acid
  MOD_RES (11)..(11): Any amino acid
  MOD_RES (13)..(13): Any amino acid
  MOD_RES (16)..(16): Any amino acid
  MOD_RES (18)..(18): Any amino acid
  MOD_RES (27)..(27): Any amino acid
  MOD_RES (32)..(32): Any amino acid
  source: /note=“See specification as filed for detailed description of substitutions and preferred embodiments”
XGCVXKCRPGXKXCCXPXKRCSRRFGXKKCKX

SEQ ID NO: 256 - protein, 41 aa, Artificial Sequence
  source: /note=“Description of Artificial Sequence Synthetic polypeptide”
  MOD_RES (1)..(1): Any amino acid
  MOD_RES (3)..(5): Any amino acid
  MOD_RES (9)..(9): Any amino acid
  MOD_RES (15)..(16): Any amino acid
  MOD_RES (21)..(22): Any amino acid
  MOD_RES (24)..(24): Any amino acid
  MOD_RES (26)..(26): Any amino acid
  MOD_RES (34)..(34): Any amino acid
  MOD_RES (39)..(41): Any amino acid
  source: /note=“See specification as filed for detailed description of substitutions and preferred embodiments”
XVXXXVKCXGSKQCXXPCKRXXGXRXGKCINKKXCKCYXXX

SEQ ID NO: 257 - protein, 45 aa, Artificial Sequence
  source: /note=“Description of Artificial Sequence Synthetic polypeptide”
  MOD_RES (1)..(3): Any amino acid
  MOD_RES (7)..(10): Any amino acid
  MOD_RES (16)..(16): Any amino acid
  MOD_RES (18)..(18): Any amino acid
  MOD_RES (21)..(21): Any amino acid
  MOD_RES (23)..(23): Any amino acid
  MOD_RES (32)..(35): Any amino acid
  MOD_RES (40)..(45): Any amino acid
  source: /note=“See specification as filed for detailed description of substitutions and preferred embodiments”
XXXGCVXXXXKCRPGXKXCCXPXKRCSRRFGXXXXKKCKXXXXXX

SEQ ID NO: 258 - protein, 40 aa, Artificial Sequence
  source: /note=“Description of Artificial Sequence Synthetic polypeptide”
  MOD_RES (3)..(3): Any amino acid, amino acid analogue or not present
  MOD_RES (5)..(6): Any amino acid, amino acid analogue or not present
  MOD_RES (9)..(9): Any amino acid, amino acid analogue or not present
  MOD_RES (15)..(15): Any amino acid, amino acid analogue or not present
  MOD_RES (20)..(22): Any amino acid, amino acid analogue or not present
  MOD_RES (24)..(24): Any amino acid, amino acid analogue or not present
  MOD_RES (26)..(26): Any amino acid, amino acid analogue or not present
  MOD_RES (30)..(30): Any amino acid, amino acid analogue or not present
  MOD_RES (37)..(40): Any amino acid, amino acid analogue or not present
GVXIXXRCXGSRQCXDPCRXXXGXRXGRCXNRRCRCXXXX

SEQ ID NO: 259 - protein, 40 aa, Artificial Sequence
  source: /note=“Description of Artificial Sequence Synthetic polypeptide”
  VARIANT (3)..(3): /replace=“Arg”
  VARIANT (5)..(5): /replace=“Asn”
  VARIANT (6)..(6): /replace=“Ile”
  VARIANT (9)..(9): /replace=“Thr” or “Arg” or “Lys”
  VARIANT (15)..(15): /replace=“Leu”
  VARIANT (20)..(20): /replace=“Arg” or “Lys”
  VARIANT (21)..(21): /replace=“Lys” or “Arg”
  VARIANT (22)..(22): /replace=“Ala”
  VARIANT (24)..(24): /replace=“Met”
  VARIANT (26)..(26): /replace=“Asn”
  VARIANT (30)..(30): /replace=“Ile”
  VARIANT (37)..(37): /replace=“Thr”
  VARIANT (38)..(38): /replace=“Pro”
  VARIANT (39)..(39): /replace=“ ”
  VARIANT (40)..(40): /replace=“ ”
  SITE (1)..(40): /note=“Variant residues given in the sequence have no preference with respect to those in the annotations for variant positions”
GVPIPVRCSGSRQCYDPCRQATGCRFGRCMNRRCRCYGCG

SEQ ID NO: 260 - protein, 39 aa, Artificial Sequence
  source: /note=“Description of Artificial Sequence Synthetic polypeptide”
  MOD_RES (1)..(4): Any amino acid, amino acid analogue or not present
  MOD_RES (6)..(6): Any amino acid, amino acid analogue or not present
  MOD_RES (8)..(8): Any amino acid, amino acid analogue or not present
  MOD_RES (15)..(15): Any amino acid, amino acid analogue or not present
  MOD_RES (19)..(20): Any amino acid, amino acid analogue or not present
  MOD_RES (24)..(26): Any amino acid, amino acid analogue or not present
  MOD_RES (29)..(30): Any amino acid, amino acid analogue or not present
  MOD_RES (32)..(32): Any amino acid, amino acid analogue or not present
XXXXIXCXGSRQCYXPCRXXTGCXXXRCXXRXCRCYGCG

SEQ ID NO: 261 - protein, 39 aa, Artificial Sequence; identical to SEQ ID NO: 109
  source: /note=“Description of Artificial Sequence Synthetic polypeptide”
  VARIANT (1)..(1): /replace=“ ”
  VARIANT (2)..(2): /replace=“ ”
  VARIANT (3)..(3): /replace=“Gly” or “ ”
  VARIANT (4)..(4): /replace=“Ser” or “ ”
  VARIANT (6)..(6): /replace=“Ser”
  VARIANT (8)..(8): /replace=“Thr”
  VARIANT (15)..(15): /replace=“Asp”
  VARIANT (19)..(19): /replace=“Arg”
  VARIANT (20)..(20): /replace=“Arg” or “Lys”
  VARIANT (24)..(24): /replace=“Pro”
  VARIANT (25)..(25): /replace=“Gln”
  VARIANT (26)..(26): /replace=“Ala”
  VARIANT (29)..(29): /replace=“Leu”
  VARIANT (30)..(30): /replace=“Gln”
  VARIANT (32)..(32): /replace=“Ser”
  SITE (1)..(39): /note=“Variant residues given in the sequence have no preference with respect to those in the annotations for variant positions”

SEQ ID NO: 262 - protein, 40 aa, Artificial Sequence
  source: /note=“Description of Artificial Sequence Synthetic polypeptide”
  MOD_RES (1)..(3): Any amino acid, amino acid analogue or not present
  MOD_RES (5)..(5): Any amino acid, amino acid analogue or not present
  MOD_RES (7)..(7): Any amino acid, amino acid analogue or not present
  MOD_RES (9)..(9): Any amino acid, amino acid analogue or not present
  MOD_RES (11)..(12): Any amino acid, amino acid analogue or not present
  MOD_RES (14)..(15): Any amino acid, amino acid analogue or not present
  MOD_RES (18)..(18): Any amino acid, amino acid analogue or not present
  MOD_RES (22)..(22): Any amino acid, amino acid analogue or not present
  MOD_RES (31)..(31): Any amino acid, amino acid analogue or not present
  MOD_RES (33)..(33): Any amino acid, amino acid analogue or not present
  MOD_RES (36)..(36): Any amino acid, amino acid analogue or not present
  MOD_RES (40)..(40): Any amino acid, amino acid analogue or not present
XXXVXIXVXCXXSXXCLXPCRXAGMRFGRCXNXRCXCTPX

SEQ ID NO: 263 - protein, 40 aa, Artificial Sequence
  source: /note=“Description of Artificial Sequence Synthetic polypeptide”
  VARIANT (1)..(1): /replace=“ ”
  VARIANT (2)..(2): /replace=“Ser” or “ ”
  VARIANT (3)..(3): /replace=“Ser” or “ ”
  VARIANT (5)..(5): /replace=“Arg”
  VARIANT (7)..(7): /replace=“Pro”
  VARIANT (9)..(9): /replace=“Lys” or “Ser”
  VARIANT (11)..(11): /replace=“Lys”
  VARIANT (12)..(12): /replace=“His”
  VARIANT (14)..(14): /replace=“Gly”
  VARIANT (15)..(15): /replace=“Gln”
  VARIANT (18)..(18): /replace=“Arg” or “Lys”
  VARIANT (22)..(22): /replace=“Arg” or “Asp”
  VARIANT (31)..(31): /replace=“Met”
  VARIANT (33)..(33): /replace=“Gly”
  VARIANT (36)..(36): /replace=“Asp”
  VARIANT (40)..(40): /replace=“Arg” or “ ”
  SITE (1)..(40): /note=“Variant residues given in the sequence have no preference with respect to those in the annotations for variant positions”
GGGVPINVRCRGSRDCLDPCRKAGMRFGRCINSRCHCTPK

SEQ ID NO: 264 - protein, 35 aa, Artificial Sequence
  source: /note=“Description of Artificial Sequence Synthetic polypeptide”
  MOD_RES (1)..(1): Any amino acid
  MOD_RES (3)..(3): Any amino acid
  MOD_RES (7)..(7): Any amino acid
  MOD_RES (13)..(13): Any amino acid
  MOD_RES (18)..(18): Any amino acid
  MOD_RES (20)..(20): Any amino acid
  MOD_RES (22)..(22): Any amino acid
  MOD_RES (30)..(30): Any amino acid
  MOD_RES (35)..(35): Any amino acid
  source: /note=“See specification as filed for detailed description of substitutions and preferred embodiments”
XVXVRCXGSRQCXPCRRXGXRXGRCINRRXCRCYX

SEQ ID NO: 265 - protein, 32 aa, Artificial Sequence
  source: /note=“Description of Artificial Sequence Synthetic polypeptide”
  MOD_RES (1)..(1): Any amino acid
  MOD_RES (5)..(5): Any amino acid
  MOD_RES (11)..(11): Any amino acid
  MOD_RES (13)..(13): Any amino acid
  MOD_RES (16)..(16): Any amino acid
  MOD_RES (18)..(18): Any amino acid
  MOD_RES (27)..(27): Any amino acid
  MOD_RES (32)..(32): Any amino acid
  source: /note=“See specification as filed for detailed description of substitutions and preferred embodiments”
XGCVXRCRPGXRXCCXPXRRCSRRFGXRRCRX

SEQ ID NO: 266 - protein, 41 aa, Artificial Sequence
  source: /note=“Description of Artificial Sequence Synthetic polypeptide”
  MOD_RES (1)..(1): Any amino acid, amino acid analogue or not present
  MOD_RES (3)..(5): Any amino acid, amino acid analogue or not present
  MOD_RES (9)..(9): Any amino acid, amino acid analogue or not present
  MOD_RES (15)..(16): Any amino acid, amino acid analogue or not present
  MOD_RES (21)..(22): Any amino acid, amino acid analogue or not present
  MOD_RES (24)..(24): Any amino acid, amino acid analogue or not present
  MOD_RES (26)..(26): Any amino acid, amino acid analogue or not present
  MOD_RES (34)..(34): Any amino acid, amino acid analogue or not present
  MOD_RES (39)..(41): Any amino acid, amino acid analogue or not present
XVXXXVRCXGSRQCXXPCRRXXGXRXGRCINRRXCRCYXXX

SEQ ID NO: 267 - protein, 45 aa, Artificial Sequence
  source: /note=“Description of Artificial Sequence Synthetic polypeptide”
  MOD_RES (1)..(3): Any amino acid, amino acid analogue or not present
  MOD_RES (7)..(10): Any amino acid, amino acid analogue or not present
  MOD_RES (16)..(16): Any amino acid, amino acid analogue or not present
  MOD_RES (18)..(18): Any amino acid, amino acid analogue or not present
  MOD_RES (21)..(21): Any amino acid, amino acid analogue or not present
  MOD_RES (23)..(23): Any amino acid, amino acid analogue or not present
  MOD_RES (32)..(35): Any amino acid, amino acid analogue or not present
  MOD_RES (40)..(45): Any amino acid, amino acid analogue or not present
XXXGCVXXXXRCRPGXRXCCXPXRRCSRRFGXXXXRRCRXXXXXX

SEQ ID NO: 268 - protein, 10 aa, Artificial Sequence
  source: /note=“Description of Artificial Sequence Synthetic peptide”
GKCINKKCKC

SEQ ID NO: 269 - protein, 4 aa, Artificial Sequence
  source: /note=“Description of Artificial Sequence Synthetic peptide”
KCIN

SEQ ID NO: 270 - protein, 4 aa, Artificial Sequence
  source: /note=“Description of Artificial Sequence Synthetic peptide”
KKCK

SEQ ID NO: 271 - protein, 4 aa, Artificial Sequence
  source: /note=“Description of Artificial Sequence Synthetic peptide”
PCKR

SEQ ID NO: 272 - protein, 6 aa, Artificial Sequence
  source: /note=“Description of Artificial Sequence Synthetic peptide”
KRCSRR

SEQ ID NO: 273 - protein, 3 aa, Artificial Sequence
  source: /note=“Description of Artificial Sequence Synthetic peptide”
KQC

SEQ ID NO: 274 - protein, 10 aa, Artificial Sequence
  source: /note=“Description of Artificial Sequence Synthetic peptide”
GRCINRRCRC

SEQ ID NO: 275 - protein, 4 aa, Artificial Sequence
  source: /note=“Description of Artificial Sequence Synthetic peptide”
RCIN

SEQ ID NO: 276 - protein, 4 aa, Artificial Sequence
  source: /note=“Description of Artificial Sequence Synthetic peptide”
RRCR

SEQ ID NO: 277 - protein, 4 aa, Artificial Sequence
  source: /note=“Description of Artificial Sequence Synthetic peptide”
PCRR

SEQ ID NO: 278 - protein, 6 aa, Artificial Sequence
  source: /note=“Description of Artificial Sequence Synthetic peptide”
RRCSRR

SEQ ID NO: 279 - protein, 3 aa, Artificial Sequence
  source: /note=“Description of Artificial Sequence Synthetic peptide”
RQC

SEQ ID NO: 280 - protein, 4 aa, Artificial Sequence
  source: /note=“Description of Artificial Sequence Synthetic peptide”
PCKK

SEQ ID NO: 281 - protein, 6 aa, Artificial Sequence
  source: /note=“Description of Artificial Sequence Synthetic peptide”
KKCSKK

SEQ ID NO: 282 - protein, 32 aa, Artificial Sequence
  source: /note=“Description of Artificial Sequence Synthetic polypeptide”
GIVCKVCKIICGMQGKKVNICKAPIKCKCKKG

SEQ ID NO: 283 - protein, 35 aa, Artificial Sequence
  source: /note=“Description of Artificial Sequence Synthetic polypeptide”
SEKDCIKHLQRCRENKDCCSKKCSRRGTNPEKRCR

SEQ ID NO: 284 - protein, 37 aa, Artificial Sequence
  source: /note=“Description of Artificial Sequence Synthetic polypeptide”
VRIPVSCKHSGQCLKPCKDAGMRFGKCMNGKCDCTPK

SEQ ID NO: 285 - protein, 37 aa, Artificial Sequence
  source: /note=“Description of Artificial Sequence Synthetic polypeptide”
GVPINVKCRGSRDCLDPCKKAGMRFGKCINSKCHCTP

SEQ ID NO: 286 - protein, 33 aa, Artificial Sequence
  source: /note=“Description of Artificial Sequence Synthetic polypeptide”
AVCVYRTCDKDCKRRGYRSGKCINNACKCYPYG

SEQ ID NO: 287 - protein, 35 aa, Artificial Sequence
  source: /note=“Description of Artificial Sequence Synthetic polypeptide”
ISCTGSKQCYDPCKRKTGCPNAKCMNKSCKCYGCG

SEQ ID NO: 288 - protein, 38 aa, Artificial Sequence
  source: /note=“Description of Artificial Sequence Synthetic polypeptide”
QVQTNVKCQGGSCASVCRREIGVAAGKCINGKCVCYRN

SEQ ID NO: 289 - protein, 37 aa, Artificial Sequence
  source: /note=“Description of Artificial Sequence Synthetic polypeptide”
EVIRCSGSKQCYGPCKQQTGCTNSKCMNKVCKCYGCG

SEQ ID NO: 290 - protein, 33 aa, Artificial Sequence
  source: /note=“Description of Artificial Sequence Synthetic polypeptide”
ACKGVFDACTPGKNECCPNRVCSDKHKWCKWKL

SEQ ID NO: 291 - protein, 38 aa, Artificial Sequence
  source: /note=“Description of Artificial Sequence Synthetic polypeptide”
QIYTSKECNGSSECYSHCEGITGKRSGKCINKKCYCYR

SEQ ID NO: 292 - protein, 35 aa, Artificial Sequence
  source: /note=“Description of Artificial Sequence Synthetic polypeptide”
GCLEFWWKCNPNDDKCCRPKLKCSKLFKLCNFSFG

SEQ ID NO: 293 - protein, 37 aa, Artificial Sequence
  source: /note=“Description of Artificial Sequence Synthetic polypeptide”
DCVRFWGKCSQTSDCCPHLACKSKWPRNICVWDGSVG

SEQ ID NO: 294 - protein, 33 aa, Artificial Sequence
  source: /note=“Description of Artificial Sequence Synthetic polypeptide”
GCFGYKCDYYKGCCSGYVCSPTWKWCVRPGPGR

SEQ ID NO: 295 - protein, 60 aa, Artificial Sequence
  source: /note=“Description of Artificial Sequence Synthetic polypeptide”
MNAKFILLLVLTTMMLLPDTKGAEVIRCSGSKQCYGPCKQQTGCTNSKCMNKVCKCYGCG

SEQ ID NO: 296 - protein, 61 aa, Artificial Sequence
  source: /note=“Description of Artificial Sequence Synthetic polypeptide”
MNAKLIYLLLVVTTMTLMFDTAQAVDIMCSGPKQCYGPCKKETGCPNAKCMNRRCKCYGCV

SEQ ID NO: 297 - protein, 62 aa, Artificial Sequence
  source: /note=“Description of Artificial Sequence Synthetic polypeptide”
MNAKLIYLLLVVTTMMLTFDTTQAGDIKCSGTRQCWGPCKKQTTCTNSKCMNGKCKCYGCVG

SEQ ID NO: 298 - protein, 61 aa, Artificial Sequence
  source: /note=“Description of Artificial Sequence Synthetic polypeptide”
MNTKFIFLLLVVTNTMMLFDTKPVEGISCTGSKQCYDPCKRKTGCPNAKCMNKSCKCYGCG

SEQ ID NO: 299 - protein, 38 aa, Artificial Sequence
  source: /note=“Description of Artificial Sequence Synthetic polypeptide”
GVPINVKCSGSRDCLEPCKKAGMRFGKCINRKCHCTPK

SEQ ID NO: 300 - protein, 38 aa, Artificial Sequence
  source: /note=“Description of Artificial Sequence Synthetic polypeptide”
GVPINVKCTGSPQCLKPCKDAGMRFGKCINGKCHCTPK

SEQ ID NO: 301 - protein, 38 aa, Artificial Sequence
  source: /note=“Description of Artificial Sequence Synthetic polypeptide”
GVIINVKCKISRQCLEPCKKAGMRFGKCMNGKCHCTPK

SEQ ID NO: 302 - protein, 38 aa, Artificial Sequence
  source: /note=“Description of Artificial Sequence Synthetic polypeptide”
GVPINVKCRGSPQCIQPCRDAGMRFGKCMNGKCHCTPQ

SEQ ID NO: 303 - protein, 38 aa, Artificial Sequence
  source: /note=“Description of Artificial Sequence Synthetic polypeptide”
GVEINVKCTGSHQCIKPCKDAGMRFGKCINRKCHCTPK

SEQ ID NO: 304 - protein, 38 aa, Artificial Sequence
  source: /note=“Description of Artificial Sequence Synthetic polypeptide”
GVEINVKCSGSPQCLKPCKDAGMRFGKCMNRKCHCTPK

SEQ ID NO: 305 - protein, 38 aa, Artificial Sequence
  source: /note=“Description of Artificial Sequence Synthetic polypeptide”
GVPTDVKCRGSPQCIQPCKDAGMRFGKCMNGKCHCTPK

SEQ ID NO: 306 - protein, 38 aa, Artificial Sequence
  source: /note=“Description of Artificial Sequence Synthetic polypeptide”
GVPINVSCTGSPQCIKPCKDAGMRFGKCMNRKCHCTPK

SEQ ID NO: 307 - protein, 38 aa, Artificial Sequence
  source: /note=“Description of Artificial Sequence Synthetic polypeptide”
GVPINVPCTGSPQCIKPCKDAGMRFGKCMNRKCHCTPK

SEQ ID NO: 308 - protein, 37 aa, Artificial Sequence
  source: /note=“Description of Artificial Sequence Synthetic polypeptide”
VGINVKCKHSGQCLKPCKDAGMRFGKCINGKCDCTPK

SEQ ID NO: 309 - protein, 37 aa, Artificial Sequence
  source: /note=“Description of Artificial Sequence Synthetic polypeptide”
VGINVKCKHSGQCLKPCKDAGMRFGKCMNGKCDCTPK

SEQ ID NO: 310 - protein, 37 aa, Artificial Sequence
  source: /note=“Description of Artificial Sequence Synthetic polypeptide”
VGIPVSCKHSGQCIKPCKDAGMRFGKCMNRKCDCTPK

SEQ ID NO: 311 - protein, 36 aa, Artificial Sequence
  source: /note=“Description of Artificial Sequence Synthetic polypeptide”
RKGCFKEGHSCPKTAPCCRPLVCKGPSPNTKKCTRP

SEQ ID NO: 312 - protein, 32 aa, Artificial Sequence
  source: /note=“Description of Artificial Sequence Synthetic polypeptide”
SFCIPFKPCKSDENCCKKFKCKTTGIVKLCRW

SEQ ID NO: 313 - protein, 36 aa, Artificial Sequence
  source: /note=“Description of Artificial Sequence Synthetic polypeptide”
LKGCLPRNRFCNALSGPRCCSGLRCKELSIWASKCL

SEQ ID NO: 314 - protein, 34 aa, Artificial Sequence
  source: /note=“Description of Artificial Sequence Synthetic polypeptide”
GNYCLRGRCLPGGRKCCNGRPCECFAKICSCKPK

SEQ ID NO: 315 - protein, 29 aa, Artificial Sequence
  source: /note=“Description of Artificial Sequence Synthetic peptide”
TVKCGGCNRKCCPGGCRSGKCINGKCQCY

SEQ ID NO: 316 - protein, 29 aa, Artificial Sequence
  source: /note=“Description of Artificial Sequence Synthetic peptide”
GCMKEYCAGQCRGKVSQDYCLKHCKCIPR

SEQ ID NO: 317 - protein, 34 aa, Artificial Sequence
  source: /note=“Description of Artificial Sequence Synthetic polypeptide”
ACLGFGEKCNPSNDKCCKSSSLVCSQKHKWCKYG

SEQ ID NO: 318 - protein, 37 aa, Artificial Sequence
  source: /note=“Description of Artificial Sequence Synthetic polypeptide”
RGGCLPHNRFCNALSGPRCCSGLRCKELSIRDSRCLG

SEQ ID NO: 319 - protein, 36 aa, Artificial Sequence
  source: /note=“Description of Artificial Sequence Synthetic polypeptide”
RGGCLPRNKFCNPSSGPRCCSGLTCKELNIWASKCL

SEQ ID NO: 320 - protein, 33 aa, Artificial Sequence
  source: /note=“Description of Artificial Sequence Synthetic polypeptide”
QRSCAKPGDMCMGIKCCDGQCGCNRGTGRCFCK

SEQ ID NO: 321 - protein, 40 aa, Artificial Sequence
  source: /note=“Description of Artificial Sequence Synthetic polypeptide”
ARGCADAYKSCNHPRTCCDGYNGYKRACICSGSNCKCKKS

SEQ ID NO: 322 - protein, 37 aa, Artificial Sequence
  source: /note=“Description of Artificial Sequence Synthetic polypeptide”
RGGCLPHNRFCNALSGPRCCSGLRCKELSIWDSRCLG

SEQ ID NO: 323 - protein, 37 aa, Artificial Sequence
  source: /note=“Description of Artificial Sequence Synthetic polypeptide”
RGGCLPHNRFCNALSGPRCCSGLKCKELSIYDSRCLG

SEQ ID NO: 324 - protein, 37 aa, Artificial Sequence
  source: /note=“Description of Artificial Sequence Synthetic polypeptide”
RGGCLPHNRFCNALSGPRCCSRLKCKELSIWDSRCLG

SEQ ID NO: 325 - protein, 37 aa, Artificial Sequence
  source: /note=“Description of Artificial Sequence Synthetic polypeptide”
RGGCLPHNRFCNALTGPRCCSRLRCKELSIWDSICLG

SEQ ID NO: 326 - protein, 34 aa, Artificial Sequence
  source: /note=“Description of Artificial Sequence Synthetic polypeptide”
SCADAYKSCDSLKCCNNRTCMCSMIGTNCTCRKK

SEQ ID NO: 327 - protein, 30 aa, Artificial Sequence
  source: /note=“Description of Artificial Sequence Synthetic polypeptide”
ERRCLPAGKTCVRGPMRVPCCGSCSQNKCT

SEQ ID NO: 328 - protein, 31 aa, Artificial Sequence
  source: /note=“Description of Artificial Sequence Synthetic polypeptide”
LCSREGEFCYKLRKCCAGFYCKAFVLHCYRN

SEQ ID NO: 329 - protein, 23 aa, Artificial Sequence
  source: /note=“Description of Artificial Sequence Synthetic peptide”
ACGSCRKKCKGSGKCINGRCKCY

SEQ ID NO: 330 - protein, 23 aa, Artificial Sequence
  source: /note=“Description of Artificial Sequence Synthetic peptide”
ACGSCRKKCKGPGKCINGRCKCY

SEQ ID NO: 331 - protein, 32 aa, Artificial Sequence
  source: /note=“Description of Artificial Sequence Synthetic polypeptide”
ACQGYMRKCGRDKPPCCKKLECSKTWRWCVWN

SEQ ID NO: 332 - protein, 30 aa, Artificial Sequence
  source: /note=“Description of Artificial Sequence Synthetic polypeptide”
GRYCQKWMWTCDSKRACCEGLRCKLWCRKI

SEQ ID NO: 333 - protein, 34 aa, Artificial Sequence
  source: /note=“Description of Artificial Sequence Synthetic polypeptide”
NAKCRGSPECLPKCKEAIGKAAGKCMNGKCKCYP

SEQ ID NO: 334 - protein, 34 aa, Artificial Sequence
  source: /note=“Description of Artificial Sequence Synthetic polypeptide”
NVKCRGSKECLPACKAAVGKAAGKCMNGKCKCYP

SEQ ID NO: 335 - protein, 34 aa, Artificial Sequence
  source: /note=“Description of Artificial Sequence Synthetic polypeptide”
NVKCRGSPECLPKCKEAIGKSAGKCMNGKCKCYP

SEQ ID NO: 336 - protein, 34 aa, Artificial Sequence
  source: /note=“Description of Artificial Sequence Synthetic polypeptide”
NAKCRGSPECLPKCKQAIGKAAGKCMNGKCKCYP

SEQ ID NO: 337 - protein, 33 aa, Artificial Sequence
  source: /note=“Description of Artificial Sequence Synthetic polypeptide”
RGYCAEKGIKCHNIHCCSGLTCKCKGSSCVCRK

SEQ ID NO: 338 - protein, 31 aa, Artificial Sequence
  source: /note=“Description of Artificial Sequence Synthetic polypeptide”
ERGCKLTFWKCKNKKECCGWNACALGICMPR

SEQ ID NO: 339 - protein, 39 aa, Artificial Sequence
  source: /note=“Description of Artificial Sequence Synthetic polypeptide”
KKKCIAKDYGRCKWGGTPCCRGRGCICSIMGTNCECKPR

SEQ ID NO: 340 - protein, 29 aa, Artificial Sequence
  source: /note=“Description of Artificial Sequence Synthetic peptide”
GCKLTFWKCKNKKECCGWNACALGICMPR

SEQ ID NO: 341 - protein, 32 aa, Artificial Sequence
  source: /note=“Description of Artificial Sequence Synthetic polypeptide”
ACKGLFVTCTPGKDECCPNHVCSSKHKWCKYK

SEQ ID NO: 342 - protein, 36 aa, Artificial Sequence
  source: /note=“Description of Artificial Sequence Synthetic polypeptide”
IACAPRGLLCFRDKECCKGLTCKGRFVNTWPTFCLV

SEQ ID NO: 343 - protein, 36 aa, Artificial Sequence
  source: /note=“Description of Artificial Sequence Synthetic polypeptide”
ACAGLYKKCGKGVNTCCENRPCKCDLAMGNCICKKK

SEQ ID NO: 344 - protein, 38 aa, Artificial Sequence
  source: /note=“Description of Artificial Sequence Synthetic polypeptide”
FTCAISCDIKVNGKPCKGSGEKKCSGGWSCKFNVCVKV

SEQ ID NO: 345 - protein, 33 aa, Artificial Sequence
  source: /note=“Description of Artificial Sequence Synthetic polypeptide”
GFCAQKGIKCHDIHCCTNLKCVREGSNRVCRKA

SEQ ID NO: 346 - protein, 42 aa, Artificial Sequence
  source: /note=“Description of Artificial Sequence Synthetic polypeptide”
CAKKRNWCGKNEDCCCPMKCIYAWYNQQGSCQSTITGLFKKC

SEQ ID NO: 347 - protein, 28 aa, Artificial Sequence
  source: /note=“Description of Artificial Sequence Synthetic peptide”
YCQKWMWTCDSARKCCEGLVCRLWCKKI

SEQ ID NO: 348 - protein, 37 aa, Artificial Sequence
  source: /note=“Description of Artificial Sequence Synthetic polypeptide”
RGGCLPHNKFCNALSGPRCCSGLKCKELTIWNTKCLE

SEQ ID NO: 349 - protein, 34 aa, Artificial Sequence
  source: /note=“Description of Artificial Sequence Synthetic polypeptide”
NVKCTGSKQCLPACKAAVGKAAGKCMNGKCKCYT

SEQ ID NO: 350 - protein, 33 aa, Artificial Sequence
  source: /note=“Description of Artificial Sequence Synthetic polypeptide”
QRSCAKPGEMCMRIKCCDGQCGCNRGTGRCFCK

SEQ ID NO: 351 - protein, 36 aa, Artificial Sequence
  source: /note=“Description of Artificial Sequence Synthetic polypeptide”
GCIPKHKRCTWSGPKCCNNISCHCNISGTLCKCRPG

SEQ ID NO: 352 - protein, 33 aa, Artificial Sequence
  source: /note=“Description of Artificial Sequence Synthetic polypeptide”
NYCVAKRCRPGGRQCCSGKPCACVGKVCKCPRD

SEQ ID NO: 353 - protein, 37 aa, Artificial Sequence
  source: /note=“Description of Artificial Sequence Synthetic polypeptide”
ERGCSGAYKRCSSSQRCCEGRPCVCSAINSNCKCRKT

SEQ ID NO: 354 - protein, 36 aa, Artificial Sequence
  source: /note=“Description of Artificial Sequence Synthetic polypeptide”
RYCPRNPEACYNYCLRTGRPGGYCGGRSRITCFCFR

SEQ ID NO: 355 - protein, 33 aa, Artificial Sequence
  source: /note=“Description of Artificial Sequence Synthetic polypeptide”
QRSCAKPGEMCMGIKCCDGQCGCNRGTGRCFCK

SEQ ID NO: 356 - protein, 36 aa, Artificial Sequence
  source: /note=“Description of Artificial Sequence Synthetic polypeptide”
RRGCFKEGKWCPKSAPCCAPLKCKGPSIKQQKCVRE

SEQ ID NO: 357 - protein, 31 aa, Artificial Sequence
  source: /note=“Description of Artificial Sequence Synthetic polypeptide”
TVKCGGCNRKCCAGGCRSGKCINGKCQCYGR

SEQ ID NO: 358 - protein, 29 aa, Artificial Sequence
  source: /note=“Description of Artificial Sequence Synthetic peptide”
ERRCEPSGKPCRPLMRIPCCGSCVRGKCA

SEQ ID NO: 359 - protein, 36 aa, Artificial Sequence
  source: /note=“Description of Artificial Sequence Synthetic polypeptide”
RGGCLPRNKFCNPSSGPRCCSGLTCKELNIWANKCL

SEQ ID NO: 360 - protein, 42 aa, Artificial Sequence
  source: /note=“Description of Artificial Sequence Synthetic polypeptide”
CAKKRNWCGKNEDCCCPMKCIYAWYNQQGSCQTTITGLFKKC

SEQ ID NO: 361 - protein, 37 aa, Artificial Sequence
  source: /note=“Description of Artificial Sequence Synthetic polypeptide”
VRIPVSCKHSGQCLKPCKDAGMRTGKCMNGKCDCTPK

SEQ ID NO: 362 - protein, 20 aa, Artificial Sequence
  source: /note=“Description of Artificial Sequence Synthetic peptide”
VKCTTSKDCWPPCKKVTGRA

SEQ ID NO: 363 - protein, 32 aa, Artificial Sequence
  source: /note=“Description of Artificial Sequence Synthetic polypeptide”
GIVCRVCRIICGMQGRRVNICRAPIRCRCRRG

SEQ ID NO: 364 - protein, 35 aa, Artificial Sequence
  source: /note=“Description of Artificial Sequence Synthetic polypeptide”
SERDCIRHLQRCRENRDCCSRRCSRRGTNPERRCR

SEQ ID NO: 365 - protein, 37 aa, Artificial Sequence
  source: /note=“Description of Artificial Sequence Synthetic polypeptide”
VRIPVSCRHSGQCLRPCRDAGMRFGRCMNGRCDCTPR

SEQ ID NO: 366 - protein, 37 aa, Artificial Sequence
  source: /note=“Description of Artificial Sequence Synthetic polypeptide”
GVPINVRCRGSRDCLDPCRRAGMRFGRCINSRCHCTP

SEQ ID NO: 367 - protein, 33 aa, Artificial Sequence
  source: /note=“Description of Artificial Sequence Synthetic polypeptide”
AVCVYRTCDRDCRRRGYRSGRCINNACRCYPYG

SEQ ID NO: 368 - protein, 35 aa, Artificial Sequence
  source: /note=“Description of Artificial Sequence Synthetic polypeptide”
ISCTGSRQCYDPCRRRTGCPNARCMNRSCRCYGCG

SEQ ID NO: 369 - protein, 38 aa, Artificial Sequence
  source: /note=“Description of Artificial Sequence Synthetic polypeptide”
QVQTNVRCQGGSCASVCRREIGVAAGRCINGRCVCYRN

SEQ ID NO: 370 - protein, 37 aa, Artificial Sequence
  source: /note=“Description of Artificial Sequence Synthetic polypeptide”
EVIRCSGSRQCYGPCRQQTGCTNSRCMNRVCRCYGCG

SEQ ID NO: 371 - protein, 33 aa, Artificial Sequence
  source: /note=“Description of Artificial Sequence Synthetic polypeptide”
ACRGVFDACTPGRNECCPNRVCSDRHRWCRWRL

SEQ ID NO: 372 - protein, 38 aa, Artificial Sequence
  source: /note=“Description of Artificial Sequence Synthetic polypeptide”
QIYTSRECNGSSECYSHCEGITGRRSGRCINRRCYCYR

SEQ ID NO: 373 - protein, 35 aa, Artificial Sequence
  source: /note=“Description of Artificial Sequence Synthetic polypeptide”
GCLEFWWRCNPNDDRCCRPRLRCSRLFRLCNFSFG

SEQ ID NO: 374 - protein, 37 aa, Artificial Sequence
  source: /note=“Description of Artificial Sequence Synthetic polypeptide”
DCVRFWGRCSQTSDCCPHLACRSRWPRNICVWDGSVG

SEQ ID NO: 375 - protein, 33 aa, Artificial Sequence
  source: /note=“Description of Artificial Sequence Synthetic polypeptide”
GCFGYRCDYYRGCCSGYVCSPTWRWCVRPGPGR

SEQ ID NO: 376 - protein, 60 aa, Artificial Sequence
  source: /note=“Description of Artificial Sequence Synthetic polypeptide”
MNARFILLLVLTTMMLLPDTRGAEVIRCSGSRQCYGPCRQQTGCTNSRCMNRVCRCYGCG

SEQ ID NO: 377 - protein, 61 aa, Artificial Sequence
  source: /note=“Description of Artificial Sequence Synthetic polypeptide”
MNARLIYLLLVVTTMTLMFDTAQAVDIMCSGPRQCYGPCRRETGCPNARCMNRRCRCYGCV

SEQ ID NO: 378 - protein, 62 aa, Artificial Sequence
  source: /note=“Description of Artificial Sequence Synthetic polypeptide”
MNARLIYLLLVVTTMMLTFDTTQAGDIRCSGTRQCWGPCRRQTTCTNSRCMNGRCRCYGCVG

SEQ ID NO: 379 - protein, 61 aa, Artificial Sequence
  source: /note=“Description of Artificial Sequence Synthetic polypeptide”
MNTRFIFLLLVVTNTMMLFDTRPVEGISCTGSRQCYDPCRRRTGCPNARCMNRSCRCYGCG

SEQ ID NO: 380 - protein, 38 aa, Artificial Sequence
  source: /note=“Description of Artificial Sequence Synthetic polypeptide”
GVPINVRCSGSRDCLEPCRRAGMRFGRCINRRCHCTPR

SEQ ID NO: 381 - protein, 38 aa, Artificial Sequence
  source: /note=“Description of Artificial Sequence Synthetic polypeptide”
GVPINVRCTGSPQCLRPCRDAGMRFGRCINGRCHCTPR

SEQ ID NO: 382 - protein, 38 aa, Artificial Sequence
  source: /note=“Description of Artificial Sequence Synthetic polypeptide”
GVIINVRCRISRQCLEPCRRAGMRFGRCMNGRCHCTPR

SEQ ID NO: 383 - protein, 38 aa, Artificial Sequence
  source: /note=“Description of Artificial Sequence Synthetic polypeptide”
GVPINVRCRGSPQCIQPCRDAGMRFGRCMNGRCHCTPQ

SEQ ID NO: 384 - protein, 38 aa, Artificial Sequence
  source: /note=“Description of Artificial Sequence Synthetic polypeptide”
GVEINVRCTGSHQCIRPCRDAGMRFGRCINRRCHCTPR

SEQ ID NO: 385 - protein, 38 aa, Artificial Sequence
  source: /note=“Description of Artificial Sequence Synthetic polypeptide”
GVEINVRCSGSPQCLRPCRDAGMRFGRCMNRRCHCTPR

SEQ ID NO: 386 - protein, 38 aa, Artificial Sequence
  source: /note=“Description of Artificial Sequence Synthetic polypeptide”
GVPTDVRCRGSPQCIQPCRDAGMRFGRCMNGRCHCTPR

SEQ ID NO: 387 - protein, 38 aa, Artificial Sequence
  source: /note=“Description of Artificial Sequence Synthetic polypeptide”
GVPINVSCTGSPQCIRPCRDAGMRFGRCMNRRCHCTPR

SEQ ID NO: 388 - protein, 38 aa, Artificial Sequence
  source: /note=“Description of Artificial Sequence Synthetic polypeptide”
GVPINVPCTGSPQCIRPCRDAGMRFGRCMNRRCHCTPR

SEQ ID NO: 389 - protein, 37 aa, Artificial Sequence
  source: /note=“Description of Artificial Sequence Synthetic polypeptide”
VGINVRCRHSGQCLRPCRDAGMRFGRCINGRCDCTPR

SEQ ID NO: 390 - protein, 37 aa, Artificial Sequence
  source: /note=“Description of Artificial Sequence Synthetic polypeptide”
VGINVRCRHSGQCLRPCRDAGMRFGRCMNGRCDCTPR

SEQ ID NO: 391 - protein, 37 aa, Artificial Sequence
  source: /note=“Description of Artificial Sequence Synthetic polypeptide”
VGIPVSCRHSGQCIRPCRDAGMRFGRCMNRRCDCTPR

SEQ ID NO: 392 - protein, 36 aa, Artificial Sequence
  source: /note=“Description of Artificial Sequence Synthetic polypeptide”
RRGCFREGHSCPRTAPCCRPLVCRGPSPNTRRCTRP

SEQ ID NO: 393 - protein, 32 aa, Artificial Sequence
  source: /note=“Description of Artificial Sequence Synthetic polypeptide”
SFCIPFRPCRSDENCCRRFRCRTTGIVRLCRW

SEQ ID NO: 394 - protein, 36 aa, Artificial Sequence
  source: /note=“Description of Artificial Sequence Synthetic polypeptide”
LRGCLPRNRFCNALSGPRCCSGLRCRELSIWASRCL

SEQ ID NO: 395 - protein, 34 aa, Artificial Sequence
  source: /note=“Description of Artificial Sequence Synthetic polypeptide”
GNYCLRGRCLPGGRRCCNGRPCECFARICSCRPR

SEQ ID NO: 396 - protein, 29 aa, Artificial Sequence
  source: /note=“Description of Artificial Sequence Synthetic peptide”
TVRCGGCNRRCCPGGCRSGRCINGRCQCY

SEQ ID NO: 397 - protein, 29 aa, Artificial Sequence
  source: /note=“Description of Artificial Sequence Synthetic peptide”
GCMREYCAGQCRGRVSQDYCLRHCRCIPR

SEQ ID NO: 398 - protein, 34 aa, Artificial Sequence
  source: /note=“Description of Artificial Sequence Synthetic polypeptide”
ACLGFGERCNPSNDRCCRSSSLVCSQRHRWCRYG

SEQ ID NO: 399 - protein, 37 aa, Artificial Sequence
  source: /note=“Description of Artificial Sequence Synthetic polypeptide”
RGGCLPHNRFCNALSGPRCCSGLRCRELSIRDSRCLG

SEQ ID NO: 400 - protein, 36 aa, Artificial Sequence
  source: /note=“Description of Artificial Sequence Synthetic polypeptide”
RGGCLPRNRFCNPSSGPRCCSGLTCRELNIWASRCL

SEQ ID NO: 401 - protein, 33 aa, Artificial Sequence
  source: /note=“Description of Artificial Sequence Synthetic polypeptide”
QRSCARPGDMCMGIRCCDGQCGCNRGTGRCFCR

SEQ ID NO: 402 - protein, 40 aa, Artificial Sequence
  source: /note=“Description of Artificial Sequence Synthetic polypeptide”
ARGCADAYRSCNHPRTCCDGYNGYRRACICSGSNCRCRRS

SEQ ID NO: 403 - protein, 37 aa, Artificial Sequence
  source: /note=“Description of Artificial Sequence Synthetic polypeptide”
RGGCLPHNRFCNALSGPRCCSGLRCRELSIWDSRCLG

SEQ ID NO: 404 - protein, 37 aa, Artificial Sequence
  source: /note=“Description of Artificial Sequence Synthetic polypeptide”
RGGCLPHNRFCNALSGPRCCSGLRCRELSIYDSRCLG

SEQ ID NO: 405 - protein, 37 aa, Artificial Sequence
  source: /note=“Description of Artificial Sequence Synthetic polypeptide”
RGGCLPHNRFCNALSGPRCCSRLRCRELSIWDSRCLG

SEQ ID NO: 406 - protein, 37 aa, Artificial Sequence
  source: /note=“Description of Artificial Sequence Synthetic polypeptide”
RGGCLPHNRFCNALTGPRCCSRLRCRELSIWDSICLG

SEQ ID NO: 407 - protein, 34 aa, Artificial Sequence
  source: /note=“Description of Artificial Sequence Synthetic polypeptide”
SCADAYKSCDSLRCCNNRTCMCSMIGTNCTCRRR

SEQ ID NO: 408 - protein, 30 aa, Artificial Sequence
  source: /note=“Description of Artificial Sequence Synthetic polypeptide”
ERRCLPAGRTCVRGPMRVPCCGSCSQNRCT

SEQ ID NO: 409 - protein, 31 aa, Artificial Sequence
  source: /note=“Description of Artificial Sequence Synthetic polypeptide”
LCSREGEFCYRLRRCCAGFYCRAFVLHCYRN

SEQ ID NO: 410 - protein, 23 aa, Artificial Sequence
  source: /note=“Description of Artificial Sequence Synthetic peptide”
ACGSCRRRCRGSGRCINGRCRCY

SEQ ID NO: 411 - protein, 23 aa, Artificial Sequence
  source: /note=“Description of Artificial Sequence Synthetic peptide”
ACGSCRRRCRGPGRCINGRCRCY

SEQ ID NO: 412 - protein, 32 aa, Artificial Sequence
  source: /note=“Description of Artificial Sequence Synthetic polypeptide”
ACQGYMRRCGRDRPPCCRRLECSRTWRWCVWN

SEQ ID NO: 413 - protein, 30 aa, Artificial Sequence
  source: /note=“Description of Artificial Sequence Synthetic polypeptide”
GRYCQRWMWTCDSRRACCEGLRCRLWCRRI

SEQ ID NO: 414 - protein, 34 aa, Artificial Sequence
  source: /note=“Description of Artificial Sequence Synthetic polypeptide”
NARCRGSPECLPRCREAIGRAAGRCMNGRCRCYP

SEQ ID NO: 415 - protein, 34 aa, Artificial Sequence
  source: /note=“Description of Artificial Sequence Synthetic polypeptide”
NVRCRGSRECLPACRAAVGRAAGRCMNGRCRCYP

SEQ ID NO: 416 - protein, 34 aa, Artificial Sequence
  source: /note=“Description of Artificial Sequence Synthetic polypeptide”
NVRCRGSPECLPRCREAIGRSAGRCMNGRCRCYP

SEQ ID NO: 417 - protein, 34 aa, Artificial Sequence
  source: /note=“Description of Artificial Sequence Synthetic polypeptide”
NARCRGSPECLPRCRQAIGRAAGRCMNGRCRCYP

SEQ ID NO: 418 - protein, 33 aa, Artificial Sequence
  source: /note=“Description of Artificial Sequence Synthetic polypeptide”
RGYCAERGIRCHNIHCCSGLTCRCRGSSCVCRR

SEQ ID NO: 419 - protein, 31 aa, Artificial Sequence
  source: /note=“Description of Artificial Sequence Synthetic polypeptide”
ERGCRLTFWRCRNRRECCGWNACALGICMPR

SEQ ID NO: 420 - protein, 39 aa, Artificial Sequence
  source: /note=“Description of Artificial Sequence Synthetic polypeptide”
RRRCIARDYGRCRWGGTPCCRGRGCICSIMGTNCECRPR

SEQ ID NO: 421 - protein, 29 aa, Artificial Sequence
  source: /note=“Description of Artificial Sequence Synthetic peptide”
GCRLTFWRCRNRRECCGWNACALGICMPR

SEQ ID NO: 422 - protein, 32 aa, Artificial Sequence
  source: /note=“Description of Artificial Sequence Synthetic polypeptide”
ACRGLFVTCTPGRDECCPNHVCSSRHRWCRYR

SEQ ID NO: 423 - protein, 36 aa, Artificial Sequence
  source: /note=“Description of Artificial Sequence Synthetic polypeptide”
IACAPRGLLCFRDRECCRGLTCRGRFVNTWPTFCLV

SEQ ID NO: 424 - protein, 36 aa, Artificial Sequence
  source: /note=“Description of Artificial Sequence Synthetic polypeptide”
ACAGLYRRCGRGVNTCCENRPCRCDLAMGNCICRRR

SEQ ID NO: 425 - protein, 38 aa, Artificial Sequence
  source: /note=“Description of Artificial Sequence Synthetic polypeptide”
FTCAISCDIRVNGRPCRGSGERRCSGGWSCRFNVCVRV

SEQ ID NO: 426 - protein, 33 aa, Artificial Sequence
  source: /note=“Description of Artificial Sequence Synthetic polypeptide”
GFCAQRGIRCHDIHCCTNLRCVREGSNRVCRRA

SEQ ID NO: 427 - protein, 42 aa, Artificial Sequence
  source: /note=“Description of Artificial Sequence Synthetic polypeptide”
CARRRNWCGRNEDCCCPMRCIYAWYNQQGSCQSTITGLFRRC

SEQ ID NO: 428 - protein, 28 aa, Artificial Sequence
  source: /note=“Description of Artificial Sequence Synthetic peptide”
YCQRWMWTCDSARRCCEGLVCRLWCRRI

SEQ ID NO: 429 - protein, 37 aa, Artificial Sequence
  source: /note=“Description of Artificial Sequence Synthetic polypeptide”
RGGCLPHNRFCNALSGPRCCSGLRCRELTIWNTRCLE

SEQ ID NO: 430 - protein, 34 aa, Artificial Sequence
  source: /note=“Description of Artificial Sequence Synthetic polypeptide”
NVRCTGSRQCLPACRAAVGRAAGRCMNGRCRCYT

SEQ ID NO: 431 - protein, 33 aa, Artificial Sequence
  source: /note=“Description of Artificial Sequence Synthetic polypeptide”
QRSCARPGEMCMRIRCCDGQCGCNRGTGRCFCR

SEQ ID NO: 432 - protein, 36 aa, Artificial Sequence
  source: /note=“Description of Artificial Sequence Synthetic polypeptide”
GCIPRHRRCTWSGPRCCNNISCHCNISGTLCRCRPG

SEQ ID NO: 433 - protein, 33 aa, Artificial Sequence
  source: /note=“Description of Artificial Sequence Synthetic polypeptide”
NYCVARRCRPGGRQCCSGRPCACVGRVCRCPRD

SEQ ID NO: 434 - protein, 37 aa, Artificial Sequence
  source: /note=“Description of Artificial Sequence Synthetic polypeptide”
ERGCSGAYRRCSSSQRCCEGRPCVCSAINSNCRCRRT

SEQ ID NO: 435 - protein, 33 aa, Artificial Sequence
  source: /note=“Description of Artificial Sequence Synthetic polypeptide”
QRSCARPGEMCMGIRCCDGQCGCNRGTGRCFCR

SEQ ID NO: 436 - protein, 36 aa, Artificial Sequence
  source: /note=“Description of Artificial Sequence Synthetic polypeptide”
RRGCFREGRWCPRSAPCCAPLRCRGPSIRQQRCVRE

SEQ ID NO: 437 - protein, 31 aa, Artificial Sequence
  source: /note=“Description of Artificial Sequence Synthetic polypeptide”
TVRCGGCNRRCCAGGCRSGRCINGRCQCYGR

SEQ ID NO: 438 - protein, 29 aa, Artificial Sequence
  source: /note=“Description of Artificial Sequence Synthetic peptide”
ERRCEPSGRPCRPLMRIPCCGSCVRGRCA

SEQ ID NO: 439 - protein, 36 aa, Artificial Sequence
  source: /note=“Description of Artificial Sequence Synthetic polypeptide”
RGGCLPRNRFCNPSSGPRCCSGLTCRELNIWANRCL

SEQ ID NO: 440 - protein, 42 aa, Artificial Sequence
  source: /note=“Description of Artificial Sequence Synthetic polypeptide”
CARRRNWCGRNEDCCCPMRCIYAWYNQQGSCQTTITGLFRRC

SEQ ID NO: 441 - protein, 37 aa, Artificial Sequence
  source: /note=“Description of Artificial Sequence Synthetic polypeptide”
VRIPVSCRHSGQCLRPCRDAGMRTGRCMNGRCDCTPR

SEQ ID NO: 442 - protein, 38 aa, Artificial Sequence
  source: /note=“Description of Artificial Sequence Synthetic polypeptide”
QKILSNRCNNSSECIPHCIRIFGTRAAKCINRKCYCYP

SEQ ID NO: 443 - protein, 32 aa, Artificial Sequence
  source: /note=“Description of Artificial Sequence Synthetic polypeptide”
AVCNLKRCQLSCRSLGLLGKCIGDKCECVKHG

SEQ ID NO: 444 - protein, 37 aa, Artificial Sequence
  source: /note=“Description of Artificial Sequence Synthetic polypeptide”
ISIGIRCSPSIDLCEGQCRIRRYFTGYCSGDTCHCSG

SEQ ID NO: 445 - protein, 33 aa, Artificial Sequence
  source: /note=“Description of Artificial Sequence Synthetic polypeptide”
GDCLPHLRRCRENNDCCSRRCRRRGANPERRCR

SEQ ID NO: 446 - protein, 34 aa, Artificial Sequence
  source: /note=“Description of Artificial Sequence Synthetic polypeptide”
SCEPGRTFRDRCNTCKCGADGRSAACTLRACPNQ

SEQ ID NO: 447 - protein, 33 aa, Artificial Sequence
  source: /note=“Description of Artificial Sequence Synthetic polypeptide”
GDCLPHLKRCKADNDCCGKKCKRRGTNAEKRCR

SEQ ID NO: 448 - protein, 33 aa, Artificial Sequence
  source: /note=“Description of Artificial Sequence Synthetic polypeptide”
GDCLPHLKRCKENNDCCSKKCKRRGTNPEKRCR

SEQ ID NO: 449 - protein, 33 aa, Artificial Sequence
  source: /note=“Description of Artificial Sequence Synthetic polypeptide”
KDCLKKLKLCKENKDCCSKSCKRRGTNIEKRCR

SEQ ID NO: 450 - protein, 33 aa, Artificial Sequence
  source: /note=“Description of Artificial Sequence Synthetic polypeptide”
GDCLPHLKRCKENNDCCSKKCKRRGANPEKRCR

SEQ ID NO: 451 - protein, 37 aa, Artificial Sequence
  source: /note=“Description of Artificial Sequence Synthetic polypeptide”
VFINVKCRGSPECLPKCKEAIGKSAGKCMNGKCKCYP

SEQ ID NO: 452 - protein, 37 aa, Artificial Sequence
  source: /note=“Description of Artificial Sequence Synthetic polypeptide”
VFINAKCRGSPECLPKCKEAIGKAAGKCMNGKCKCYP

SEQ ID NO: 453 - protein, 36 aa, Artificial Sequence
  source: /note=“Description of Artificial Sequence Synthetic polypeptide”
VIINVKCKISRQCLEPCKKAGMRFGKCMNGKCHCTP

SEQ ID NO: 454 - protein, 36 aa, Artificial Sequence
  source: /note=“Description of Artificial Sequence Synthetic polypeptide”
VPTDVKCRGSPQCIQPCKDAGMRFGKCMNGKCHCTP

SEQ ID NO: 455 - protein, 36 aa, Artificial Sequence
  source: /note=“Description of Artificial Sequence Synthetic polypeptide”
VRIPVSCKHSGQCLKPCKDAGMRFGKCMNGKCDCTP

SEQ ID NO: 456 - protein, 36 aa, Artificial Sequence
  source: /note=“Description of Artificial Sequence Synthetic polypeptide”
VRIPVSCRHSGQCLRPCRDAGMRFGRCMNGRCDCTP

SEQ ID NO: 457 - protein, 33 aa, Artificial Sequence
  source: /note=“Description of Artificial Sequence Synthetic polypeptide”
TNVSCTTSKECWSVCQRLHNTSRGKCMNKKCRC

SEQ ID NO: 458 - protein, 32 aa, Artificial Sequence
  source: /note=“Description of Artificial Sequence Synthetic polypeptide”
NVKCTGSKQCLPACKAAVGKAAGKCMNGKCKC

SEQ ID NO: 459 - protein, 37 aa, Artificial Sequence
  source: /note=“Description of Artificial Sequence Synthetic polypeptide”
GVPINVRCRGSRDCLDPCRGAGERHGRCGNSRCHCTP

SEQ ID NO: 460 - protein, 37 aa, Artificial Sequence
  source: /note=“Description of Artificial Sequence Synthetic polypeptide”
VRIPVSCRHSGQCLRPCRDAGERHGRCGGGRCDCTPR

SEQ ID NO: 461 - protein, 38 aa, Artificial Sequence
  source: /note=“Description of Artificial Sequence Synthetic polypeptide”
QVQTNVRCQGGSCGSVCRREGGGAGGGCGNGRCGCYRN

SEQ ID NO: 462 - protein, 32 aa, Artificial Sequence
  source: /note=“Description of Artificial Sequence Synthetic polypeptide”
IKCSESYQCFPVCKSRFGKTNGRCVNGFCDCF

SEQ ID NO: 463 - protein, 33 aa, Artificial Sequence
  source: /note=“Description of Artificial Sequence Synthetic polypeptide”
VKCSSPQQCLKPCKAAFGISAGGKCINGKCKCY

SEQ ID NO: 464 - protein, 32 aa, Artificial Sequence
  source: /note=“Description of Artificial Sequence Synthetic polypeptide”
VSCSASSQCWPVCKKLFGTYRGKCMNSKCRCY

SEQ ID NO: 465 - protein, 32 aa, Artificial Sequence
  source: /note=“Description of Artificial Sequence Synthetic polypeptide”
ESCTASNQCWSICKRLHNTNRGKCMNKKCRCY

SEQ ID NO: 466 - protein, 32 aa, Artificial Sequence
  source: /note=“Description of Artificial Sequence Synthetic polypeptide”
VSCTTSKECWSVCEKLYNTSRGKCMNKKCRCY

SEQ ID NO: 467 - protein, 32 aa, Artificial Sequence
  source: /note=“Description of Artificial Sequence Synthetic polypeptide”
MRCKSSKECLVKCKQATGRPNGKCMNRKCKCY

SEQ ID NO: 468 - protein, 32 aa, Artificial Sequence
  source: /note=“Description of Artificial Sequence Synthetic polypeptide”
IKCTLSKDCYSPCKKETGCPRAKCINRNCKCY

SEQ ID NO: 469 - protein, 32 aa, Artificial Sequence
  source: /note=“Description of Artificial Sequence Synthetic polypeptide”
IRCSGSRDCYSPCMKQTGCPNAKCINKSCKCY

SEQ ID NO: 470 - protein, 32 aa, Artificial Sequence
  source: /note=“Description of Artificial Sequence Synthetic polypeptide”
IRCSGTRECYAPCQKLTGCLNAKCMNKACKCY

SEQ ID NO: 471 - protein, 32 aa, Artificial Sequence
  source: /note=“Description of Artificial Sequence Synthetic polypeptide”
ISCTNPKQCYPHCKKETGYPNAKCMNRKCKCF

SEQ ID NO: 472 - protein, 32 aa, Artificial Sequence
  source: /note=“Description of Artificial Sequence Synthetic polypeptide”
ASCRTPKDCADPCRKETGCPYGKCMNRKCKCN

SEQ ID NO: 473 - protein, 31 aa, Artificial Sequence
  source: /note=“Description of Artificial Sequence Synthetic polypeptide”
TSCISPKQCTEPCRAKGCKHGKCMNRKCHCM

SEQ ID NO: 474 - protein, 31 aa, Artificial Sequence
  source: /note=“Description of Artificial Sequence Synthetic polypeptide”
KECTGPQHCTNFCRKNKCTHGKCMNRKCKCF

SEQ ID NO: 475 - protein, 32 aa, Artificial Sequence
  source: /note=“Description of Artificial Sequence Synthetic polypeptide”
IKCRTPKDCADPCRKQTGCPHAKCMNKTCRCH

SEQ ID NO: 476 - protein, 32 aa, Artificial Sequence
  source: /note=“Description of Artificial Sequence Synthetic polypeptide”
VKCTTSKECWPPCKAATGKAAGKCMNKKCKCQ

SEQ ID NO: 477 - protein, 32 aa, Artificial Sequence
  source: /note=“Description of Artificial Sequence Synthetic polypeptide”
LECGASRECYDPCFKAFGRAHGKCMNNKCRCY

SEQ ID NO: 478 - protein, 32 aa, Artificial Sequence
  source: /note=“Description of Artificial Sequence Synthetic polypeptide”
EKCFATSQCWTPCKKAIGSLQSKCMNGKCKCY

SEQ ID NO: 479 - protein, 32 aa, Artificial Sequence
  source: /note=“Description of Artificial Sequence Synthetic polypeptide”
VRCYASRECWEPCRRVTGSAQAKCQNNQCRCY

SEQ ID NO: 480 - protein, 32 aa, Artificial Sequence
  source: /note=“Description of Artificial Sequence Synthetic polypeptide”
VKCSASRECWVACKKVTGSGQGKCQNNQCRCY

SEQ ID NO: 481 - protein, 32 aa, Artificial Sequence
  source: /note=“Description of Artificial Sequence Synthetic polypeptide”
VKCISSQECWIACKKVTGRFEGKCQNRQCRCY

SEQ ID NO: 482 - protein, 32 aa, Artificial Sequence
  source: /note=“Description of Artificial Sequence Synthetic polypeptide”
VRCYDSRQCWIACKKVTGSTQGKCQNKQCRCY

SEQ ID NO: 483 - protein, 32 aa, Artificial Sequence
  source: /note=“Description of Artificial Sequence Synthetic polypeptide”
VDCTVSKECWAPCKAAFGVDRGKCMGKKCKCY

SEQ ID NO: 484 - protein, 32 aa, Artificial Sequence
  source: /note=“Description of Artificial Sequence Synthetic polypeptide”
AKCRGSPECLPKCKEAIGKAAGKCMNGKCKCY

SEQ ID NO: 485 - protein, 31 aa, Artificial Sequence
  source: /note=“Description of Artificial Sequence Synthetic polypeptide”
KKCQGGSCASVCRRVIGVAAGKCINGRCVCY

SEQ ID NO: 486 - protein, 32 aa, Artificial Sequence
  source: /note=“Description of Artificial Sequence Synthetic polypeptide”
KKCSNTSQCYKTCEKVVGVAAGKCMNGKCICY

SEQ ID NO: 487 - protein, 32 aa, Artificial Sequence
  source: /note=“Description of Artificial Sequence Synthetic polypeptide”
VKCSGSSKCVKICIDRYNTRGAKCINGRCTCY

SEQ ID NO: 488 - protein, 32 aa, Artificial Sequence
  source: /note=“Description of Artificial Sequence Synthetic polypeptide”
NRCNNSSECIPHCIRIFGTRAAKCINRKCYCY

SEQ ID NO: 489 - protein, 32 aa, Artificial Sequence
  source: /note=“Description of Artificial Sequence Synthetic polypeptide”
KECNGSSECYSHCEGITGKRSGKCINKKCYCY

SEQ ID NO: 490 - protein, 29 aa, Artificial Sequence
  source: /note=“Description of Artificial Sequence Synthetic peptide”
AFCNLRRCELSCRSLGLLGKCIGEECKCV

SEQ ID NO: 491 - protein, 29 aa, Artificial Sequence
  source: /note=“Description of Artificial Sequence Synthetic peptide”
AVCNLKRCQLSCRSLGLLGKCIGDKCECV

SEQ ID NO: 492 - protein, 30 aa, Artificial Sequence
  source: /note=“Description of Artificial Sequence Synthetic polypeptide”
AACYSSDCRVKCVAMGFSSGKCINSKCKCY

SEQ ID NO: 493 - protein, 27 aa, Artificial Sequence
  source: /note=“Description of Artificial Sequence Synthetic peptide”
AICATDADCSRKCPGNPPCRNGFCACT

SEQ ID NO: 494 - protein, 27 aa, Artificial Sequence
  source: /note=“Description of Artificial Sequence Synthetic peptide”
TECQIKNDCQRYCQSVKECKYGKCYCN

SEQ ID NO: 495 - protein, 27 aa, Artificial Sequence
  source: /note=“Description of Artificial Sequence Synthetic peptide”
TQCQSVRDCQQYCLTPDRCSYGTCYCK

SEQ ID NO: 496 - protein, 32 aa, Artificial Sequence
  source: /note=“Description of Artificial Sequence Synthetic polypeptide”
VSCRYGSDCAEPCKRLKCLLPSKCINGKCTCY

SEQ ID NO: 497 - protein, 32 aa, Artificial Sequence
  source: /note=“Description of Artificial Sequence Synthetic polypeptide”
IKCRYPADCHIMCRKVTGRAEGKCMNGKCTCY

SEQ ID NO: 498 - protein, 32 aa, Artificial Sequence
  source: /note=“Description of Artificial Sequence Synthetic polypeptide”
IKCSSSSSCYEPCRGVTGRAHGKCMNGRCTCY

SEQ ID NO: 499 - protein, 32 aa, Artificial Sequence
  source: /note=“Description of Artificial Sequence Synthetic polypeptide”
VKCTGSKQCLPACKAAVGKAAGKCMNGKCKCY

SEQ ID NO: 500 - protein, 31 aa, Artificial Sequence
  source: /note=“Description of Artificial Sequence Synthetic polypeptide”
VSCKHSGQCIKPCKDAGMRFGKCMNRKCDCT

SEQ ID NO: 501 - protein, 31 aa, Artificial Sequence
  source: /note=“Description of Artificial Sequence Synthetic polypeptide”
VKCRGSPQCIQPCRDAGMRFGKCMNGKCHCT

SEQ ID NO: 502 - protein, 33 aa, Artificial Sequence
  source: /note=“Description of Artificial Sequence Synthetic polypeptide”
VKCTSPKQCLPPCKAQFGIRAGAKCMNGKCKCY

SEQ ID NO: 503 - protein, 33 aa, Artificial Sequence
  source: /note=“Description of Artificial Sequence Synthetic polypeptide”
VKCTSPKQCSKPCKELYGSSAGAKCMNGKCKCY

SEQ ID NO: 504 - protein, 33 aa, Artificial Sequence
  source: /note=“Description of Artificial Sequence Synthetic polypeptide”
VKCTSPKQCLPPCKEIYGRHAGAKCMNGKCHCS

SEQ ID NO: 505 - protein, 32 aa, Artificial Sequence
  source: /note=“Description of Artificial Sequence Synthetic polypeptide”
VKCTGSKQCWPVCKQMFGKPNGKCMNGKCRCY

SEQ ID NO: 506 - protein, 31 aa, Artificial Sequence
  source: /note=“Description of Artificial Sequence Synthetic polypeptide”
VKCRGSRDCLDPCKKAGMRFGKCINSKCHCT

SEQ ID NO: 507 - protein, 27 aa, Artificial Sequence
  source: /note=“Description of Artificial Sequence Synthetic peptide”
VRCVTDDDCFRKCPGNPSCKRGFCACK

SEQ ID NO: 508 - protein, 32 aa, Artificial Sequence
  source: /note=“Description of Artificial Sequence Synthetic polypeptide”
VPCNNSRPCVPVCIREVNNKNGKCSNGKCLCY

SEQ ID NO: 509 - protein, 40 aa, Artificial Sequence
  source: /note=“Description of Artificial Sequence Synthetic polypeptide”
GSAEIIRCSGTRECYAPCQRLTGCLNARCMNRACRCYGCV

SEQ ID NO: 510 - protein, 38 aa, Artificial Sequence
  source: /note=“Description of Artificial Sequence Synthetic polypeptide”
AEIIRCSGTRECYAPCQRLTGCLNARCMNRACRCYGCV

SEQ ID NO: 511 - protein, 4 aa, Artificial Sequence
  source: /note=“Description of Artificial Sequence Synthetic peptide”
GAGA

SEQ ID NO: 512 - protein, 40 aa, Artificial Sequence
  source: /note=“Description of Artificial Sequence Synthetic polypeptide”
CGSGVPINVRCRGSRDCLDPCRRAGMRFGRCINSRCHCTP

SEQ ID NO: 513 - protein, 40 aa, Artificial Sequence
  source: /note=“Description of Artificial Sequence Synthetic polypeptide”
  MOD_RES (1)..(1): Beta-Ala
XGSGVPINVRCRGSRDCLDPCRRAGMRFGRCINSRCHCTP

(19) United States
(12) Patent Application Publication
(10) Pub. No.: US 2021/0252159 A1
(43) Pub. Date: Aug. 19, 2021

(54) CONJUGATES OF CARTILAGE-HOMING PEPTIDES

(71) Applicant: FRED HUTCHINSON CANCER RESEARCH CENTER, Seattle, WA (US)

(72) Inventors: Gene Gregory HOPPING, Seattle, WA (US); Julian A. SIMON, Seattle, WA (US); Chunfeng YIN, Redmond, WA (US); Christopher MEHLIN, Seattle, WA (US); Michelle L. COOK SANGAR, Kenmore, WA (US); Andrew James MHYRE, Kenmore, WA (US); Joseph Michael BEATY, Seattle, WA (US); James OLSON, Seattle, WA (US)

(21) Appl. No.: 17/049,148

(22) PCT Filed: Apr. 19, 2019
(86) PCT No.: PCT/US2019/028406

§ 371 Date: Oct. 20, 2020

Related U.S. Application Data
(60) Provisional application No. 62/661,577, filed on Apr. 23, 2018.

(51) Int. Cl. A61K 47/64; A61K 47/54; A61K 31/573; A61K 31/58; A61P 19/02; A61P 37/06

(52) U.S. Cl. CPC A61K 47/64; A61K 47/542; A61P 37/06; A61K 31/58; A61P 19/02; A61K 31/573

(57) ABSTRACT

Compositions such as pharmaceutical compositions and uses for peptide-drug conjugates are disclosed. Such compositions can deliver a drug, a peptide, or a conjugate thereof to a target region, tissue, structure or cell in cartilage.

### CROSS-REFERENCE

This application claims the benefit of U.S. Provisional Application No. 62/661,577, filed Apr. 23, 2018, which application is incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Apr. 19, 2019, is named 44189-722_601_SL.txt and is 285,551 bytes in size.

### BACKGROUND

Cartilage comprises chondrocytes, a specialized cell-type which produces components of the extracellular matrix, mainly collagen, proteoglycans (e.g., aggrecan), and elastic fibers. The extracellular matrix proteins provide support, cushion, and durability to cartilage-rich portions of the body such as joints, ears, nose and windpipe. Cartilage is one of few tissues in the body which does not contain blood vessels and is considered an avascular tissue. Unlike many cells in the body which rely on a combination of blood flow and diffusion, chondrocytes rely on diffusion. Because it does not have a direct blood supply, compared to other connective tissues, cartilage grows and repairs much more slowly. As a result, cartilage disorders are particularly difficult to treat.

### SUMMARY

In various aspects, the present disclosure provides a conjugate, wherein the conjugate comprises: an anti-arthritic agent; a cystine-dense peptide, wherein upon administration to a subject the cystine-dense peptide homes, targets, migrates to, accumulates in, binds to, is retained by, or is directed to a cartilage of the subject; and a linker, wherein the linker comprises a cyclic carboxylic acid, a cyclic dicarboxylic acid, an aromatic dicarboxylic acid, or an amino acid, and wherein the linker conjugates the anti-arthritic agent and the cystine-dense peptide via an ester bond, a carbamate bond, a carbonate bond, or an amide bond. In some aspects, the anti-arthritic agent is an anti-inflammatory agent. In some aspects, the anti-inflammatory agent is a glucocorticoid or an NSAID. In some aspects, the anti-inflammatory agent is the glucocorticoid that is dexamethasone, budesonide, triamcinolone, triamcinolone acetonide, beclomethasone, betamethasone, butixicort, cortisol (hydrocortisone), clobetasol, estriol, diflorasone, diflucortolone, difluprednate, des-ciclesonide, desisobutyryl-ciclesonide, hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof. In some aspects, the glucocorticoid is dexamethasone. In some aspects, the glucocorticoid is des-ciclesonide. In some aspects, the glucocorticoid is budesonide. In some aspects, the glucocorticoid is triamcinolone acetonide. In some aspects, the cyclic carboxylic acid, the cyclic dicarboxylic acid, or the aromatic dicarboxylic acid is monocyclic, bicyclic, tricyclic, or any combination thereof. In some aspects, the cyclic carboxylic acid, the cyclic dicarboxylic acid, or the aromatic dicarboxylic acid comprises a 4, 5, 6, 7, or 8 membered ring, or a combination thereof. In some aspects, the linker comprises the cyclic carboxylic acid. In some aspects, the cyclic carboxylic acid comprises

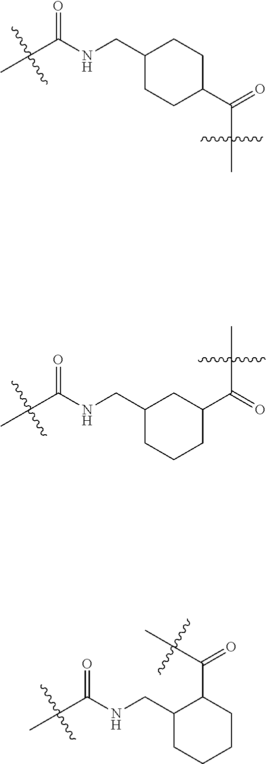 [SMILES: *C(=O)NCC1CCC(C(*)=O)CC1 ; *C(=O)NCC1CCCC(C(*)=O)C1 ; *C(=O)NCC1CCCCC1C(*)=O]

or a substituted analog or a stereoisomer thereof. In some aspects, the cyclic carboxylic acid comprises

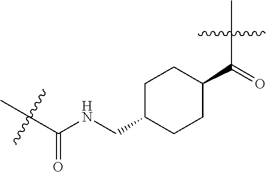 [SMILES: *C(=O)NC[C@H]1CC[C@H](C(*)=O)CC1]

or a substituted analog or a stereoisomer thereof. In some aspects, the linker comprises the cyclic dicarboxylic acid. In some aspects, the cyclic dicarboxylic acid comprises one of the following groups:

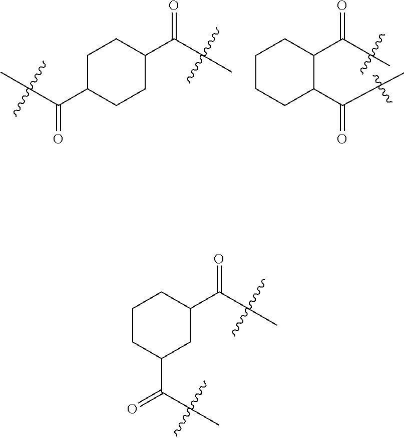 [SMILES: *C(=O)C1CCC(C(*)=O)CC1 ; *C(=O)C1CCCCC1C(*)=O ; *C(=O)C1CCCC(C(*)=O)C1]

or a substituted analog or a stereoisomer thereof. In some aspects, the cyclic dicarboxylic acid comprises one of the following groups:

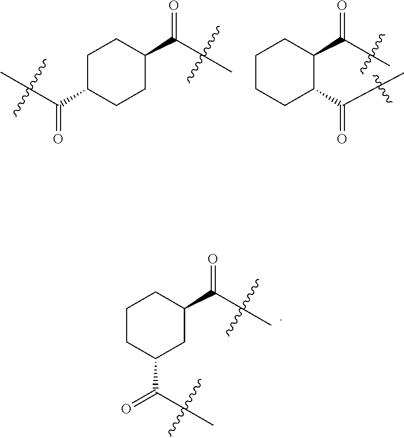 [SMILES: *C(=O)[C@H]1CC[C@H](C(*)=O)CC1 ; *C(=O)[C@@H]1CCCC[C@H]1C(*)=O ; *C(=O)[C@@H]1CCC[C@@H](C(*)=O)C1]

In some aspects, the cyclic dicarboxylic acid comprises

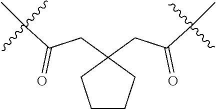 [SMILES: *C(=O)CC1(CC(*)=O)CCCC1]

or a substituted analog or a stereoisomer thereof. In some aspects, the linker comprises the aromatic dicarboxylic acid. In some aspects, the aromatic dicarboxylic acid comprises

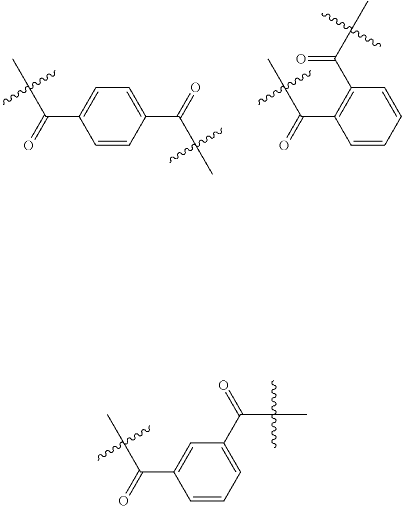 [SMILES: *C(=O)c1ccc(C(*)=O)cc1 ; *C(=O)c1ccccc1C(*)=O ; *C(=O)c1cccc(C(*)=O)c1]

or a substituted analog thereof. In some aspects, the linker comprises the amino acid. In some aspects, the amino acid comprises

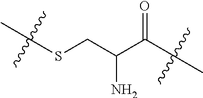 [SMILES: *SCC(N)C(*)=O]

or a substituted analog or a stereoisomer thereof. In some aspects, the linker comprises at least one of compound 2-17 listed in TABLE 2.

In various aspects, the present disclosure provides a conjugate, wherein the conjugate comprises: a glucocorticoid, wherein the glucocorticoid is not budesonide or dexamethasone; a cystine-dense peptide, wherein upon administration to a subject the cystine-dense peptide homes, targets, migrates to, accumulates in, binds to, is retained by, or is directed to a cartilage of the subject; and a linker, wherein the linker comprises a linear dicarboxylic acid, and wherein the linker conjugates the glucocorticoid and the cystine-dense peptide via an ester bond, a carbamate bond, or an amide bond. In some aspects, the glucocorticoid is triamcinolone acetonide, triamcinolone, beclomethasone, betamethasone, butixicort, cortisol (hydrocortisone), clobetasol, estriol, diflorasone, diflucortolone, difluprednate, des-ciclesonide, desisobutyryl-ciclesonide hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof.

In various aspects, the present disclosure provides a conjugate, wherein the conjugate comprises: a glucocorticoid, wherein the glucocorticoid is triamcinolone acetonide, triamcinolone, beclomethasone, betamethasone, budesonide, butixicort, cortisol (hydrocortisone), clobetasol, estriol, diflorasone, diflucortolone, difluprednate, des-ciclesonide, desisobutyryl-ciclesonide, hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof; a cystine-dense peptide, wherein upon administration to a subject the cystine-dense peptide homes, targets, migrates to, accumulates in, binds to, is retained by, or is directed to a cartilage of the subject; and a linker, wherein the linker comprises a linear dicarboxylic acid, and wherein the linker conjugates the glucocorticoid and the cystine-dense peptide via an ester bond, a carbamate bond, a carbonate bond, or an amide bond. In some aspects, the glucocorticoid is triamcinolone acetonide. In some aspects, the linear dicarboxylic acid comprises one of the following groups:

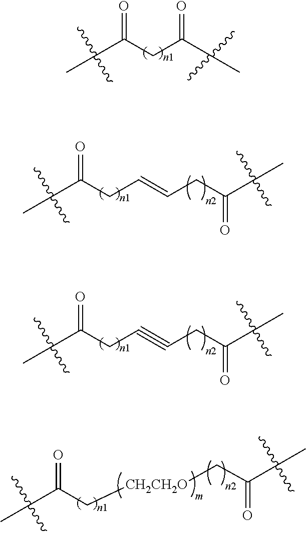

or a substituted analog or a stereoisomer thereof,
wherein each n1, n2, and m is independently a value from 1 to 10, and wherein m is a value from 0 to 10. In some aspects, the linear dicarboxylic acid comprises one of the following groups:

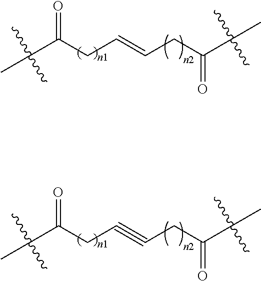

or a substituted analog or a stereoisomer thereof, wherein each n1 and n2 is independently a value from 1 to 10. In some aspects, the linear dicarboxylic acid is functionalized using a multiple bond of the linear dicarboxylic acid. In some aspects, the functionalization comprises attaching at least one molecule to the linear dicarboxylic acid. In some aspects, the functionalization via the multiple bond of the linear dicarboxylic acid comprises one or more of an addition reaction, a substitution reaction, a cycloaddition, or any combination thereof. In some aspects, the addition reaction is a nucleophilic or an electrophilic addition reaction. In some aspects, the addition reaction comprises the use of hydrogen bromide. In some aspects, the functionalization further comprises a nucleophilic substitution reaction. In some aspects, the nucleophilic substitution reaction occurs after the addition reaction. In some aspects, the cycloaddition is a 1,3-dipolar cycloaddition. In some aspects, the at least one molecule is an active agent or a detectable agent. In some aspects, the at least one molecule alters (i) the uptake of the conjugate in a cartilage; (ii) the retention of the conjugate in a cartilage; (iii) the hydrolysis rate of the conjugate, or any combination thereof. In some aspects, the linear dicarboxylic acid is one of the following groups:

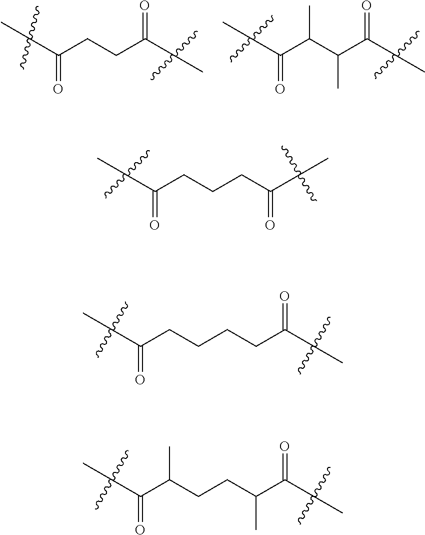

or a substituted analog or a stereoisomer thereof. In some aspects, the linker comprises at least one of compounds 18-22 listed in TABLE 2. In some aspects, the linker is stable. In some aspects, the linker is cleavable. In some aspects, the linker is cleavable by hydrolysis, an enzyme, a pH change, a reduction, a self-immolation, radiation, or a chemical reaction. In some aspects, less than 50% of the conjugates are cleaved within 24 hours, 32 hours, 56 hours, or 100 hours, at 20° C. to 37° C. or to 40° C. in a phosphate buffered saline, human plasma, or rat plasma as measured by LC/MS. In some aspects, less than 50% of the conjugates are cleaved within 10 hours, 30 hours, or 60 hours, at 20° C. to 37° C. or to 40° C. in a human plasma or rat plasma as measured by LC/MS. In some aspects, the linker of the conjugate comprises a carbamate bond. In some aspects, less than 50% of the conjugates are cleaved after 32 hours at 20° C. to 40° C. in a phosphate buffered saline, human plasma, or rat plasma as measured by LC/MS. In some aspects, less than 50% of the conjugates are cleaved after 32 hours at 20° C. to 40° C. in a human plasma as measured by LC/MS. In some aspects, less than 50% of the conjugates are cleaved after 32 hours at 20° C. to 40° C. in a rat plasma as measured by LC/MS. In some aspects, the linker comprises one of the following groups:

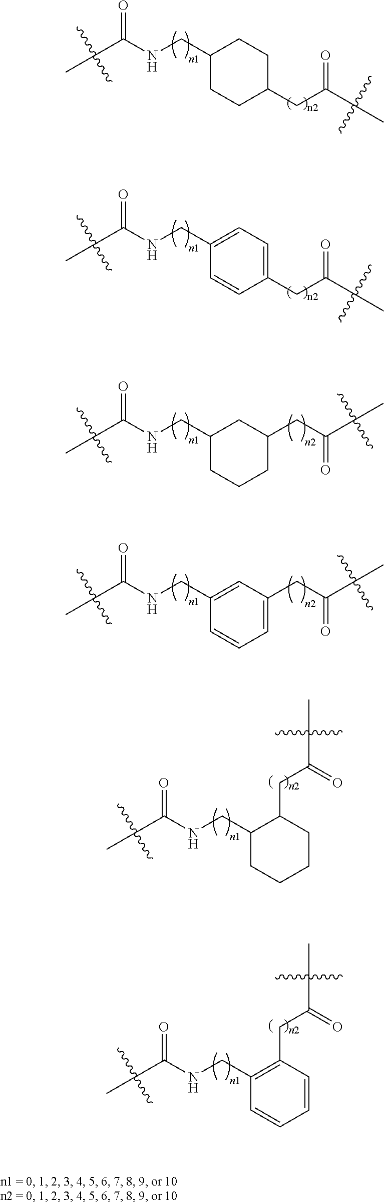

or a substituted analog or a stereoisomer thereof. In some aspects, the linker comprises one of the following groups:

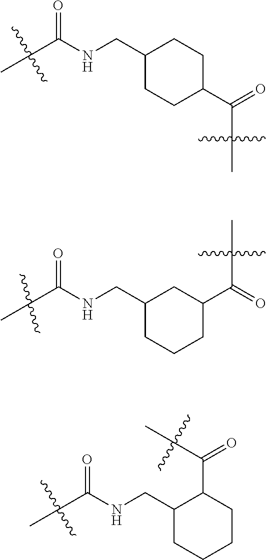 [SMILES: *C(=O)NCC1CCC(C(*)=O)CC1 ; *C(=O)NCC1CCCC(C(*)=O)C1 ; *C(=O)NCC1CCCCC1C(*)=O]

or a substituted analog or a stereoisomer thereof. In some aspects, the linker comprises:

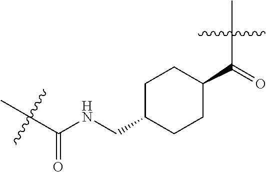 [SMILES: *C(=O)NC[C@H]1CC[C@H](C(*)=O)CC1]

or a substituted analog or a stereoisomer thereof. In some aspects, the linker comprises one of the following groups

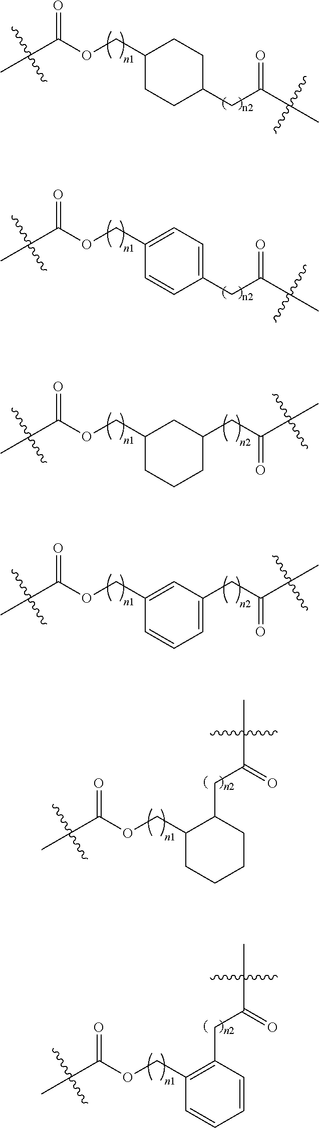

or a substituted analog or a stereoisomer thereof, and wherein n1 and n2 are each independently 0, 1, 2, 3, 4, 5, 6, 7, 8, 9, or 10. In some aspects, 50/6-100% of the conjugates are cleaved within 10-30 hours or 10-40 hours at 20° C. to 37° C. or to 40° C. in a human plasma as measured by LC/MS. In some aspects, at least 50% of the conjugates are cleaved within 0.5 to 100 hours, 1-50 hours, 1-20 hours, or 2-10 hours, at 20° C. to 37° C. or to 40° C. in a phosphate buffered saline, human plasma, or rat plasma as measured b LC/MS. In some aspects, the linker comprises

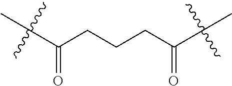 [SMILES: *C(=O)CCCC(*)=O]

or a substituted analog thereof,
and wherein 50%-100% of the conjugates are cleaved within 1-8 hours at 37° C. in a human plasma or rat plasma as measured by LC/MS. In some aspects, the linker comprises

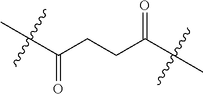 [SMILES: *C(=O)CCC(*)=O]

or a substituted analog thereof,
and wherein 50%/6-100% of the conjugates are cleaved within 1-8 hours at 37° C. in a phosphate buffered saline as measured by LC/MS. In some aspects, the linker comprises

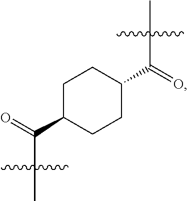 [SMILES: *C(=O)[C@H]1CC[C@H](C(*)=O)CC1]

or a substituted analog or a stereoisomer thereof, and wherein 50%-100% of the conjugates are cleaved within 1-8 hours at 37° C. in a rat plasma as measured by LC/MS. In some aspects, the linker comprises

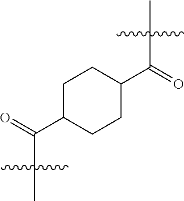 [SMILES: *C(=O)C1CCC(C(*)=O)CC1]

or a substituted analog or a stereoisomer thereof,
and wherein 25%-50% of the conjugates are cleaved within 1-8 hours at 37° C. in a human plasma as measured by LC/MS. In some aspects, the linker comprises

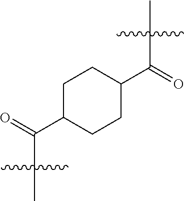 [SMILES: *C(=O)C1CCC(C(*)=O)CC1]

or a substituted analog or a stereoisomer thereof,
and wherein 50%-100% of the conjugates are cleaved within 10-30 hours at 37° C. in a human plasma as measured by LC/MS. In some aspects, the linker comprises

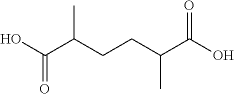

or a substituted analog or a stereoisomer thereof,
and wherein 5%-50% of the conjugates are cleaved within 1-8 hours at 37° C. in a rat plasma as measured b LC/MS. In some aspects, the linker comprises

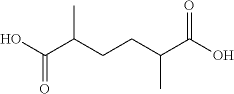

or a substituted analog or a stereoisomer thereof,
and wherein 2%-25% of the conjugates are cleaved within 1-8 hours at 37° C. in a human plasma as measured by LC/MS. In some aspects, the linker comprises

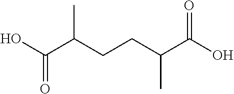

or a substituted analog or a stereoisomer thereof,
and wherein 50%-100% of the conjugates are cleaved within 10-40 hours at 37° C. in a human plasma as measured by LC/MS. In some aspects, the linker comprises

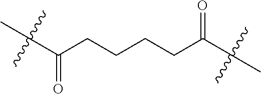 [SMILES: *C(=O)CCCCC(*)=O]

or a substituted analog thereof,
and wherein 75%-100% of the conjugates are cleaved within 1-8 hours at 37° C. in a rat plasma as measured by LC/MS. In some aspects, the linker comprises

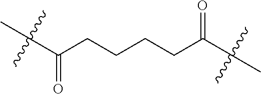 [SMILES: *C(=O)CCCCC(*)=O]

or a substituted analog thereof,
and wherein greater than 50% of the conjugates are cleaved by 10, 30, or 60 hours at 20° C. to 37° C. in rat plasma or a human plasma as measured by LC/MS. In some aspects, the linker comprises:

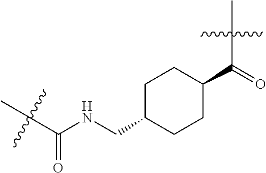 [SMILES: *C(=O)NC[C@H]1CC[C@H](C(*)=O)CC1]

or a substituted analog or a stereoisomer thereof,
and wherein less than 50% of the conjugates are cleaved within 1-8 hours at 37° C. in a phosphate buffered saline, human plasma, or rat plasma as measured by LC/MS. In some aspects, the linker comprises

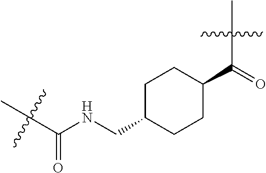 [SMILES: *C(=O)NC[C@H]1CC[C@H](C(*)=O)CC1]

or a substituted analog or a stereoisomer thereof,
and wherein less than 50% of the conjugates are cleaved within 8-32 hours at 37° C. in a phosphate buffered saline, human plasma, or rat plasma as measured by LC/MS. In some aspects, the conjugates are cleaved in vivo. In some aspects, the conjugates are cleaved when administered to an animal. In some aspects, the conjugates are cleaved when administered to a human. In some aspects, the conjugates are cleaved by hydrolysis. In some aspects, the conjugates are cleaved by a pH change, reduction, self-immolation, radiation or chemical reaction. In some aspects, ein the conjugate further comprises an amino acid sequence cleavable by enzymatic proteinase activity. In some aspects, the conjugate comprises a cleavage site for a matrix metalloproteinase (MMP). In some aspects, the MMP is MMP13. In some aspects, the conjugate comprises a cleavage site for cathepsin. In some aspects, the conjugate comprises a cathepsin cleavable linker. In some aspects, the cathepsin cleavable linker is a valine-citrulline linker. In some aspects, the cathepsin is cathepsin K. In some aspects, the conjugate comprises a cleavage site for urokinase-type plasminogen activator. In some aspects, the conjugate comprises a cleavage site for thrombin. In some aspects, the cystine-dense peptide comprises a disulfide through a disulfide knot. In some aspects, the cystine-dense peptide comprises a plurality of disulfide bridges formed between cysteine residues. In some aspects, the cystine-dense peptide comprises three or more disulfide bridges formed between cysteine residues, wherein one of the disulfide bridges passes through a loop formed by two other disulfide bridges. In some aspects, the cystine-dense peptide comprises 4 or more cysteine residues. In some aspects, the cystine-dense peptide comprises 6 or more basic residues and 2 or fewer acidic residues. In some aspects, the cystine-dense peptide comprises a 4-19 amino acid residue fragment containing at least 2 cysteine residues, and at least 2 positively charged amino acid residues. In some aspects, the cystine-dense peptide comprises a 20-70 amino acid residue fragment containing at least 2 cysteine residues, no more than 2 basic residues and at least 2 positively charged amino acid residues. In some aspects, the cystine-dense peptide comprises at least 3 positively charged amino acid residues. In some aspects, the positively charged amino acid residues are selected from K, R, or a combination thereof. In some aspects, the cystine-dense peptide comprises an amino acid sequence that has at least 80%, at least 85%, at least 90%, at least 95%, at least 96%, at least 97%, at least 98%, or at least 99% sequence identity with the amino acid sequence of any one of SEQ ID NO: 21-SEQ ID NO: 247 or SEQ ID NO: 282-SEQ ID NO: 510, or a fragment thereof. In some aspects, the cystine-dense peptide comprises an amino acid sequence of any one of SEQ ID NO: 1-SEQ ID NO: 20, SEQ ID NO: 248-SEQ ID NO: 267, or a fragment thereof. In some aspects, the cystine-dense peptide comprises the amino acid sequence set forth in SEQ ID NO: 103. In some aspects, the cystine-dense peptide comprises the amino acid sequence set forth in SEQ ID NO: 184. In some aspects, the cystine-dense peptide comprises the amino acid sequence set forth in SEQ ID NO: 105. In some aspects, the conjugate comprises any one of compounds 23, 26-31, 34, 36, 38, 40 43, 45-46, or 49-56. In some aspects, the conjugate comprises any one of compounds 23, 26-28, or 40-46. In some aspects, the conjugate comprises any one of compounds 45-46, or 49-56. In some aspects, the conjugate comprises any one of compounds 29-31, 34, or 36. In some aspects, the conjugate comprises any one of compounds 44 or 49-56. In some aspects, the conjugate comprises any one of compounds 32, 33, 35, 46, or 49-56. In some aspects, the cystine-dense peptide comprises the amino acid sequence set forth in any one of SEQ ID NO: 103, SEQ ID NO: 105, or SEQ ID NO: 184. In some aspects, the conjugate comprises any one of compounds 46, or 49-56.

In various aspects, the present disclosure provides a pharmaceutical composition that comprises a conjugate as described herein or a pharmaceutically acceptable salt thereof, and a pharmaceutically acceptable carrier. In some aspects, the pharmaceutical composition is formulated for inhalation, intranasal administration, oral administration, topical administration, intravenous administration, subcutaneous administration, intra-articular administration, intramuscular administration, intraperitoneal administration, intra-joint administration, or any combination thereof. In some aspects, the pharmaceutical composition is in a single unit dose. In some aspects, the pharmaceutical composition is a liquid. In some aspects, the pharmaceutical composition is a solid dosage form. In some aspects, the pharmaceutical composition is lyophilized. In various aspects, the present disclosure provides a kit that comprises a conjugate of the present disclosure or the pharmaceutical composition as described herein in a container and instructions for use thereof.

In various aspects, the present disclosure provides a method comprising administering to a subject in need thereof a conjugate of the present disclosure or a pharmaceutical composition of the present disclosure. In some aspects, the method provides the subject with reduction or prevention of an anti-arthritic agent-associated adverse effect, compared to that provided by a corresponding administration of the anti-arthritic agent alone. In some aspects, the method reduces occurrence of the adverse effect in the subject, compared to the administration of the anti-arthritic agent alone. In some aspects, the method reduces intensity of the adverse effect in the subject, compared to the administration of the anti-arthritic agent alone. In some aspects, the method reduces the occurrence or intensity of the adverse effect by at least 10-20%. In some aspects, the method reduces the occurrence or intensity of the adverse effect or both by at least 10%-50%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some aspects, the reduction is measured at 1, 2, 3, 6, 9, 12, 18, or 24 months following the administration. In some aspects, the reduction is measured at 1, 2, 3, 4, 5, 6, 7, 8, 9, 10, 11, 12, 13, 14, or 15 days following the administration. In some aspects, the reduction is measured at 1, 2, 3, 4, 5, 6, 7, 8, 9, or 10 weeks following the administration. In some aspects, the reduction is measured at 1, 2, 3, 4, 5, 6, 7, 8, 9, 10, 11, or 12 months following the administration. In some aspects, the adverse effect comprises: body weight loss, immunosuppression, skin thinning, purpura, Cushingoid appearance, cataract or glaucoma in an eye, osteoporosis or bone fractures, hypothalamic-pituitary-adrenal (HPA) axis suppression, hyperglycemia and diabetes, increased incidence of serious cardiovascular events, dyslipidemia, myopathy, gastritis, gastrointestinal ulcers and bleeding, psychiatric disturbance, increased blood glucose, decreased serum cortisol or corticosterone, atrophy of adrenal gland, thymus, or spleen, reduction in circulating lymphocytes, decreased cellularity of bone marrow, muscular atrophy, decreased muscle function, pain, muscular pain, arthritic pain, joint pain, joint deformity, decreased mobility, decreased range of motion in a joint, decreased flexibility, decreased strength, decreased balance, impaired glucose tolerance, loss of appetite, decreased bone metabolism, impaired immunity, nephrotic syndrome, fatigability, fungal infection, viral infection, bacterial infection, GI perforation, behavioral and mood disturbances, secondary adrenocortical insufficiency, water retention, cataracts, glaucoma, elevated blood pressure, osteoporosis, suppression of growth in children, increased insulin requirements, weight gain, nausea, Cushing's syndrome, malfunctions of the musculoskeletal, gastrointestinal, dermatologic, neurologic, endocrine, ophthalmic, metabolic, or cardiovascular systems, or any combination thereof. In some aspects, the adverse effect is the body weight loss. In some aspects, the method results in less than 5% reduction of a total body weight of the subject over 12 days following the administration, compared to the administration of the anti-arthritic agent alone. In some aspects, the administration of the conjugate results in less than 10% reduction of a total body weight of the subject over 13 days following the administration, compared to the administration of the anti-arthritic agent alone. In some aspects, the adverse effect comprises immunosuppression that is characterized by decreased function or numbers of neutrophils, lymphocytes, monocytes, macrophages, or any combination thereof. In some aspects, the adverse effect comprises immunosuppression that is characterized by T cell deficiency, humoral immune deficiency, neutropenia, or any combination thereof. In some aspects, the method results in lower toxicity to the subject, compared to a corresponding administration of the anti-arthritic agent alone. In some aspects, the conjugate is therapeutically effective at a lower dosage compared to the anti-arthritic agent alone. In some aspects, the conjugate is therapeutically effective at a less dosing frequency compared to the anti-arthritic agent alone. In some aspects, the conjugate is released within 15-60 minutes following the administration. In some aspects, the conjugate is released within 15-30 minutes following the administration. In some aspects, the conjugate has a half-life greater than: 1, 3, 6, 12, 24, or 32 hours. In some aspects, the conjugate accumulates in a target cartilage or joint within 1-3 hours. In some aspects, the conjugate is cleaved at a target cartilage or joint after the administration. In some aspects, the administration is by inhalation, intranasally, orally, topically, intravenously, subcutaneously, intra-articularly, intramuscularly administration, intraperitoneally, or any combination thereof. In some aspects, the method treats or prevents a condition associated with a function of cartilage in the subject. In some aspects, the method provides the subject with increased amelioration of a condition associated with a function of cartilage compared to that provided by a corresponding administration of the anti-arthritic agent alone. In some aspects, the condition is an inflammation, a cancer, a degradation, a growth disturbance, a genetic disease, a tear, an infection, or an injury. In some aspects, the condition is a chondrodystrophy. In some aspects, the condition is a traumatic rupture or detachment. In some aspects, the condition is a costochondritis. In some aspects, the condition is a herniation. In some aspects, the condition is a polychondritis. In some aspects, the condition is a chordoma. In some aspects, the condition is a type of arthritis. In some aspects, the type of arthritis is rheumatoid arthritis. In some aspects, the type of arthritis is osteoarthritis. In some aspects, the type of arthritis is ankylosing spondylitis. In some aspects, the type of arthritis is psoriatic arthritis. In some aspects, the type of arthritis is gout. In some aspects, the condition is achondroplasia. In some aspects, the condition is benign chondroma or malignant chondrosarcoma. In some aspects, the condition is a lupus nephritis, lupus arthritis, or systemic lupus erythematosus. In some aspects, the condition is bursitis, tendinitis, gout, pseudogout, an arthropathy, or an infection. In some aspects, the condition is an injury, damaged tissue from an injury, or pain caused by an injury. In some aspects, the condition is a tear or damaged tissue from a tear. In some aspects, the administration occurs 1, 2, 3, or 4 times yearly. In some aspects, the administration occurs 1, 2, 3, 4, 5, 6, 7, or 8 times daily. In some aspects, the administration occurs 1, 2, 3, 4, 5, 6, or 7 times weekly. In some aspects, the administration occurs 1, 2, 3, 4, 5, 6, 7, 8, 9, or 10 times monthly. In some aspects, the conjugate is administered at 0.2-20 mg/kg, 0.01-0.2 mg/kg, 0.0001-0.001 mg/kg, or 0.001-0.01 mg/kg per body weight of the subject. In some aspects, the subject is a human

In various aspects, the present disclosure provides a method of making a conjugate as described herein, the method comprising: mixing the linker and the anti-arthritic agent to form an ester bond, a carbamate bond, or an amide bond; and adding the cystine-dense peptide to form an ester bond, a carbamate bond, or an amide bond with the linker. In some aspects, the method further comprises activating a conjugating site of the anti-arthritic agent before step a). In some aspects, the method further comprises activating a functional group of the linker before step b).

In various aspects, the present disclosure provides a method of lowering a side effect in a patient undergoing treatment with an anti-arthritic agent, comprising administering to the patient a conjugate as described herein or a pharmaceutical composition as described herein. In some aspects, the side effect comprises: body weight loss, immunosuppression, skin thinning, purpura, Cushingoid appearance, cataract or glaucoma in an eye, osteoporosis or bone fractures, hypothalamic-pituitary-adrenal (HPA) axis suppression, hyperglycemia and diabetes, increased incidence of serious cardiovascular events, dyslipidemia, myopathy, gastritis, gastrointestinal ulcers and bleeding, psychiatric disturbance, increased blood glucose, decreased serum cortisol or corticosterone, atrophy of adrenal gland, thymus, or spleen, reduction in circulating lymphocytes, decreased cellularity of bone marrow, muscular atrophy, decreased muscle function, pain, muscular pain, arthritic pain, joint pain, joint deformity, decreased mobility, decreased range of motion in a joint, decreased flexibility, decreased strength, decreased balance, impaired glucose tolerance, loss of appetite, decreased bone metabolism, impaired immunity, nephrotic syndrome, fatigability, fungal infection, viral infection, bacterial infection, GI perforation, behavioral and mood disturbances, secondary adrenocortical insufficiency, water retention, cataracts, glaucoma, elevated blood pressure, osteoporosis, suppression of growth in children, increased insulin requirements, weight gain, nausea, Cushing's syndrome, malfunctions of the musculoskeletal, gastrointestinal, dermatologic, neurologic, endocrine, ophthalmic, metabolic, or cardiovascular systems, or any combination thereof.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a method of manufacturing a peptide component of a peptide-drug conjugate of the present disclosure.

FIG. 2 illustrates hydrolysis rates of peptide-dexamethasone conjugates in PBS, rat plasma, and human plasma. The graphs illustrate hydrolysis assay measurements for release of the drug dexamethasone (“Dex”) from 5 different peptide-drug conjugates with different linkers (as noted) conjugated to a peptide having the amino acid sequence set forth in SEQ ID NO: 105. Percent hydrolysis was calculated using the average area under the curve (AUC) for dexamethasone at the final time-point as the value for maximal drug release. Hydrolysis half-life of peptides incubated in PBS, rat plasma, and human plasma was determined. Peptides were incubated in PBS, rat plasma, or human plasma at 37° C. Samples were removed at regular intervals, processed by solvent extraction, and analyzed by LC/MS to quantitate the release of free dexamethasone (i.e., Dex). Each assay comprised at least 9 time-points (ranging from 2 min to 32 hours) with 3 replicate samples per time-point. The peptide-drug conjugate (peptide(SEQ ID NO; 105)-dimethyladipic acid-Dex; abbreviated as peptide(SEQ ID NO; 105)-DMA-Dex) was monitored for up to 56 hrs in human plasma.

FIG. 2A illustrates hydrolysis rates of peptide-drug conjugate peptide(SEQ ID NO:105)-glutaric acid-Dex in human plasma, rat plasma, and PBS, respectively.

FIG. 2B illustrates hydrolysis rates of peptide-drug conjugate peptide(SEQ ID NO:105)-trans-1,4-cyclohexyl-Dex in human plasma, rat plasma, and PBS, respectively.

FIG. 2C illustrates hydrolysis rates of peptide-drug conjugate peptide(SEQ ID NO:105)-DMA-Dex in human plasma, rat plasma, and PBS, respectively.

FIG. 2D illustrates hydrolysis rates of peptide-drug conjugate peptide(SEQ ID NO: 105)-[cyclohexyl-(N-4-aminomethyl-trans-1-carbamoyl)]-Dex in human plasma, rat plasma, and PBS, respectively.

FIG. 2E illustrates hydrolysis rate in rat plasma only of peptide-drug conjugate peptide(SEQ ID NO:105)-adipic acid-Dex.

FIG. 3 illustrates a group of representative autoradiograph images at 3 hrs after administration of the radiolabeled peptide-drug conjugate peptide(SEQ ID NO: 105)-¹⁴C-Cys-triamicinolone acetonide (peptide(SEQ ID NO: 105)-¹⁴C-Cys-TAA) (“peptide(SEQ ID NO: 105)-¹⁴C-Cys” is disclosed as SEQ ID NO: 512) wherein the peptide within the peptide-drug conjugate comprises the amino acid sequence SEQ ID NO: 105, or the radiolabeled drug-only control ¹⁴C-Cys-triamicinolone acetonide (¹⁴C-Cys-TAA). The peptide-drug conjugate Peptide(SEQ ID NO: 105)-¹⁴C-Cys-TAA (“peptide(SEQ ID NO: 105)-¹⁴C-Cys” is disclosed as SEQ ID NO: 512) signal is shown to home, target, be directed to, are be retained by, accumulate in, migrate to, and/or bind to cartilage of the knee (labeled), costal cartilages, intervertebral discs (IVDs), and trachea in these sections. There is no observable signal for the ¹⁴C-Cys-TAA control in cartilage. ¹⁴C-Cys-TAA signal is apparent in the bone marrow of the vertebrae and long bones.

FIG. 3A and FIG. 3B illustrate biodistribution and cartilage uptake of radiolabeled peptide-drug conjugate peptide(SEQ ID NO: 105)-¹⁴C-Cys-TAA (“peptide(SEQ ID NO: 105)-¹⁴C-Cys” is disclosed as SEQ ID NO: 512) 3 hrs after administration.

FIG. 3C and FIG. 3D illustrate biodistribution and cartilage uptake of ¹⁴C-Cys-TAA control (without the peptide) 3 hrs after administration.

FIG. 4 illustrates quantification of peptide-drug conjugate peptide(SEQ ID NO: 105)-¹⁴C-Cys-TAA (“peptide(SEQ ID NO: 105)-¹⁴C-Cys” is disclosed as SEQ ID NO: 512) accumulation in knee and intervertebral disc (IVD) (as labeled) at 3 hrs (n=6) and 24 hrs (n=6) after administration of radiolabeled drug-only control ¹⁴C-Cys-TAA or radiolabeled peptide-drug conjugate peptide(SEQ ID NO: 105)-¹⁴C-Cys-TAA (“peptide(SEQ ID NO: 105)-¹⁴C-Cys” is disclosed as SEQ ID NO: 512) described in FIG. 3. (“***” denotes treatment groups that were significantly different with each other based upon a two-tailed t-test (p<0.0001)).

FIG. 4A illustrates quantification of peptide-drug conjugate peptide(SEQ ID NO: 105)-¹⁴C-Cys-TAA (“peptide(SEQ ID NO: 105)-¹⁴C-Cys” is disclosed as SEQ ID NO: 512) and ¹⁴C-Cys-TAA control accumulation in knee and IVD (as labeled) at 3 hrs (n=6) post injection.

FIG. 4B illustrates quantification of peptide-drug conjugate peptide(SEQ ID NO: 105)-¹⁴C-Cys-TAA (“peptide(SEQ ID NO: 105)-¹⁴C-Cys” is disclosed as SEQ ID NO: 512) and ¹⁴C-Cys-TAA control accumulation in knee and IVD (as labeled) at 24 hrs (n=6) post injection.

FIG. 5 illustrates a graph showing change in ankle diameter (in mm) between day 9 (asymptomatic) and the day of administration of radiolabeled peptide-only control ¹⁴C-peptide(SEQ ID NO: 105), radiolabeled peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or radiolabeled drug-only control ¹⁴C-Cys-Dex, respectively, for collagen-induced arthritis (CIA) rats in a biodistribution study.

FIG. 6 illustrates autoradiograph images comparing accumulation in knee and ankle in the CIA model shown in FIG. 5. Ankle joints of animals that received radiolabeled peptide-only control ¹⁴C-peptide(SEQ ID NO: 105) or radiolabeled peptide-drug conjugate peptide(SEQ ID NO: 105)-¹⁴C-Cys-Dex (“peptide(SEQ ID NO: 105)-¹⁴C-Cys” is disclosed as SEQ ID NO: 512) demonstrate similar pattern of cartilage homing, targeting, retention, binding and/or accumulation at 3 hours. There is no observable accumulation of the radiolabeled drug-only control ¹⁴C-Cys-Dex in cartilage of knee or ankle. “Dex” is an abbreviation of dexamethasone. The ankle joints demonstrated similar patterns of cartilage accumulation as compared to knees when they were captured in the same section. There is accumulation of both the radiolabeled peptide control ¹⁴C-peptide(SEQ ID NO: 105) and the peptide-drug conjugate peptide(SEQ ID NO: 105)-¹⁴C-Cys-Dex (“peptide(SEQ ID NO: 105)-¹⁴C-Cys” is disclosed as SEQ ID NO: 512) in cartilage of the knee and ankle, but there is no detectable signal for drug (¹⁴C-Cys-Dex) alone in cartilage in either joint.

FIG. 6A illustrates an autoradiograph image of the knee showing accumulation of peptide-only control ¹⁴C-peptide(SEQ ID NO: 105).

FIG. 6B illustrates an autoradiograph image of the ankle showing accumulation of peptide-only control ¹⁴C-peptide(SEQ ID NO: 105).

FIG. 6C illustrates an autoradiograph image of the knee showing accumulation of peptide-drug conjugate peptide(SEQ ID NO: 105)-¹⁴C-Cys-Dex (“peptide(SEQ ID NO: 105)-¹⁴C-Cys” is disclosed as SEQ ID NO: 512).

FIG. 6D illustrates an autoradiograph image of the ankle showing accumulation of peptide-drug conjugate peptide(SEQ ID NO: 105)-¹⁴C-Cys-Dex (“peptide(SEQ ID NO: 105)-¹⁴C-Cys” is disclosed as SEQ ID NO: 512).

FIG. 6E illustrates an autoradiograph image of the knee showing lack of cartilage accumulation of drug-only control ¹⁴C-Cys-Dex.

FIG. 6F illustrates an autoradiograph image of the ankle showing lack of cartilage accumulation of drug-only control ¹⁴C-Cys-Dex.

FIG. 7 shows in vivo biodistribution of radiolabeled peptide-only control ¹⁴C-peptide(SEQ ID NO: 105), radiolabeled peptide-drug conjugate peptide(SEQ ID NO: 105)-¹⁴C-Cys-Dex (“peptide(SEQ ID NO: 105)-¹⁴C-Cys” is disclosed as SEQ ID NO: 512), and radiolabeled drug-only control ¹⁴C-Cys-Dex in knee cartilage for each treatment at 1 hr, 3 hrs, and 24 hrs post-injection (p.i.) by WBA. Black Arrows in the 1 hr images highlight regions of cartilage, black stars indicate bone marrow space.

FIG. 7A shows accumulation of peptide-only control ¹⁴C-peptide(SEQ ID NO: 105) in knee cartilage at 1 hr p.i.

FIG. 7B shows accumulation of peptide-only control ¹⁴C-peptide(SEQ ID NO: 105) in knee cartilage at 3 hr p.i.

FIG. 7C shows accumulation of peptide-only control ¹⁴C-peptide(SEQ ID NO: 105) in knee cartilage at 24 hrs p.i.

FIG. 7D shows accumulation of peptide-drug conjugate peptide(SEQ ID NO: 105)-¹⁴C-Cys-Dex (“peptide(SEQ ID NO: 105)-¹⁴C-Cys” is disclosed as SEQ ID NO: 512) in knee cartilage at 1 h p.i.

FIG. 7E shows accumulation of peptide-drug conjugate peptide(SEQ ID NO: 105)-¹⁴C-Cys-Dex (“peptide(SEQ ID NO: 105)-¹⁴C-Cys” is disclosed as SEQ ID NO: 512) in knee cartilage at 3 h p.i.

FIG. 7F shows accumulation of peptide-drug conjugate peptide(SEQ ID NO: 105)-¹⁴C-Cys-Dex (“peptide(SEQ ID NO: 105)-¹⁴C-Cys” is disclosed as SEQ ID NO: 512) in knee cartilage at 24 h p.i.

FIG. 7G shows lack of accumulation of drug-only control ¹⁴C-Cys-Dex in knee cartilage at 1 h p.i.

FIG. 7H shows lack of accumulation of drug-only control ¹⁴C-Cys-Dex in knee cartilage at 3 h p.i.

FIG. 7I shows lack of accumulation of drug-only control ¹⁴C-Cys-Dex in knee cartilage at 24 h p.i.

FIG. 8 illustrates graphs showing quantitation of signal in knee and IVD using QWBA at 1 hr, 3 hr, and 24 hrs, in animals that received radiolabeled peptide-only control ¹⁴C-peptide(SEQ ID NO: 105), radiolabeled peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or radiolabeled drug-only control ¹⁴C-Cys-Dex.

FIG. 8A illustrates quantitation of signal obtained from peptide-only control ¹⁴C-peptide(SEQ ID NO: 105), peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or drug-only control ¹⁴C-Cys-Dex in knee and IVD using QWBA at 1 hr post injection.

FIG. 8B illustrates quantitation of signal obtained from peptide-only control ¹⁴C-peptide(SEQ ID NO: 105), peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or drug-only control ¹⁴C-Cys-Dex in knee and IVD using QWBA at 3 hrs post injection.

FIG. 8C illustrates quantitation of signal obtained from peptide-only control ¹⁴C-peptide(SEQ ID NO: 105), peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or drug-only control ¹⁴C-Cys-Dex in knee and IVD using QWBA at 24 hrs post injection.

FIG. 9 illustrates graphs showing quantitation of signal in kidney, liver, blood, muscle, and bone marrow using QWBA at 1 hr, 3 hrs, and 24 hrs in animals that received radiolabeled peptide-only control (¹⁴C-peptide(SEQ ID NO: 105), radiolabeled peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or radiolabeled drug-only control ¹⁴C-Cys-Dex.

FIG. 9A shows quantitation of signal obtained from peptide-only control ¹⁴C-peptide(SEQ ID NO: 105), peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or drug-only control ¹⁴C-Cys-Dex in kidney, liver, blood, muscle, and bone marrow using QWBA at 1 hr post injection.

FIG. 9B shows quantitation of signal obtained from peptide-only control ¹⁴C-peptide(SEQ ID NO: 105), peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or drug-only control ¹⁴C-Cys-Dex in kidney, liver, blood, muscle, and bone marrow using QWBA at 3 hrs post injection.

FIG. 9C shows quantitation of signal obtained from peptide-only control ¹⁴C-peptide(SEQ ID NO: 105), peptide-drug conjugate peptide(SEQ ID NO: 105)-¹⁴C-Cys-Dex (“peptide(SEQ ID NO: 105)-¹⁴C-Cys” is disclosed as SEQ ID NO: 512), or drug-only control ¹⁴C-Cys-Dex in kidney, liver, blood, muscle, and bone marrow using QWBA at 24 hrs post injection.

FIG. 10 is a graph showing change in ankle joint diameter in millimeter (mm) between treatment initiation (day 11) and euthanasia (Day 13) for animals in 0.2 mg/kg treatment groups in the CIA model (0.2 mg/kg refers to the mass of Dex dosed and does not include the mass of peptide or linker). Each data point represents the change in ankle diameter (median day 13-day 11) for one rat in a treatment group. The bars represent the mean+/−SD for the group. “*” denotes treatment groups that were significantly different than vehicle based upon a one-tailed t-test (p<0.05). All 4 peptide drug conjugates (conjugates with different linkers listed on the x-axis of this figure) abrogated progression of disease as compared to vehicle. The graph label “1,4-cyclohexyl” refers to the peptide-drug conjugate peptide(SEQ ID NO: 105)-trans-1,4-cylcohexyl-Dex; the graph label “carbamate” refers to the peptide-drug conjugate peptide(SEQ ID NO: 105)-[cyclohexyl-(N-4-aminomethyl-trans-1-carbamoyl)]-Dex; the graph label “glutaric acid” refers to the peptide-drug conjugate peptide(SEQ ID NO: 105)-glutaric acid-Dex; and the graph label “dimethyladipic acid” refers to the peptide-drug conjugate peptide(SEQ ID NO: 105)-DMA-Dex; “Dex” is dexamethasone alone (not conjugated to a peptide).

FIG. 11 is a set of graphs comparing joint retention time in knee and IVD of the radiolabeled peptides ¹⁴C-peptide(SEQ ID NO: 105), ¹⁴C-peptide(SEQ ID NO: 103), and ¹⁴C-peptide(SEQ ID NO: 184). Joint retention was shown for all three peptides but longer joint retention was observed with the latter two peptides using the dosing regimen tested and detection method used (radiolabel on N-terminus).

FIG. 11A shows the joint retention time in knee of the radiolabeled peptides having the amino acid sequences set forth in SEQ ID NO: 105, SEQ ID NO: 103, and SEQ ID NO: 184, respectively.

FIG. 11B shows the joint retention time in IVD of the radiolabeled peptides having the amino acid sequences set forth in SEQ ID NO: 105, SEQ ID NO: 103, and SEQ ID NO: 184, respectively.

FIG. 12 shows systemic markers (including thymus weight, spleen weight, total white blood cell (WBC) count, lymphocyte count, ALT (alanine aminotransferase)) of glucocorticoid exposure or no exposure in normal rats (vehicle control, Dex×2=after 2 days of dosing, Dex×4=after 4 days of dosing with dexamethasone respectively) and in comparison to untreated CIA rats at various stages of disease progression (days 10, 12, or 14 denoted as “CIA d10”, “CIA d12” and “CIA d14” respectively). n=5 for vehicle and normal rats, n=3-4 for CIA rats. Statistical significance was assessed only for certain groups for example Dex×2 vs CIA day 12 and Dex×4 vs CIA d14. (*** p<0.0001, ** p=0.0011, * p=0.0034, statistical significance based upon a two-tailed t-test).

FIG. 12A shows the thymus weight for the various cohorts tested.

FIG. 12B shows the spleen weight for the various cohorts tested.

FIG. 12C shows the total WBC count for the various cohorts tested.

FIG. 12D shows the lymphocyte count for the various cohorts tested.

FIG. 12E shows the ALT level for the various cohorts tested.

FIG. 13 shows the effect of peptide-drug conjugates on ankle joint diameter (measured in millimeters) and systemic markers of Dex exposure in CIA rats (“*” denotes treatment groups that were significantly different than vehicle based upon a one-tailed t-test (p<0.05) in the 0.2 mg/kg cohort). The graph label “1,4-cyclohexyl” refers to the peptide-drug conjugate peptide(SEQ ID NO: 105)-trans-1,4-cyclohexyl-Dex; the graph label “carbamate” refers to the peptide-drug conjugate peptide(SEQ ID NO: 105)-[cyclohexyl-(N-4-aminomethyl-trans-1-carbamoyl)]-Dex; the graph label “glutaric acid” refers to the peptide-drug conjugate peptide(SEQ ID NO: 105)-glutaric acid-Dex; and the graph label “dimethyladipic acid” refers to the peptide-drug conjugate peptide(SEQ ID NO: 105)-DMA-Dex comprising a 2,5-dimethyl adipic acid (DMA) linker; “Dex” is dexamethasone alone (not conjugated to a peptide). The mass dose (0.2 or 0.5 mg/kg) refers to the mass of Dex dosed and does not include the mass of peptide or linker.

FIG. 13A shows ankle diameter measurements in millimeters over time for rats in the 0.2 mg/kg and 0.5 mg/kg dose treatment arms. Dose denotes mass of active agent Dex administration, not including the mass of the peptide. Arrows indicate days when treatment was administered. Data are presented at the group mean+/−SD.

FIG. 13B shows the change in ankle joint diameter in millimeters between treatment initiation (day 11) and euthanasia (Day 13) for animals in the 0.2 mg/kg and 0.5 mg/kg treatment groups, respectively, for each the peptide-drug conjugate. The median ankle diameter for each rat was calculated on day 11 and day 13 (6 total ankle measurements per day). Each data point represents the change in ankle diameter (median day 13-day 11) for one rat in a treatment group. The bars represent the mean+/−SD for the group.

FIG. 13C shows the percent change in total body weight between days 11 and 13 for animals in the 0.2 mg/kg and 0.5 mg/kg treatment groups, respectively.

FIG. 13D shows the thymus weight at euthanasia for animals in the 0.2 mg/kg and 0.5 mg/kg treatment groups, respectively.

FIG. 13E shows the spleen weight at euthanasia for animals in the 0.2 mg/kg and 0.5 mg/kg treatment groups, respectively.

FIG. 14 shows the concentration of Dex in plasma over time following a single intravenous (IV) dose of either (i) the peptide-drug conjugate peptide(SEQ ID NO: 105)-DMA-Dex or (ii) unconjugated dexamethasone sodium phosphate (denoted as “DexSP” in FIG. 14A and FIG. 14B) in non-diseased female Lewis rats. Data are presented as mean+/−SD. Each animal received a single intravenous (IV) dose of 0.2 mg/kg of dexamethasone (either as DexSP or as the peptide-drug conjugate), and the concentration of dexamethasone drug released (denoted “Free Dex”) from the peptide-drug conjugate or DexSP was measured. In addition “Total Dex” was also determined (i.e., released Free Dex plus Dex present in the peptide-drug conjugate) for the peptide-drug conjugate by taking plasma from treated rats and subjecting it to ex vivo forced hydrolysis to measure both free Dex and Dex conjugated to peptide.

FIG. 14A shows the concentration of Free Dex measured in plasma at each time-point in rats treated with either the peptide-drug conjugate peptide(SEQ ID NO: 105)-DMA-Dex (denoted as “peptide(SEQ ID NO: 105)-DMA-Dex (free Dex)”) or DexSP.

FIG. 14B shows the concentration of Total Dex (released Free-Dex plus Dex present in the peptide-drug conjugate, denoted as “free and peptide-bound”)) measured in plasma from (i) peptide-drug conjugate peptide(SEQ ID NO: 105)-DMA-Dex treated rats (denoted as “peptide(SEQ ID NO: 105)-DMA-Dex (Total Dex)”) or (ii) from rats treated with DexSP.

FIG. 14C shows the plasma concentration (in nM) of Total Dex and free Dex and calculated concentration of intact peptide-drug conjugate (intact PDC) following treatment with peptide(SEQ ID NO: 105)-DMA-Dex.

FIG. 15 shows measures of systemic exposure to Dex following a single IV administration of the peptide-drug conjugate peptide(SEQ ID NO: 105)-DMA-Dex, or DexSP. Data are presented as mean+/−SD, n=4 per time-point. Lymphocyte counts were unavailable for 1 rat each in the Dex treatment group at 6 h and 12 h, and the peptide(SEQ ID NO: 105)-DMA-Dex group at 12 h due to cell counts that were too low to permit an accurate differential.

FIG. 15A shows total white blood cell (WBC) counts over time (*p=0.0293).

FIG. 15B shows absolute lymphocyte counts over time (*(2 h) p=0.0316, (12 h) p=0.0210, unpaired t-test, two-tailed, Dex (denoted as “DexSP”) vs peptide(SEQ ID NO: 105)-DMA-Dex conjugate).

FIG. 15C shows the thymus weight over time.

FIG. 15D shows the spleen weight over time.

FIG. 16 shows the chemical structures of the peptide-drug conjugates peptide(SEQ ID NO: 105)-DMA-Dex (27) and peptide(SEQ ID NO: 105)-carbamate-Dex (28) as well as the function of effect on ankle diameter in CIA rats and markers of systemic Dex exposure in CIA rats following treatment with peptide-drug conjugate peptide(SEQ ID NO: 105)-DMA-Dex, peptide-drug conjugate peptide(SEQ ID NO: 105)-carbamate-Dex, drug-only control DexSP or vehicle. Each point represents a single animal, lines represent the mean+/−SD. (**p=0.0078 (DexSP 0.05 mg/kg), p=0.0014 (DMA 0.05), *p=0.0119 (DexSP 0.05), p=0.0132 (DMA 0.05), NS=not significant (unpaired t-test, two-tailed)). Arrows indicate days on which treatment was administered. Ankle measurements were recorded until study day 7 but euthanasia of severely affected animals between days 5-7 resulted in alterations to the mean ankle diameter of the group that were unrelated to treatment effects, therefore days 6 and 7 are omitted from the graphs. The indicated dose (0.2 or 0.05 mg/kg) refers to the mass of Dex dosed and does not include the mass of peptide or linker.

FIG. 16A shows the chemical structures of peptide-drug conjugates peptide(SEQ ID NO: 105)-DMA-Dex (27) and peptide(SEQ ID NO: 105)-carbamate-Dex (28).

FIG. 16B shows the change in ankle diameter over time following treatment with 0.2 mg/kg of peptide-drug conjugate peptide(SEQ ID NO: 105)-carbamate-Dex (denoted “Carbamate 0.2 mg/kg”), or 0.2 mg/kg DexSP (denoted “DexSP 0.2 mg/kg”), or vehicle. Data are presented as mean+/−SEM, with n=5 per group. Vehicle group was reduced to n=3 on day 3 and carbamate was reduced to n=3 on day 5 due to euthanasia of animals with severe arthritis.

FIG. 16C shows the thymus weight in rats euthanized 24 h after the last dose of 0.2 mg/kg of peptide-drug conjugate peptide(SEQ ID NO: 105)-carbamate-Dex, (denoted “carbamate 0.2 mg/kg”), or 0.2 mg/kg DexSP (denoted “DexSP 0.2 mg/kg”), or vehicle, n=3 per group. Each point represents a single animal, lines represent the mean+/−SD. (**p=0.0014 (thymus), p=0.0015 (spleen), *p=0.018′7, NS=not significant (unpaired t-test, two-tailed)).

FIG. 16D shows the spleen weight in rats euthanized 24 h after the last dose of 0.2 mg/kg of peptide-drug conjugate peptide(SEQ ID NO: 105)-carbamate-Dex (denoted “carbamate 0.2 mg/kg”), or 0.2 mg/kg DexSP (denoted “DexSP 0.2 mg/kg”), or vehicle, n=3 per group. Each point represents a single animal, lines represent the mean+/−SD. **p=0.0014 (thymus), p=0.0015 (spleen), * p=0.0187, NS=not significant (unpaired t-test, two-tailed).

FIG. 16E shows the change in ankle diameter over time following treatment with 0.05 mg/kg of peptide-drug conjugate peptide(SEQ ID NO: 105)-DMA-Dex (denoted “DMA 0.05 mg/kg”), or 0.05 mg/kg DexSP (denoted “DexSP 0.05 mg/kg”), or vehicle. Data are presented as mean+/−SEM, with n=5 per group. Vehicle group is the same as used in the 0.02 mg/kg experiment (see e.g., FIG. 16B).

FIG. 16F shows the thymus weight in rats euthanized 24 h after the last dose of 0.05 mg/kg of peptide-drug conjugate peptide(SEQ ID NO: 105)-DMA-Dex (denoted “DMA 0.05 mg/kg”), or 0.05 mg/kg DexSP (denoted “DexSP 0.05 mg/kg”), or vehicle, n=3 per group.

FIG. 16G shows the spleen weight in rats euthanized 24 h after the last dose of 0.05 mg/kg of peptide-drug conjugate peptide(SEQ ID NO: 105)-DMA-Dex (denoted “DMA 0.05 mg/kg”), or 0.05 mg/kg DexSP (denoted “DexSP 0.05 mg/kg”), or vehicle, n=3 per group.

FIG. 17 shows the effect of either DexSP or peptide-drug conjugate peptide(SEQ ID NO: 105)-DMA-Dex comprising synthetically produced peptide (also denoted “PDC”) on ankle swelling in the CIA rate arthritis model at four different dose levels (0.001 mg/kg, 0.005 mg/kg, 0.01 mg/kg, and 0.05 mg/kg for each of DexSP and PDC, and an additional dose level for 0.05 mg/kg for PDC comprising recombinantly produced peptide (denoted “rPDC”); the mass dose refers to the mass of Dex dosed and does not include the mass of peptide or linker). Data are presented as the group mean+/−SD. Vehicle group mean is shown at each dose level for comparison (black solid line). The standard deviation for the vehicle group is indicated by dotted lines. The mean+/−SD for the recombinant peptide-drug conjugate group is also shown at 0.05 mg/kg. (*p=0.0352 versus vehicle (unpaired t-test, two-tailed)). Vehicle group is the same in FIG. 17A and FIG. 17B. Data are presented as group mean+/−SEM, n=5 per group except recombinantly expresses peptide-drug conjugate (rPDC) which had n=3. A single ankle is analyzed for each rat due to a high incidence of unilateral disease. In cases of bilateral disease the ankle that demonstrated the smallest change in ankle diameter over time was included in the analysis.

FIG. 17A shows ankle diameter measurements over time following treatment with DexSP (graph labeled “Dexamethasone”) at 4 different dose levels, dosed daily for 7 days.

FIG. 17B shows ankle diameter measurements over time following treatment with peptide-drug conjugate peptide(SEQ ID NO: 105)-DMA-Dex (graph labeled “Peptide (SEQ ID NO: 105)-DMA-Dex”) at 4 different dose levels as denoted (peptide was synthetically produced), and peptide-drug conjugate peptide(SEQ ID NO: 105)-DMA-Dex at 0.05 mg/kg (“rPDC”) peptide was recombinantly produced), dosed daily for 7 days.

FIG. 17C shows a change in ankle diameter in millimeters between study day 6 and study day 0 at 4 different dose levels of DexSP (denoted “DexSP”) or peptide-drug conjugate peptide(SEQ ID NO: 105)-DMA-Dex (for conjugates comprising both synthetically (PDC) and recombinantly (rPDC) produced peptides as denoted).

FIG. 18 shows systemic measures of Dex exposure and general health status in CIA rats following 7 days of treatment with DexSP or peptide-drug conjugate peptide(SEQ ID NO: 105)-DMA-Dex (“PDC”) at four different dose levels (0.001 mg/kg, 0.005 mg/kg, 0.01 mg/kg, and 0.05 mg/kg for DexSP and PDC, and 0.05 mg/kg for PDC comprising recombinantly produced peptide (“rPDC”); the dose refers to the mass of Dex dosed and does not include the mass of peptide or linker). Vehicle group mean+/−SD is shown at each dose level for comparison (black solid line). Recombinant PDC group mean+/−SD is shown at 0.05 mg/kg dose. Tissues were collected 3 h after the final dose. P values are compared to vehicle

FIG. 18A shows the thymus weight on study day 6. (*p=0.0343, **p=0.0053, *** p=0.0006 (Dex 0.05), p=0.0002 (PDC 0.05)).

FIG. 18B shows the spleen weight on study day 6. (*p=0.0209, ***p=0.0005, **** p<0.0001).

FIG. 18C shows lymphocyte counts on study day 6. (*p=0.0469 (PDC 0.001), p=0.0156 (Dex 0.01), p=0.0173 (rPDC), **p=0.00′78, ***p=0.0009, ****p<0.0001, all unpaired two-tailed t-tests as compared to vehicle).

FIG. 18D shows the adrenal gland weight on study day 6.

FIG. 18E shows the percent (%) change in body weight between study day 6 and study day 0.

FIG. 19 shows the tissue accumulation and retention of peptides used in peptide-drug conjugates of the disclosure comprising the amino acid sequences set forth in SEQ ID NO: 105, SEQ ID NO: 103, and SEQ ID NO: 184 in athymic nude mice. Synthetic peptides were radiolabeled by reductive methylation of the N-terminus with ¹⁴C formaldehyde. 2 mice per time point were injected IV with 12.4 μCi of peptide (approximately 100 nmol or 18 mg/kg) and then euthanized at 0.08, 0.5, 1, 3, 8, 24, 48, 72, or 96 hours, followed by quantitative whole body autoradiography analysis.

FIG. 19A shows the accumulation and retention of the peptides comprising the amino acid sequences set forth in SEQ ID NO: 105, SEQ ID NO: 103, and SEQ ID NO: 184 in the knee using a linear time scale.

FIG. 19B shows the accumulation and retention of the peptides comprising the amino acid sequences set forth in SEQ ID NO: 105, SEQ ID NO: 103, and SEQ ID NO: 184 in the knee using a logarithmic time scale.

FIG. 19C shows the accumulation and retention of the peptides comprising the amino acid sequences set forth in SEQ ID NO: 105, SEQ ID NO: 103, and SEQ ID NO: 184 in the intervertebral disc (IVD) using a linear time scale.

FIG. 19D shows the accumulation and retention of the peptides comprising the amino acid sequences set forth in SEQ ID NO: 105, SEQ ID NO: 103, and SEQ ID NO: 184 in the IVD using a logarithmic time scale.

FIG. 20 shows immunohistochemistry results using anti-peptide(SEQ ID NO: 105) antibodies or anti-Dex antibodies on knee sections obtained from C57Bl/6 mice treated with either peptide-only control (peptide(SEQ ID NO: 105)), drug-only control (Cys-Dex), peptide-drug conjugate (peptide(SEQ ID NO: 105)-Cys-Dex) (“peptide(SEQ ID NO: 105)-Cys” is disclosed as SEQ ID NO: 512), or vehicle, respectively. Sections from each treatment group were stained with anti-peptide(SEQ ID NO: 105) antibody (first column), anti-Dex antibody (second column), and toluidine blue (third column). Antigen retrieval was performed using Protease 3 endopeptidase (Roche, 760-2020). Primary antibodies were diluted in antibody diluent with casein (Roche, 760-219) at a dilution of 1:200 (Dex Ab) and 1:100 (anti-peptide(SEQ ID NO: 105)-Ab). Antigens were detected using anti-rabbit-HQ (hapten) (Roche, 760-4815), anti-HQ-HRP (horseradish peroxidase)(Roche, 760-4820), and ChromoMap DAB substrate (Roche, 760-159).

FIG. 20A shows a knee section using immunohistochemistry with an anti-peptide(SEQ ID NO; 105) antibody after treatment with peptide-only control peptide(SEQ ID NO: 105), and shows localization of the peptide in the cartilage.

FIG. 20B shows a knee section using immunohistochemistry with an anti-Dex antibody after treatment with peptide-only control peptide(SEQ ID NO: 105), confirming that the anti-Dex antibody does not bind to the peptide.

FIG. 20C shows a knee section using staining with toluidine blue after treatment with peptide-only control peptide(SEQ ID NO: 105). Toluidine blue stains the proteoglycans in cartilage.

FIG. 20D shows a knee section using immunohistochemistry with an anti-peptide(SEQ ID NO: 105) antibody after treatment with drug-only control Cys-Dex, confirming that the anti-peptide antibody does not bind to the drug (i.e., Dex).

FIG. 20E shows a knee section using immunohistochemistry with an anti-Dex antibody after treatment with Cys-Dex, showing that the drug alone did not accumulate in cartilage.

FIG. 20F shows a knee section using staining with toluidine blue after treatment with Cys-Dex.

FIG. 20G shows a knee section using immunohistochemistry with an anti-peptide(SEQ ID NO: 105) antibody after treatment with peptide-drug conjugate peptide(SEQ ID NO: 105)-Cys-Dex (“peptide(SEQ ID NO: 105)-Cys” is disclosed as SEQ ID NO: 512), showing that the peptide-drug conjugate accumulated in cartilage.

FIG. 20H shows a knee section using immunohistochemistry with an anti-Dex antibody after treatment with peptide-drug conjugate peptide(SEQ ID NO: 105)-Cys-Dex (“peptide(SEQ ID NO: 105)-Cys” is disclosed as SEQ ID NO: 512), showing that the anti-Dex antibody binds the drug and peptide-drug conjugate accumulated in cartilage, indicating that the peptide delivered the drug (i.e., Dex) to cartilage.

FIG. 20I shows a knee section using staining with toluidine blue after treatment with peptide(SEQ ID NO: 105)-Cys-Dex (“peptide(SEQ ID NO: 105)-Cys” is disclosed as SEQ ID NO: 512). Toluidine blue stains the proteoglycans in cartilage.

FIG. 20J shows a knee section using immunohistochemistry with an anti-peptide(SEQ ID NO: 105) antibody after treatment with vehicle, showing that the anti-peptide antibody is not non-specifically binding to cartilage.

FIG. 20K shows a knee section using immunohistochemistry with an anti-Dex antibody after treatment with vehicle, showing that the anti-Dex antibody antibody is not non-specifically binding to cartilage.

FIG. 20L shows a knee section using staining with toluidine blue after treatment with vehicle. Toluidine blue stains the proteoglycans in cartilage, and this FIG. 20L, together with FIG. 20C, FIG. 20I, and FIG. 20F show consistent proteoglycan content in knee sections from all treatment groups.

FIG. 21 shows distribution of staining in knee sections obtained from animals treated with peptide-drug conjugate peptide(SEQ ID NO: 105)-Cys-Dex (“peptide(SEQ ID NO: 105)-Cys” is disclosed as SEQ ID NO: 512). Top row (FIG. 21A): anti-peptide(SEQ ID NO: 105)-antibody staining; Middle row (FIG. 21B): anti-Dex antibody staining; Bottom row (FIG. 21C): H&E staining.

FIG. 21A shows distribution of staining in knee sections obtained from animals treated with peptide-drug conjugate peptide(SEQ ID NO: 105)-Cys-Dex (“peptide(SEQ ID NO: 105)-Cys” is disclosed as SEQ ID NO: 512) using anti-peptide(SEQ ID NO: 105)-antibody staining.

FIG. 21B shows distribution of staining in knee sections obtained from animals treated with peptide-drug conjugate peptide(SEQ ID NO: 105)-Cys-Dex (“peptide(SEQ ID NO: 105)-Cys” is disclosed as SEQ ID NO: 512) using anti-Dex-antibody staining.

FIG. 21C shows distribution of staining in knee sections obtained from animals treated with peptide-drug conjugate peptide(SEQ ID NO: 105)-Cys-Dex (“peptide(SEQ ID NO: 105)-Cys” is disclosed as SEQ ID NO: 512) using H&E staining. Black Arrows indicate tide-mark visible in the H&E section that demarcates the boundary between non-calcified and calcified cartilage.

FIG. 22 shows a QWBA analysis of radiolabeled peptide-drug conjugate peptide(SEQ ID NO: 105)-¹⁴C-Cys-Dex (“peptide(SEQ ID NO: 105)-¹⁴C-Cys” is disclosed as SEQ ID NO: 512) or radiolabeled drug-only control ¹⁴C-Cys-Dex accumulation in cartilage (knee and IVD, FIG. 22A) and other tissues (blood, muscle, liver, kidney, and bone marrow, FIG. 22B) shown as “nmol compound/g tissue”. “+” denotes cases in which data from one animal was excluded in analysis of the tissue either due to quantitated value less than LLOQ, or absence of the tissue in sections from that animal. ++ denotes cases in which both animals in the peptide-drug conjugate peptide(SEQ ID NO: 105)-¹⁴C-Cys-Dex-treated group (“peptide(SEQ ID NO: 105)-¹⁴C-Cys” is disclosed as SEQ ID NO: 512) were excluded in the analysis of a tissue due to quantitated value less than LLOQ, or absence of the tissue in sections from that animal. A dotted line is inserted to indicate which tissues have a tissue to knee cartilage ratio greater than 1.

FIG. 22A shows accumulation in cartilage of the knee and IVD at 1 h, 3 h, and 24 h following IV administration of drug-only control ¹⁴C-Cys-Dex or peptide-drug conjugate peptide(SEQ ID NO: 105)-¹⁴C-Cys-Dex (“peptide(SEQ ID NO: 105)-¹⁴C-Cys” is disclosed as SEQ ID NO: 512).

FIG. 22B shows accumulation in various other tissues (blood, muscle, liver, kidney, bone marrow) at 1 h, 3 h, and 24 h following IV administration of drug-only control ¹⁴C-Cys-Dex or peptide-drug conjugate peptide(SEQ ID NO: 105)-¹⁴C-Cys-Dex (“peptide(SEQ ID NO: 105)-¹⁴C-Cys” is disclosed as SEQ ID NO: 512).

FIG. 22C shows the ratio (tissue-to knee cartilage) of drug-only control ¹⁴C-Cys-Dex or peptide-drug conjugate peptide(SEQ ID NO: 105)-¹⁴C-Cys-Dex (“peptide(SEQ ID NO: 105)-¹⁴C-Cys” is disclosed as SEQ ID NO: 512) accumulation in various tissues to accumulation in cartilage of the knee at 1 hr, 3 hrs, and 24 hrs after administration.

FIG. 23 shows the synthesis and in vitro hydrolysis of the peptide-drug conjugate peptide(SEQ ID NO: 105)-DMA-dCIC conjugate (46).

FIG. 23A shows the synthesis of the peptide-drug conjugate peptide(SEQ ID NO: 105)-DMA-dCIC (46). Briefly, dimethyladipic acid (DMA) was obtained via hydrolysis of the dimethyl ester with lithium hydroxide. This was reacted with dCIC in the presence of N′-ethylcarbodiimide hydrochloride (EDC.HCl) and N,N′-dimethylamino pyridine (DMAP) in dichloromethane overnight with LC-MS monitoring. The resulting carboxylic acid was activated as the sulfo-N-hydroxysuccinimide ester (sulfo-NHS) prior to reacting with the N-terminus of the peptide in DMSO under mildly basic conditions to provide the final compound which was purified by preparative-HPLC, frozen and lyophilized to give the product as a white foam.

FIG. 23B shows the in vitro hydrolysis rate of the peptide-drug conjugate peptide(SEQ ID NO: 105)-DMA-dCIC conjugate in rat plasma. Peptide-drug conjugate peptide(SEQ ID NO: 105)-DMA-dCIC was incubated in rat plasma at 37° C. Samples were removed at regular intervals, processed by solvent extraction, and analyzed by LC/MS to quantitate free dCIC. 11 time-points were measured between 5 min and 56 hours with 3 replicates per time point. The assay was repeated two times. The first assay was conducted using the same hydrolysis procedure as for the peptide-drug conjugate peptide(SEQ ID NO: 105)-DMA-Dex described above in FIG. 2, using acetonitrile extraction of dCIC, and the half-life was calculated as 8.5 h. The second assay used an optimized recovery protocol with ethyl acetate extraction of dCIC and the half-life was calculated as 5 h.

FIG. 24 shows the assessment of the PD inflammatory marker IL-6 in the knee joints of rats challenged with IL-1β. Terminal synovial fluid was collected from all animals and analyzed by Luminex 200 for IL-6 cytokine concentrations using EMD Milliplex MAP Rat Cytokine/Chemokine Magnetic Panel (RECYTMAG-65K). IL-6 levels were normalized to total protein levels (measured as IL-6 ([pg]) to total protein amp) measured by Bradford assay (Pierce Coomassie Plus Assay Kit (ThermoFisher Scientific, 23236). All treatments were administered intravenously via the tail vein (1.67 ml/kg), except for one group that received intra-articular injection of dCIC at one dose (denoted as “1274 #”). The treatment groups included the following test articles: (i) no IL-1β negative control (denoted “No IL-1b”); (ii) vehicle only control for the peptide-drug conjugate (denoted as “vehicle (PDC)”, which was 5% DMSO in PBS); (iii) vehicle only control for dCIC (40% propylene glycol, 5% DMSO in PBS, denoted as “vehicle (dCIC)”); (iv) 2 dose levels of Dexamethasone sodium phosphate, i.e, DexSP (denoted as “Dex”) at 1274 nmol/kg and 127 nmol/kg (denoted over “Dex” as “1274” and “127” respectively); (v) 3 dose levels of dCIC, 1274 nmol/kg IA, 1274 nmol/kg IV, 127 nmol/kg IV (denoted over “dCIC” as “1274 #”, “1274” and “127” respectively); and (vi) 3 dose levels of peptide(SEQ ID NO: 105)-DMA-dCIC (127 nmol/kg, 51 nmol/kg, and 13 nmol/kg) (denoted over “Peptide(SEQ ID NO: 105)-DMA-dCIC” as “127”, “51” and “13” respectively), each of (i)-(vi) with n=5 animals per dose group. Doses are listed as nmol/kg.

FIG. 25 shows the assessment of biomarkers of steroid exposure in blood of rats as part of a dose-ranging study. All treatments were administered intravenously via the tail vein (1.67 ml/kg), except for one group that received intra-articular injection of dCIC (denoted as “1274 #”). The treatment groups included the following test articles: (i) no IL-1β negative control (denoted “No IL-1b”); (ii) vehicle only control for the peptide-drug conjugate (denoted as “vehicle (PDC)”, which was 5% DMSO in PBS); (iii) vehicle only control for dCIC (40% propylene glycol, 5% DMSO in PBS, denoted as “vehicle (dCIC)”); (iv) 2 dose levels of Dexamethasone sodium phosphate, i.e, DexSP (denoted as “Dex”) at 1274 nmol/kg and 127 nmol/kg (denoted over “Dex” as “1274” and “127” respectively); (v) 3 dose levels of dCIC, 1274 nmol/kg IA, 1274 nmol/kg IV, 127 nmol/kg IV (denoted over “dCIC” as “1274 #”, “1274” and “127” respectively); and (vi) 3 dose levels of peptide(SEQ ID NO: 105)-DMA-dCIC (127 nmol/kg, 51 nmol/kg, and 13 nmol/kg) (denoted over “Peptide(SEQ ID NO: 105)-DMA-dCIC” as “127”, “51” and “13” respectively), each of (i)-(vi) with n=5 animals per dose group. Doses are listed as nmol/kg

FIG. 25A shows lymphocyte counts measured by CBC analysis at 2.75 hr post-treatment with the respective test articles.

FIG. 25B shows lymphocyte counts measured by CBC analysis at euthanasia (4 hours post IA injection of IL-1β, 7 hours post-treatment with the respective test articles).

FIG. 26 shows neutrophil and monocyte counts measured by CBC analysis at 2.75 hours post-treatment (# denotes IA injection. All other treatments were administered IV) using the dose levels shown in TABLE 16 in EXAMPLE 31. All treatments were administered intravenously via the tail vein (1.67 ml/kg), except for one group that received intra-articular injection of dCIC (denoted as “1274 #”). The treatment groups included the following test articles: (i) no IL-1β negative control (denoted “No IL-1b”); (ii) vehicle only control for the peptide-drug conjugate (denoted as “vehicle (PDC)”, which was 5% DMSO in PBS); (iii) vehicle only control for dCIC (40% propylene glycol, 5% DMSO in PBS, denoted as “vehicle (dCIC)”); (iv) 2 dose levels of Dexamethasone sodium phosphate, i.e, DexSP (denoted as “Dex”) at 1274 nmol/kg and 127 nmol/kg (denoted over “Dex” as “1274” and “127” respectively); (v) 3 dose levels of dCIC, 1274 nmol/kg IA, 1274 nmol/kg IV, 127 nmol/kg IV (denoted over “dCIC” as “1274 #”, “1274” and “127” respectively); and (vi) 4 dose levels of peptide(SEQ ID NO: 105)-DMA-dCIC (510 nmol/kg, 127 nmol/kg, 51 nmol/kg, and 13 nmol/kg) (denoted over “Peptide(SEQ ID NO: 105)-DMA-dCIC” as “510”, “127”, “51” and “13” respectively), each of (i)-(vi) with n=5 animals per dose group. Doses are listed as nmol/kg.

FIG. 26A shows neutrophil counts measured by CBC analysis at 2.75 hours post-treatment with the respective test articles.

FIG. 26B shows monocyte counts measured by CBC analysis at 2.75 hours post-treatment with the respective test articles.

FIG. 27 shows the assessment of biomarkers of steroid exposure in blood of rats as part of a PD study (# denotes dose administered by IA injection. All other treatments were administered IV). The treatment groups included the following test articles: (i) no IL-1β negative control (denoted “No IL-1b”); (ii); vehicle only control for the peptide-drug conjugate (denoted as “vehicle”), which was (5% DMSO in PBS); (iii) the peptide-only control peptide(SEQ ID NO: 105) at 127 nmol/kg (denoted over “Peptide” as “127”); (iv) 3 dose levels of dCIC, 1274 nmol/kg IA, 127 nmol/kg IV, 51 nmol/kg IV (denoted over “dCIC” as “1274 #”, “127” and “51” respectively); (v) and 2 dose levels of peptide(SEQ ID NO: 105)-DMA-dCIC (51 nmol/kg, 13 nmol/kg) (denoted over “Peptide(SEQ ID NO: 105)-DMA-dCIC” as “51” and “13” respectively), each of (i)-(v) with n=10 animals per dose group, except for no Il-1β control and peptide-only control arms which had n=6 animals per dose group. Doses are listed as nmol/kg. Statistical analysis was conducted using Wilcoxon rank-sum test with correction for multiple comparisons using Holm's method. (*p≤0.05, **p≤0.005, ***p≤0.0005, see EXAMPLE 31).

FIG. 27A shows total white blood cell counts measured by CBC analysis at 2.75 hours post-treatment with the respective test articles.

FIG. 27B shows lymphocyte counts measured by CBC analysis at 2.75 hours post-treatment with the respective test articles.

FIG. 27C shows monocyte counts measured by CBC analysis at 2.75 hours post-treatment with the respective test articles.

### DETAILED DESCRIPTION

In some cases, the present disclosure provides conjugates (e.g., pharmaceutical conjugates), compositions thereof, and methods for cartilage therapy and for diseases manifested in, by, or near tissues in proximity to cartilage. In some embodiments, a conjugate can be a pharmaceutical conjugate, a therapeutic conjugate, or used as a detecting agent or carrier. In some embodiments, the active agent of the conjugate is an anti-arthritic agent such as an anti-inflammatory agent, for example, a glucocorticoid or non-steroidal anti-inflammatory drug (NSAID). The conjugates as disclosed herein have many advantages over administration of the active agent or anti-arthritic agent such as anti-inflammatory agent alone. For example, the active agent or anti-arthritic agent such as anti-inflammatory agent of the conjugate is targeted to cartilage and/or joints. In some instances, the active agent or anti-arthritic agent such as anti-inflammatory agent of the conjugate is released and accumulates in cartilage and joints. The active agent may accumulate in higher levels or be present over longer time frames when administered as a conjugate than when administered alone. Additionally, for example as compared with traditional glucocorticoid therapy, which is associated with a wide range of adverse effects in humans and the incidence of adverse effects increases with dose level and cumulative duration of use, administration of the conjugate comprising a peptide-glucocorticoid conjugate can result in a decrease in adverse effects. This can be because higher relative amounts of drug or active agent are delivered to the target tissue (cartilage and structures nearby) and a lower relative amount is delivered to non-target tissues throughout the body and/or is present in circulation. The conjugates described herein can allow treatment of multiple joints from a single systemic injection (such as intravenous or subcutaneous), whereas administering the active agent alone may require injection directly into the joint (intraarticularly) in order to achieve therapeutic levels in the joint with acceptable side effect profiles and requiring direct injection into multiple joints in order to treat multiple joints. In addition, the peptide-glucocorticoid conjugate can also be advantageous when some joints are not amenable to direct injection.

In some embodiments, a conjugate disclosed herein provides a subject with reduction or prevention of a glucocorticoid-associated adverse effect in occurrence and/or intensity, compared to that provided by a corresponding administration of the glucocorticoid alone. The adverse effect can comprise body weight loss; immunosuppression; skin thinning; purpura; Cushingoid appearance; cataract or glaucoma in an eye; osteoporosis or bone fractures; hypothalamic-pituitary-adrenal axis suppression; hyperglycemia and diabetes; increased incidence of serious cardiovascular events; dyslipidemia; myopathy; gastritis, gastrointestinal ulcers and bleeding; psychiatric disturbance; increased blood glucose; decreased serum cortisol or corticosterone; atrophy of adrenal gland, thymus, or spleen; reduction of weight of the spleen or thymus; reduction in circulating white blood cells, including lymphocytes or monocytes; decreased cellularity of bone marrow; or any combination thereof. In some embodiments, the adverse effect comprises immunosuppression that is characterized by decreased function or numbers of neutrophils, lymphocytes, monocytes, macrophages, or any combination thereof. In some embodiments, the adverse effect comprises immunosuppression that is characterized by T cell deficiency, humoral immune deficiency, neutropenia, or any combination thereof. In some embodiments, the adverse effect comprises changes in alanine aminotransferase (ALT) levels and/or aspartate aminotransferase (AST) levels in the serum of a subject.

In some embodiments, a conjugate disclosed herein is therapeutically effective at a lower dosage or a lower dosing frequency as compared to the active agent or anti-arthritic agent such as anti-inflammatory agent alone.

In some embodiments, a conjugate disclosed herein exhibits lower toxicity or no toxicity to the subject compared to a corresponding administration of the active agent or anti-arthritic agent such as anti-inflammatory agent alone.

In some embodiments, an active agent or anti-arthritic agent such as anti-inflammatory agent of the conjugate disclosed herein can be released immediately following administration to a subject in need thereof and accumulate in a target cartilage or joint within hours. In some embodiments, the conjugate accumulates in the target cartilage or joint, and the accumulated conjugate can be cleaved, hydrolyzed, or degraded chemically or by enzymes, to release the active agent or anti-arthritic agent such as anti-inflammatory agent and the peptide at the target cartilage or joint. In some embodiments, the conjugate has a longer half-life compared to the active agent or anti-arthritic agent such as anti-inflammatory agent alone.

In some embodiments, the compositions and methods described herein utilize peptides that home, target, are directed to, are retained by, accumulate in, migrate to, and/or bind to cartilage following administration to a subject. In some embodiments, the cartilage homing peptides of the present disclosure are used to deliver an active agent or anti-arthritic agent such as anti-inflammatory agent to cartilage or tissue or cell thereof. The active agent or anti-arthritic agent such as anti-inflammatory agent can exert a therapeutic effect on cartilage or tissue or cell thereof. For example, in certain embodiments, the conjugate allows for the localized delivery of the active agent or anti-arthritic agent such as anti-inflammatory agent to cartilage or tissue or cell thereof. In certain embodiments, the peptide itself induces therapeutic responses. In some embodiments, other tissues are targeted that are near the cartilage, such as synovium, synovial fibroblasts, ligaments, or tendons. Active agent release from the cartilage depot can result in higher concentrations of the active agent in these nearby tissues.

Cartilage disorders are particularly difficult to treat. A direct route for drug administration can be intravenously, intra-articularly, or orally. However, cartilage can be avascular thus intravenous administration of drugs can fail to reach the cartilage. Drugs for cartilage diseases, such as osteoarthritis, can be injected directly locally into the affected area, for example, directly injected into the joint. Few drugs aimed at treating cartilage disorders have proved therapeutically viable with lack of access to target tissue being a primary reason for failure. The lack of access to the target tissue can also lead to administration of doses that are higher than would be necessary if a drug could home, target, or be directed to, is retained by, and/or binds to a target region, tissue, structure or cell. Thus, treatment of cartilage conditions often requires the use of high concentrations of non-specific drugs, such as glucocorticoids or NSAIDS. In addition, a number of therapeutics are of interest in treating joint disorders, but are problematic because of the level of side effects caused by systemic administration of the drug (Dancevic and McCulloch, Arthritis Research & Therapy, 16:429 (2014)).

Specific and potent drugs that are capable of contacting the cartilage can counteract the non-specificity of many treatments by selectively targeting and delivering compounds to specific regions, tissues, cells and structures. Such drugs can also be useful to modulate ion channels, protein-protein interactions, extracellular matrix remodeling (i.e., protease inhibition), and the like. Such targeted therapy can allow for lower dosing, reduced side effects, improved patient compliance, and improvement in therapeutic outcomes, which would be advantageous not only in acute disease of the cartilage, but in chronic conditions as well.

The present disclosure describes conjugates comprising a class of peptides derived from cystine-dense peptides that can effectively contact and/or accumulate in cartilage and be used with anti-arthritic agents such as anti-inflammatory agents to treat a cartilage condition. The conjugates of the disclosure can be used to treat the symptoms of various conditions. The peptides of the conjugates of the disclosure can bind to chondrocytes, to cartilage, to extracellular matrix, to collagen, hyaluranon, aggrecan (also known as cartilage-specific proteoglycan core protein (CSPCP)), or other components of the extracellular matrix, or to other components in joints and cartilaginous tissues.

Also described herein are conjugates comprising peptides that selectively home, target, are directed to, migrate to, are retained by, or accumulate in and/or bind to specific regions, tissues, structures or cells of the cartilage that aid in managing, decreasing, ablating or reducing pain (e.g., joint pain) due to chronic disease or cartilage injury or other therapeutic indications as described herein. A conjugate that homes, targets, migrates to, is directed to, is retained by, or accumulates in and/or binds to one or more specific regions, tissues, structures or cells of the cartilage can have fewer off-target and potentially negative effects, for example, side effects that often limit use and efficacy of pain drugs. In addition, such peptides can reduce dosage and increase the efficacy of existing drugs by directly targeting them to a specific region, tissue, structure or cell of the cartilage and helping the contact the cartilage or increasing the local concentration of agent. The peptide itself can modulate pain or it can be conjugated to an agent that modulates pain. Such pain modulation may operate by various mechanisms such as modulating inflammation, autoimmune responses, direct or indirect action on pain receptors, cell killing, or programmed cell death (whether via an apoptotic and/or non-apoptotic pathway of diseased cells or tissues, and the like (Tait et al. J Cell Sci, 127(Pt 10): 2135-44 (2014)).

Peptides of the conjugates of this disclosure that home, target, are directed to, migrate to, are retained by, accumulate in, or bind to specific regions, tissues, structures or cells of the cartilage can do so with different degrees of efficiency. The peptides can have a higher concentration in cartilage than in other locations, such as blood, muscle, bone marrow, spleen, thymus, skin, pancreas, or other organs. The peptides can be recorded as having a signal in cartilage as a percentage of signal in blood. The peptides can be recorded as having a signal in cartilage as a tissue-to-cartilage ratio (e.g., blood-to-cartilage ratio).

The selectively homing, targeting, directing to, migrating to, being retained by, or accumulating in and/or binding to specific regions, tissues, structures or cells of the cartilage by the peptides of the conjugates can occur after administration of the conjugate to a subject. A subject can be a human or a non-human animal.

The conjugates disclosed herein can be used with detecting agents such a fluorophores for imaging and/or to carry active agents such as anti-arthritic agents for example anti-inflammatory agents to the joint to treat inflammation.

The peptides of the conjugates disclosed herein can be used to bind cartilage explants ex vivo. Cartilage explants can be from any subject, such as a human or an animal. Assessment of peptide binding to cartilage explants can be used to screen peptides that may efficiently home to cartilage in vivo.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

As used herein, the terms “comprising” and “having” can be used interchangeably. For example, the terms “a peptide comprising an amino acid sequence of SEQ ID NO: 1” and “a peptide having an amino acid sequence of SEQ ID NO: 1” can be used interchangeably.

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

### Peptides of the Conjugates

Cystine-dense peptides are a class of peptides, usually ranging from about 13 to about 81 amino acids in length that are often folded into a compact structure. Cystine-dense peptides are typically assembled into a complex tertiary structure that is characterized by a number of intramolecular disulfide (cystine) crosslinks and may contain beta strands and other secondary structures. The presence of the disulfide bonds can give cystine-dense peptides remarkable environmental stability, allowing them to withstand extremes of temperature and pH and to resist the proteolytic enzymes of the blood stream.

A wider examination of the sequence structure and homology of cystine-dense peptides reveals that they have arisen by convergent evolution in all kinds of animals and plants. In animals, they are typically found in venoms, for example, the venoms of spiders and scorpions and have been implicated in the modulation of ion channels. Many of this class of peptide can be protease inhibitors, and as such can both home to cartilage and inhibit collagenase or a matrix metalloprotease that breaks down cartilage (e.g., matrix metalloprotease 13 (MMP13)). The cystine-dense proteins of plants can inhibit the proteolytic enzymes of animals or have antimicrobial activity, suggesting that cystine-dense peptides can function in the native defense of plants. Many of this class of peptides can have antimicrobial activity, and as such one of these can both home to cartilage and treat microbial infections. Some cystine-dense peptides can interact with ion channels, and as such can home to cartilage and interact (bind, block, activate) with ion channels such as those in chondrocytes that are known to effect proliferation, mechanotransduction, and other functions (Potassium Ion Channels in Articular Chondrocytes, Ali Mobasheri, in Mechanosensitive Ion Channels Mechanosensitivity in Cells and Tissues Volume 1, 2008, pp 157-178).

The cystine-dense peptides, of the present disclosure provide certain advantages. For instance, the presence of the disulfide bonds can give cystine-dense peptides remarkable environmental stability, allowing them to withstand extremes of temperature and pH and to resist the proteolytic enzymes of the blood stream, the gastrointestinal tract, and elsewhere in the body. The resistance of cystine-dense peptides to degradation can be beneficial in terms of reducing immunogenicity. The rigidity of knotted peptides can also allow them to bind to targets without paying the “entropic penalty” that a floppy peptide accrues upon binding a target. The knotted peptides can bind targets with antibody-like affinity. The knotted peptides can modulate the activity of a plurality of cartilage regions, tissues, structures or cells. Some of the cartilage regions, tissues, structures include: (a) elastic cartilage; (b) hyaline cartilage, such as articular cartilage and physeal cartilage; (c) fibrocartilage; and (d) any cells or cell types in (a)-(c) above. Some of the areas where the cystine-dense peptide can home to cartilage include joints such as knees, hips, or digits, nasal cartilage, spinal cartilage, tracheal cartilage, and rib cartilage. In various aspects, cartilage components include aggrecan and type II collagen. Additionally, in some embodiments, knotted peptides can penetrate into cells. In other embodiments, knotted peptides exhibit more rapid clearance and cellular uptake compared to other types of molecules.

The present disclosure provides peptides that comprise or are derived from these cystine-dense peptides. In some cases, the term “cystine-dense peptide” is considered to be interchangeable with the terms “knotted peptide,” “knottin” and “peptide,” or with the terms “hitchin,” and “knottin.”

The peptides of the conjugates of present disclosure can comprise cysteine amino acid residues. In some cases, the peptide has at least 4 cysteine amino acid residues. In some cases, the peptide has at least 6 cysteine amino acid residues. In other cases, the peptide has at least 8 cysteine amino acid residues, at least 10 cysteine amino acid residues, at least 12 cysteine amino acid residues, at least 14 cysteine amino acid residues or at least 16 cysteine amino acid residues.

A knotted peptide can comprise disulfide bridges. A knotted peptide can be a peptide wherein 5% or more of the residues are cysteines forming intramolecular disulfide bonds. A knotted peptide can be a peptide where at least 3 cystine intramolecule double bonds are present. A disulfide-linked peptide can be a drug scaffold. In some embodiments, the disulfide bridges form an inhibitor knot. A disulfide bridge can be formed between cysteine residues, for example, between cysteines 1 and 4, 2 and 5, or, 3 and 6. In some cases, one disulfide bridge passes through a loop formed by the other two disulfide bridges, for example, to form the inhibitor knot. In other cases, the disulfide bridges can be formed between any two cysteine residues.

The conjugates of the present disclosure further includes peptide scaffolds that, e.g., can be used as a starting point for generating additional peptides that can target and home to cartilage. In some embodiments, these scaffolds can be derived from a variety of knotted peptides or cystine-dense peptides. In certain embodiments, knotted peptides are assembled into a complex tertiary structure that is characterized by a number of intramolecular disulfide crosslinks, and optionally contain beta strands and other secondary structures such as an alpha helix. For example, knotted peptides include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures can include peptide having two parallel helices linked by two disulfide bridges without β-sheets (e.g., hefutoxin). In some embodiments, the knot can have different topologies in terms of which cystine is the knotting cystine. In some embodiments, the knot can have different disulfide connectivities in terms of which cysteine residues are bonded to each other. In some embodiments, the peptide can have at least 3 intramolecular cystine bonds but they do not form a knot.

A knotted peptide of a conjugate can comprise at least one amino acid residue in an L configuration. A knotted peptide can comprise at least one amino acid residue in a D configuration. In some embodiments, a knotted peptide is 15-40 amino acid residues long. In other embodiments, a knotted peptide is 11-57 amino acid residues long. In further embodiments, a knotted peptide is at least 20 amino acid residues long.

These kinds of peptides can be derived from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Grammostola rosea or another suitable genus or species of scorpion. In some cases, a peptide can be derived from a Buthus martensii Karsh (scorpion) toxin.

In some embodiments, the peptides of the conjugates are members of the pfam00451:toxin_2 family. The pfam00451:toxin_2 structural class family can include a peptide of any one of SEQ ID NO: 462-SEQ ID NO: 510. A cartilage homing peptide of this disclosure can be a variant of any peptide members of the pfam00451:toxin_2 family. In some embodiments, an exemplary cartilage homing peptide of this disclosure that is a variant of the pfam00451:toxin_2 structural class family is a peptide of SEQ ID NO: 24. In other embodiments, an exemplary cartilage homing peptide of this disclosure that is a variant of the pfam00451:toxin_2 structural class family is a peptide of SEQ ID NO: 105. In other embodiments, the variant peptides are at least 30% identical to a peptide of the structural class pfam00451:toxin_2 family. In some embodiments, the variant peptides are 30%, 40%, 50%, 60%, 80%, 90% or 95% identical to a peptide of the structural class pfam00451:toxin_2 family. In some embodiments, the variant peptides are at least 30%, at least 40%, at least 50%, at least 60%, at least 80%, at least 90% or at least 95% identical to a peptide of the structural class pfam00451:toxin_2 family. The pfam00451:toxin_2 family comprises peptide family members found as portions of various scorpion toxins, often functioning to block potassium channels. Features of the pfam00451:toxin_2 family include, but are not limited to, a features associated with members of a cystine-dense peptide 1 (CL0054) clan, which has at least 120 family members. For example, the average family member amino acid residue lengths is 31.4 amino acid residues, the average identity of family member sequence homology to the consensus sequence is 46%, and family members are derived from at least the following organisms: Tityus costatus, Centruroides noxius, Tityus serrulatus, Mesobuthus gibbosus, Centruroides elegans, Hottentotta judaicus, Mesobuthus eupeus, Parabuthus transvaalicus, Isometroides vescus, Hottentotta tamulus sindicus, Centruroides margaritatus, Centruroides suffusus suffusus, Buthus occitanus israelis, Centruroides limpidus limpidus, Leiurus quinquestriatus hebraeus, Odontobuthus doriae, Mesobuthus tamulus, Tityus stigmurus, Lychas mucronatus, Androctonus australis, Orthochirus scrobiculosus, Mesobuthus martensii, Androctonus mauretanicus mauretanicus, Centruroides limbatus, Isometrus maculatus, Tityus discrepans, Androctonus amoreuxi, Buthus occitanus tunetanus, Tityus trivittatus and Tityus obscurus (Amazonian scorpion).

In some embodiments, cartilage homing peptides of the conjugates are members of family with the sequence GSXVXXXVKCXGSKQCXXPCKRXXGXRXGKCINKKXCKCYXXX (SEQ ID NO: 9) or XVXXXVKCXGSKQCXXPCKRXXGXRXGKCINKKXCKCYXXX (SEQ ID NO: 256), in which this sequence is based on the most common elements found in the following sequences: GSGVPINVKCRGSRDCLDPCKKA-GMRFGKCINSK-CHCTP- - (SEQ ID NO: 24), GS-VRIPVSCKHSGQCLKPCKDA-GMRFGKCMNGK-CDCTPK- (SEQ ID NO: 23), GSQVQTNVKCQGGS-CASVCRREIGVAAGKCINGK-CVCYRN- (SEQ ID NO: 27), GS- - - - -ISCTGSKQCYDPCKRKTGCPNAKCMNKS-CKCYGCG (SEQ ID NO: 26), GSEV- - -IRCSGSKQCYGPCKQQTGCTNSKCMNKV-CKCYGCG (SEQ ID NO: 28), GSAVCVYRT- - - - - -CDKDCKRR-GYRSGKCINNA-CKCYPYG (SEQ ID NO: 25), GS- - - -GIVC- - -KVCKIICGMQ-GKKVNICKAPIKCKCKKG- (SEQ ID NO: 21), and GSQIYTSKECNGSSECYSHCEGITGKRSGKCINKK-CYCYR- - (SEQ ID NO: 30), where the following residues may be independently interchanged in the sequences: K and R; M, I, L, and V; G and A; S and T; Q and N; and X can independently be any number of any amino acid or no amino acid. The N-terminal GS sequence can be included or excluded between the peptides of the present disclosure.

In other embodiments, peptides of the conjugates are members of family with the sequence GSXXXGCVXXXXKCRPGXKXCCXPXKRCSRRFGXXXXKKCKXXXXXX (SEQ ID NO: 10) or XXXGCVXXXXKCRPGXKXCCXPXKRCSRRFGXXXXKKCKXXXXXX (SEQ ID NO: 257), in which the sequence is based on the most common elements found in the following sequences: GS- - -ACKGVFDACTPGKNECC-PNRVCSDK-H- - - -KWCKWKL- - - (SEQ ID NO: 29), GS- - -GCLEFWWKCNPNDDKCCRPKLKCSKLF- - - - -KLCNFSFG- - (SEQ ID NO: 31), GSSEKDCIKHLQRCR-ENKDCC- -SKKCSRR-GTNPEKRCR- - - - - - (SEQ ID NO: 22), and GS- - -GCFGY- -KCDYY-KGCCSGYV-CSPTW- - - - -KWCVRPGPGR (SEQ ID NO: 33), where the following residues may be independently interchanged in the sequences: K and R; M, I, L, and V; G and A; S and T; Q and N; and X can independently be any number of any amino acid or no amino acid. The N-terminal GS sequence can be included or excluded between the peptides of the present disclosure.

In some embodiments, a peptide of a conjugate comprises the sequence GSGVX¹IX²X³KCX⁴GSKQCX⁵DPCKX⁶X⁷X⁸GX⁹RX¹⁰GKCX¹¹NKKCKCX¹²X¹³X¹⁴X¹⁵ (SEQ ID NO: 1) or GVX¹IX²X³KCX⁴GSKQCX⁵DPCKX⁶X⁷X⁸GX⁹RX¹⁰GKCX¹¹NKKCKCX¹²X¹³X¹⁴X¹⁵ (SEQ ID NO: 248), wherein X¹, X², X³, X⁴, X⁵, X⁶, X⁷, X⁸, X⁹, X¹⁰, X¹¹, X¹², X¹³, X¹⁴ and X¹⁵ are each individually any amino acid or amino acid analogue or null. In some cases, the peptide of the conjugate comprises the sequence GSGVX¹IX²X³KCX⁴GSKQCX⁵DPCKX⁶X⁷X⁸GX⁹RX¹⁰GKCX¹¹NKKCKCX¹²X¹³X¹⁴X¹⁵ (SEQ ID NO: 2) or GVX¹IX²X³KCX⁴GSKQCX⁵DPCKX⁶X⁷X⁸GX⁹RX¹⁰GKCX¹¹NKKCKCX¹²X¹³X¹⁴X¹⁵ (SEQ ID NO: 249), where X¹ is selected from P or R, wherein X² is selected from P or N, wherein X³ is selected from V or I, wherein X⁴ is selected from S, T, R or K, wherein X⁵ is selected from Y or L, wherein X⁶ is selected from Q, R or K, wherein X⁷ is selected from A, K or R, wherein X⁸ is selected from T or A, wherein X⁹ is selected from C or M, wherein X¹⁰ is selected from F or N, wherein X¹¹ is selected from M or I, wherein X¹² is selected from Y or T, wherein X¹³ is selected from G or P, wherein X¹⁴ is selected from C or null, and wherein X¹⁵ is selected from G or null.

In some embodiments, a peptide of a conjugate comprises the sequence GSX¹X²X³X⁴IX⁵CX⁶GSKQCYX⁷PCKX⁸X⁹TGCX¹⁰X¹¹X¹²KCX¹³X¹⁴KX¹⁵CKCYGCG (SEQ ID NO: 3) or X¹X²X³X⁴IX⁵CX⁶GSKQCYX⁷PCKX⁸X⁹TGCX¹⁰X¹¹X¹²KCX¹³X¹⁴KX¹⁵CKCYGCG (SEQ ID NO: 250), wherein X¹, X², X³, X⁴, X⁵, X⁶, X⁷, X⁸, X⁹, X¹⁰, X¹¹, X¹², X¹³, X¹⁴, and X¹⁵ are each individually any amino acid or amino acid analogue or null. In some cases, the peptide comprises the sequence GSX¹X²X³X⁴IX⁵CX⁶GSKQCYX⁷PCKX⁸X⁹TGCX¹⁰X¹¹X¹²KCX¹³X¹⁴KX¹⁵CKCYGCG, (SEQ ID NO: 4) or X¹X²X³X⁴IX⁵CX⁶GSKQCYX⁷PCKX⁸X⁹TGCX¹⁰X¹¹X¹²KCX¹³X¹⁴KX¹⁵CKCYGCG (SEQ ID NO: 251), where X¹ is selected from G or null, wherein X² is selected from S or null, wherein X³ is selected from E, G or null, wherein X⁴ is selected from V, S, or null, wherein X⁵ is selected from R or S, wherein X⁶ is selected from S or T, wherein X⁷ is selected from G or D, wherein X⁸ is selected from Q or R, wherein X⁹ is selected from Q or K, wherein X¹⁰ is selected from T or P, wherein X¹¹ is selected from N or Q, wherein X¹² is selected from S or A, wherein X¹³ is selected from M or L, wherein X¹⁴ is selected from N or Q, and wherein X¹⁵ is selected from V or S.

In some embodiments, a peptide of a conjugate comprises the sequence GSX¹X²X³VX⁴IX⁵VX⁶CX⁷X⁸SX⁹X¹⁰CLX¹¹PCKX¹²AGMRFGKCX¹³NX¹⁴KCX¹⁵CTPX¹⁶ (SEQ ID NO: 5) or X¹X²X³VX⁴IX⁵VX⁶CX⁷X⁸SX⁹X¹⁰CLX¹¹PCKX¹²AGMRFGKCX¹³NX¹⁴KCX¹⁵CTPX¹⁶ (SEQ ID NO: 252), wherein X¹, X², X³, X⁴, X⁵, X⁶, X⁷, X⁸, X⁹, X¹⁰, X¹¹, X¹², X¹³, X¹⁴, X¹⁵, X¹⁶ are each individually any amino acid or amino acid analogue or null. In some cases, the peptide of the conjugate comprises the sequence GSX¹X²X³VX⁴IX⁵VX⁶CX⁷X⁸SX⁹X¹⁰CLX¹¹PCKX¹²AGMRFGKCX¹³NX¹⁴KCX¹⁵CTPX¹⁶ (SEQ ID NO: 6) or X¹X²X³VX⁴IX⁵VX⁶CX⁷X⁸SX⁹X¹⁰CLX¹¹PCKX¹²AGMRFGKCX¹³NX¹⁴KCX¹⁵CTPX¹⁶ (SEQ ID NO: 253), where X¹ is selected from G or null, wherein X² is selected from G, S or null, wherein X³ is selected from G, S or null, wherein X⁴ is selected from P or R, wherein X⁵ is selected from N or P, wherein X⁶ is selected from K or S, wherein X⁷ is selected from R or K, wherein X⁸ is selected from G or H, wherein X⁹ is selected from R or G, wherein X¹⁰ is selected from D or Q, wherein X¹¹ is selected from D or K, wherein X¹² is selected from K or D, wherein X¹³ is selected from I or M, wherein X¹⁴ is selected from S or G, wherein X¹⁵ is selected from H or D, and wherein X¹⁶ is selected from K or null.

In some embodiments, a peptide of a conjugate comprises the sequence GSXVXVKCXGSKQCXPCKRXGXRXGKCINKKXCKCYX (SEQ ID NO: 7), XVXVKCXGSKQCXPCKRXGXRXGKCINKKXCKCYX (SEQ ID NO: 254), GSXGCVXKCRPGXKXCCXPXKRCSRRFGXKKCKX (SEQ ID NO: 8), or XGCVXKCRPGXKXCCXPXKRCSRRFGXKKCKX (SEQ ID NO: 255) wherein each X is each individually any amino acid or amino acid analogue, no amino acid, or a 1-10 amino acid long peptide fragment wherein each amino acid within such peptide fragment can in each case be any amino acid or amino acid analogue.

In some embodiments, a peptide of a conjugate comprises the sequence GSGVX¹X²X³RCX⁴GSRQCX⁵DPCRX⁶X⁷X⁸GX⁹RX¹⁰GRCX¹¹NRRCRCX¹²X¹³X¹⁴X¹⁵ (SEQ ID NO: 11) or GVX¹IX²X³RCX⁴GSRQCX⁵DPCRX⁶X⁷X⁸GX⁹RX¹⁰GRCX¹¹NRRCRCX¹²X¹³X¹⁴X¹⁵ (SEQ ID NO: 258), wherein X¹, X², X³, X⁴, X⁵, X⁶, X⁷, X⁸, X⁹, X¹⁰, X¹¹, X¹², X¹³, X¹⁴ and X¹⁵ are each individually any amino acid or amino acid analogue or null. In some cases, the peptide comprises the sequence GSGVX¹IX²X³RCX⁴GSRQCX⁵DPCRX⁶X⁷X⁸GX⁹RX¹⁰GRCX¹¹NRRCRCX¹²X¹³X¹⁴X¹⁵ (SEQ ID NO: 12) or GVX¹IX²X³RCX⁴GSRQCX⁵DPCRX⁶X⁷X⁸GX⁹RX¹⁰GRCX¹¹NRRCRCX¹²X¹³X¹⁴X¹⁵ (SEQ ID NO: 259), where X¹ is selected from P or R, wherein X² is selected from P or N, wherein X³ is selected from V or I, wherein X⁴ is selected from S, T, R or K, wherein X⁵ is selected from Y or L, wherein X⁶ is selected from Q, R or K, wherein X⁷ is selected from A, K or R, wherein X⁸ is selected from T or A, wherein X⁹ is selected from C or M, wherein X¹⁰ is selected from F or N, wherein X¹¹ is selected from M or I, wherein X¹² is selected from Y or T, wherein X¹³ is selected from G or P, wherein X¹⁴ is selected from C or null, and wherein X¹⁵ is selected from G or null.

In some embodiments, a peptide of a conjugate comprises the sequence GSX¹X²X³X⁴IX⁵CX⁶GSRQCYX⁷PCRX⁸X⁹TGCX¹⁰X¹¹X¹²RCX¹³X¹⁴RX¹⁵CRCYGCG (SEQ ID NO: 13) or X¹X²X³X⁴IX⁵CX⁶GSRQCYX⁷PCRX⁸X⁹TGCX¹⁰X¹¹X¹²RCX¹³X¹⁴RX¹⁵CRCYGCG (SEQ ID NO: 260), wherein X¹, X², X³, X⁴, X⁵, X⁶, X⁷, X⁸, X⁹, X¹⁰, X¹¹, X¹², X¹³, X¹⁴, and X¹⁵ are each individually any amino acid or amino acid analogue or null. In some cases, the peptide comprises the sequence GSX¹X²X³X⁴IX⁵CX⁶GSRQCYX⁷PCRX⁸X⁹TGCX¹⁰X¹¹X¹²RCX¹³X¹⁴RX¹⁵CRCYGCG, (SEQ ID NO: 14) or X¹X²X³X⁴IX⁵CX⁶GSRQCYX⁷PCRX⁸X⁹TGCX¹⁰X¹¹X¹²RCX¹³X¹⁴RX¹⁵CRCYGCG (SEQ ID NO: 261), where X¹ is selected from G or null, wherein X² is selected from S or null, wherein X³ is selected from E, G or null, wherein X⁴ is selected from V, S, or null, wherein X⁵ is selected from R or S, wherein X⁶ is selected from S or T, wherein X⁷ is selected from G or D, wherein X⁸ is selected from Q or R, wherein X⁹ is selected from Q, R, or K, wherein X¹⁰ is selected from T or P, wherein X¹¹ is selected from N or Q, wherein X¹² is selected from S or A, wherein X¹³ is selected from M or L, wherein X¹⁴ is selected from N or Q, and wherein X¹⁵ is selected from V or S.

In some embodiments, a peptide comprises the sequence GSX¹X²X³VX⁴IX⁵VX⁶CX⁷X⁸SX⁹X¹⁰CLX¹¹PCRX¹²AGMRFGRCX¹³NX¹⁴RCX¹⁵CTPX¹⁶ (SEQ ID NO: 15) or X¹X²X³VX⁴IX⁵VX⁶CX⁷X⁸SX⁹X¹⁰CLX¹¹PCRX¹²AGMRFGRCX¹³NX¹⁴RCX¹⁵CTPX¹⁶ (SEQ ID NO: 262), wherein X¹, X², X³, X⁴, X⁵, X⁶, X⁷, X⁸, X⁹, X¹⁰, X¹¹, X¹², X¹³, X¹⁴, X¹⁵, X¹⁶ are each individually any amino acid or amino acid analogue or null. In some cases, the peptide comprises the sequence GSX¹X²X³VX⁴IX⁵VX⁶CX⁷X⁸SX⁹X¹⁰CLX¹¹PCRX¹²AGMRFGRCX¹³NX¹⁴RCX¹⁵CTPX¹⁶ (SEQ ID NO: 16) or X¹X²X³VX⁴IX⁵VX⁶CX⁷X⁸SX⁹X¹⁰CLX¹¹PCRX¹²AGMRFGRCX¹³NX¹⁴RCX¹⁵CTPX¹⁶ (SEQ ID NO: 263), where X¹ is selected from G or null, wherein X² is selected from G, S or null, wherein X³ is selected from G, S or null, wherein X⁴ is selected from P or R, wherein X⁵ is selected from N or P, wherein X⁶ is selected from R, K or S, wherein X⁷ is selected from R or K, wherein X⁸ is selected from G or H, wherein X⁹ is selected from R or G, wherein X¹⁰ is selected from D or Q, wherein X¹¹ is selected from D, R, or K, wherein X¹² is selected from K, R, or D, wherein X¹³ is selected from I or M, wherein X¹⁴ is selected from S or G, wherein X¹⁵ is selected from H or D, and wherein X¹⁶ is selected from K, R, or null.

In some embodiments, a peptide of a conjugate comprises the sequence GSXVXVRCXGSRQCXPCRRXGXRXGRCINRRXCRCYX (SEQ ID NO: 17), XVXVRCXGSRQCXPCRRXGXRXGRCINRRXCRCYX (SEQ ID NO: 264), GSXGCVXRCRPGXRXCCXPXRRCSRRFGXRRCRX (SEQ ID NO: 18), or XGCVXRCRPGXRXCCXPXRRCSRRFGXRRCRX (SEQ ID NO: 265), wherein each letter is each individually any amino acid or amino acid analogue and where X is no amino acid or a 1-10 amino acid long peptide fragment wherein each amino acid within such peptide fragment can in each case be any amino acid or amino acid analogue.

In some embodiments, a peptide comprises the sequence GSXVXXXVRCXGSRQCXXPCRRXXGXRXGRCINRRXCRCYXXX (SEQ ID NO: 19), XVXXXVRCXGSRQCXXPCRRXXGXRXGRCINRRXCRCYXXX (SEQ ID NO: 266), GSXXXGCVXXXXRCRPGXRXCCXPXRRCSRRFGXXXXRRCRXXXXXX (SEQ ID NO: 20), or XXXGCVXXXXRCRPGXRXCCXPXRRCSRRFGXXXXRRCRXXXXXX (SEQ ID NO: 267) wherein X is no amino acid, any amino acid, or any amino acid analogue.

In some embodiments, a peptide of a conjugate comprises one or more of the following peptide fragments: GKCINKKCKC (SEQ ID NO: 268); KCIN (SEQ ID NO: 269); KKCK (SEQ ID NO: 270); PCKR (SEQ ID NO: 271); KRCSRR (SEQ ID NO: 272); KQC (SEQ ID NO: 273); GRCINRRCRC (SEQ ID NO: 274); RCIN (SEQ ID NO:275); RRCR (SEQ ID NO: 276); PCRR (SEQ ID NO: 277); RRCSRR (SEQ ID NO: 278); RQC (SEQ ID NO: 279); PCKK (SEQ ID NO: 280); and KKCSKK (SEQ ID NO: 281).

TABLE 1 lists some exemplary peptides of a conjugate according to the present disclosure.

TABLE 1

| SEQ ID NO: | Amino Acid Sequence |
|---|---|
| SEQ ID NO: 21 | GSGIVCKVCKIICGMQGKKVNICKAPIKCKCKKG |
| SEQ ID NO: 22 | GSSEKDCIKHLQRCRENKDCCSKKCSRRGTNPEKRCR |
| SEQ ID NO: 23 | GSVRIPVSCKHSGQCLKPCKDAGMRFGKCMNGKCDCTPK |
| SEQ ID NO: 24 | GSGVPINVKCRGSRDCLDPCKKAGMRFGKCINSKCHCTP |
| SEQ ID NO: 25 | GSAVCVYRTCDKDCKRRGYRSGKCINNACKCYPYG |
| SEQ ID NO: 26 | GSISCTGSKQCYDPCKRKTGCPNAKCMNKSCKCYGCG |
| SEQ ID NO: 27 | GSQVQTNVKCQGGSCASVCRREIGVAAGKCINGKCVCYRN |
| SEQ ID NO: 28 | GSEVIRCSGSKQCYGPCKQQTGCTNSKCMNKVCKCYGCG |
| SEQ ID NO: 29 | GSACKGVFDACTPGKNECCPNRVCSDKHKWCKWKL |
| SEQ ID NO: 30 | GSQIYTSKECNGSSECYSHCEGITGKRSGKCINKKCYCYR |
| SEQ ID NO: 31 | GSGCLEFWWKCNPNDDKCCRPKLKCSKLFKLCNFSFG |
| SEQ ID NO: 32 | GSDCVRFWGKCSQTSDCCPHLACKSKWPRNICVWDGSVG |
| SEQ ID NO: 33 | GSGCFGYKCDYYKGCCSGYVCSPTWKWCVRPGPGR |
| SEQ ID NO: 34 | GSMNAKFILLLVLTTMMLLPDTKGAEVIRCSGSKQCYGPCKQQT |
| | GCTNSKCMNKVCKCYGCG |
| SEQ ID NO: 35 | GSMNAKLIYLLLVVTTMTLMFDTAQAVDIMCSGPKQCYGPCKKE |
| | TGCPNAKCMNRRCKCYGCV |
| SEQ ID NO: 36 | GSMNAKLIYLLLVVTTMMLTFDTTQAGDIKCSGTRQCWGPCKKQ |
| | TTCTNSKCMNGKCKCYGCVG |
| SEQ ID NO: 37 | GSMNTKFIFLLLVVTNTMMLFDTKPVEGISCTGSKQCYDPCKRK |
| | TGCPNAKCMNKSCKCYGCG |
| SEQ ID NO: 38 | GSGVPINVKCSGSRDCLEPCKKAGMRFGKCINRKCHCTPK |
| SEQ ID NO: 39 | GSGVPINVKCTGSPQCLKPCKDAGMRFGKCINGKCHCTPK |
| SEQ ID NO: 40 | GSGVIINVKCKISRQCLEPCKKAGMRFGKCMNGKCHCTPK |
| SEQ ID NO: 41 | GSGVPINVKCRGSPQCIQPCRDAGMRFGKCMNGKCHCTPQ |
| SEQ ID NO: 42 | GSGVEINVKCTGSHQCIKPCKDAGMRFGKCINRKCHCTPK |
| SEQ ID NO: 43 | GSGVEINVKCSGSPQCLKPCKDAGMRFGKCMNRKCHCTPK |
| SEQ ID NO: 44 | GSGVPTDVKCRGSPQCIQPCKDAGMRFGKCMNGKCHCTPK |
| SEQ ID NO: 45 | GSGVPINVSCTGSPQCIKPCKDAGMRFGKCMNRKCHCTPK |
| SEQ ID NO: 46 | GSGVPINVPCTGSPQCIKPCKDAGMRFGKCMNRKCHCTPK |
| SEQ ID NO: 47 | GSVGINVKCKHSGQCLKPCKDAGMRFGKCINGKCDCTPK |
| SEQ ID NO: 48 | GSVGINVKCKHSGQCLKPCKDAGMRFGKCMNGKCDCTPK |
| SEQ ID NO: 49 | GSVGIPVSCKHSGQCIKPCKDAGMRFGKCMNRKCDCTPK |
| SEQ ID NO: 50 | GSRKGCFKEGHSCPKTAPCCRPLVCKGPSPNTKKCTRP |
| SEQ ID NO: 51 | GSSFCIPFKPCKSDENCCKKFKCKTTGIVKLCRW |
| SEQ ID NO: 52 | GSLKGCLPRNRFCNALSGPRCCSGLRCKELSIWASKCL |
| SEQ ID NO: 53 | GSGNYCLRGRCLPGGRKCCNGRPCECFAKICSCKPK |
| SEQ ID NO: 54 | GSTVKCGGCNRKCCPGGCRSGKCINGKCQCY |
| SEQ ID NO: 55 | GSGCMKEYCAGQCRGKVSQDYCLKHCKCIPR |
| SEQ ID NO: 56 | GSACLGFGEKCNPSNDKCCKSSSLVCSQKHKWCKYG |
| SEQ ID NO: 57 | GSRGGCLPHNRFCNALSGPRCCSGLRCKELSIRDSRCLG |
| SEQ ID NO: 58 | GSRGGCLPRNKFCNPSSGPRCCSGLTCKELNIWASKCL |
| SEQ ID NO: 59 | GSQRSCAKPGDMCMGIKCCDGQCGCNRGTGRCFCK |
| SEQ ID NO: 60 | GSARGCADAYKSCNHPRTCCDGYNGYKRACICSGSNCKCKKS |
| SEQ ID NO: 61 | GSRGGCLPHNRFCNALSGPRCCSGLRCKELSIWDSRCLG |
| SEQ ID NO: 62 | GSRGGCLPHNRFCNALSGPRCCSGLKCKELSIYDSRCLG |
| SEQ ID NO: 63 | GSRGGCLPHNRFCNALSGPRCCSRLKCKELSIWDSRCLG |
| SEQ ID NO: 64 | GSRGGCLPHNRFCNALTGPRCCSRLRCKELSIWDSICLG |
| SEQ ID NO: 65 | GSSCADAYKSCDSLKCCNNRTCMCSMIGTNCTCRKK |
| SEQ ID NO: 66 | GSERRCLPAGKTCVRGPMRVPCCGSCSQNKCT |
| SEQ ID NO: 67 | GSLCSREGEFCYKLRKCCAGFYCKAFVLHCYRN |
| SEQ ID NO: 68 | GSACGSCRKKCKGSGKCINGRCKCY |
| SEQ ID NO: 69 | GSACGSCRKKCKGPGKCINGRCKCY |
| SEQ ID NO: 70 | GSACQGYMRKCGRDKPPCCKKLECSKTWRWCVWN |
| SEQ ID NO: 71 | GSGRYCQKWMWTCDSKRACCEGLRCKLWCRKI |
| SEQ ID NO: 72 | GSNAKCRGSPECLPKCKEAIGKAAGKCMNGKCKCYP |
| SEQ ID NO: 73 | GSNVKCRGSKECLPACKAAVGKAAGKCMNGKCKCYP |
| SEQ ID NO: 74 | GSNVKCRGSPECLPKCKEAIGKSAGKCMNGKCKCYP |
| SEQ ID NO: 75 | GSNAKCRGSPECLPKCKQAIGKAAGKCMNGKCKCYP |
| SEQ ID NO: 76 | GSRGYCAEKGIKCHNIHCCSGLTCKCKGSSCVCRK |
| SEQ ID NO: 77 | GSERGCKLTFWKCKNKKECCGWNACALGICMPR |
| SEQ ID NO: 78 | GSKKKCIAKDYGRCKWGGTPCCRGRGCICSIMGTNCECKPR |
| SEQ ID NO: 79 | GSGCKLTFWKCKNKKECCGWNACALGICMPR |
| SEQ ID NO: 80 | GSACKGLFVTCTPGKDECCPNHVCSSKHKWCKYK |
| SEQ ID NO: 81 | GSIACAPRGLLCFRDKECCKGLTCKGRFVNTWPTFCLV |
| SEQ ID NO: 82 | GSACAGLYKKCGKGVNTCCENRPCKCDLAMGNCICKKK |
| SEQ ID NO: 83 | GSFTCAISCDIKVNGKPCKGSGEKKCSGGWSCKFNVCVKV |
| SEQ ID NO: 84 | GSGFCAQKGIKCHDIHCCTNLKCVREGSNRVCRKA |
| SEQ ID NO: 85 | GSCAKKRNWCGKNEDCCCPMKCIYAWYNQQGSCQSTITGLFKKC |
| SEQ ID NO: 86 | GSYCQKWMWTCDSARKCCEGLVCRLWCKKI |
| SEQ ID NO: 87 | GSRGGCLPHNKFCNALSGPRCCSGLKCKELTIWNTKCLE |
| SEQ ID NO: 88 | GSNVKCTGSKQCLPACKAAVGKAAGKCMNGKCKCYT |
| SEQ ID NO: 89 | GSQRSCAKPGEMCMRIKCCDGQCGCNRGTGRCFCK |
| SEQ ID NO: 90 | GSGCIPKHKRCTWSGPKCCNNISCHCNISGTLCKCRPG |
| SEQ ID NO: 91 | GSNYCVAKRCRPGGRQCCSGKPCACVGKVCKCPRD |
| SEQ ID NO: 92 | GSERGCSGAYKRCSSSQRCCEGRPCVCSAINSNCKCRKT |
| SEQ ID NO: 93 | GSRYCPRNPEACYNYCLRTGRPGGYCGGRSRITCFCFR |
| SEQ ID NO: 94 | GSQRSCAKPGEMCMGIKCCDGQCGCNRGTGRCFCK |
| SEQ ID NO: 95 | GSRRGCFKEGKWCPKSAPCCAPLKCKGPSIKQQKCVRE |
| SEQ ID NO: 96 | GSTVKCGGCNRKCCAGGCRSGKCINGKCQCYGR |
| SEQ ID NO: 97 | GSERRCEPSGKPCRPLMRIPCCGSCVRGKCA |
| SEQ ID NO: 98 | GSRGGCLPRNKFCNPSSGPRCCSGLTCKELNIWANKCL |
| SEQ ID NO: 99 | GSCAKKRNWCGKNEDCCCPMKCIYAWYNQQGSCQTTITGLFKKC |
| SEQ ID NO: 100 | GSVRIPVSCKHSGQCLKPCKDAGMRTGKCMNGKCDCTPK |
| SEQ ID NO: 101 | GSVKCTTSKDCWPPCKKVTGRA |
| SEQ ID NO: 102 | GSGIVCRVCRIICGMQGRRVNICRAPIRCRCRRG |
| SEQ ID NO: 103 | GSSERDCIRHLQRCRENRDCCSRRCSRRGTNPERRCR |
| SEQ ID NO: 104 | GSVRIPVSCRHSGQCLRPCRDAGMRFGRCMNGRCDCTPR |
| SEQ ID NO: 105 | GSGVPINVRCRGSRDCLDPCRRAGMRFGRCINSRCHCTP |
| SEQ ID NO: 106 | GSAVCVYRTCDRDCRRRGYRSGRCINNACRCYPYG |
| SEQ ID NO: 107 | GSISCTGSRQCYDPCRRRTGCPNARCMNRSCRCYGCG |
| SEQ ID NO: 108 | GSQVQTNVRCQGGSCASVCRREIGVAAGRCINGRCVCYRN |
| SEQ ID NO: 109 | GSEVIRCSGSRQCYGPCRQQTGCTNSRCMNRVCRCYGCG |
| SEQ ID NO: 110 | GSACRGVFDACTPGRNECCPNRVCSDRHRWCRWRL |
| SEQ ID NO: 111 | GSQIYTSRECNGSSECYSHCEGITGRRSGRCINRRCYCYR |
| SEQ ID NO: 112 | GSGCLEFWWRCNPNDDRCCRPRLRCSRLFRLCNFSFG |
| SEQ ID NO: 113 | GSDCVRFWGRCSQTSDCCPHLACRSRWPRNICVWDGSVG |
| SEQ ID NO: 114 | GSGCFGYRCDYYRGCCSGYVCSPTWRWCVRPGPGR |
| SEQ ID NO: 115 | GSMNARFILLLVLTTMMLLPDTRGAEVIRCSGSRQCYGPCRQQT |
| | GCTNSRCMNRVCRCYGCG |
| SEQ ID NO: 116 | GSMNARLIYLLLVVTTMTLMFDTAQAVDIMCSGPRQCYGPCRRE |
| | TGCPNARCMNRRCRCYGCV |
| SEQ ID NO: 117 | GSMNARLIYLLLVVTTMMLTFDTTQAGDIRCSGTRQCWGPCRRQ |
| | TTCTNSRCMNGRCRCYGCVG |
| SEQ ID NO: 118 | GSMNTRFIFLLLVVTNTMMLFDTRPVEGISCTGSRQCYDPCRRR |
| | TGCPNARCMNRSCRCYGCG |
| SEQ ID NO: 119 | GSGVPINVRCSGSRDCLEPCRRAGMRFGRCINRRCHCTPR |
| SEQ ID NO: 120 | GSGVPINVRCTGSPQCLRPCRDAGMRFGRCINGRCHCTPR |
| SEQ ID NO: 121 | GSGVIINVRCRISRQCLEPCRRAGMRFGRCMNGRCHCTPR |
| SEQ ID NO: 122 | GSGVPINVRCRGSPQCIQPCRDAGMRFGRCMNGRCHCTPQ |
| SEQ ID NO: 123 | GSGVEINVRCTGSHQCIRPCRDAGMRFGRCINRRCHCTPR |
| SEQ ID NO: 124 | GSGVEINVRCSGSPQCLRPCRDAGMRFGRCMNRRCHCTPR |
| SEQ ID NO: 125 | GSGVPTDVRCRGSPQCIQPCRDAGMRFGRCMNGRCHCTPR |
| SEQ ID NO: 126 | GSGVPINVSCTGSPQCIRPCRDAGMRFGRCMNRRCHCTPR |
| SEQ ID NO: 127 | GSGVPINVPCTGSPQCIRPCRDAGMRFGRCMNRRCHCTPR |
| SEQ ID NO: 128 | GSVGINVRCRHSGQCLRPCRDAGMRFGRCINGRCDCTPR |
| SEQ ID NO: 129 | GSVGINVRCRHSGQCLRPCRDAGMRFGRCMNGRCDCTPR |
| SEQ ID NO: 130 | GSVGIPVSCRHSGQCIRPCRDAGMRFGRCMNRRCDCTPR |
| SEQ ID NO: 131 | GSRRGCFREGHSCPRTAPCCRPLVCRGPSPNTRRCTRP |
| SEQ ID NO: 132 | GSSFCIPFRPCRSDENCCRRFRCRTTGIVRLCRW |
| SEQ ID NO: 133 | GSLRGCLPRNRFCNALSGPRCCSGLRCRELSIWASRCL |
| SEQ ID NO: 134 | GSGNYCLRGRCLPGGRRCCNGRPCECFARICSCRPR |
| SEQ ID NO: 135 | GSTVRCGGCNRRCCPGGCRSGRCINGRCQCY |
| SEQ ID NO: 136 | GSGCMREYCAGQCRGRVSQDYCLRHCRCIPR |
| SEQ ID NO: 137 | GSACLGFGERCNPSNDRCCRSSSLVCSQRHRWCRYG |
| SEQ ID NO: 138 | GSRGGCLPHNRFCNALSGPRCCSGLRCRELSIRDSRCLG |
| SEQ ID NO: 139 | GSRGGCLPRNRFCNPSSGPRCCSGLTCRELNIWASRCL |
| SEQ ID NO: 140 | GSQRSCARPGDMCMGIRCCDGQCGCNRGTGRCFCR |
| SEQ ID NO: 141 | GSARGCADAYRSCNHPRTCCDGYNGYRRACICSGSNCRCRRS |
| SEQ ID NO: 142 | GSRGGCLPHNRFCNALSGPRCCSGLRCRELSIWDSRCLG |
| SEQ ID NO: 143 | GSRGGCLPHNRFCNALSGPRCCSGLRCRELSIYDSRCLG |
| SEQ ID NO: 144 | GSRGGCLPHNRFCNALSGPRCCSRLRCRELSIWDSRCLG |
| SEQ ID NO: 145 | GSRGGCLPHNRFCNALTGPRCCSRLRCRELSIWDSICLG |
| SEQ ID NO: 146 | GSSCADAYKSCDSLRCCNNRTCMCSMIGTNCTCRRR |
| SEQ ID NO: 147 | GSERRCLPAGRTCVRGPMRVPCCGSCSQNRCT |
| SEQ ID NO: 148 | GSLCSREGEFCYRLRRCCAGFYCRAFVLHCYRN |
| SEQ ID NO: 149 | GSACGSCRRRCRGSGRCINGRCRCY |
| SEQ ID NO: 150 | GSACGSCRRRCRGPGRCINGRCRCY |
| SEQ ID NO: 151 | GSACQGYMRRCGRDRPPCCRRLECSRTWRWCVWN |
| SEQ ID NO: 152 | GSGRYCQRWMWTCDSRRACCEGLRCRLWCRRI |
| SEQ ID NO: 153 | GSNARCRGSPECLPRCREAIGRAAGRCMNGRCRCYP |
| SEQ ID NO: 154 | GSNVRCRGSRECLPACRAAVGRAAGRCMNGRCRCYP |
| SEQ ID NO: 155 | GSNVRCRGSPECLPRCREAIGRSAGRCMNGRCRCYP |
| SEQ ID NO: 156 | GSNARCRGSPECLPRCRQAIGRAAGRCMNGRCRCYP |
| SEQ ID NO: 157 | GSRGYCAERGIRCHNIHCCSGLTCRCRGSSCVCRR |
| SEQ ID NO: 158 | GSERGCRLTFWRCRNRRECCGWNACALGICNIPR |
| SEQ ID NO: 159 | GSRRRCIARDYGRCRWGGTPCCRGRGCICSIMGTNCECRPR |
| SEQ ID NO: 160 | GSGCRLTFWRCRNRRECCGWNACALGICMPR |
| SEQ ID NO: 161 | GSACRGLFVTCTPGRDECCPNHVCSSRHRWCRYR |
| SEQ ID NO: 162 | GSIACAPRGLLCFRDRECCRGLTCRGRFVNTWPTFCLV |
| SEQ ID NO: 163 | GSACAGLYRRCGRGVNTCCENRPCRCDLAMGNCICRRR |
| SEQ ID NO: 164 | GSFTCAISCDIRVNGRPCRGSGERRCSGGWSCRFNVCVRV |
| SEQ ID NO: 165 | GSGFCAQRGIRCHDIHCCTNLRCVREGSNRVCRRA |
| SEQ ID NO: 166 | GSCARRRNWCGRNEDCCCPMRCIYAWYNQQGSCQSTITGLFRRC |
| SEQ ID NO: 167 | GSYCQRWMWTCDSARRCCEGLVCRLWCRRI |
| SEQ ID NO: 168 | GSRGGCLPHNRFCNALSGPRCCSGLRCRELTIWNTRCLE |
| SEQ ID NO: 169 | GSNVRCTGSRQCLPACRAAVGRAAGRCMNGRCRCYT |
| SEQ ID NO: 170 | GSQRSCARPGEMCMRIRCCDGQCGCNRGTGRCFCR |
| SEQ ID NO: 171 | GSGCIPRHRRCTWSGPRCCNNISCHCNISGTLCRCRPG |
| SEQ ID NO: 172 | GSNYCVARRCRPGGRQCCSGRPCACVGRVCRCPRD |
| SEQ ID NO: 173 | GSERGCSGAYRRCSSSQRCCEGRPCVCSAINSNCRCRRT |
| SEQ ID NO: 174 | GSQRSCARPGEMCMGIRCCDGQCGCNRGTGRCFCR |
| SEQ ID NO: 175 | GSRRGCFREGRWCPRSAPCCAPLRCRGPSIRQQRCVRE |
| SEQ ID NO: 176 | GSTVRCGGCNRRCCAGGCRSGRCINGRCQCYGR |
| SEQ ID NO: 177 | GSERRCEPSGRPCRPLMRIPCCGSCVRGRCA |
| SEQ ID NO: 178 | GSRGGCLPRNRFCNPSSGPRCCSGLTCRELNIWANRCL |
| SEQ ID NO: 179 | GSCARRRNWCGRNEDCCCPMRCIYAWYNQQGSCQTTITGLFRRC |
| SEQ ID NO: 180 | GSVRIPVSCRHSGQCLRPCRDAGMRTGRCMNGRCDCTPR |
| SEQ ID NO: 181 | GSQKILSNRCNNSSECIPHCIRIFGTRAAKCINRKCYCYP |
| SEQ ID NO: 182 | GSAVCNLKRCQLSCRSLGLLGKCIGDKCECVKHG |
| SEQ ID NO: 183 | GSISIGIRCSPSIDLCEGQCRIRRYFTGYCSGDTCHCSG |
| SEQ ID NO: 184 | GSGDCLPHLRRCRENNDCCSRRCRRRGANPERRCR |
| SEQ ID NO: 185 | GSSCEPGRTFRDRCNTCKCGADGRSAACTLRACPNQ |
| SEQ ID NO: 186 | GSGDCLPHLKRCKADNDCCGKKCKRRGTNAEKRCR |
| SEQ ID NO: 187 | GSGDCLPHLKRCKENNDCCSKKCKRRGTNPEKRCR |
| SEQ ID NO: 188 | GSKDCLKKLKLCKENKDCCSKSCKRRGTNIEKRCR |
| SEQ ID NO: 189 | GSGDCLPHLKRCKENNDCCSKKCKRRGANPEKRCR |
| SEQ ID NO: 190 | GSVFINVKCRGSPECLPKCKEAIGKSAGKCMNGKCKCYP |
| SEQ ID NO: 191 | GSVFINAKCRGSPECLPKCKEAIGKAAGKCMNGKCKCYP |
| SEQ ID NO: 192 | GSVIINVKCKISRQCLEPCKKAGMRFGKCMNGKCHCTP |
| SEQ ID NO: 193 | GSVPTDVKCRGSPQCIQPCKDAGMRFGKCMNGKCHCTP |
| SEQ ID NO: 194 | GSVRIPVSCKHSGQCLKPCKDAGMRFGKCMNGKCDCTP |
| SEQ ID NO: 195 | GSVRIPVSCRHSGQCLRPCRDAGMRFGRCMNGRCDCTP |
| SEQ ID NO: 196 | GSTNVSCTTSKECWSVCQRLHNTSRGKCMNKKCRC |
| SEQ ID NO: 197 | GSNVKCTGSKQCLPACKAAVGKAAGKCMNGKCKC |
| SEQ ID NO: 198 | GSGVPINVRCRGSRDCLDPCRGAGERHGRCGNSRCHCTP |
| SEQ ID NO: 199 | GSVRIPVSCRHSGQCLRPCRDAGERHGRCGGGRCDCTPR |
| SEQ ID NO: 200 | GSQVQTNVRCQGGSCGSVCRREGGGAGGGCGNGRCGCYRN |
| SEQ ID NO: 201 | GSIKCSESYQCFPVCKSRFGKTNGRCVNGFCDCF |
| SEQ ID NO: 202 | GSVKCSSPQQCLKPCKAAFGISAGgKCINGKCKCY |
| SEQ ID NO: 203 | GSVSCSASSQCWPVCKKLFGTYRGKCMNSKCRCY |
| SEQ ID NO: 204 | GSESCTASNQCWSICKRLHNTNRGKCMNKKCRCY |
| SEQ ID NO: 205 | GSVSCTTSKECWSVCEKLYNTSRGKCMNKKCRCY |
| SEQ ID NO: 206 | GSMRCKSSKECLVKCKQATGRPNGKCMNRKCKCY |
| SEQ ID NO: 207 | GSIKCTLSKDCYSPCKKETGCPRAKCINRNCKCY |
| SEQ ID NO: 208 | GSIRCSGSRDCYSPCMKQTGCPNAKCINKSCKCY |
| SEQ ID NO: 209 | GSIRCSGTRECYAPCQKLTGCLNAKCMNKACKCY |
| SEQ ID NO: 210 | GSISCTNPKQCYPHCKKETGYPNAKCMNRKCKCF |
| SEQ ID NO: 211 | GSASCRTPKDCADPCRKETGCPYGKCMNRKCKCN |
| SEQ ID NO: 212 | GSTSCISPKQCTEPCRAKGCKHGKCMNRKCHCM |
| SEQ ID NO: 213 | GSKECTGPQHCTNFCRKN-KCTHGKCMNRKCKCF |
| SEQ ID NO: 214 | GSIKCRTPKDCADPCRKQTGCPHAKCMNKTCRCH |
| SEQ ID NO: 215 | GSVKCTTSKECWPPCKAATGKAAGKCMNKKCKCQ |
| SEQ ID NO: 216 | GSLECGASRECYDPCFKAFGRAHGKCMNNKCRCY |
| SEQ ID NO: 217 | GSEKCFATSQCWTPCKKAIGSLQSKCMNGKCKCY |
| SEQ ID NO: 218 | GSVRCYASRECWEPCRRVTGSAQAKCQNNQCRCY |
| SEQ ID NO: 219 | GSVKCSASRECWVACKKVTGSGQGKCQNNQCRCY |
| SEQ ID NO: 220 | GSVKCISSQECWIACKKVTGRFEGKCQNRQCRCY |
| SEQ ID NO: 221 | GSVRCYDSRQCWIACKKVTGSTQGKCQNKQCRCY |
| SEQ ID NO: 222 | GSVDCTVSKECWAPCKAAFGVDRGKCMGKKCKCY |
| SEQ ID NO: 223 | GSAKCRGSPECLPKCKEAIGKAAGKCMNGKCKCY |
| SEQ ID NO: 224 | GSKKCQGGSCASVCRRVIGVAAGKCINGRCVCY |
| SEQ ID NO: 225 | GSKKCSNTSQCYKTCEKVVGVAAGKCMNGKCICY |
| SEQ ID NO: 226 | GSVKCSGSSKCVKICIDRYNTRGAKCINGRCTCY |
| SEQ ID NO: 227 | GSNRCNNSSECIPHCIRIFGTRAAKCINRKCYCY |
| SEQ ID NO: 228 | GSKECNGSSECYSHCEGITGKRSGKCINKKCYCY |
| SEQ ID NO: 229 | GSAFCNLRRCELSCRSLGLLGKCIGEECKCV |
| SEQ ID NO: 230 | GSAVCNLKRCQLSCRSLGLLGKCIGDKCECV |
| SEQ ID NO: 231 | GSAACYSS-DCRVKCVAMGFSSGKCINSKCKCY |
| SEQ ID NO: 232 | GSAICATDADCSRKCPGNPPCRNGFCACT |
| SEQ ID NO: 233 | GSTECQIKNDCQRYCQSVKECKYGKCYCN |
| SEQ ID NO: 234 | GSTQCQSVRDCQQYCLTPDRCSYGTCYCK |
| SEQ ID NO: 235 | GSVSCRYGSDCAEPCKRLKCLLPSKCINGKCTCY |
| SEQ ID NO: 236 | GSIKCRYPADCHIMCRKVTGRAEGKCMNGKCTCY |
| SEQ ID NO: 237 | GSIKCSSSSSCYEPCRGVTGRAHGKCMNGRCTCY |
| SEQ ID NO: 238 | GSVKCTGSKQCLPACKAAVGKAAGKCMNGKCKCY |
| SEQ ID NO: 239 | GSVSCKHSGQCIKPCKDA-GMRFGKCMNRKCDCT |
| SEQ ID NO: 240 | GSVKCRGSPQCIQPCRDA-GMRFGKCMNGKCHCT |
| SEQ ID NO: 241 | GSVKCTSPKQCLPPCKAQFGIRAGAKCMNGKCKCY |
| SEQ ID NO: 242 | GSVKCTSPKQCSKPCKELYGSSAGAKCMNGKCKCY |
| SEQ ID NO: 243 | GSVKCTSPKQCLPPCKEIYGRHAGAKCMNGKCHCS |
| SEQ ID NO: 244 | GSVKCTGSKQCWPVCKQMFGKPNGKCMNGKCRCY |
| SEQ ID NO: 245 | GSVKCRGSRDCLDPCKKAGMRFGKCINSKCHCT |
| SEQ ID NO: 246 | GSVRCVTDDDCFRKCPGNPSCKRGFCACK |
| SEQ ID NO: 247 | GSVPCNNSRPCVPVCIREVNNKNGKCSNGKCLCY |
| SEQ ID NO: 282 | GIVCKVCKIICGMQGKKVNICKAPIKCKCKKG |
| SEQ ID NO: 283 | SEKDCIKHLQRCRENKDCCSKKCSRRGTNPEKRCR |
| SEQ ID NO: 284 | VRIPVSCKHSGQCLKPCKDAGMRFGKCMNGKCDCTPK |
| SEQ ID NO: 285 | GVPINVKCRGSRDCLDPCKKAGMRFGKCINSKCHCTP |
| SEQ ID NO: 286 | AVCVYRTCDKDCKRRGYRSGKCINNACKCYPYG |
| SEQ ID NO: 287 | ISCTGSKQCYDPCKRKTGCPNAKCMNKSCKCYGCG |
| SEQ ID NO: 288 | QVQTNVKCQGGSCASVCRREIGVAAGKCINGKCVCYRN |
| SEQ ID NO: 289 | EVIRCSGSKQCYGPCKQQTGCTNSKCMNKVCKCYGCG |
| SEQ ID NO: 290 | ACKGVFDACTPGKNECCPNRVCSDKHKWCKWKL |
| SEQ ID NO: 291 | QIYTSKECNGSSECYSHCEGITGKRSGKCINKKCYCYR |
| SEQ ID NO: 292 | GCLEFWWKCNPNDDKCCRPKLKCSKLFKLCNFSFG |
| SEQ ID NO: 293 | DCVRFWGKCSQTSDCCPHLACKSKWPRNICVWDGSVG |
| SEQ ID NO: 294 | GCFGYKCDYYKGCCSGYVCSPTWKWCVRPGPGR |
| SEQ ID NO: 295 | MNAKFILLLVLTTMMLLPDTKGAEVIRCSGSKQCYGPCKQQTGC |
| | TNSKCMNKVCKCYGCG |
| SEQ ID NO: 296 | MNAKLIYLLLVVTTMTLMFDTAQAVDIMCSGPKQCYGPCKKETG |
| | CPNAKCMNRRCKCYGCV |
| SEQ ID NO: 297 | MNAKLIYLLLVVTTMMLTFDTTQAGDIKCSGTRQCWGPCKKQTT |
| | CTNSKCMNGKCKCYGCVG |
| SEQ ID NO: 298 | MNTKFIFLLLVVTNTMMLFDTKPVEGISCTGSKQCYDPCKRKTG |
| | CPNAKCMNKSCKCYGCG |
| SEQ ID NO: 299 | GVPINVKCSGSRDCLEPCKKAGMRFGKCINRKCHCTPK |
| SEQ ID NO: 300 | GVPINVKCTGSPQCLKPCKDAGMRFGKCINGKCHCTPK |
| SEQ ID NO: 301 | GVIINVKCKISRQCLEPCKKAGMRFGKCMNGKCHCTPK |
| SEQ ID NO: 302 | GVPINVKCRGSPQCIQPCRDAGMRFGKCMNGKCHCTPQ |
| SEQ ID NO: 303 | GVEINVKCTGSHQCIKPCKDAGMRFGKCINRKCHCTPK |
| SEQ ID NO: 304 | GVEINVKCSGSPQCLKPCKDAGMRFGKCMNRKCHCTPK |
| SEQ ID NO: 305 | GVPTDVKCRGSPQCIQPCKDAGMRFGKCMNGKCHCTPK |
| SEQ ID NO: 306 | GVPINVSCTGSPQCIKPCKDAGMRFGKCMNRKCHCTPK |
| SEQ ID NO: 307 | GVPINVPCTGSPQCIKPCKDAGMRFGKCMNRKCHCTPK |
| SEQ ID NO: 308 | VGINVKCKHSGQCLKPCKDAGMRFGKCINGKCDCTPK |
| SEQ ID NO: 309 | VGINVKCKHSGQCLKPCKDAGMRFGKCMNGKCDCTPK |
| SEQ ID NO: 310 | VGIPVSCKHSGQCIKPCKDAGMRFGKCMNRKCDCTPK |
| SEQ ID NO: 311 | RKGCFKEGHSCPKTAPCCRPLVCKGPSPNTKKCTRP |
| SEQ ID NO: 312 | SFCIPFKPCKSDENCCKKFKCKTTGIVKLCRW |
| SEQ ID NO: 313 | LKGCLPRNRFCNALSGPRCCSGLRCKELSIWASKCL |
| SEQ ID NO: 314 | GNYCLRGRCLPGGRKCCNGRPCECFAKICSCKPK |
| SEQ ID NO: 315 | TVKCGGCNRKCCPGGCRSGKCINGKCQCY |
| SEQ ID NO: 316 | GCMKEYCAGQCRGKVSQDYCLKHCKCIPR |
| SEQ ID NO: 317 | ACLGFGEKCNPSNDKCCKSSSLVCSQKHKWCKYG |
| SEQ ID NO: 318 | RGGCLPHNRFCNALSGPRCCSGLRCKELSIRDSRCLG |
| SEQ ID NO: 319 | RGGCLPRNKFCNPSSGPRCCSGLTCKELNIWASKCL |
| SEQ ID NO: 320 | QRSCAKPGDMCMGIKCCDGQCGCNRGTGRCFCK |
| SEQ ID NO: 321 | ARGCADAYKSCNHPRTCCDGYNGYKRACICSGSNCKCKKS |
| SEQ ID NO: 322 | RGGCLPHNRFCNALSGPRCCSGLRCKELSIWDSRCLG |
| SEQ ID NO: 323 | RGGCLPHNRFCNALSGPRCCSGLKCKELSIYDSRCLG |
| SEQ ID NO: 324 | RGGCLPHNRFCNALSGPRCCSRLKCKELSIWDSRCLG |
| SEQ ID NO: 325 | RGGCLPHNRFCNALTGPRCCSRLRCKELSIWDSICLG |
| SEQ ID NO: 326 | SCADAYKSCDSLKCCNNRTCMCSMIGTNCTCRKK |
| SEQ ID NO: 327 | ERRCLPAGKTCVRGPMRVPCCGSCSQNKCT |
| SEQ ID NO: 328 | LCSREGEFCYKLRKCCAGFYCKAFVLHCYRN |
| SEQ ID NO: 329 | ACGSCRKKCKGSGKCINGRCKCY |
| SEQ ID NO: 330 | ACGSCRKKCKGPGKCINGRCKCY |
| SEQ ID NO: 331 | ACQGYMRKCGRDKPPCCKKLECSKTWRWCVWN |
| SEQ ID NO: 332 | GRYCQKWMWTCDSKRACCEGLRCKLWCRKI |
| SEQ ID NO: 333 | NAKCRGSPECLPKCKEAIGKAAGKCMNGKCKCYP |
| SEQ ID NO: 334 | NVKCRGSKECLPACKAAVGKAAGKCMNGKCKCYP |
| SEQ ID NO: 335 | NVKCRGSPECLPKCKEAIGKSAGKCMNGKCKCYP |
| SEQ ID NO: 336 | NAKCRGSPECLPKCKQAIGKAAGKCMNGKCKCYP |
| SEQ ID NO: 337 | RGYCAEKGIKCHNIHCCSGLTCKCKGSSCVCRK |
| SEQ ID NO: 338 | ERGCKLTFWKCKNKKECCGWNACALGICMPR |
| SEQ ID NO: 339 | KKKCIAKDYGRCKWGGTPCCRGRGCICSIMGTNCECKPR |
| SEQ ID NO: 340 | GCKLTFWKCKNKKECCGWNACALGICMPR |
| SEQ ID NO: 341 | ACKGLFVTCTPGKDECCPNHVCSSKHKWCKYK |
| SEQ ID NO: 342 | IACAPRGLLCFRDKECCKGLTCKGRFVNTWPTFCLV |
| SEQ ID NO: 343 | ACAGLYKKCGKGVNTCCENRPCKCDLAMGNCICKKK |
| SEQ ID NO: 344 | FTCAISCDIKVNGKPCKGSGEKKCSGGWSCKFNVCVKV |
| SEQ ID NO: 345 | GFCAQKGIKCHDIHCCTNLKCVREGSNRVCRKA |
| SEQ ID NO: 346 | CAKKRNWCGKNEDCCCPMKCIYAWYNQQGSCQSTITGLFKKC |
| SEQ ID NO: 347 | YCQKWMWTCDSARKCCEGLVCRLWCKKI |
| SEQ ID NO: 348 | RGGCLPHNKFCNALSGPRCCSGLKCKELTIWNTKCLE |
| SEQ ID NO: 349 | NVKCTGSKQCLPACKAAVGKAAGKCMNGKCKCYT |
| SEQ ID NO: 350 | QRSCAKPGEMCMRIKCCDGQCGCNRGTGRCFCK |
| SEQ ID NO: 351 | GCIPKHKRCTWSGPKCCNNISCHCNISGTLCKCRPG |
| SEQ ID NO: 352 | NYCVAKRCRPGGRQCCSGKPCACVGKVCKCPRD |
| SEQ ID NO: 353 | ERGCSGAYKRCSSSQRCCEGRPCVCSAINSNCKCRKT |
| SEQ ID NO: 354 | RYCPRNPEACYNYCLRTGRPGGYCGGRSRITCFCFR |
| SEQ ID NO: 355 | QRSCAKPGEMCMGIKCCDGQCGCNRGTGRCFCK |
| SEQ ID NO: 356 | RRGCFKEGKWCPKSAPCCAPLKCKGPSIKQQKCVRE |
| SEQ ID NO: 357 | TVKCGGCNRKCCAGGCRSGKCINGKCQCYGR |
| SEQ ID NO: 358 | ERRCEPSGKPCRPLMRIPCCGSCVRGKCA |
| SEQ ID NO: 359 | RGGCLPRNKFCNPSSGPRCCSGLTCKELNIWANKCL |
| SEQ ID NO: 360 | CAKKRNWCGKNEDCCCPMKCIYAWYNQQGSCQTTITGLFKKC |
| SEQ ID NO: 361 | VRIPVSCKHSGQCLKPCKDAGMRTGKCMNGKCDCTPK |
| SEQ ID NO: 362 | VKCTTSKDCWPPCKKVTGRA |
| SEQ ID NO: 363 | GIVCRVCRIICGMQGRRVNICRAPIRCRCRRG |
| SEQ ID NO: 364 | SERDCIRHLQRCRENRDCCSRRCSRRGTNPERRCR |
| SEQ ID NO: 365 | VRIPVSCRHSGQCLRPCRDAGMRFGRCMNGRCDCTPR |
| SEQ ID NO: 366 | GVPINVRCRGSRDCLDPCRRAGMRFGRCINSRCHCTP |
| SEQ ID NO: 367 | AVCVYRTCDRDCRRRGYRSGRCINNACRCYPYG |
| SEQ ID NO: 368 | ISCTGSRQCYDPCRRRTGCPNARCMNRSCRCYGCG |
| SEQ ID NO: 369 | QVQTNVRCQGGSCASVCRREIGVAAGRCINGRCVCYRN |
| SEQ ID NO: 370 | EVIRCSGSRQCYGPCRQQTGCTNSRCMNRVCRCYGCG |
| SEQ ID NO: 371 | ACRGVFDACTPGRNECCPNRVCSDRHRWCRWRL |
| SEQ ID NO: 372 | QIYTSRECNGSSECYSHCEGITGRRSGRCINRRCYCYR |
| SEQ ID NO: 373 | GCLEFWWRCNPNDDRCCRPRLRCSRLFRLCNFSFG |
| SEQ ID NO: 374 | DCVRFWGRCSQTSDCCPHLACRSRWPRNICVWDGSVG |
| SEQ ID NO: 375 | GCFGYRCDYYRGCCSGYVCSPTWRWCVRPGPGR |
| SEQ ID NO: 376 | MNARFILLLVLTTMMLLPDTRGAEVIRCSGSRQCYGPCRQQTGC |
| | TNSRCMNRVCRCYGCG |
| SEQ ID NO: 377 | MNARLIYLLLVVTTMTLMFDTAQAVDIMCSGPRQCYGPCRRETG |
| | CPNARCMNRRCRCYGCV |
| SEQ ID NO: 378 | MNARLIYLLLVVTTMMLTFDTTQAGDIRCSGTRQCWGPCRRQTT |
| | CTNSRCMNGRCRCYGCVG |
| SEQ ID NO: 379 | MNTRFIFLLLVVTNTMMLFDTRPVEGISCTGSRQCYDPCRRRTG |
| | CPNARCMNRSCRCYGCG |
| SEQ ID NO: 380 | GVPINVRCSGSRDCLEPCRRAGMRFGRCINRRCHCTPR |
| SEQ ID NO: 381 | GVPINVRCTGSPQCLRPCRDAGMRFGRCINGRCHCTPR |
| SEQ ID NO: 382 | GVIINVRCRISRQCLEPCRRAGMRFGRCMNGRCHCTPR |
| SEQ ID NO: 383 | GVPINVRCRGSPQCIQPCRDAGMRFGRCMNGRCHCTPQ |
| SEQ ID NO: 384 | GVEINVRCTGSHQCIRPCRDAGMRFGRCINRRCHCTPR |
| SEQ ID NO: 385 | GVEINVRCSGSPQCLRPCRDAGMRFGRCMNRRCHCTPR |
| SEQ ID NO: 386 | GVPTDVRCRGSPQCIQPCRDAGMRFGRCMNGRCHCTPR |
| SEQ ID NO: 387 | GVPINVSCTGSPQCIRPCRDAGMRFGRCMNRRCHCTPR |
| SEQ ID NO: 388 | GVPINVPCTGSPQCIRPCRDAGMRFGRCMNRRCHCTPR |
| SEQ ID NO: 389 | VGINVRCRHSGQCLRPCRDAGMRFGRCINGRCDCTPR |
| SEQ ID NO: 390 | VGINVRCRHSGQCLRPCRDAGMRFGRCMNGRCDCTPR |
| SEQ ID NO: 391 | VGIPVSCRHSGQCIRPCRDAGMRFGRCMNRRCDCTPR |
| SEQ ID NO: 392 | RRGCFREGHSCPRTAPCCRPLVCRGPSPNTRRCTRP |
| SEQ ID NO: 393 | SFCIPFRPCRSDENCCRRFRCRTTGIVRLCRW |
| SEQ ID NO: 394 | LRGCLPRNRFCNALSGPRCCSGLRCRELSIWASRCL |
| SEQ ID NO: 395 | GNYCLRGRCLPGGRRCCNGRPCECFARICSCRPR |
| SEQ ID NO: 396 | TVRCGGCNRRCCPGGCRSGRCINGRCQCY |
| SEQ ID NO: 397 | GCMREYCAGQCRGRVSQDYCLRHCRCIPR |
| SEQ ID NO: 398 | ACLGFGERCNPSNDRCCRSSSLVCSQRHRWCRYG |
| SEQ ID NO: 399 | RGGCLPHNRFCNALSGPRCCSGLRCRELSIRDSRCLG |
| SEQ ID NO: 400 | RGGCLPRNRFCNPSSGPRCCSGLTCRELNIWASRCL |
| SEQ ID NO: 401 | QRSCARPGDMCMGIRCCDGQCGCNRGTGRCFCR |
| SEQ ID NO: 402 | ARGCADAYRSCNHPRTCCDGYNGYRRACICSGSNCRCRRS |
| SEQ ID NO: 403 | RGGCLPHNRFCNALSGPRCCSGLRCRELSIWDSRCLG |
| SEQ ID NO: 404 | RGGCLPHNRFCNALSGPRCCSGLRCRELSIYDSRCLG |
| SEQ ID NO: 405 | RGGCLPHNRFCNALSGPRCCSRLRCRELSIWDSRCLG |
| SEQ ID NO: 406 | RGGCLPHNRFCNALTGPRCCSRLRCRELSIWDSICLG |
| SEQ ID NO: 407 | SCADAYKSCDSLRCCNNRTCMCSMIGTNCTCRRR |
| SEQ ID NO: 408 | ERRCLPAGRTCVRGPMRVPCCGSCSQNRCT |
| SEQ ID NO: 409 | LCSREGEFCYRLRRCCAGFYCRAFVLHCYRN |
| SEQ ID NO: 410 | ACGSCRRRCRGSGRCINGRCRCY |
| SEQ ID NO: 411 | ACGSCRRRCRGPGRCINGRCRCY |
| SEQ ID NO: 412 | ACQGYMRRCGRDRPPCCRRLECSRTWRWCVWN |
| SEQ ID NO: 413 | GRYCQRWMWTCDSRRACCEGLRCRLWCRRI |
| SEQ ID NO: 414 | NARCRGSPECLPRCREAIGRAAGRCMNGRCRCYP |
| SEQ ID NO: 415 | NVRCRGSRECLPACRAAVGRAAGRCMNGRCRCYP |
| SEQ ID NO: 416 | NVRCRGSPECLPRCREAIGRSAGRCMNGRCRCYP |
| SEQ ID NO: 417 | NARCRGSPECLPRCRQAIGRAAGRCMNGRCRCYP |
| SEQ ID NO: 418 | RGYCAERGIRCHNIHCCSGLTCRCRGSSCVCRR |
| SEQ ID NO: 419 | ERGCRLTFWRCRNRRECCGWNACALGICMPR |
| SEQ ID NO: 420 | RRRCIARDYGRCRWGGTPCCRGRGCICSIMGTNCECRPR |
| SEQ ID NO: 421 | GCRLTFWRCRNRRECCGWNACALGICMPR |
| SEQ ID NO: 422 | ACRGLFVTCTPGRDECCPNHVCSSRHRWCRYR |
| SEQ ID NO: 423 | IACAPRGLLCFRDRECCRGLTCRGRFVNTWPTFCLV |
| SEQ ID NO: 424 | ACAGLYRRCGRGVNTCCENRPCRCDLAMGNCICRRR |
| SEQ ID NO: 425 | FTCAISCDIRVNGRPCRGSGERRCSGGWSCRFNVCVRV |
| SEQ ID NO: 426 | GFCAQRGIRCHDIHCCTNLRCVREGSNRVCRRA |
| SEQ ID NO: 427 | CARRRNWCGRNEDCCCPMRCIYAWYNQQGSCQSTITGLFRRC |
| SEQ ID NO: 428 | YCQRWMWTCDSARRCCEGLVCRLWCRRI |
| SEQ ID NO: 429 | RGGCLPHNRFCNALSGPRCCSGLRCRELTIWNTRCLE |
| SEQ ID NO: 430 | NVRCTGSRQCLPACRAAVGRAAGRCMNGRCRCYT |
| SEQ ID NO: 431 | QRSCARPGEMCMRIRCCDGQCGCNRGTGRCFCR |
| SEQ ID NO: 432 | GCIPRHRRCTWSGPRCCNNISCHCNISGTLCRCRPG |
| SEQ ID NO: 433 | NYCVARRCRPGGRQCCSGRPCACVGRVCRCPRD |
| SEQ ID NO: 434 | ERGCSGAYRRCSSSQRCCEGRPCVCSAINSNCRCRRT |
| SEQ ID NO: 435 | QRSCARPGEMCMGIRCCDGQCGCNRGTGRCFCR |
| SEQ ID NO: 436 | RRGCFREGRWCPRSAPCCAPLRCRGPSIRQQRCVRE |
| SEQ ID NO: 437 | TVRCGGCNRRCCAGGCRSGRCINGRCQCYGR |
| SEQ ID NO: 438 | ERRCEPSGRPCRPLMRIPCCGSCVRGRCA |
| SEQ ID NO: 439 | RGGCLPRNRFCNPSSGPRCCSGLTCRELNIWANRCL |
| SEQ ID NO: 440 | CARRRNWCGRNEDCCCPMRCIYAWYNQQGSCQTTITGLFRRC |
| SEQ ID NO: 441 | VRIPVSCRHSGQCLRPCRDAGMRTGRCMNGRCDCTPR |
| SEQ ID NO: 442 | QKILSNRCNNSSECIPHCIRIFGTRAAKCINRKCYCYP |
| SEQ ID NO: 443 | AVCNLKRCQLSCRSLGLLGKCIGDKCECVKHG |
| SEQ ID NO: 444 | ISIGIRCSPSIDLCEGQCRIRRYFTGYCSGDTCHCSG |
| SEQ ID NO: 445 | GDCLPHLRRCRENNDCCSRRCRRRGANPERRCR |
| SEQ ID NO: 446 | SCEPGRTFRDRCNTCKCGADGRSAACTLRACPNQ |
| SEQ ID NO: 447 | GDCLPHLKRCKADNDCCGKKCKRRGTNAEKRCR |
| SEQ ID NO: 448 | GDCLPHLKRCKENNDCCSKKCKRRGTNPEKRCR |
| SEQ ID NO: 449 | KDCLKKLKLCKENKDCCSKSCKRRGTNIEKRCR |
| SEQ ID NO: 450 | GDCLPHLKRCKENNDCCSKKCKRRGANPEKRCR |
| SEQ ID NO: 451 | VFINVKCRGSPECLPKCKEAIGKSAGKCMNGKCKCYP |
| SEQ ID NO: 452 | VFINAKCRGSPECLPKCKEAIGKAAGKCMNGKCKCYP |
| SEQ ID NO: 453 | VIINVKCKISRQCLEPCKKAGMRFGKCMNGKCHCTP |
| SEQ ID NO: 454 | VPTDVKCRGSPQCIQPCKDAGMRFGKCMNGKCHCTP |
| SEQ ID NO: 455 | VRIPVSCKHSGQCLKPCKDAGMRFGKCMNGKCDCTP |
| SEQ ID NO: 456 | VRIPVSCRHSGQCLRPCRDAGMRFGRCMNGRCDCTP |
| SEQ ID NO: 457 | TNVSCTTSKECWSVCQRLHNTSRGKCMNKKCRC |
| SEQ ID NO: 458 | NVKCTGSKQCLPACKAAVGKAAGKCMNGKCKC |
| SEQ ID NO: 459 | GVPINVRCRGSRDCLDPCRGAGERHGRCGNSRCHCTP |
| SEQ ID NO: 460 | VRIPVSCRHSGQCLRPCRDAGERHGRCGGGRCDCTPR |
| SEQ ID NO: 461 | QVQTNVRCQGGSCGSVCRREGGGAGGGCGNGRCGCYRN |
| SEQ ID NO: 462 | IKCSESYQCFPVCKSRFGKTNGRCVNGFCDCF |
| SEQ ID NO: 463 | VKCSSPQQCLKPCKAAFGISAGGKCINGKCKCY |
| SEQ ID NO: 464 | VSCSASSQCWPVCKKLFGTYRGKCMNSKCRCY |
| SEQ ID NO: 465 | ESCTASNQCWSICKRLHNTNRGKCMNKKCRCY |
| SEQ ID NO: 466 | VSCTTSKECWSVCEKLYNTSRGKCMNKKCRCY |
| SEQ ID NO: 467 | MRCKSSKECLVKCKQATGRPNGKCMNRKCKCY |
| SEQ ID NO: 468 | IKCTLSKDCYSPCKKETGCPRAKCINRNCKCY |
| SEQ ID NO: 469 | IRCSGSRDCYSPCMKQTGCPNAKCINKSCKCY |
| SEQ ID NO: 470 | IRCSGTRECYAPCQKLTGCLNAKCMNKACKCY |
| SEQ ID NO: 471 | ISCTNPKQCYPHCKKETGYPNAKCMNRKCKCF |
| SEQ ID NO: 472 | ASCRTPKDCADPCRKETGCPYGKCMNRKCKCN |
| SEQ ID NO: 473 | TSCISPKQCTEPCRAKGCKHGKCMNRKCHCM |
| SEQ ID NO: 474 | KECTGPQHCTNFCRKN-KCTHGKCMNRKCKCF |
| SEQ ID NO: 475 | IKCRTPKDCADPCRKQTGCPHAKCMNKTCRCH |
| SEQ ID NO: 476 | VKCTTSKECWPPCKAATGKAAGKCMNKKCKCQ |
| SEQ ID NO: 477 | LECGASRECYDPCFKAFGRAHGKCMNNKCRCY |
| SEQ ID NO: 478 | EKCFATSQCWTPCKKAIGSLQSKCMNGKCKCY |
| SEQ ID NO: 479 | VRCYASRECWEPCRRVTGSAQAKCQNNQCRCY |
| SEQ ID NO: 480 | VKCSASRECWVACKKVTGSGQGKCQNNQCRCY |
| SEQ ID NO: 481 | VKCISSQECWIACKKVTGRFEGKCQNRQCRCY |
| SEQ ID NO: 482 | VRCYDSRQCWIACKKVTGSTQGKCQNKQCRCY |
| SEQ ID NO: 483 | VDCTVSKECWAPCKAAFGVDRGKCMGKKCKCY |
| SEQ ID NO: 484 | AKCRGSPECLPKCKEAIGKAAGKCMNGKCKCY |
| SEQ ID NO: 485 | KKCQGGSCASVCRRVIGVAAGKCINGRCVCY |
| SEQ ID NO: 486 | KKCSNTSQCYKTCEKVVGVAAGKCMNGKCICY |
| SEQ ID NO: 487 | VKCSGSSKCVKICIDRYNTRGAKCINGRCTCY |
| SEQ ID NO: 488 | NRCNNSSECIPHCIRIFGTRAAKCINRKCYCY |
| SEQ ID NO: 489 | KECNGSSECYSHCEGITGKRSGKCINKKCYCY |
| SEQ ID NO: 490 | AFCNLRRCELSCRSLGLLGKCIGEECKCV |
| SEQ ID NO: 491 | AVCNLKRCQLSCRSLGLLGKCIGDKCECV |
| SEQ ID NO: 492 | AACYSS-DCRVKCVAMGFSSGKCINSKCKCY |
| SEQ ID NO: 493 | AICATDADCSRKCPGNPPCRNGFCACT |
| SEQ ID NO: 494 | TECQIKNDCQRYCQSVKECKYGKCYCN |
| SEQ ID NO: 495 | TQCQSVRDCQQYCLTPDRCSYGTCYCK |
| SEQ ID NO: 496 | VSCRYGSDCAEPCKRLKCLLPSKCINGKCTCY |
| SEQ ID NO: 497 | IKCRYPADCHIMCRKVTGRAEGKCMNGKCTCY |
| SEQ ID NO: 498 | IKCSSSSSCYEPCRGVTGRAHGKCMNGRCTCY |
| SEQ ID NO: 499 | VKCTGSKQCLPACKAAVGKAAGKCMNGKCKCY |
| SEQ ID NO: 500 | VSCKHSGQCIKPCKDA-GMRFGKCMNRKCDCT |
| SEQ ID NO: 501 | VKCRGSPQCIQPCRDA-GMRFGKCMNGKCHCT |
| SEQ ID NO: 502 | VKCTSPKQCLPPCKAQFGIRAGAKCMNGKCKCY |
| SEQ ID NO: 503 | VKCTSPKQCSKPCKELYGSSAGAKCMNGKCKCY |
| SEQ ID NO: 504 | VKCTSPKQCLPPCKEIYGRHAGAKCMNGKCHCS |
| SEQ ID NO: 505 | VKCTGSKQCWPVCKQMFGKPNGKCMNGKCRCY |
| SEQ ID NO: 506 | VKCRGSRDCLDPCKKAGMRFGKCINSKCHCT |
| SEQ ID NO: 507 | VRCVTDDDCFRKCPGNPSCKRGFCACK |
| SEQ ID NO: 508 | VPCNNSRPCVPVCIREVNNKNGKCSNGKCLCY |
| SEQ ID NO: 509 | GSAEIIRCSGTRECYAPCQRLTGCLNARCMNRACRCYGCV |
| SEQ ID NO: 510 | AEIIRCSGTRECYAPCQRLTGCLNARCMNRACRCYGCV |

In any of SEQ ID NO: 1-SEQ ID NO: 510 or fragment thereof, any one or more K residues can be replaced by an R residue or any one or more R residues can be replaced by a K residue. In any of SEQ ID NO: 1-SEQ ID NO: 510 or any fragment thereof, any one or more M residues can be replaced by any one of I, L, or V residues, any one or more L residues can be replaced by any one of V, I, or M residues, any one or more I residues can be replaced by any one of M, L, or V residues, or any one or more V residues can be replaced by any one of I, L, or M residues. In any embodiment, at least one of the amino acids alone or in combination can be interchanged in the peptides or peptide fragments as follows: K/R, M/I/L/V, G/A, S/T, Q/N, and D/E wherein each letter is each individually any amino acid or amino acid analogue. In some instances, the peptide can contain only one lysine residue, or no lysine residue. In any of SEQ ID NO: 1-SEQ ID NO: 510 or any fragment thereof, X can independently be any number of any amino acid or no amino acid. In some cases, a peptide can include the first two N-terminal amino acids GS, as with peptides of SEQ ID NO: 1-SEQ ID NO: 247, or such N-terminal amino acids (GS) can be substituted by any other one or two amino acids. In other cases, a peptide does not include the first two N-terminal amino acids GS, as with peptides of SEQ ID NO: 248-SEQ ID NO: 510. In some cases, the N-terminus of the peptide is blocked, such as by an acetyl group; in other instances the C-terminus of the peptide is blocked, such as by an amide group.

In some instances, the peptide of the conjugate is any one of SEQ ID NO: 1-SEQ ID NO: 510 or a functional fragment thereof. In other embodiments, the peptide of the conjugate of the disclosure further comprises a peptide with 100%, 99%, 98%, 97%, 96%, 95%, 90%, 85%, or 80% homology to any one of SEQ ID NO: 1-SEQ ID NO: 510. In further embodiments, the peptide fragment of the conjugate comprises a contiguous fragment of any one of SEQ ID NO: 1-SEQ ID NO: 510 that is at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46 residues long, wherein the peptide fragment is selected from any portion of the peptide. In some embodiments, such peptide fragments contact the cartilage and exhibit properties of those described herein for peptide and peptide-active agent conjugates.

In some embodiments, a cystine-dense peptide of the present disclosure comprises an amino acid sequence that has at least 80%, at least 85%, at least 90%, at least 95%, at least 96%, at least 97%, at least 98%, at least 99%, or at least 100% sequence identity with the amino acid sequence of any one of SEQ ID NO: 1-SEQ ID NO: 510, or a fragment thereof. In some embodiments, a cystine-dense peptide of the present disclosure comprises an amino acid sequence that has at least 80%, at least 85%, at least 90%, at least 95%, at least 96%, at least 97%, at least 98%, at least 99%, or at least 100% sequence identity with the amino acid sequence of any one of SEQ ID NO: 21-SEQ ID NO: 247 or SEQ ID NO: 282-SEQ ID NO: 510, or a fragment thereof. In other embodiments, the cystine-dense peptide of the present disclosure comprises an amino acid sequence that has at least 99.1%, at least 99.2%, at least 99.3%, at least 99.4%, at least 99.5%, at least 99.66%, at least 99.7%, at least 99.8%, or at least 99.9% sequence identity with the amino acid sequence of any one of (i) SEQ ID NO: 1-SEQ ID NO: 510 or a fragment thereof, or (ii) SEQ ID NO: 21-SEQ ID NO: 247 or SEQ ID NO: 282-SEQ ID NO: 510 or a fragment thereof.

The peptides of the conjugates of the present disclosure can further comprise negative amino acid residues. In some cases, the peptide has 2 or fewer negative amino acid residues. In other cases, the peptide has 4 or fewer negative amino acid residues, 3 or fewer negative amino acid residues, or 1 or fewer negative amino acid residues. The negative amino acid residues can be selected from any negative charged amino acid residues. The negative amino acid residues can selected from either E or D, or a combination of both E and D.

The peptides of the conjugates of the present disclosure can further comprise basic amino acid residues. In some embodiments, basic residues are added to the peptide sequence to increase the charge at physiological pH. The added basic residues can be any basic amino acid. The added basic residues can be selected from K or R, or a combination of K or R.

In some embodiments, the peptide of the conjugate has a charge distribution comprising an acidic region and a basic region. An acidic region can be a nub. A nub is a portion of a peptide extending out of the peptide's three-dimensional structure. A basic region can be a patch. A patch is a portion of a peptide that does not designate any specific topology characteristic of the peptide's three-dimensional structure. In further embodiments, a knotted peptide can be 6 or more basic residues and 2 or fewer acidic residues.

The peptides of the conjugates of the present disclosure can further comprise positively charged amino acid residues. In some cases, the peptide has at least 2 positively charged residues. In other cases, the peptide has at least 3 positively charged residues, at least 4 positively charged residues, at least 5 positively charged residues, at least 6 positively charged residues, at least 7 positively charged residues, at least 8 positively charged residues or at least 9 positively charged residues. The positively charged residues can be selected from any positively charged amino acid residues. The positively charged residues can be selected from either K or R, or a combination of K and R.

In addition, the peptides of the conjugates herein can comprise a 4-19 amino acid residue fragment of any of the above sequences containing at least 2 cysteine residues, and at least 2 or 3 positively charged amino acid residues (for example, arginine, lysine or histidine, or any combination of arginine, lysine or histidine). In other embodiments, the peptides herein is a 20-70 amino acid residue fragment of any of the above sequences containing at least 2 cysteine residues, no more than 2 basic residues, and at least 2 or 3 positively charged amino acid residues (for example, arginine, lysine or histidine, or any combination of arginine, lysine or histidine). In some embodiments, such peptide fragments contact the cartilage and exhibit properties of those described herein for peptide and peptide-active agent conjugates.

In some embodiments, the peptide of a conjugate contains one or more disulfide bonds and has a positive net charge at neutral pH. At physiological pH, peptides can have a net charge, for example, of −5, −4, −3, −2, −1, 0, +1, +2, +3, +4, or +5. When the net charge is zero, the peptide can be uncharged or zwitterionic. In some instances, the peptide can have a positive charge at physiological pH. In some instances, the peptide can have a charge ≥+2 at physiological pH, ≥+3.5 at physiological pH, ≥+4.5 at physiological pH. In some embodiments, the peptide contains one or more disulfide bonds and has a positive net charge at neutral pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10. In some cases, the engineering of one or more mutations within a peptide yields a peptide with an altered isoelectric point, charge, surface charge, or rheology at physiological pH. Such engineering of a mutation to a peptide derived from a scorpion or spider can change the net charge of the complex, for example, by decreasing the net charge by 1, 2, 3, 4, or 5, or by increasing the net charge by 1, 2, 3, 4, or 5. In such cases, the engineered mutation may facilitate the ability of the peptide to contact the cartilage. Suitable amino acid modifications for improving the rheology and potency of a peptide can include conservative or non-conservative mutations. A peptide can comprises at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin that the peptide is derived from. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom or toxin that the peptide is derived from. In some embodiments, mutations can be engineered within a peptide to provide a peptide that has a desired charge or stability at physiological pH.

In some embodiments, charge can play a role in cartilage homing of the peptide of the conjugate. The interaction of a peptide of this disclosure in solution and in vivo can be influenced by the isoelectric point (pI) of the cystine-dense peptide and/or the pH of the solution or the local environment it is in. The charge of a peptide in solution can impact the solubility of the protein as well as parameters such as biodistribution, bioavailability, and overall pharmacokinetics. Additionally, positively charged molecules can interact with negatively charged molecules. Positively charged molecules such as the peptides disclosed herein can interact and bind with negatively charged molecules such as the negatively charged extracellular matrix molecules in the cartilage including hyaluranon and aggrecan. Positively charged residues can also interact with specific regions of other proteins and molecules, such as negatively charged residues of receptors or electronegative regions of an ion channel pore on cell surfaces. As such, the pI of a peptide can influence whether a peptide of this disclosure can efficiently home to cartilage. Identifying a correlation between pI and cartilage homing can be an important strategy in identifying lead peptide candidates of the present disclosure. The pI of a peptide can be calculated using a number of different methods including the Expasy pI calculator and the Sillero method. The Expasy pI can be determined by calculating pKa values of amino acids as described in Bjellqvist et al., which were defined by examining polypeptide migration between pH 4.5 to pH 7.3 in an immobilized pH gradient gel environment with 9.2M and 9.8M urea at 15° C. or 25° C. (Bjellqvist et al. Electrophoresis. 14(10):1023-31 (1993)). The Sillero method of calculating pI can involve the solution of a polynomial equation and the individual pKas of each amino acid. This method does not use denaturing conditions (urea) (Sillero et al. 179(2): 319-35 (1989)) Using these pI calculation methods and quantifying the cartilage to blood ratio of peptide signal after administration to a subject can be a strategy for identifying a trend or correlation in charge and cartilage homing. In some embodiments, a peptide with a pI above biological pH (˜pH 7.4) can exhibit efficient homing to cartilage. In some embodiments, a peptide with a pI of at least 8, at least 9, at least 10, or at least 11 can efficiently home to cartilage. In other embodiments, a peptide with a pI of 11-12 can home most efficiently to cartilage. In certain embodiments, a peptide can have a pI of about 9. In other embodiments, a peptide can have a pI of 8-10. In some embodiments, more basic peptides can home more efficiently to cartilage. In other embodiments, a high pI alone may not be sufficient to cause cartilage homing of a peptide.

In some embodiments, the tertiary structure and electrostatics of a peptide of the conjugate of the disclosure can impact cartilage homing. Structural analysis or analysis of charge distribution can be a strategy to predict residues important in biological function, such as cartilage homing. For example, several peptides of this disclosure that home to cartilage can be grouped into a structural class defined herein as “hitchins,” and can share the properties of disulfide linkages between C1-C4, C2-C5, and C3-C6. The folding topologies of peptides knotted through three disulfide linkages (C1-C4, C2-C5, and C3-C6), can be broken down into structural families based on the three-dimensional arrangement of the disulfides. Knottins can have the C3-C6 disulfide linkage passing through the macrocycle formed by the C1-C4 and C2-C5 disulfide linkages, hitchins have the C2-C5 disulfide linkage passing through the macrocycle formed by the C1-C4 and C3-C6 disulfide linkages, and yet other structural families have the C1-C4 disulfide linkage passing through the macrocycle formed by the C2-C5 and C3-C6 disulfide linkages. Variants of “hitchin” class peptides with preserved disulfide linkages at these cysteine residues, primary sequence identity, and/or structural homology can be a method of identifying or predicting other potential cystine-dense peptide candidates that can home to cartilage. Additionally, members and related members of the calcin family of peptides can also home to cartilage, despite having a distinct tertiary structure from the “hitchin” class of peptides. Calcin peptides are structurally a subset of the knottins, with knottin disulfide connectivity and topology, but are further classified on the basis of functioning to bind and activate ryanodine receptors (RyRs). These receptors are calcium channels that act to regulate the influx and efflux of calcium in muscle (Schwartz et al. Br J Pharmacol 157(3):392-403. (2009)). Variants of the calcin family of peptides with preserved key residues can be one way to predict promising candidates that can home to cartilage. In some embodiments, structural analysis of a peptide of this disclosure can be determined by evaluating peptides for resistance to degradation in buffers with various proteases or reducing agents. Structural analysis of the distribution of charge density on the surface of a peptide can also be a strategy for predicting promising candidates that can home to cartilage. Peptides with large patches of positive surface charge (when at pH 7.5) can home to cartilage.

The NMR solution structures, x-ray crystallography, or crystal structures of related structural homologs can be used to inform mutational strategies that can improve the folding, stability, and manufacturability, while maintaining the ability of a peptide of a conjugate to home to cartilage. They can be used to predict the 3D pharmacophore of a group of structurally homologous scaffolds, as well as to predict possible graft regions of related proteins to create chimeras with improved properties. For example, this strategy can be used to identify important amino acid positions and loops that can be used to design drugs with improved properties or to correct deleterious mutations that complicate folding and manufacturability for the peptides. These key amino acid positions and loops can be retained while other residues in the peptide sequences can be mutated to improve, change, remove, or otherwise modify function, homing, and activity of the peptide.

Additionally, the comparison of the primary sequences and the tertiary sequences of two or more peptides can be used to reveal sequence and 3D folding patterns that can be leveraged to improve the peptides and parse out the biological activity of these peptides. For example, comparing two different peptide scaffolds that home to cartilage can lead to the identification of conserved pharmacophores that can guide engineering strategies, such as designing variants with improved folding properties. Important pharmacophore, for example, can comprise aromatic residues or basic residues, which can be important for binding.

Improved peptides of the conjugates can also be engineered based upon immunogenicity information, such as immunogenicity information predicted by TEPITOPE and TEPITOPEpan. TEPITOPE is a computational approach which uses position specific scoring matrix to provide prediction rules for whether a peptide will bind to 51 different HLA-DR alleles, and TEPITOPEpan is method that uses TEPITOPE to extrapolate from HLA-DR molecules with known binding specificities to HLA-DR molecules with unknown binding specificities based on pocket similarity. For example, TEPITOPE and TEPITOPEpan can be used to determine immunogenicity of peptides that home to cartilage. Comparison of peptides with high immunogenicity to peptides with low immunogenicity can guide engineering strategies for designing variants with decreased immunogenicity.

A peptide of a conjugate of this disclosure can bind to sodium channels. The peptide can bind to calcium channels. The peptide can block potassium channels and/or sodium channels. The peptide can block calcium channels. In some embodiments, the peptide can activate potassium channels and/or sodium channels. In other embodiments, the peptide can activate calcium channels. In still other embodiments, the peptide can be a potassium channel agonist, a potassium channel antagonist, a portion of a potassium channel, a sodium channel agonist, a sodium channel antagonist, a calcium channel agonist, a calcium channel antagonist, a hadrucalcin, a theraphotoxin, a huwentoxin, a kaliotoxin, a cobatoxin or a lectin. In some embodiments, the lectin can be SHL-Ib2. In some embodiments, the peptide can interact with, binds, inhibits, inactivates, or alters expression of ion channels or chloride channels. In some embodiments, the peptide can interact with an Nav1.7 ion channel. In some embodiments, the peptide can interact with a Kv 1.3 ion channel. In still other embodiments, the peptide interacts with proteases, matrix metalloproteinase, inhibits cancer cell migration or metastases, has antimicrobial activity, or has antitumor activity. In addition to acting on matrix metalloproteinases, the peptide can interact with other possible proteases (e.g., elastases).

In some embodiments, the peptide of the conjugate has other therapeutic effects on the cartilage or structures thereof or nearby. Beta defensin expression in articular cartilage can be correlated with immunomodulatory functions as well as osteoarthritis, autoimmune rheumatic disorders such as systemic lupus erythematosus and rheumatoid arthritis (Vordenbaumen and Schneider 2011, Varoga 2004 and Varoga 2005). In some embodiments, the peptides or their mutants inhibit beta defensins, supplement beta defensins, are competitive inhibitors of beta defensins, active or block activation of beta defensin targets, and are used as immune modulators, or to treat autoimmune, arthritis, infections, and other articular disorders. In some cases, the condition is a chondrodystrophy, a traumatic rupture or detachment, pain following surgery in regions of the body containing cartilage, costochondritis, herniation, polychondritis, arthritis, osteoarthritis, rheumatoid arthritis, ankylosing spondylitis (AS), a Lupus disease (e.g., Systemic Lupus Erythematosus, also referred to herein as“SLE” or “Lupus”), Psoriatic Arthritis (PsA), gout, achondroplasia, or another suitable condition. In some cases, the condition is associated with a cancer or tumor of the cartilage. In some cases, the condition is a type of chondroma or chondrosarcoma, whether metastatic or not, or another suitable condition. In some embodiments, such as those associated with cancers, the imaging may be associated with surgical removal of the diseased region, tissue, structure or cell of a subject. In other aspects, the condition is a chordoma. In some aspects, the condition is a type of arthritis. In some aspects, the type of arthritis is rheumatoid arthritis. In some aspects, the type of arthritis is osteoarthritis. In some aspects, the type of arthritis is lupus arthritis. In some aspects, the condition is Systemic lupus erythematosus. In some aspects, the condition is achondroplasia. In some aspects, the condition is benign chondroma or malignant chondrosarcoma. In some aspects, the condition is bursitis, tendinitis, gout, pseudogout, an arthropathy, psoriatic arthritis, ankylosing spondylitis, or an infection. In some aspects, the peptide active agent complex, peptide, or pharmaceutical composition is administered to treat the injury, to repair a tissue damaged by the injury, or to treat a pain caused by the injury. In some aspects, the peptide active agent complex, peptide, or pharmaceutical composition is administered to treat the tear or to repair a tissue damaged by the tear. In some aspects, the peptide active agent complex, peptide, or pharmaceutical composition homes, targets, or migrates to a kidney of the subject following administration. In some aspects, the condition is associated with a kidney. In some aspects, the condition is lupus nephritis, acute kidney injury (AKI), chronic kidney disease (CKD), hypertensive kidney damage, diabetic nephropathy, lupus nephritis, or renal fibrosis.

The present disclosure can also encompass multimers of the various peptides described herein in a conjugate. Examples of multimers include dimers, trimers, tetramers, pentamers, hexamers, heptamers, and so on. A multimer can be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The conjugates of the present disclosure can further include peptide scaffolds that, e.g., can be used as a starting point for generating additional peptides. In some embodiments, these scaffolds can be derived from a variety of knotted peptides or cystine-dense peptides. Some suitable peptides for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, μ-PIIIA, ω-CVID, χ-MrIA, ρ-TIA, conantokin G, contulakin G, GsMTx4, margatoxin, shK, toxin K, chymotrypsin inhibitor (CTI), and EGF epiregulin core.

In some embodiments, the peptide sequences of the disclosure are flanked by additional amino acids. One or more additional amino acids can, for example, confer a desired in vivo charge, isoelectric point, chemical conjugation site, stability, or physiologic property to a peptide.

Identifying sequence homology can be important for determining key residues that preserve cartilage homing function of the peptide of the conjugate. For example, in some embodiments identification of conserved positively charged residues can be important in preserving cartilage homing in any homologous variants that are made. In other embodiments, identification of basic or aromatic dyads, can be important in preserving interaction and activity with Kv ion channels in homologous variants.

Two or more peptides can share a degree of homology and share similar properties in vivo. For instance, a peptide can share a degree of homology with a peptide of the present disclosure. In some cases, a peptide of the disclosure can have up to about 20% pairwise homology, up to about 25% pairwise homology, up to about 30% pairwise homology, up to about 35% pairwise homology, up to about 40% pairwise homology, up to about 45% pairwise homology, up to about 50% pairwise homology, up to about 55% pairwise homology, up to about 60% pairwise homology, up to about 65% pairwise homology, up to about 70% pairwise homology, up to about 75% pairwise homology, up to about 80% pairwise homology, up to about 85% pairwise homology, up to about 90% pairwise homology, up to about 95% pairwise homology, up to about 96% pairwise homology, up to about 97% pairwise homology, up to about 98% pairwise homology, up to about 99% pairwise homology, up to about 99.5% pairwise homology, or up to about 99.9% pairwise homology with a second peptide. In some cases, a peptide of the disclosure can have at least about 20% pairwise homology, at least about 25% pairwise homology, at least about 30% pairwise homology, at least about 35% pairwise homology, at least about 40% pairwise homology, at least about 45% pairwise homology, at least about 50% pairwise homology, at least about 55% pairwise homology, at least about 60% pairwise homology, at least about 65% pairwise homology, at least about 70% pairwise homology, at least about 75% pairwise homology, at least about 80% pairwise homology, at least about 85% pairwise homology, at least about 90% pairwise homology, at least about 95% pairwise homology, at least about 96% pairwise homology, at least about 97% pairwise homology, at least about 98% pairwise homology, at least about 99% pairwise homology, at least about 99.5% pairwise homology, at least about 99.9% pairwise homology with a second peptide. Various methods and software programs can be used to determine the homology between two or more peptides, such as NCBI BLAST, Clustal W, MAFFT, Clustal Omega, AlignMe, Praline, or another suitable method or algorithm.

In still other instances, the variant nucleic acid molecules of a peptide of any one of SEQ ID NO: 21-SEQ ID NO: 247 or SEQ ID NO: 282-SEQ ID NO: 510 can be identified by either a determination of the sequence identity or homology of the encoded peptide amino acid sequence with the amino acid sequence of any one of SEQ ID NO: 21-SEQ ID NO: 247 or SEQ ID NO: 282-SEQ ID NO: 510, or by a nucleic acid hybridization assay. Such peptide variants can include nucleic acid molecules (1) that remain hybridized with a nucleic acid molecule having the nucleotide sequence of any one of SEQ ID NO: 21-SEQ ID NO: 247 or SEQ ID NO: 282-SEQ ID NO: 510 (or any complement of the previous sequences) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one SEQ ID NO: 21-SEQ ID NO: 247 or SEQ ID NO: 282-SEQ ID NO: 510. Alternatively, peptide variants of any SEQ ID NO: 21-SEQ ID NO: 247 or SEQ ID NO: 282-SEQ ID NO: 510 can be characterized as nucleic acid molecules (1) that remain hybridized with a nucleic acid molecule having the nucleotide sequence of any one SEQ ID NO: 21-SEQ ID NO: 247 or SEQ ID NO: 282-SEQ ID NO: 510 (or any complement of the previous sequences) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 21-SEQ ID NO: 247 or SEQ ID NO: 282-SEQ ID NO: 510.

Percent sequence identity or homology can be determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, for example from three to six, or for example three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present disclosure. Although it is possible to design amino acid substitutions based solely upon chemical properties (as discussed above), the language “conservative amino acid substitution” can refer to a substitution represented by a BLOSUM62 value of greater than −1. For example, an amino acid substitution is conservative if the substitution is characterized by a BLOSUM62 value of 0, 1, 2, or 3. According to this system, some conservative amino acid substitutions are characterized by a BLOSUM62 value of at least 1 (e.g., 1, 2 or 3), while some conservative amino acid substitutions are characterized by a BLOSUM62 value of at least 2 (e.g., 2 or 3).

Determination of amino acid residues within regions or domains that are important to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments determination of structure can typically be accompanied by evaluating activity of modified molecules.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

### Chemical Modifications

A peptide of a conjugate can be chemically modified one or more of a variety of ways. In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of a conjugate of the disclosure. In some embodiments, the peptide can be modified by methylation on free amines. Free amines can be N-terminal amino group(s) and/or an amino group of an amino acid side chain such as lysine. For example, full methylation (e.g., the replacement of each hydrogen atom of an amino group with a methyl group) can be accomplished through the use of reductive methylation with formaldehyde and sodium cyanoborohydride.

A chemical modification can, for instance, extend the half-life of a peptide of a conjugate or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. The chemical modification of a peptide with an Fc region can be a fusion Fc-peptide. A polyamino acid can include, for example, a polyamino acid sequence with repeated single amino acids (e.g., polyglycine), and a polyamino acid sequence with mixed polyamino acid sequences (e.g., gly-ala-gly-ala (SEQ ID NO: 511)) that can or can not follow a pattern, or any combination of the foregoing.

In some embodiments, the peptides of the conjugates of the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide of the present disclosure. In other embodiments, the peptide of the present disclosure can include post-translational modifications (e.g., methylation and/or amidation), which can affect, e.g., serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof. In other embodiments, the peptides of the present disclosure are coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin.

In some embodiments, the first two N-terminal amino acids (GS) of SEQ ID NO: 1-SEQ ID NO: 247 can serve as a spacer or linker in order to facilitate conjugation or fusion to another molecule, as well as to facilitate cleavage of the peptide from such conjugated or fused molecules. In some embodiments, the fusion proteins or peptides of the present disclosure can be conjugated to other moieties that, e.g., can modify or effect changes to the properties of the peptides.

### Active Agent Conjugates

Peptides of the conjugates according to the present disclosure can be conjugated or fused to an active agent for use in the treatment of cartilage diseases, disorders, or injuries. Generally, any peptide disclosed herein can be conjugated to an active agent. Such a peptide can comprise an amino acid having at least 80%, at least 90%, at least 95%, at least 96%, at least 97%, at least 98%, at least 99%, or at least 100% sequence identity to any one or more of the amino acid sequences set forth in SEQ ID NO: 1-SEQ ID NO: 510, or a fragment thereof. The active agent that such a peptide can be conjugated to can be any active agent described herein. In some embodiments, a peptide-active agent conjugate comprises a peptide (e.g., a peptide having an amino acid sequence set forth in any one of SEQ ID NO: 21-SEQ ID NO: 247 or SEQ ID NO: 282-SEQ ID NO: 510), a linker (e.g., any one of the linkers listed in TABLE 2), and an active agent as described herein, wherein the linker conjugates the peptide to the active agent. An active agent can be an anti-arthritic agent such as an anti-inflammatory agent. For example, an anti-inflammatory agent is a glucocorticoid or an NSAID.

As described herein, the terms “peptide-active agent conjugate”, “peptide-drug conjugate”, and “PDC” can be used interchangeably herein. Certain non-limiting exemplary “peptide-active agent conjugates”, “peptide-drug conjugates”, and “PDCs” are annotated herein with respect to an agent drug or drug class, for example, “peptide-glucocorticoid conjugate”, “peptide-dexamethasone conjugate”, and “peptide-des-ciclesonide conjugate” and/or non-limiting exemplary linker, for example, “peptide-DMA-drug conjugate”, “peptide-DMA-Dex” or “peptide-DMA-dCIC”

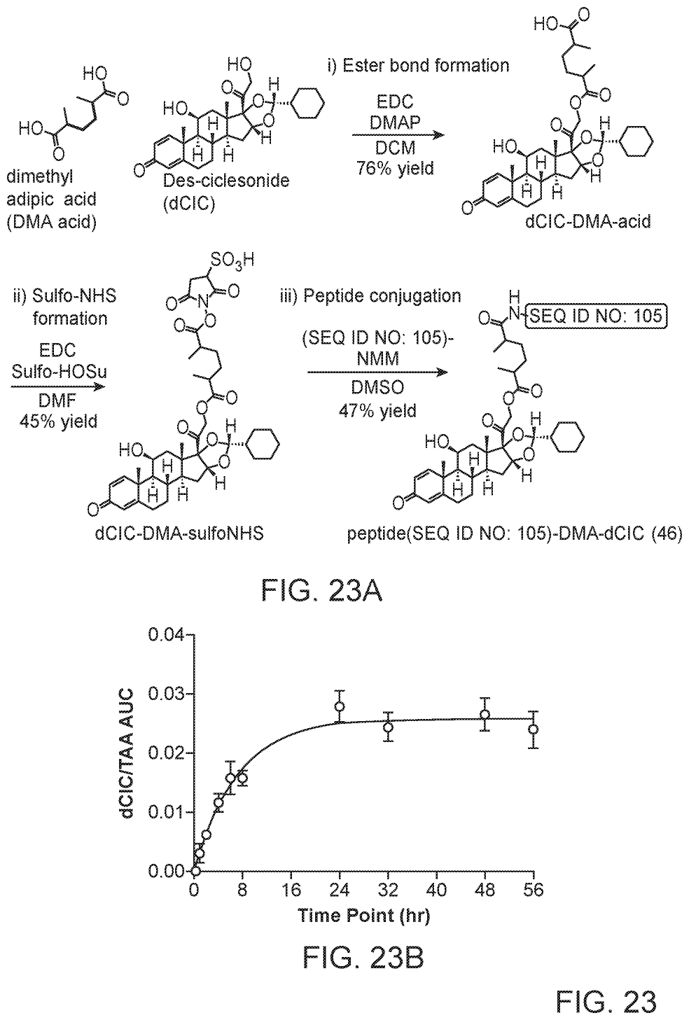

Generally, when chemical structures of peptide-drug conjugates are displayed, it is noted that the amine (e.g., the “—NH—”) of the peptide-linker bond (e.g., an amide bond) immediately adjacent to the denoted peptide is part of the peptide. FIG. 23A, for example, illustrates that “—NH-SEQ ID NO: 105” defines the peptide, wherein an amino group of the peptide (e.g., the N-terminus) is used to attach the peptide to the linker (DMA in this case) via, in this case, an amide bond (this linker comprises an ester bond on the dCIC side and an amide bond on the peptide side).

As used herein, the nomenclature “peptide(SEQ ID NO: 105)”, for example, is an abbreviation and refers to a peptide having the amino acid sequence set forth in SEQ ID NO: 105. This nomenclature can be used for any peptide and any peptide-drug conjugate (PDC) disclosed herein.

In certain embodiments, a peptide of a conjugate as described herein can be fused to another molecule, such as an active agent that provides a functional capability. The peptide can be fused with an active agent through expression of a vector containing the sequence of the peptide with the sequence of the active agent. In various embodiments, the sequence of the peptide and the sequence of the active agent are expressed from the same Open Reading Frame (ORF). In various embodiments, the sequence of the peptide and the sequence of the active agent can comprise a contiguous sequence. The peptide and the active agent can each retain similar functional capabilities in the fusion peptide compared with their functional capabilities when expressed separately.

Furthermore, for example, in certain embodiments, the peptides of the conjugates described herein are attached to another molecule, such as an active agent that provides a functional capability. In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, aa chemokine, a neurotransmitter, an ion channel inhibitor, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-arthritic agent such as an anti-inflammatory agent, a glucocorticoid, e.g., budesonide (i.e., BUD), dexamethasone (i.e., Dex), triamcinolone, triamcinolone acetonide (i.e., TAA), des-ciclesonide (can also be referred to herein as dCIC or desisobutyryl-ciclesonide), beclomethasone, betamethasone, butixicort, cortisol (hydrocortisone), clobetasol, estriol, diflorasone, diflucortolone, difluprednate, hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof, an immune modulator, a complement fixing peptide or protein, a tumor necrosis factor inhibitor, a tumor necrosis factor activator, a tumor necrosis factor receptor family agonist, a tumor necrosis receptor antagonist, a tumor necrosis factor (TNF) soluble receptor or antibody, caspase protease activator or inhibitor, an NF-κB a RIPK1 and/or RIPK3 inhibitor or activator (e.g., through Toll-like receptors (TLRs) TLR-3 and/or TLR-4, or T-cell receptor (TCR) and the like), a death-receptor ligand (E.g., Fas ligand) activator or inhibitor, TNF receptor family (e.g., TNFR1, TNFR2, lymphotoxin β receptor/TNFRS3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRS10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TACl/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17), GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27, ectodysplasin 1, and anhidrotic receptor, a TNF receptor superfamily ligand including—TNF alpha, lymphotoxin-α, tumor necrosis factor membrane form, tumor necrosis factor shed form, LIGHT, lymphotoxin β₂α₁ heterotrimer, OX-40 ligand, compound 1 [PMID: 24930776], CD40 ligand, Fas ligand, TL1A, CD70, CD30 ligand, TRAF1, TRAF2, TRAF3, TRAIL, RANK ligand, APRIL, BAFF, B and T lymphocyte attenuator, NGF, BDNF, neurotrophin-3, neurotrophin-4, TL6, ectodysplasin A2, ectodysplasin A1—a TIMP-3 inhibitor, a BCL-2 family inhibitor, an IAP disruptor, a protease inhibitor, an amino sugar, a chemotherapeutic (whether acting through an apoptotic or non-apoptotic pathway) (Ricci et al. Oncologist 11(4):342-57 (2006)), a cytotoxic chemical, a toxin, a tyrosine kinase inhibitor (e.g. imatinib mesylate), protons, bevacuzimab (antivascular agent), erlotinib (EGFR inhibitor), an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc domain or an Fc region, or an active fragment or a modification thereof. Any combination of the above active agents can be co-delivered with conjugates of this disclosure. Additionally, in some embodiments, other co-therapies such as proton therapy or ablative radiotherapy can be administered to a subject in need thereof along with the conjugates of this disclosure. In some embodiments, the peptide is covalently or non-covalently linked to an active agent or an anti-arthritic agent such as an anti-inflammatory agent, e.g., directly or via a linker. TNF blockers suppress the immune system by blocking the activity of TNF, a substance in the body that can cause inflammation and lead to immune-system diseases, such as Crohn's disease, ulcerative colitis, rheumatoid arthritis, ankylosing spondylitis, psoriatic arthritis and plaque psoriasis. The drugs in this class include Remicade (infliximab), Enbrel (etanercept), Humira (adalimumab), Cimzia (certolizumab pegol) and Simponi (golimumab). The peptide disclosed herein can be used to home, distribute to, target, directed to, is retained by, accumulate in, migrate to, and/or bind to cartilage, and thus also be used for localizing the attached or fused active agent or anti-arthritic agent such as anti-inflammatory agent. Furthermore, the knotted chlorotoxin peptide can be internalized in cells (Wiranowska, M., Cancer Cell Int., 11: 27 (2011)). Therefore, cellular internalization, subcellular localization, and intracellular trafficking after internalization of the active agent peptide conjugate or fusion peptide can be important factors in the efficacy of an active agent conjugate or fusion. (Ducry, L., Antibody Drug Conjugates (2013); and Singh, S. K., Pharm Res. 32(11): 3541-3571 (2015)). Exemplary linkers suitable for use with the embodiments herein are discussed in further detail below.

The peptides or fusion peptides of the conjugates of the present disclosure can also be conjugated to other moieties that can serve other roles, such as providing an affinity handle (e.g., biotin) for retrieval of the peptides from tissues or fluids. For example, peptides or fusion peptides of the conjugates of the present disclosure can also be conjugated to biotin. In addition to extension of half-life, biotin could also act as an affinity handle for retrieval of peptides, fusion peptides, or conjugates from tissues or other locations. In some embodiments, fluorescent biotin conjugates that can act both as a detectable label and an affinity handle can be used. Non limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates could include chemiluminescent compounds, colloidal metals, luminescent compounds, enzymes, radioisotopes, and paramagnetic labels. In some embodiments, the peptide of the conjugates described herein can be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

Additionally, more than one peptide amino acid sequence derived from a toxin or venom can be present on or fused with a particular peptide of a conjugate. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

### Detectable Agent Conjugates

A peptide of a conjugate can be conjugated to an agent used in imaging, research, therapeutics, theranostics, chemotherapy, chelation therapy, targeted drug delivery, and radiotherapy. The agent can be a detectable agent. Generally, any peptide disclosed herein can be conjugated to a detectable agent. Such a peptide can comprise an amino acid having at least 80%, at least 90%, at least 95%, at least 96%, at least 97%, at least 98%, at least 99%, or at least 100% sequence identity to any one or more of the amino acid sequences set forth in SEQ ID NO: 1-SEQ ID NO: 510, or a fragment thereof. The detectable agent that such a peptide can be conjugated to can be any detectable agent described herein. In some embodiments, a peptide-detectable agent conjugate comprises a peptide (e.g., a peptide having an amino acid sequence set forth in any one of SEQ ID NO: 21-SEQ ID NO: 247 or SEQ ID NO: 282-SEQ ID NO: 510), a linker (e.g., any one of the linkers listed in TABLE 2), and a detectable agent as described herein, wherein the linker conjugates the peptide to the detectable agent.

In some embodiments, a cystine-dense peptide is conjugated to detectable agents, such as a metal, a radioisotope, a dye, fluorophore, or another suitable material that can be used in imaging. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the fluorophore is a fluorescent agent emitting electromagnetic radiation at a wavelength between 650 nm and 4000 nm, such emissions being used to detect such agent. In some embodiments the fluorophore is a fluorescent agent is selected from the group consisting of fluorescent dyes that could be used as a conjugating molecule in the present disclosure include DyLight-680, DyLight-750, VivoTag-750, DyLight-800, IRDye-800, VivoTag-680, Cy5.5, or an indocyanine green (ICG). In some embodiments, near infrared dyes often include cyanine dyes. Additional non-limiting examples of fluorescent dyes for use as a conjugating molecule in the present disclosure include acradine orange or yellow, Alexa Fluors and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iodine, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Other embodiments of the present disclosure provide conjugates comprising peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is covalently or non-covalently linked to the agent, e.g., directly or via a linker. Exemplary linkers suitable for use with the embodiments herein are discussed in further detail below.

### Linkers in Conjugates

Peptides of the conjugates according to the present disclosure that home, target, migrate to, are retained by, accumulate in, and/or bind to, or are directed to the cartilage can be attached to another moiety (e.g., an active agent or anti-arthritic agent such as anti-inflammatory agent), such as a small molecule such as a glucocorticoid, a second peptide, a protein, an antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent described herein through a linker, or directly in the absence of a linker.

A peptide of a conjugate can be directly attached to another molecule by a covalent attachment. The attachment can be via an amide bond, an ester bond, an ether bond, a carbamate bond, a carbonate bond, a carbon-nitrogen bond, a triazole, a macrocycle, an oxime bond, a hydrazone bond, an azo bond, a carbon-carbon single double or triple bond, a disulfide bond, or a thioether bond. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbonate bond, a carbon-nitrogen bond, a triazole, a macrocycle, an oxime bond, a hydrazone bond, a carbon-carbon single double or triple bond, a disulfide bond, or a thioether bond, or linker as described herein) can be used to link other molecules. In some embodiments, the peptide is attached to a terminus of the amino acid sequence of a larger polypeptide or peptide molecule, or is attached to a side chain, such as the side chain of a lysine, serine, threonine, cysteine, tyrosine, aspartic acid, a non-natural amino acid residue, or glutamic acid residue.

Attachment via a linker can involve incorporation of a linker moiety between the other molecule (e.g., an active agent and/or a detectable agent) and the peptide (e.g., a peptide having an amino acid sequence set forth in any one of SEQ ID NO: 21-SEQ ID NO: 247 or SEQ ID NO: 282-SEQ ID NO: 510). The peptide and the other molecule can both be covalently attached to the linker. The linker can be cleavable, stable, self-immolating, hydrophilic, or hydrophobic. The linker can have bulky side groups or chains that sterically limit access of enzymes, water, or other chemicals to the linking group. The linker can have at least two functional groups (e.g., carboxylic acids, carbamic acids, carbonic acids, amines, thiols, ester etc.) with one bonded to the peptide (e.g., an ester in the linker bonded to an amine of the peptide, such as to form an amide bond), the other bonded to the other molecule (e.g., a carboxylic acid, a carbamic acid, a carbonic acid, etc. on the linker bonded to a hydroxyl group in the other molecule to form a bond such as an ester, carbamate, or carbonate bond), and a linking portion between the two functional groups. The other molecule(s) may be an active agent (e.g., a glucocorticoid) and/or a detectable agent.

Non-limiting examples of the functional groups for attachment can include functional groups capable of forming an amide bond, an ester bond, an ether bond, a carbonate bond, a carbamate bond, or a thioether bond. Non-limiting examples of functional groups capable of forming such bonds can include amino groups; carboxyl groups; hydroxyl groups; aldehyde groups; azide groups; alkyne and alkene groups; ketones; hydrazides; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; hydroxyl groups; sulfhydryl groups; and molecules possessing, for example, alkyl, alkenyl, alkynyl, allylic, or benzylic leaving groups, such as halides, mesylates, tosylates, nosylates (e.g., para-nitrophenylsulfonates), triflates, epoxides, phosphate esters, sulfate esters, and besylates.

Non-limiting examples of the linking portion can include alkylene, alkenylene, alkynylene, polyether, such as polyethylene glycol (PEG), hydroxy carboxylic acids, polyester, polyamide, polyamino acids, polypeptides, cleavable peptides, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, and ester groups.

In some cases, a linker can comprise a cyclic group, such as an organic nonaromatic or aromatic ring, optionally with 3-10 carbons in the ring, optionally built from a carboxylic acid,

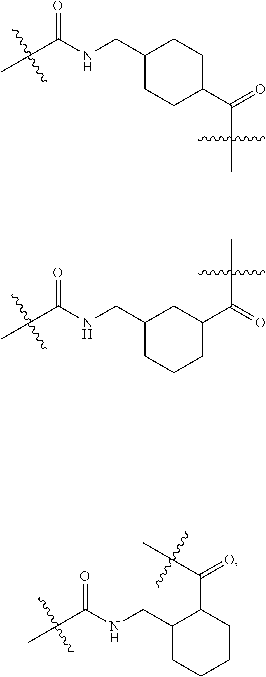 [SMILES: *C(=O)NCC1CCC(C(*)=O)CC1 ; *C(=O)NCC1CCCC(C(*)=O)C1 ; *C(=O)NCC1CCCCC1C(*)=O]

for example trans-4-(aminomethyl) cyclohexane carboxylic acid,

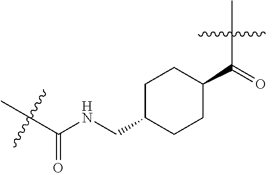 [SMILES: *C(=O)NC[C@H]1CC[C@H](C(*)=O)CC1]

or a substituted analog or a stereoisomer thereof. This linker can optionally be used to form a carbamate linkage. In some cases, a carbamate linkage can be more resistant to cleavage, such as by hydrolysis, enzymes such as esterases, or other chemical reactions, than an ester linkage.

In some cases, a linker can comprise a cyclic carboxylic acid, for example a cyclic dicarboxylic acid, for example one of the following groups: 1,4-cyclohexane dicarboxylic acid, 1,2-cyclohexane dicarboxylic acid, or 1,3-cyclohexane dicarboxylic acid, 1,1-cyclopentanediacetic acid,

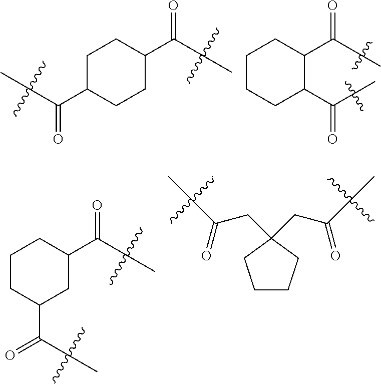

or a substituted analog or a stereoisomer thereof. For example, the linker can comprise one of the following groups:

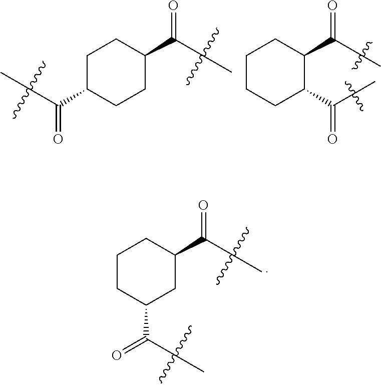

In some instances, the linker can optionally be used to form an ester linkage. In some cases, a cyclic ester linkage can be more sterically resistant to cleavage, such as by hydrolysis by water, enzymes such as esterases, or other chemical reactions, than a noncyclic or linear ester linkage.

In some cases, a linker can comprise an aromatic dicarboxylic acid, for example terephthalic acid, isophthalic acid, phthalic acid

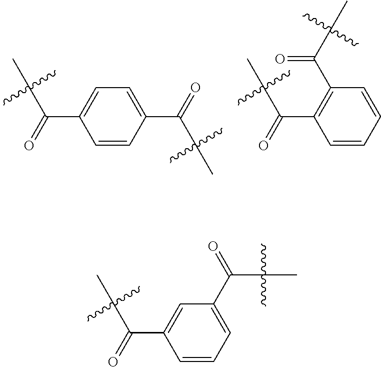 [SMILES: *C(=O)c1ccc(C(*)=O)cc1 ; *C(=O)c1ccccc1C(*)=O ; *C(=O)c1cccc(C(*)=O)c1]

or a substituted analog thereof.

In some cases, a linker can comprise a natural or non-natural amino acid, for example cysteine,

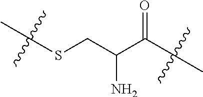 [SMILES: *SCC(N)C(*)=O]

or a substituted analog or a stereoisomer thereof. In some instances, a linker can comprise alanine (A, Ala); arginine (R, Arg); asparagine (N, Asn); aspartic acid (D, Asp); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val); or any plurality or combination thereof. In some embodiments, the non-natural amino acid can comprise one or more functional groups, e.g., alkene or alkyne, that can be used as functional handles.

In some cases, a linker can comprise one of the following groups:

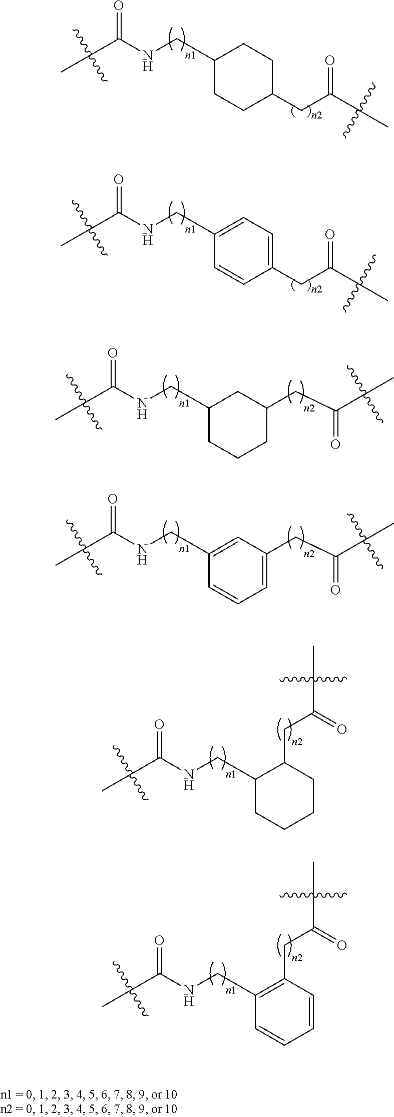

or a substituted analog or a stereoisomer thereof. In some instances, the linker is selected from one of the following groups:

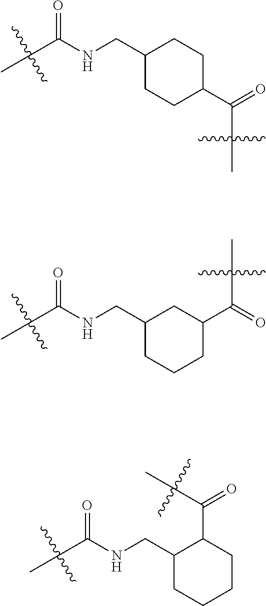 [SMILES: *C(=O)NCC1CCC(C(*)=O)CC1 ; *C(=O)NCC1CCCC(C(*)=O)C1 ; *C(=O)NCC1CCCCC1C(*)=O]

or a substituted analog or a stereoisomer thereof.

In some cases, a linker can comprise one of the following groups:

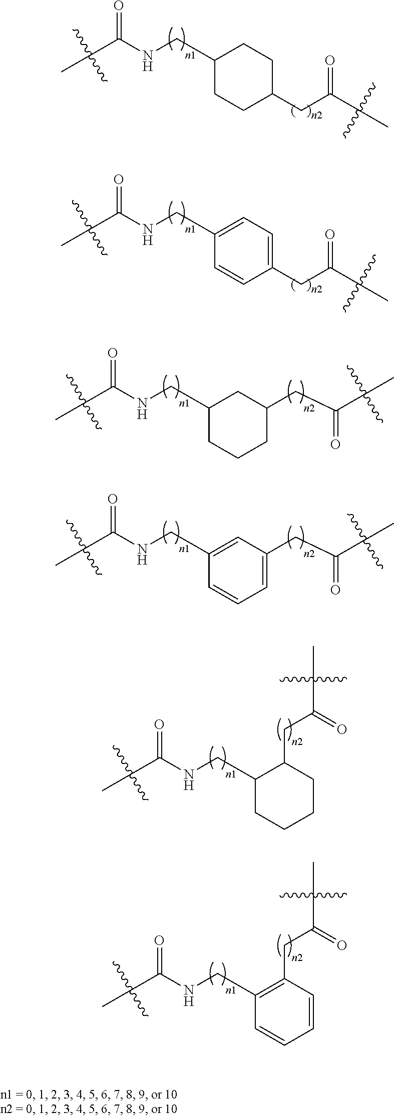

or a substituted analog or a stereoisomer thereof. In some instances, the linker is selected from one of the following groups:

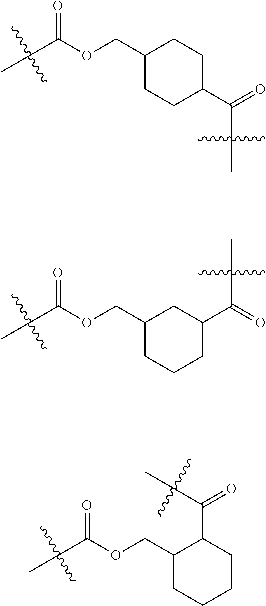 [SMILES: *C(=O)OCC1CCC(C(*)=O)CC1 ; *C(=O)OCC1CCCC(C(*)=O)C1 ; *C(=O)OCC1CCCCC1C(*)=O]

or a substituted analog or a stereoisomer thereof.

In some cases, a substituted analog or a stereoisomer is a structural analog of a compound disclosed herein, for which one or more hydrogen atoms of the compound can be substituted by one or more groups of halo (e.g., Cl, F, Br), alkyl (e.g., methyl, ethyl, propyl), alkenyl, alkynyl, arylalkyl, cycloalkyl, cycloalkylalkyl, heteroalkyl, heterocycloalkyl, or any combination thereof. In some cases, a stereoisomer can be an enantiomer, a diastereomer, a cis or trans stereoisomer, a E or Z stereoisomer, or a R or S stereoisomer.

Non-limiting examples of linear linkers include:

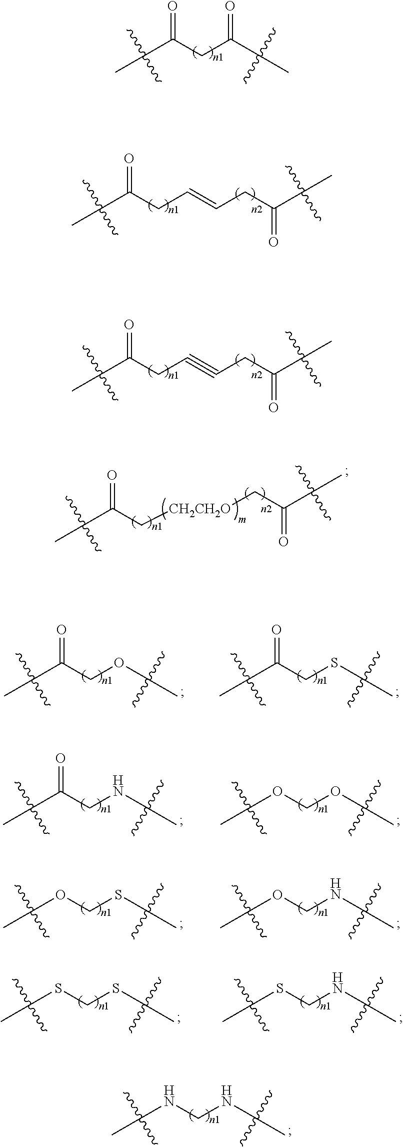

wherein each n1, n2 or m is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some instances, the linker can comprise a linear dicarboxylic acid, e.g., one of the following groups: succinic acid, 2,3-dimethyl succinic acid, glutaric acid, adipic acid, 2,5-dimethyladipic acid,

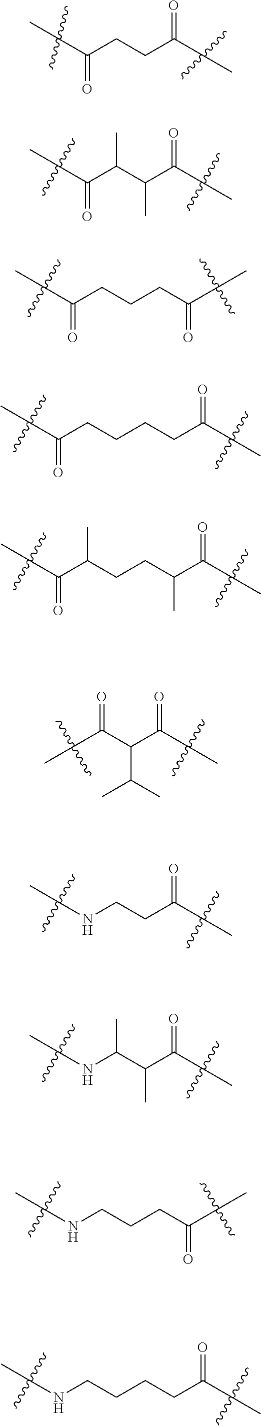

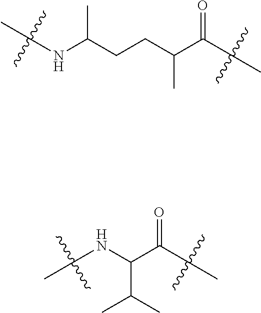

or a substituted analog or a stereoisomer thereof. In some cases, the linker can be used to form a carbamate linkage. In some embodiments, the carbamate linkage can be more resistant to cleavage, such as by hydrolysis, enzymes such as esterases, or other chemical reactions, than an ester linkage. In some cases, the linker can be used to form a linear ester linkage. In some embodiments, the linear ester linkage can be more susceptible to cleavage, such as by hydrolysis, enzymes such as esterases, or other chemical reactions, than a cyclic ester or carbamate linkage. Side chains such as methyl groups on the linear ester linkage can optionally make the linkage less susceptible to cleavage than without the side chains.

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some cases, a drug can be attached to a peptide using any one or more of the linkers shown below in TABLE 2, or any isomer, stereoisomer, or derivative thereof.

TABLE 2
Exemplary Linkers for Use in Peptide-Drug Conjugates

| Compound | | |
|---|---|---|
| number | Chemical structure | Linker is based on: |
| 1 | 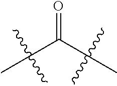 [SMILES: *C(*)=O] | Carbonic acid |
| 2 | 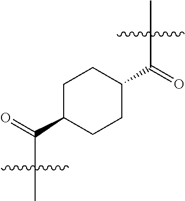 [SMILES: *C(=O)[C@H]1CC[C@H](C(*)=O)CC1] | trans-1,4- cyclohexanedicarboxylic acid |
| 3 | 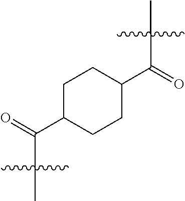 [SMILES: *C(=O)C1CCC(C(*)=O)CC1] | 1,4-cyclohexanedicarboxylic acid |
| 4 | 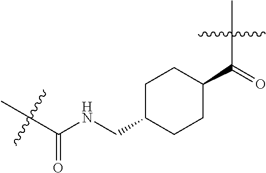 [SMILES: *C(=O)NC[C@H]1CC[C@H](C(*)=O)CC1] | trans-1-aminomethylamine- cyclohexane-4-carboxylic acid (carbamate) |
| 5 | 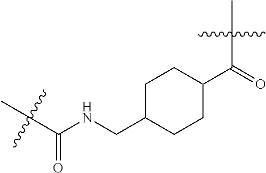 [SMILES: *C(=O)NCC1CCC(C(*)=O)CC1] | 1-aminomethylamine- cyclohexane-4-carboxylic acid |
| 6 | 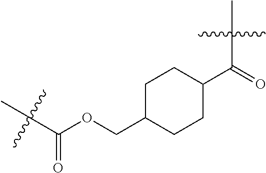 [SMILES: *C(=O)OCC1CCC(C(*)=O)CC1] | 4-(carboxyoxy)-methyl- cyclohexane-1-carboxylic acid |
| 7 | 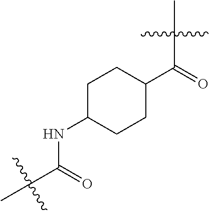 [SMILES: *C(=O)NC1CCC(C(*)=O)CC1] | 4-(carboxyamino)- cyclohexane-1-carboxylic acid |
| 8 | 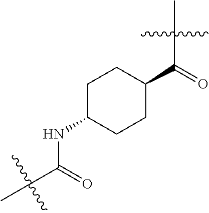 [SMILES: *C(=O)N[C@H]1CC[C@H](C(*)=O)CC1] | (1r,4r)-4-(carboxyamino)- cyclohexane-1-carboxylic acid |
| 9 | 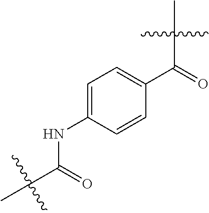 [SMILES: *C(=O)Nc1ccc(C(*)=O)cc1] | 4-(carboxyamino)benzoic acid |
| 10 | 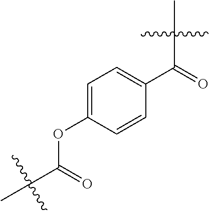 [SMILES: *C(=O)Oc1ccc(C(*)=O)cc1] | 4-(carboxyoxy)benzoic acid |
| 11 | 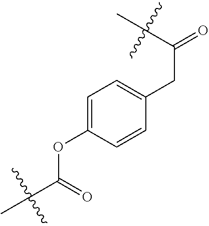 [SMILES: *C(=O)Cc1ccc(OC(*)=O)cc1] | 2-(4- (carboxyoxy)phenyl)acetic acid |
| 12 | 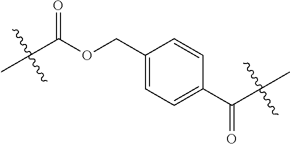 [SMILES: *C(=O)OCc1ccc(C(*)=O)cc1] | 4- ((carboxyoxy)methyl)benzoic acid |
| 13 | 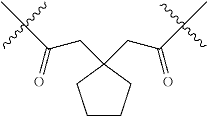 [SMILES: *C(=O)CC1(CC(*)=O)CCCC1] | 2,2′-(cyclopentane-1,1- diyl)diacetic acid |
| 14 | 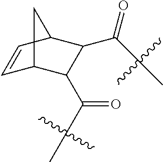 [SMILES: *C(=O)C1C2C=CC(C2)C1C(*)=O] | cis-5-norborene-endo-2,3- dicarboxylic acid |
| 15 | 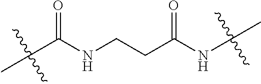 [SMILES: *NC(=O)CCNC(*)=O] | (3-amino-3- oxypropyl)carbamic acid |
| 16 | 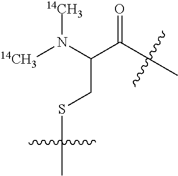 [SMILES: *SCC(C(*)=O)N([14CH3])[14CH3]] | [¹⁴C]-Cysteine |
| 17 | 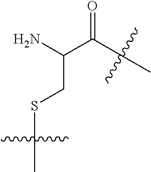 [SMILES: *SCC(N)C(*)=O] | Cysteine |
| 18 | 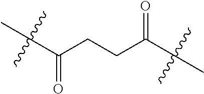 [SMILES: *C(=O)CCC(*)=O] | Succinic acid |
| 19 | 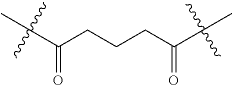 [SMILES: *C(=O)CCCC(*)=O] | Glutaric acid |
| 20 | 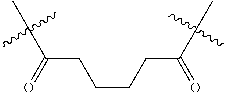 [SMILES: *C(=O)CCCCC(*)=O] | Adipic acid |
| 21 | 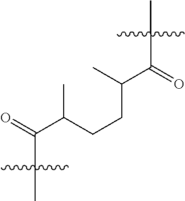 [SMILES: *C(=O)C(C)CCC(C)C(*)=O] | 2,5-dimethyladipic acid (DMA) |
| 22 | 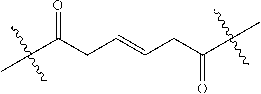 [SMILES: *C(=O)C/C=C/CC(*)=O] | trans-β-hydromuconic acid |

In some cases, a drug molecule is attached to a linker wherein a nucleophilic functional group (e.g., a hydroxyl group) of the drug molecule acts as the nucleophile and replaces a leaving group on the linker moiety, thereby attaching the drug to the linker. For instance, EXAMPLE 5 shows that a primary alcohol (e.g., a hydroxyl group) of the drug molecule (e.g., dexamethasone also abbreviated herein as “Dex”) reacts with a carboxylic acid of a linker (e.g., dimethyl adipic acid also abbreviated herein as “DMA”) in the presence of the activating agents DMAP and EDC to form an ester linkage that attaches the drug to the linker.

In other cases, a drug molecule is attached to a linker wherein a nucleophilic functional group (e.g., thiol group, amine group, etc) of the linker replaces a leaving group on the drug molecule, thereby attaching the drug to the linker. For instance, EXAMPLE 8 shows that a thiol group of a linker (e.g., cysteine, or ¹⁴C-labeled cysteine) can be the nucleophile and can react with a leaving group of the drug molecule dexamethasone. Such leaving group (or functional group that may be converted into a leaving group) may be a primary alcohol to form a thioether bond, thereby attaching the drug to the linker. A primary alcohol can be converted into a leaving group such as a mesylate, a tosylate, or a nosylate in order to accelerate the nucleophilic substitution reaction.

The peptide-drug conjugates of the present disclosure can comprise a drug, a linker, and/or a peptide of the present disclosure. A general connectivity between these three components can be drug-linker-peptide, such that the linker is attached to both the drug and the peptide. In most cases, the peptide is attached to a linker via an amide bond. Amide bonds can be relatively stable (e.g., in vivo) compared to other bonds described herein, such as esters, carbonates, etc. The amide bond between the peptide and the linker may thus provide advantageous properties due to its in vivo stability if the drug is sought to be cleaved from a peptide-drug-conjugate without the linker being attached to the drug after such in vivo cleavage. Thus, in various cases, a drug is attached to the linker-peptide moiety via linkages such as ester, carbonate, carbamate, etc., wherein the peptide is attached to the linker via an amide bond. This can allow for selective cleavage of the drug-linker bond (as opposed to the linker-peptide bond) allowing the drug to be released without a linker moiety attached to it after cleavage. The use of such different drug-linker bonds or linkages can allow the modulation of drug release in vivo, e.g., in order to achieve a therapeutic function while minimizing off-target effects (e.g., reduction of drug release during circulation).

In some cases, the use of a stable linker is suitable for a specific application of a peptide-drug conjugate. In other cases, a cleavable linker is suitable for a specific application of a peptide-drug conjugate, e.g., to release an active and/or detectable agent at a target site, cell, organ, or tissue (e.g., cartilage). Thus, the linker moieties used in the herein described peptide-drug conjugates can be a cleavable or a stable linker. The use of a cleavable linker permits release of the conjugated moiety (e.g., a therapeutic agent) from the peptide, e.g., after targeting to the cartilage. In some cases the linker is enzyme cleavable, e.g., a valine-citrulline linker that can be cleavable by cathepsin, or an ester linker that can be cleavable by esterase. In some embodiments, the linker contains a self-immolating portion. In other embodiments, the linker includes one or more cleavage sites for a specific protease, such as a cleavage site for matrix metalloproteases (MMPs), thrombin, urokinase-type plasminogen activator, or cathepsin (e.g., cathepsin K).

Thus, in some cases, a peptide-active agent conjugate of the present disclosure can comprise one or more, about two or more, about three or more, about five or more, about ten or more, or about 15 or more amino acids that can form an amino acid sequence cleavable by an enzyme. Such enzymes can include proteinases. A peptide-drug conjugate can comprise an amino acid sequence that can be cleaved by a Cathepsin, a Chymotrypsin, an Elastase, a Subtilisin, a Thrombin I, or a Urokinas, or any combination thereof.

Alternatively or in combination, the cleavable linker can be cleaved, dissociated, or broken by other mechanisms, such as via pH, reduction, or hydrolysis. Hydrolysis can occur directly due to water reaction, or be facilitated by an enzyme, or be facilitated by presence of other chemical species. A hydrolytically labile linker, (amongst other cleavable linkers described herein) can be advantageous in terms of releasing active agents from the peptide. For example, an active agent in a conjugate form with the peptide may not be active, but upon release from the conjugate after targeting to the cartilage, the active agent is active. The cleaved active agent may retain the chemical structure of the native active agent before cleavage, or may be modified. In some embodiments, a stable linker may optionally not cleave in buffer over extended periods of time (e.g., hours, days, or weeks). In some embodiments, a stable linker may optionally not cleave in body fluids such as plasma or synovial fluid over extended periods of time (e.g., hours, days, or weeks). In some embodiments, a stable linker optionally may cleave, such as after exposure to enzymes, reactive oxygen species, other chemicals or enzymes that may be present in cells (such as macrophages), cellular compartments (such as endosomes and lysosomes), inflamed areas of the body (such as inflamed joints), or tissues or body compartments. In some embodiments, a stable linker may optionally not cleave in vivo but present an active agent that is still active when conjugated to the peptide.

The rate of hydrolysis of the linker (e.g., a linker of a peptide conjugate) can be tuned. For example, the rate of hydrolysis of linkers with unhindered esters is faster compared to the hydrolysis of linkers with bulky groups next an ester carbonyl. A bulky group can be a methyl group, an ethyl group, a phenyl group, a ring, or an isopropyl group, or any group that provides steric bulk. In another example, the rate of hydrolysis can be faster with hydrophilic groups, such as alcohols, acids, or ethers, or near an ester carbonyl. In another example, hydrophobic groups present as side chains or by having a longer hydrocarbon linker can slow cleavage of the ester. In some embodiments, cleavage of a carbamate group can also be tuned by hindrance, hydrophobicity, and the like. In another example, using a less labile linker, such as a carbamate rather than an ester, can slow the cleavage rate of the linker. In some cases, the steric bulk can be provided by the drug itself, such as by ketorolac when conjugated via its carboxylic acid. The rate of hydrolysis of the linker can be tuned according to the residency time of the conjugate in the cartilage, according to how quickly the peptide accumulates in the cartilage, or according to the desired time frame for exposure to the active agent in the cartilage. For example, when a peptide is cleared from the cartilage relatively quickly, the linker can be tuned to rapidly hydrolyze. In contrast, for example, when a peptide has a longer residence time in the cartilage, a slower hydrolysis rate can allow for extended delivery of an active agent. This can be important when the peptide is used to deliver a drug to the cartilage. “Programmed hydrolysis in designing paclitaxel prodrug for nanocarrier assembly” Sci Rep 2015, 5, 12023 Fu et al., provides an example of modified hydrolysis rates. In some embodiments, rates of cleavage can vary by species, body compartment, and disease state. For instance, cleavage by esterases may be more rapid in rat or mouse plasma than in human plasma, such as due to different levels of carboxyesterases. In some embodiments, a linker may be tuned for different cleavage rates for similar cleavage rates in different species.

The rate of hydrolysis of the linker (e.g., a linker of a peptide conjugate) can be measured. Such measurements can include determining free active agent in plasma, or synovial fluid, or other fluid or tissue of a subject in vivo and/or by incubating a linker or a peptide conjugate comprising a linker of the present disclosure with a buffer (e.g., PBS) or blood plasma from a subject (e.g., rat plasma, human plasma, etc.) or synovial fluid or other fluids or tissues ex vivo. The methods for measuring hydrolysis rates can include taking samples during incubation or after administration and determine free active agent, free peptide, or any other parameter indicate of hydrolysis, including also measuring total peptide, total active agent, or conjugated active agent-peptide. The results of such measurements can then be used to determine a hydrolysis half-life of a given linker or peptide conjugate comprising the linker. A hydrolysis half-life of a linker can differ depending on the plasma or fluid or species or other conditions used to determine such half-life. This can be due to certain enzymes or other compounds present in a certain plasma (e.g., rat plasma). For instance, different fluids (such as plasma or synovial fluid) can contain different amounts of enzymes such as esterases, and these levels of these compounds can also vary depending on species (such as rat versus human) as well as disease state (such as normal versus arthritic).

The conjugates of the present disclosure can be described as having a modular structure comprising various components, wherein each of the components (e.g., peptide, linker, active agent and/or detectable agent) can be selected dependently or independently of any other component. For example, a conjugate for use in the treatment of arthritis can comprise a cartilage-targeting peptide of the present disclosure (e.g., those having the amino acid sequence of any one of SEQ ID NO: 21-SEQ ID NO: 247 or SEQ ID NO: 282-SEQ ID NO: 510), a linker (e.g., any linker described in TABLE 2) and an active agent (e.g., a glucocorticoid). The linker, for example, can be selected and/or modified to achieve a certain active agent release (e.g., a certain release rate) via a certain mechanism (e.g., via hydrolysis, such as enzyme and/or pH-dependent hydrolysis) at the target site (e.g., in the cartilage) and/or to minimize systemic exposure to the active agent. During the testing of a conjugate any one or more of the components of the conjugate can be modified and/or altered to achieve certain in vivo properties of the conjugate, e.g., pharmacokinetic (e.g., clearance time, bioavailability, uptake and retention in various organs) and/or pharmacodynamic (e.g., target engagement) properties. Thus, the conjugates of the present disclosure can be modulated to prevent, treat, and/or diagnose a variety of diseases and conditions, while reducing side effects (e.g., side effects that occur if such active agents are administered alone (i.e., not conjugated to a peptide)).

In some embodiments, a conjugate as described herein comprises one or more non-natural amino acid and/or one or more linkers. Such one or more non-natural amino acid and/or one or more linkers can comprise one or more functional groups, e.g., alkene or alkyne (e.g., non-terminal alkenes and alkynes), which can be used as functional handles. For example, a multiple bond of such functional groups can be used to add one or more molecules to the conjugate. The one or more molecules can be added using various synthetic strategies, some of which may include addition and/or substitution chemistries, cycloadditions, etc. For example, an addition reaction using a multiple bond can comprise the use of hydrogen bromide (e.g., via hydrohalogenation reactions), wherein the bromide substituent, once attached, can act as a leaving group and thus be substituted with various moieties comprising a nucleophilic functional groups, e.g., active agents, detectable agents, agents. As another example, a multiple bond can be used as a functional handle in a cycloaddition reaction. Cycloaddition reactions can comprise 1,3-dipolar cycloadditions, [2+2]-cycloadditions (e.g., photocatalyzed), Diels-Alder reactions, Huisgen cycloadditions, nitrone-olefin cycloadditions, etc. Such cycloaddition reactions can be used to attached various functional groups, functional moieties, active agents, detectable agents, and so forth to the conjugate. For example, a 1,3-dipolar cycloaddition reaction can be used to attach a molecule to a conjugate, wherein the molecule comprises a 1,3-dipole that can react with, e.g., an alkyne to form a 5-membered ring, thereby attaching said molecule to the conjugate.

The addition of such agents or molecules (e.g., via nucleophilic or electrophilic addition followed by nucleophilic substitution) can have various application. For example, attaching such molecule or agent can modify or alter the pharmacokinetic (e.g., plasma half-life, retention and/or uptake in cartilage or biodistribution) and/or pharmacodynamic (e.g., hydrolysis rate such as an enzymatic hydrolysis rate) properties of the conjugate. Attaching such molecule or agent can also alter (e.g., increase) the depot effect of a conjugate, or provide functionality for in vivo tracking, e.g., using fluorescence or other types of detectable agents.

In some cases, a conjugate of the present disclosure can comprise one or more non-terminal alkenes and/or alkynes. In some cases, a conjugate of the present disclosure can comprise a linker comprising one or more of the following groups:

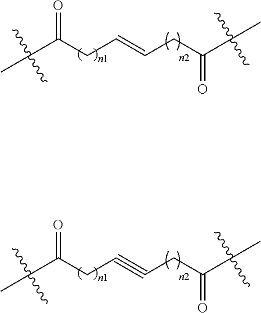

or a substituted analog or a stereoisomer thereof, wherein each n1 and n2 is independently a value from 1 to 10. Such a group can be used as a handle to attach one or more molecules to a conjugate, e.g., to alter the pharmacokinetic (e.g., plasma half-life, retention and/or uptake in cartilage) and/or pharmacodynamic properties of the conjugate. Functionalization of such a group can occur using one or more multiple bonds (e.g., double bonds, triple bonds, etc.) of the groups (e.g., non-terminal alkenes and alkynes). Such functionalization can comprise addition and/or substitution chemistries and cycloaddition reactions as described herein. For example, a functional group of a linker, such as a double bond, can be converted into a single bond (e.g., via an addition reaction such as a nucleophilic/electrophilic addition reaction), wherein one or both of the carbon atoms of the newly formed single bond can have a leaving group (e.g., a bromide) attached to them. Such a leaving group can then be used (e.g., via nucleophilic substitution reaction) to attach a specific molecule (e.g., an active agent, a detectable agent, etc.) to that carbon atom(s) of the linker. As another example, a multiple bond can be used as a functional handle in a cycloaddition reaction. Cycloaddition reactions can comprise 1,3-dipolar cycloadditions, [2+2]-cycloadditions (e.g., photocatalyzed), Diels-Alder reactions, Huisgen cycloadditions, nitrone-olefin cycloadditions, etc. Such cycloaddition reactions can be used to attached various functional groups, functional moieties, active agents, detectable agents, and so forth to the conjugate. For example, a 1,3-dipolar cycloaddition reaction can be used to attach a molecule to a conjugate, wherein the molecule comprises a 1,3-dipole that can react with, e.g., an alkyne to form a 5-membered ring, thereby attaching said molecule (e.g., active agent, detectable agent, etc.) to the conjugate. In some cases, molecules may be attached to a conjugate to e.g., modulate the half-life, increase the depot effect, or provide new functionality of a conjugate, such as fluorescence for tracking.

### Peptide Stability

A peptide or a conjugate of the present disclosure can be stable in various biological conditions. For example, any peptide of SEQ ID NO: 1-SEQ ID NO: 510 or a conjugate comprising thereof can exhibit resistance to reducing agents, proteases, oxidative conditions, or acidic conditions.

In some cases, biologic molecules (such as peptides and proteins) can provide therapeutic functions, but such therapeutic functions are decreased or impeded by instability caused by the in vivo environment. (Moroz et al. Adv Drug Deliv Rev 101:108-21 (2016), Mitragotri et al. Nat Rev Drug Discov 13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g. pH˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they may be unable to provide a therapeutic function on intracellular targets. Therefore, conjugates disclosed herein can comprise peptides that are resistant to reducing agents, proteases, and low pH (e.g., pH 1-2 or pH 1-3). In some cases, the conjugates may be able to provide enhanced therapeutic effects or enhance the therapeutic efficacy of co-formulated or conjugated active agents in vivo.

Additionally, oral delivery of drugs can be desirable in order to target certain areas of the body despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides and peptides of conjugates without nullifying their therapeutic function.

### Peptide Resistance to Reducing Agents

Peptides of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body and in cells, and can reduce disulfide bonds. As another example, a peptide can become reduced upon cellular internalization during trafficking of a peptide across the gastrointestinal epithelium after oral administration. Peptides can be reduced by exposure to blood. A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life including cartilage homing and accumulation. Reduction of the disulfide bonds can also lead to increased susceptibility of the peptide to subsequent degradation by proteases, resulting in rapid loss of intact peptide (or peptide conjugate) after administration. In some embodiments, a peptide that is resistant to reduction can remain intact and can impart a functional activity for a longer period of time in various compartments of the body and in cells, as compared to a peptide that is more readily reduced.

In certain embodiments, the peptides of the conjugates of this disclosure can be analyzed for the characteristic of resistance to reducing agents to identify stable peptides. In some embodiments, the peptides of this disclosure can remain intact after being exposed to different molarities of reducing agents such as 0.00001M-0.0001M, 0.0001M-0.001M, 0.001M-0.01M, 0.01 M-0.05 M, 0.05 M-0.1 M, for greater 15 minutes or more. In some embodiments, the reducing agent used to determine peptide stability can be dithiothreitol (DTT), Tris (2-carboxyethyl)phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent.

### Peptide Resistance to Proteases

The stability of peptides of the conjugates of this disclosure can be determined by resistance to degradation by proteases. Proteases, also referred to as peptidases or proteinases, can be enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, esterases (including carboxyesterases), serum proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, peptides of conjugates of this disclosure can resist degradation by any class of protease. In certain embodiments, peptides of this disclosure resist degradation by pepsin (which can be found in the stomach), trypsin (which can be found in the duodenum), serum proteases, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, elafin), or any combination thereof. In some embodiments, the proteases used to determine peptide stability can be pepsin, trypsin, chymotrypsin, or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%400% of the peptide remains intact after exposure to a protease. Peptides of SEQ ID NO: 196, SEQ ID NO: 24, and SEQ ID NO: 105 can have particular structural qualities, which make them more resistant to protease degradation. For example, peptide of SEQ ID NO: 24 and SEQ ID NO: 106 exhibit the “hitchin” topology as described previously, which can be associated with resistance to protease and chemical degradation.

### Peptide Stability in Acidic Conditions

Conjugates comprising peptides of this disclosure can be administered in biological environments that are acidic. For example, after oral administration, the conjugates or peptides can experience acidic environmental conditions in the gastric fluids of the stomach and gastrointestinal (GI) tract. The pH of the stomach can range from ˜1-4 and the pH of the GI tract ranges from acidic to normal physiological pH descending from the upper GI tract to the colon. In addition, the vagina, late endosomes, and lysosomes can also hav acidic pH values, such as less than pH 7. These acidic conditions can lead to denaturation of peptides and proteins into unfolded states. Unfolding of peptides and proteins can lead to increased susceptibility to subsequent digestion by other enzymes as well as loss of biological activity of the peptide.

In certain embodiments, the peptides of conjugates of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5-10%, at least 10%-20%, at least 20%-30%, at least 30%-40%, at least 40%-50%, at least 50%-60%, at least 60%-70%, at least 70%-80%, at least 80%-90%, or at least 90-100% of the peptide remains intact after exposure to simulated gastric fluid. In some embodiments, low pH solutions such as simulated gastric fluid or citrate buffers can be used to determine peptide stability.

### Peptide Stability at High Temperatures

Conjugate comprising peptides of this disclosure can be administered in biological environments with high temperatures. For example, after oral administration, the conjugates or peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

### Pharmacokinetics of Conjugates

The pharmacokinetics of conjugates of this disclosure can be determined after administration of the conjugate via different routes of administration. For example, the pharmacokinetic parameters of a conjugate of this disclosure can be quantified after intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, intra-articular, peritoneal, buccal, synovial, or topical administration. Conjugates of the present disclosure can be analyzed by using tracking agents such as radiolabels or fluorophores. For example, a conjugate comprising a radiolabeled peptide of this disclosure can be administered via various routes of administration. Peptide or conjugate concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein can relate to pharmacokinetics of a conjugate administered via any route to a subject. Pharmacokinetics can be described using methods and models, for example, compartmental models or noncompartmental methods. Compartmental models include but are not limited to monocompartmental model, the two compartmental model, the multicompartmental model or the like. Models can be divided into different compartments and can be described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion can be grouped into one compartment referred to as the elimination compartment. For example, liberation can include liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition is often biphasic, including, for example an alpha phase and a beta phase, occasionally a gamma, delta or other phase is observed.

Pharmacokinetics includes determining at least one parameter associated with administration of a conjugate to a subject. In some aspects, parameters include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach Cmax (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

In certain embodiments, conjugates comprising the peptides of any of SEQ ID NO: 1-SEQ ID NO: 510 exhibit optimal pharmacokinetic parameters after oral administration. In other embodiments, the peptides of any of SEQ ID NO: 1-SEQ ID NO: 510 exhibit optimal pharmacokinetic parameters after any route of administration, such as oral administration, inhalation, intranasal administration, topical administration, intravenous administration, subcutaneous administration, intra-articular administration, intramuscular administration, intraperitoneal administration, or any combination thereof.

In some embodiments any peptide of SEQ ID NO: 1-SEQ ID NO: 510 exhibits an average Tₘₐₓ of 0.5-12 hours, or 1-48 hours at which the Cₘₐₓ is reached, an average bioavailability in serum of 0.1%-10% in the subject after administering the conjugate to the subject by an oral route, an average bioavailability in serum of less than 0.1% after oral administration to a subject for delivery to the GI tract, an average bioavailability in serum of 10-100% after parenteral administration, an average t_{1/2} of 0.1 hours-168 hours, or 0.25 hours-48 hours in a subject after administering the conjugate to the subject, an average clearance (CL) of 0.5-100 L/hour or 0.5-50 L/hour of the conjugate after administering the conjugate to a subject, an average volume of distribution (V_{d}) of 200-20,000 mL in the subject after systemically administering the conjugate to the subject, or optionally no systemic uptake, any combination thereof.

Pharmacokinetic parameters such as biodistribution, organ clearance and/or clearance from circulation, and organ distribution, uptake and/or retention can be determined for any peptide and/or peptide conjugate described herein. Such parameters can be determined using various techniques in vivo and/or ex vivo. Pharmacokinetic parameters of a peptide or peptide conjugate can be determined using, for example, a detectable agent that is attached to the peptide or peptide conjugate. As further described herein, such detectable agent can be a fluorophore or a radioactive isotope. In some cases, a peptide or peptide conjugate is labeled with ¹⁴C to determine one or more pharmacokinetic parameters. In such cases, pharmacokinetic parameters can be determined in vivo and ex vivo by using, for example, quantitative whole body autoradiography (QWBA) and/or scintillation counting, respectively. In some cases, a peptide or peptide conjugate is labeled and/or conjugated to a fluorophore, allowing for the determination of peptide or peptide conjugate distribution in a subject, organ, and/or tissue in vivo and ex vivo.

### Methods of Manufacture

### Production of Peptides

Various expression vector/host systems can be utilized for the production of the recombinant expression of peptides of the conjugates described herein. Non-limiting examples of such systems include microorganisms such as bacteria (e.g., E. coli) transformed with recombinant bacteriophage DNA, plasmid DNA or cosmid DNA expression vectors containing a nucleic acid sequence encoding peptides or peptide fusion proteins/chimeric proteins described herein, yeast (e.g., pichia) transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal such as mammalian cell systems (e.g., CHO or HEK) infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secretes minimal amounts of proteolytic enzymes.

In some instances, the peptide is secreted from the cells into media and can be harvested. In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, Peptides can also be synthesized in a cell-free system using a variety of known techniques employed in protein and peptide synthesis.

In some cases, a peptide of the present disclosure is produced recombinantly alone or as a fusion peptide or fusion protein. In some embodiments, disclosed herein are methods that can express the peptide as a C-terminal fusion to a larger protein. In some cases, a peptide is expressed as a C-terminal fusion protein comprising siderocalin as an additional protein. In some cases, such fusions direct the fusion peptide or protein through a mammalian secretory pathway. In some cases, such processes ensure proper formation of the disulfide bond structure of a cystine-dense peptide of the present disclosure. Upon expression, the C-terminal fusion protein (e.g., siderocalin) can be cleaved from the peptide by an optimized TEV enzyme. In some cases, co-expression of a protease or chemical cleavage is used. Expressed fusion proteins can be purified using various methods. Such methods can include Ni-NTA capture of the His tag encoded upstream of the C-terminal fusion protein (e.g., siderocalin), which can be followed by TEV cleavage and peptide purification using, for example, chromatography (e.g., size-exclusion chromatography, reversed-phase (RP) high-pressure liquid chromatography (HPLC), and/or RP-fast protein liquid chromatography (FPLC)). Different purification techniques can be advantageous for manufacturing where the His-tag may be absent and where use of organic solvents may not be used. As an example, the two peptides having the amino acid sequences set forth in SEQ ID NO: 105 and SEQ ID NO: 184 can be expressed in CHO—S cells by transient transfection. Each peptide can be expressed as a siderocalin fusion or as the peptide alone. The peptide may or may not contain any predicted glycosylation sites or other post-translational modifications.

In some cases, a host cell produces a peptide that has an attachment point for a drug. An attachment point could comprise a lysine residue, an N-terminus, a cysteine residue, a cysteine disulfide bond, or a non-natural amino acid. The peptide could also be produced synthetically, such as by solid-phase peptide synthesis, or solution-phase peptide synthesis. The peptide could be folded (formation of disulfide bonds) during synthesis or after synthesis or both. Peptide fragments could be produced synthetically or recombinantly and then joined together synthetically, recombinantly, or via an enzyme.

In some aspects, the peptides of conjugates of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000) or Boc solid phase peptide synthesis or by solution phase peptide synthesis. The peptide can be folded with disulfide bond formation during peptide synthesis, after peptide synthesis, before or after cleavage from the resin or polymer, or stepwise in both. Disulfide bond formation can occur by exposure to air, oxidants, catalysts, or reduced/oxidized pairs, or in a buffer.

In some aspects, chemical synthesis can be used to incorporate unnatural amino acids with functional groups (e.g., alkenes, alkynes, leaving groups, etc.) into a peptide of the disclosure. This functional group can be used as a functional handle. For example, a multiple bond of such functional groups can be used to add one or more molecules to the conjugate. The one or more molecules can be added using various synthetic strategies, some of which may include addition and/or substitution chemistries. For example, an addition reaction using a multiple bond can comprise the use of hydrogen bromide, wherein the bromide can act as a leaving group and thus be substituted with various moieties comprising a nucleophilic functional group, e.g., active agents, detectable agents, agents that can modify or alter the pharmacokinetic (e.g., plasma half-life, retention and/or uptake in cartilage) and/or pharmacodynamic properties of the conjugate, or molecules with other suitable properties.

### Synthesis of Peptide-Drug Conjugates

Peptide-drug conjugates (PDC) of the present disclosure can comprise one or more of any peptide described herein (e.g., those listed in TABLE 1 and/or those having the amino acid sequences set forth in SEQ ID NO: 1-SEQ ID NO: 510), any linker described herein (e.g., those listed in TABLE 2), and any active and/or detectable agent described herein.

Generally, a synthetic method for producing a peptide-drug conjugate as described herein can comprise attaching a drug molecule to a linker, followed by the attachment of a peptide to the drug-linker conjugate. Solvents, reaction conditions, and/or additional reagents used during synthesis may be selected such that yield and purity allow scaling the production of a peptide-drug conjugate (PDC) for preclinical and/or clinical studies while retaining the biological activity of the peptide and/or the active or detectable agent used in the PDC.

Peptide-drug conjugates (PDC) of the present disclosure can be synthesized using various synthetic strategies. Examples of such synthetic strategies are shown in EXAMPLE 5-EXAMPLE 19, and EXAMPLE 29. PDC's of the present disclosure can be synthesized using various chemical reactions and/or chemical transformations. In some embodiments, a PDC is synthesized using hydrolysis, ester bond formation(s), NHS ester formation(s), amide formation(s), peptide conjugation(s), a carbamate formation(s), mesylate formation(s), sulfur alkylation(s), reductive amination(s), deprotection(s), or any combination thereof. A peptide conjugation reaction can include but is not limited to an amide bond formation, a carbamate formation, a carbonate formation, an ester bond formation, or a combination thereof. Synthetic approaches for producing PDCs can comprise use of one or more protecting groups (e.g., Boc, Fmoc, MOM, etc), and, as such, can include one or more protection and/or deprotection steps. In some cases, a PDC synthesis comprises the formation of activated carboxylic acids such as a conversion of a carboxylic acid into an activated ester such as an NHS ester.

In some cases, the synthesis of a PDC comprises a radiolabeling step. Radiolabeling can comprise attaching one or more radionuclides to a PDC, e.g., the linker, and/or the peptide. The radionuclide can be ¹⁴C. Radiolabeling of peptides with ¹⁴C can comprise reductive amination. Such radiolabeling using ¹⁴C can comprise adding one or more ¹⁴CH₃ groups to a molecule such as a drug, a peptide, a linker, or a PDC. For example, a cysteine linker can be labeled with one or two ¹⁴CH₃ groups via its free amino group. For example, the term “¹⁴C-Cys-Dex” can comprise cysteine-Dex conjugates wherein the cysteine contains one or more ¹⁴CH₃ groups. Thus, in some cases, ¹⁴C-Cys-Dex comprises two ¹⁴CH₃ groups attached to its amino group. This principle is applicable to any molecule described herein.

Radiolabeling of PDCs with other radionuclides as described herein can comprise use of different synthetic strategies and/or use of chelator moieties. In some embodiments, a peptide and/or a peptide-drug conjugate is radiolabeled. A peptide and/or a peptide-drug conjugate can be radiolabeled with various radionuclides suitable for determining pharmacokinetic and/or pharmacodynamic (PD) parameters such as plasma half-life, organ and/or tissue uptake and/or retention, target engagement, etc.

In some embodiments, a peptide conjugate comprising a 2,5-dimethyladipic acid (DMA) linker and dexamethasone (also referred to herein as “Dex”) that can be synthesized using any one or more of hydrolysis, ester bond formation, NHS ester formation, and peptide conjugation. For example, a peptide conjugate comprising a peptide having the amino acid sequence set forth in SEQ ID NO: 105, a (carbamate) trans-1-aminomethyl-cyclohexyl-4-carboxylic linker and dexamethasone can be synthesized using any one or more of carbamate formation, NHS ester formation, peptide conjugation, or any combination thereof.

In some embodiments, a synthesis of a PDC of the present disclosure comprises any one or more of mesylate formation, sulfur alkylation, NHS ester formation, peptide conjugation, Boc deprotection, reductive amination, or any combination thereof. For example, a peptide-drug conjugate comprising a cysteine linker and triamcinolone acetonide (TAA) can be synthesized using mesylate formation, sulfur alkylation, NHS ester formation, peptide conjugation, Boc deprotection, reductive amination, or any combination thereof.

In some embodiments, a PDC comprising any one of the peptides which amino acid sequence is set forth in any one of SEQ ID NO: 1-SEQ ID NO: 510, a drug (e.g., glucocorticoid such as dexamethasone, TAA, or des ciclesonide), and any one of a glutaric acid linker, 3,3-tetramethylene-glutaric linker, a trans-1,2-cyclohexyl linker, a 1,3-cyclohexyl linker, a terephthalic acid linker, a 2,3-dimethyl-succinic acid linker, a succinic acid linker, a adipic acid linker, a trans-1,4-cyclohexyl linker can be produced using any one or more of ester bond formation, NHS ester formation, peptide conjugation, or any combination thereof. In some embodiments, a peptide-drug conjugate of the present disclosure comprises a peptide of the present disclosure linked to a glucocorticoid via a linker. In some cases, the peptide comprises an amino acid sequence set forth in any one of SEQ ID NO: 103, SEQ ID NO: 105, SEQ ID NO: 184, or SEQ ID NO: 509. In some cases, the glucocorticoid is dexamethasone or des-Ciclesonide. In some cases, the linker is any linker listed in TABLE 2 having compound number 1-22. The PCD comprising the peptide having the amino acid sequence set forth in SEQ ID NO: 105 (peptide(SEQ ID NO: 105)) linked to dexamethasone (i.e., Dex) via a cysteine linker (Cys), peptide(SEQ ID NO: 105)-Cys-Dex (“peptide(SEQ ID NO: 105)-Cys” is disclosed as SEQ ID NO: 512), can be synthesized using any one or more of mesylate formation, sulfur alkylation, NHS ester formation, peptide conjugation, Boc deprotection, reductive amination (see e.g., EXAMPLE 8). As another example, the PDC peptide(SEQ ID NO: 105)-DMA-dCIC, peptide(SEQ ID NO: 103)-DMA-dCIC, and/or peptide(SEQ ID NO: 184)-DMA-dCIC can be synthesized using the following three synthetic steps of ester bond formation, sulfo-NHS ester formation, and peptide conjugation as described herein.

### Pharmaceutical Compositions of Conjugates

A pharmaceutical composition of the disclosure can be a combination of any conjugate described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a conjugate described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, intraarticular, and topical administration. A pharmaceutical composition can be administered in a local or systemic manner, for example, via injection directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a conjugate described herein in water soluble form. Suspensions of conjugates described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such conjugates described herein to allow for the preparation of highly concentrated solutions. Alternatively, the conjugates described herein can be lyophilized or in powder form for re-constitution with a suitable vehicle, e.g., sterile pyrogen-free water, before use. In some embodiments, a purified conjugate is administered intravenously.

A conjugate of the disclosure can be applied directly to an organ, or an organ tissue or cells, such as brain or brain tissue or cancer cells, during a surgical procedure. The conjugate described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the conjugate described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a conjugate described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes before conjugation to an active agent or anti-arthritic agent such as anti-inflammatory agent. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the conjugate described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Administration of Pharmaceutical Compositions

In various aspects, the present disclosure provides a pharmaceutical composition comprising any of the conjugate disclosed herein or a salt thereof, and a pharmaceutically acceptable carrier.

In further aspects, the pharmaceutical composition is formulated for administration to a subject. In still further aspects, the pharmaceutical composition is formulated for inhalation, intranasal administration, oral administration, topical administration, intravenous administration, subcutaneous administration, intra-articular administration, intramuscular administration, intraperitoneal administration, or a combination thereof.

A pharmaceutical composition of the disclosure can be a combination of any conjugate described herein with other chemical components, such as carriers, stabilizers, diluents, dispersing agents, suspending agents, thickening agents, and/or excipients. The pharmaceutical composition facilitates administration of a conjugate described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, inhalation, dermal, intra-articular, intrathecal, intranasal, and topical administration. A pharmaceutical composition can be administered in a local or systemic manner, for example, via injection directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a conjugate described herein in water-soluble form. Suspensions of conjugates described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such conjugates described herein to allow for the preparation of highly concentrated solutions. Alternatively, the conjugates described herein can be lyophilized or in powder form for re-constitution with a suitable vehicle, e.g., sterile pyrogen-free water, before use. In some embodiments, a purified conjugate is administered intravenously. A conjugate described herein can be administered to a subject, home, target, migrates to, is retained by, and/or binds to, or be directed to an organ, e.g., the cartilage.

A conjugate of the disclosure can be applied directly to an organ, or an organ tissue or cells, such as cartilage or cartilage tissue or cells, during a surgical procedure. The conjugate described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the conjugate described herein described herein are administered in pharmaceutical compositions to a subject suffering from a condition. In some instances the pharmaceutical composition can affect the physiology of the animal, such as the immune system, inflammatory response, or other physiologic affect. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions that comprise a conjugate comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of the pharmaceutical compositions described herein include formulating the conjugate described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Use of Conjugates in Imaging and Surgical Methods

The present disclosure generally relates to conjugates that home, target, migrate to, are retained by, accumulate in, and/or bind to, or are directed to specific regions, tissues, structures, or cells within the body and methods of using such conjugates. These conjugates have the ability to contact the cartilage, which makes them useful for a variety of applications. In particular, the conjugates can have applications in site-specific modulation of biomolecules to which the peptides are directed to. End uses of such conjugates can include, for example, imaging, research, therapeutics, theranostics, pharmaceuticals, chemotherapy, chelation therapy, targeted drug delivery, and radiotherapy. Some uses can include targeted drug delivery and imaging.

In some embodiments, the present disclosure provides a method for detecting a cancer, cancerous tissue, or tumor tissue, the method comprising the steps of contacting a tissue of interest with a conjugate of the present disclosure, wherein the conjugate is further conjugated to a detectable agent and measuring the level of binding of the peptide of the conjugate, wherein an elevated level of binding, relative to normal tissue, is indicative that the tissue is a cancer, cancerous tissue or tumor tissue.

In some embodiments, the disclosure provides a method of imaging an organ or body region or region, tissue or structure of a subject, the method comprising administrating to the subject the conjugate further conjugated to a detectable agent or a pharmaceutical composition disclosed herein and imaging the subject. In some embodiments such imaging is used to detect a condition associated with a function of the cartilage. In some cases the condition is an inflammation, a cancer, a degradation, a growth disturbance, genetic, a tear or an injury, or another suitable condition. In some cases the condition is a chondrodystrophy, a traumatic rupture or detachment, pain following surgery in regions of the body containing cartilage, costochondritis, herniation, polychondritis, arthritis, osteoarthritis, rheumatoid arthritis, ankylosing spondylitis (AS), Systemic Lupus Erythematosus (SLE or “Lupus”), Psoriatic Arthritis (PsA), gout, achondroplasia, or another suitable condition. In some case the condition is associated with a cancer or tumor of the cartilage. In some cases the condition is a type of chondroma or chondrosarcoma, whether metastatic or not, or another suitable condition. In some embodiments, such as those associated with cancers, the imaging may be associated with surgical removal of the diseased region, tissue, structure or cell of a subject.

Furthermore, the present disclosure provides methods for intraoperative imaging and resection of a diseased or inflamed tissue, cancer, cancerous tissue, or tumor tissue using a conjugate of the present disclosure further conjugated with a detectable agent. In some embodiments, the diseased or inflamed tissue, cancer, cancerous tissue, or tumor tissue is detectable by fluorescence imaging that allows for intraoperative visualization of the cancer, cancerous tissue, or tumor tissue using a conjugate of the present disclosure. In some embodiments, the conjugate of the present disclosure is further conjugated to one or more detectable agents. In a further embodiment, the detectable agent comprises a fluorescent moiety coupled to the peptide of a conjugate herein. In another embodiment, the detectable agent comprises a radionuclide. In some embodiments, imaging is achieved during open surgery. In further embodiments, imaging is accomplished using endoscopy or other non-invasive surgical techniques.

### Treatment of Cartilage Disorders

In various aspects, the present disclosure provides a method of treating a condition in a subject in need thereof, the method comprising: administering to the subject a conjugate as described herein or any of the above pharmaceutical compositions.

In some aspects, the conjugate or pharmaceutical composition is administered by inhalation, intranasally, orally, topically, intravenously, subcutaneously, intra-articularly, intramuscularly administration, intraperitoneally, or a combination thereof. In further aspects, the composition homes, targets, or migrates to cartilage of the subject following administration.

In some aspects, the condition is associated with a function of cartilage. In some aspects, the condition is an inflammation, a cancer, a degradation, a growth disturbance, genetic, a tear, an infection, or an injury. In other aspects, the condition is a chondrodystrophy. In still other aspects, the condition is a traumatic rupture or detachment. In some aspects, the condition is a costochondritis. In other aspects, the condition is a herniation. In still other aspects, the condition is a polychondritis.

In other aspects, the condition is a chordoma. In some aspects, the condition is a type of arthritis. In further aspects, the type of arthritis is rheumatoid arthritis. In other aspects, the type of arthritis is osteoarthritis. In some aspects, the condition is achondroplasia. In other aspects, the type of arthritis is ankylosing spondylitis or psoriatic arthritis. In some aspects, the cancer is benign chondroma or malignant chondrosarcoma. In other aspects, the condition is bursitis, tendinitis, gout, pseudogout, an arthropathy, or an infection.

In some aspects, the conjugate or pharmaceutical composition is administered to treat the injury, to repair a tissue damaged by the injury, or to treat a pain caused by the injury. In further aspects, the conjugate or pharmaceutical composition is administered to treat the tear or to repair a tissue damaged by the tear.

In various aspects, the present disclosure provides a method of imaging an organ or body region of a subject, the method comprising: administering to the subject composition of any one of conjugates previously described conjugated to a detectable agent or a pharmaceutical composition as previously described; and imaging the subject.

In some aspects, the method further comprises detecting a cancer or diseased region, tissue, structure or cell. In further aspects, the method further comprises performing surgery on the subject. In some aspects, the method further comprises treating the cancer.

In other aspects, the surgery comprises removing the cancer or the diseased region, tissue, structure or cell of the subject. In still other aspects, the method further comprises imaging the cancer or diseased region, tissue, structure, or cell of the subject after surgical removal.

The term “effective amount,” as used herein, can refer to a sufficient amount of an active agent, an anti-arthritic agent such as anti-inflammatory agent, or a compound being administered which will relieve to some extent one or more of the symptoms of the disease or condition being treated. The result can be reduction and/or alleviation of the signs, symptoms, or causes of a disease, or any other desired alteration of a biological system. Compositions containing such active agents, anti-arthritic agents such as anti-inflammatory agents, or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal or a lab animal afflicted with a disease or condition) with a conjugate of the disclosure. In treating a disease, the conjugate or the peptide of the conjugate can contact the cartilage of a subject. The subject can be a human. A subject can be a human; a non-human primate such as a chimpanzee, or other ape or monkey species; a farm animal such as a cattle, horse, sheep, goat, swine; a domestic animal such as a rabbit, dog, and cat; a laboratory animal including a rodent, such as a rat, mouse and guinea pig, or the like. A subject can be of any age. A subject can be, for example, an elderly adult, adult, adolescent, pre-adolescence, child, toddler, infant, or fetus in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial. A treatment can comprise administering to a subject a conjugate or pharmaceutical composition, such as one or more of the conjugates or pharmaceutical compositions described throughout the disclosure. A treatment can comprise a once daily dosing. A treatment can comprise delivering a conjugate or pharmaceutical composition of the disclosure to a subject, either intravenously, subcutaneously, intramuscularly, by inhalation, dermally, intra-articular injection, orally, intrathecally, transdermally, intranasally, via a peritoneal route, or directly onto or into a joint, e.g., via topical, intra-articular injection route or injection route of application. A treatment can comprise administering a conjugate to a subject, either intravenously, intra-articular injection, parenterally, orally, via a peritoneal route, or directly onto, near or into the cartilage.

Types of cartilage diseases or conditions that can be treated with a conjugate of the disclosure can include inflammation, pain management, anti-infective, pain relief, anti-cytokine, cancer, injury, degradation, genetic basis, remodeling, hyperplasia, surgical injury/trauma, or the like. Examples of cartilage diseases or conditions that can be treated with a conjugate of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosi s, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis-nephritis, Coloboma of Alar-nasal cartilages with telecanthus, Alar cartilages hypoplasia-coloboma-telecanthus, Pierre Robin syndrome-fetal chondrodysplasia, Dysspondyloenchondromatosis, Achondroplasia regional-dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia-ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or another cartilage or joint disease or condition.

In some embodiments, a conjugate of this disclosure can be administered to a subject in order to target, an anti-arthritic joint. In other embodiments, a peptide or peptide conjugate of this disclosure can be administered to a subject in order to treat an anti-arthritic joint.

In some embodiments, the present disclosure provides methods for determining a therapeutic effect of a peptide conjugate in a subject (e.g., for dose finding, dose testing, and/or dose escalation studies). Such methods include size, diameter, and/or weight measurements of various organs and/or tissues. For example, the effects of a peptide conjugate on arthritis can be determined by measuring ankle and/or joint diameters and weights (e.g., measurements of reduced ankle inflammation and/or ankle diameter compared to a control cohort can show efficacy of a peptide or peptide conjugate). Such methods also include determining uptake and/or retention of peptide or peptide conjugate in such organs and/or tissues, e.g., using in vivo imaging or visualization methods (e.g., autoradiography, nuclear imaging, etc) and/or ex vivo tissue staining (e.g., haemotoxylin and eosin (H&E) staining and/or staining with anti-drug (e.g., anti-dexamethasone) and/or anti-peptide (e.g., anti-peptide(SEQ ID NO: 105) antibodies) in conjunction with microscopy. Organs and tissues that can be measured to determine therapeutic efficacy and/or side effects can include the knee, intervertebral disc (IVD), joints, ankles, blood, muscle, kidney, liver, spleen, bone, thymus, and bone marrow.

In some embodiments, the present disclosure provides methods for determining an efficacious dose range of an active agent, peptide or peptide conjugate as described herein (e.g., for dose finding, dose testing, and/or dose escalation studies). Such methods include measuring certain parameters that may be indicative of therapeutic efficacy (e.g., reduction in joint or ankle inflammation and/or swelling, pain reduction, increase in mobility and/or appetite, overall health, reduced inflammatory marker secretion such as cytokines such as IL-6, modulation of transcription factors due to receptor activation, gene activation or repression, etc.) after administration of different doses of the active agent, peptide, or peptide conjugate to a subject. Additional parameters measured can include those indicative of systemic and/or prolonged exposure to an active agent. Such parameters can include blood cell viability and/or concentration (e.g., total white blood cell (WBC) count, lymphocyte count, etc.), organ weights (e.g., those of the knee, IVD, joints, ankles, blood, muscle, kidney, liver, spleen, bone, thymus, and/or bone marrow) and/or overall body weights, and other observable parameters such as mobility, loss of appetite, or the swelling of joints and ankles.

In some embodiments, the present disclosure provides methods for determining one or more side effects of an active agent, either when administered alone or as a peptide conjugate (e.g., for dose finding, dose testing, and/or dose escalation studies). Such side effects can be due to systemic exposure to an active agent (e.g., a glucocorticoid). The present disclosure provides markers that can be used to assess systemic exposure to an active agent (e.g., a glucocorticoid) and methods for determining such markers. The methods can include measuring blood cell viability and/or concentration (e.g., total white blood cell (WBC) count, lymphocyte count, etc.), organ weights (e.g., those of the knee, IVD, joints, ankles, blood, muscle, kidney, liver, spleen, bone, thymus, and/or bone marrow) and/or overall body weights, and other observable parameters such as mobility, loss of appetite, or the swelling of joints and ankles. Additional markers that can be measured to determine systemic exposure to an active agent can include enzyme levels, such as alanine aminotransferase (ALT) and/or aspartate aminotransferase (AST) levels, or levels of hormones or other signaling molecules such as chemokines (e.g., cytokines and interleukins (e.g., IL-2, IL-6, IL-10, etc.).

In some embodiments, the present disclosure provides a method for determining immunogenicity of a peptide or peptide conjugate. Such methods can include determining binding alleles of a peptide or peptide conjugate, e.g., MHC class II binding alleles. Such methods include in silico screening methods using, for example, computer programs and/or neural networks, such as the neural network program NetMHCII version 2.2. This can enable evaluation of all possible 15mer peptides in each sequence against each major MHC allele group. In some cases, a peptide or peptide conjugate has two or fewer strong binding alleles. In some cases, a peptide or peptide conjugate has one or fewer strong binding alleles. In some cases, a peptide or peptide conjugate has no strong binding alleles when tested using the methods described herein. Other methods include measuring ex vivo T cell activation or in vivo immunogenicity in any species (e.g., rodent, human, etc.) or animal model.

In some embodiments, the present disclosure provides a method for treating a cancer, the method comprising administering to a subject in need thereof an effective amount of a conjugate of the present disclosure.

In some embodiments, the present disclosure provides a method for treating a cancer, the method comprising administering to a patient in need thereof an effective amount of a pharmaceutical composition comprising a conjugate of the present disclosure and a pharmaceutically acceptable carrier.

In some embodiments, the conjugates of the present disclosure can be used to treat chondrosarcoma. Chondrosarcoma is a cancer of cartilage producing cells and is often found in bones and joints. It falls within the family of bone and soft-tissue sarcomas. In certain embodiments, administration of a peptide or peptide conjugate of the present disclosure can be used to image and diagnose or target and treat a subject with chondrosarcoma. The administration of a conjugate of the present disclosure can be used in combination with ablative radiotherapy or proton therapy to treat chondrosarcoma. The subject can be a human or an animal.

In some embodiments, a conjugate of this disclosure can be used to treat Chordoma. In certain embodiments, administration of a conjugate of the present disclosure can be used to image and diagnose or target and treat a subject with chordoma. The administration of a conjugate of the present disclosure can be used in combination with a tyrosine kinase inhibitor, such as imatinib mesylate, and ablative radiotherapy or proton therapy to treat chordoma. The administration of a conjugate of the present disclosure can be used in combination with an antivascular agent such as bevacizumab and an epidermal growth factor receptor inhibitor such as erlotinib to treat chordoma. The subject can be a human or an animal.

In some embodiments, the present disclosure provides a method for inhibiting invasive activity of cells, the method comprising administering an effective amount of a conjugate of the present disclosure to a subject.

In some embodiments, the conjugates of the present disclosure are further conjugated to one or more therapeutic agents. In further embodiments, the therapeutic agent is a chemotherapeutic, anti-cancer drug, or anti-cancer agent selected from, but are not limited to: anti-arthritis, anti-inflammatories, such as for example a glucocorticoid, a corticosteroid, a protease inhibitor, such as for example collagenase inhibitor or a matrix metalloprotease inhibitor (i.e., MMP-13 inhibitor), an amino sugar, vitamin (e.g., Vitamin D), and antibiotics, antiviral, or antifungal, a statin, an immune modulator, radioisotopes, toxins, enzymes, sensitizing drugs, nucleic acids, including interfering RNAs, antibodies, anti-angiogenic agents, cisplatin, anti-metabolites, mitotic inhibitors, growth factor inhibitors, paclitaxel, temozolomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, and their equivalents, as well as photo-ablation. Some of these active agents induce programmed cell death such as apoptosis in target cells and thereby improve symptoms or ameliorate disease. Apoptosis can be induced by many active agents, including, for example, chemotherapeutics, anti-arthritic agents, anti-inflammatories, corticosteroids, NSAIDS, tumor necrosis factor alpha (TNF-α) modulators, tumor necrosis factor receptor (TNFR) family modulators. In some embodiments, conjugates of this disclosure can be used to target active agents to pathways of cell death or cell killing, such as caspases, apoptosis activators and inhibitors, XBP-1, Bcl-2, Bcl-Xl, Bcl-w, and other disclosed herein. In other embodiments, the therapeutic agent is any nonsteroidal anti-inflammatory drug (NSAID). The NSAID can be any heterocyclic acetic acid derivatives such as ketorolac, indomethacin, etodolac, or tolemetin, any propionic acid derivatives such as naproxen, any enolic acid derivatives, any anthranilic acid derivatives, any selective COX-2 inhibitors such as celecoxib, any sulfonanilides, any salicylates, aceclofenac, nabumetone, sulindac, diclofenac, or ibuprofen. In other embodiments, the therapeutic agent is any steroid, such as dexamethasone, budesonide, triamcinolone, cortisone, prednisone, rednisolone, triamcinolone hexacetonide, or methylprednisolone. In other embodiments, the therapeutic agent is a pain reliever, such as acetaminophen, opioids, local anesthetics, anti-depressants, glutamate receptor antagonists, adenosine, or neuropetides. In some embodiments, a treatment consists of administering a combination of any of the above therapeutic agents and a peptide conjugate, such as a treatment in which both a dexamethasone-peptide conjugate and an NSAID are administered to a patient. Conjugates of the current disclosure that target the cartilage can be used to treat the diseases conditions as described herein, for example, any diseases or conditions including tears, injuries (i.e., sports injuries), genetic factors, degradation, thinning, inflammation, cancer or any other disease or condition of the cartilage or to target therapeutically-active substances to treat these diseases amongst others. In other cases, a conjugate of the disclosure can be used to treat traumatic rupture, detachment, chostochondritis, spinal disc herniation, relapsing and non-relapsing polychondritis, injury to the articular cartilage, osteoarthritis, arthritis or achondroplasia. In some cases, the peptide or peptide-active agent conjugate can be used to target cancer in the cartilage, for example benign chondroma or malignant chondrosarcoma, by contacting the cartilage by diffusion into chondrocytes and then having antitumor function, targeted toxicity, inhibiting metastases, etc. As well, such peptide or peptide-active agent conjugate can be used to label, detect, or image such cartilage lesions, including tumors and metastases amongst other lesions, which may be removed through various surgical techniques or by targeting with peptide-active agent conjugates that induce programmed cell death or kill cells.

Conjugates described herein can be administered for prophylactic and/or therapeutic treatments. In therapeutic applications, the conjugate can be administered to a subject already suffering from a disease or condition, in an amount sufficient to cure or at least partially arrest the symptoms of the disease or condition, or to cure, heal, improve, or ameliorate the condition. Such conjugates described herein can also be administered to prevent (either in whole or in part), lessen a likelihood of developing, contracting, or worsening a condition. Amounts effective for this use can vary based on the severity and course of the disease or condition, previous therapy, the subject's health status, weight, response to the drugs, and the judgment of the treating physician. Conjugates and pharmaceutical compositions described herein can allow for targeted homing of the peptide and local delivery of any conjugate. For example, a peptide conjugated to a steroid allows for local delivery of the steroid, which is significantly more effective and less toxic than traditional systemic steroids. A peptide conjugated to an NSAID is another example. In this case, the peptide conjugated to an NSAID allows for local delivery of the NSAID, which allows for administration of a lower NSAID dose and is subsequently less toxic. By delivering an active agent to the joint, pain relief can be more rapid, may be more long lasting, and can be obtained with a lower systemic dose and off-site undesired effects than with systemic dosing without targeting.

Conjugates of the current disclosure that target the cartilage can be used to treat or manage pain associated with a cartilage injury or disorder, or any other cartilage or joint condition as described herein. For example, since ion channels can be associated with pain and can be activated in disease states such as arthritis, the conjugates comprising peptides that interact with ion channels can be used directly to reduce pain. In another embodiment, the peptide of the conjugate is conjugated to an active agent with anti-arthritic or anti-inflammatory activity, in which the peptide acts as a carrier for the local delivery of the active agent to reduce pain.

In some embodiments, the conjugate described herein provide a method of treating a cartilage condition of a subject, the method comprising administering to the subject a therapeutically-effective amount of a conjugate comprising the sequence SEQ ID NO: 1 or fragment thereof conjugated to an anti-arthritic agent such as an anti-inflammatory agent. In some embodiments, the conjugates described herein provide a method of treating a cartilage condition of a subject, the method comprising administering to the subject a conjugate comprising a peptide of any one of SEQ ID NO: 2-SEQ ID NO: 510 or fragment thereof conjugated to an anti-arthritic agent such as an anti-inflammatory agent.

In some embodiments, the present disclosure provides methods and compositions comprising peptide-drug conjugates that can reduce the occurrence and/or intensity of an adverse effect in a subject (e.g., adverse effects associated with the administration of the drug alone). In some cases, the present disclosure provides methods and compositions comprising peptide-drug conjugates that can reduce the occurrence and/or intensity of the adverse effect or both by at least 10%-50%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Multiple conjugates described herein can be administered in any order or simultaneously. If simultaneously, the multiple conjugates described herein can be provided in a single, unified form, such as an intravenous injection, or in multiple forms, such as subsequent intravenous dosages.

Conjugates can be packaged as a kit. In some embodiments, a kit includes written instructions on the use or administration of the conjugates.

### Methods

Manufacture of Peptides.

In some cases, a peptide of the present disclosure is produced recombinantly alone or as a fusion peptide or fusion protein. In some embodiments, disclosed herein are methods that can express the peptide as a C-terminal fusion to a larger protein. In some cases, a peptide is expressed as a C-terminal fusion protein comprising siderocalin as an additional protein. In some cases, such fusions direct the fusion peptide or protein through a mammalian secretory pathway. In some cases, such processes ensure proper formation of the disulfide bond structure of a cystine-dense peptide of the present disclosure. Upon expression, the C-terminal fusion protein (e.g., siderocalin) can be cleaved from the peptide by an optimized TEV enzyme. In some cases, co-expression of a protease or chemical cleavage is used. Expressed fusion proteins can be purified using various methods. Such methods can include Ni-NTA capture of the His tag encoded upstream of the C-terminal fusion protein (e.g., siderocalin), which can be followed by TEV cleavage and peptide purification using, for example, chromatography (e.g., size-exclusion chromatography, reversed-phase (RP) high-pressure liquid chromatography (HPLC), and/or RP-fast protein liquid chromatography (FPLC)). Different purification techniques can be advantageous for manufacturing where the His-tag may be absent and where use of organic solvents may not be used. As an example, the two peptides having the amino acid sequences set forth in SEQ ID NO: 105 and SEQ ID NO: 184 can be expressed in CHO—S cells by transient transfection. Each peptide can be expressed as a siderocalin fusion or as the peptide alone. The peptide may or may not contain any predicted glycosylation sites or other post-translational modifications.

In some embodiments, peptides of the present disclosure (e.g., those derived from cystine-dense peptides) can be synthesized using Fmoc solid-phase SPPS according to cGMP guidelines. Subsequent to SPPS, the peptides can be cleaved from the resin followed by removal of protecting groups. The multiple disulfide bonds of each peptide can then be formed by a single oxidative folding step in solution. The folded peptides can be purified by using any reversed-phase chromatography method and isolated as lyophilized TFA salts. Identity of each peptide synthesized having the amino acid sequences set forth in SEQ ID NO: 105, SEQ ID NO: 103, or SEQ ID NO: 184 can be verified by mass spectrometry (e.g., ESI-MS), chromatography (e.g., RP-HPLC), and/or nuclear magnetic resonance spectroscopy (e.g., NMR).

Collagen Induced Arthritis (CIA) Model.

In some embodiments, prevention, treatment, and/or diagnosis of arthritis are performed in a collagen-induced arthritis (CIA) model system in a non-human animal. Such non-human animal can be a rodent such as a rat or a mouse. In some cases, the non-human animal is a rat. In such a model, arthritis can be induced in 9-week-old female Lewis rats (Envigo or Charles River Laboratories) by intradermal injection while anesthetized of 400 ug bovine type II collagen (Chondrex Inc, Redmond Wash.) in 2 adjacent 200 ul (1 mg/ml) doses on day 0. Collagen can be prepared for injection by dissolving at 2 mg/ml in 0.01N glacial acetic acid in sterile water and rocking at 4° C. overnight then emulsifying in Freund's incomplete adjuvant (IFA, Sigma Aldrich). To emulsify, equal volumes of collagen solution and IFA can be drawn up into separate syringes, which are joined by a 3-way stopcock. While on ice, the collagen and IFA can be mixed by pressing between the two syringes rapidly for 10 minutes. Quality of the emulsification is tested after 10 minutes of mixing by dropping a small amount of mixture in water. A properly emulsified solution can remain as a discrete droplet and not disperse in the water. On day 7, rats are challenged with a second intradermal injection of 100 ug of collagen in 100 ul, 1 mg/ml, collagen in IFA solution prepared fresh. For monitoring arthritis progression, body weight and ankle diameter measurements, for example, can be recorded daily from day 7 through the end of the study. Ankle diameter can be measures by a single researcher using a Fowler Digitrix 2 micrometer. Three measurements of each ankle can be taken of the lateral dimension at the tarsus of lightly anesthetized rats.

Hydrolysis Measurements.

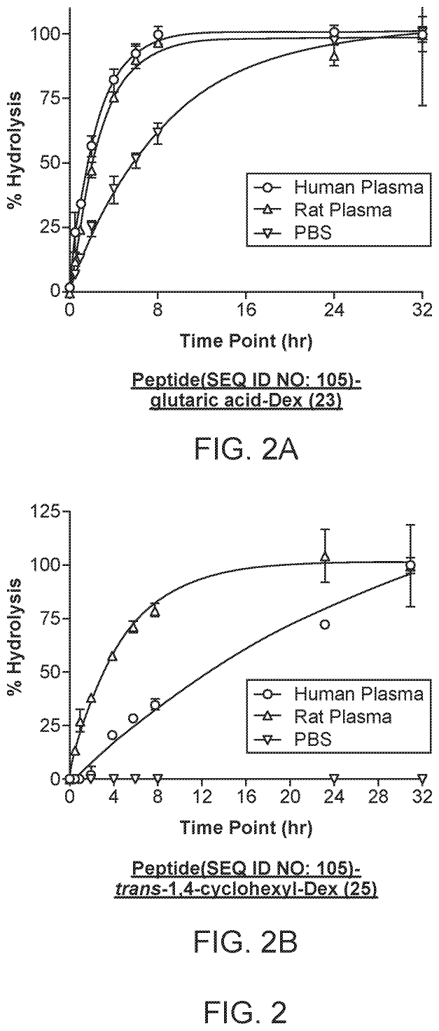

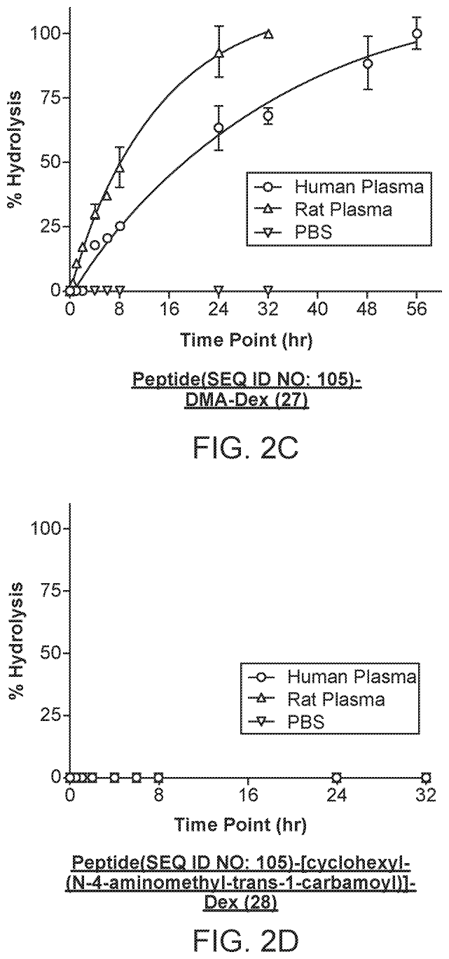

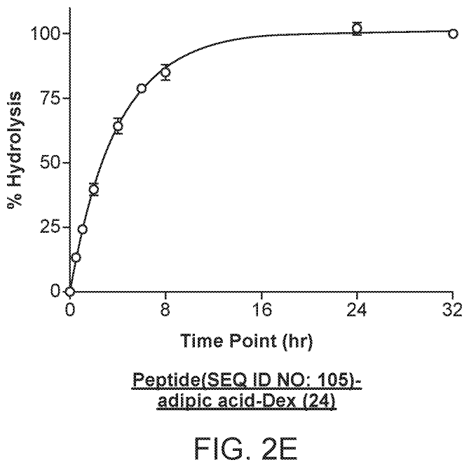

In order to determine hydrolysis rates, conjugates can be diluted in PBS, rat plasma, or human plasma and incubated at 37 degrees. Samples can be taken at designated time-points (e.g., 1, 2, 3, 5, 10, 15, 20, 25, 30, 45, 60, or 90 minutes and/or hours after administration). Internal standards such as triamcinolone acetonide (TAA) can be added as an internal standard, and then active agent/TAA can be extracted using acetonitrile, for example. The samples can be dried down, reconstituted, and analyzed by LC/MS. Data can be normalized using Dex AUC/TAA AUC. Percent hydrolysis can be calculated using the average ratio for active agent AUC/TAA AUC at a time point of maximal drug release. Exemplary hydrolysis measurements are shown in FIG. 2A-FIG. 2E. A general method can include using a lyophilized, purified peptide-drug conjugates reconstituted in DMSO following production to generate stock solution at 20 mg/mL. Each conjugate stock can be brought to 0.25 mg/mL in each hydrolysis condition (1×DPBS, human plasma and rat plasma) in triplicate and rocked at 37° C. To quantitate free active agent, 100 μl of the hydrolyzed conjugate solution can be transferred into 1 mL acetonitrile at 4° C. at which time all plasma proteins and intact conjugates can precipitate. This can be performed repeatedly to generate a series of time points (0 hr, 0.5 hr, 1 hr, 2 hr, 4 hr, 6 hr, 8 hr, 24 hr, 32 hr). The acetonitrile solution can be spun at 17,000 g for 5 minutes, and the supernatant removed and added to a 96 well block. The pellet can then be resuspended in 500 μl acetonitrile and re-pelleted as before. The supernatant can be removed and added to the same well in the block. Samples can be prepared for analysis by drying under a nitrogen stream followed by reconstitution in 110 μl 45:55 ACN:Citrate 10 mM pH 5.5. To ensure optimal recovery the block can be shaken via plate shaker at 7,000 rpm for 5 minutes during the reconstitution step. Prior to LC/MS the samples can be transferred to a 96 well plate and centrifuged at 6,500 g for 15 minutes. Analysis can be performed on an Agilent 1260 Infinity series HPLC with inline 6120 single quad MS using acetonitrile, water (+0.1% TFA) gradient (shown below) on an InfinityLab Poroshell SB-C18 column. Quantification of free active agent (or drug) can be achieved through integration of active agent peaks of the TIC.

Synthesis of Peptide-Active Agent Conjugates.

Peptide-active agent conjugates (or peptide-drug conjugates, i.e., PDC) of the present disclosure can comprise one or more of any peptide described herein (e.g., those listed in TABLE 1 and/or those having the amino acid sequences set forth in SEQ ID NO: 1-SEQ ID NO: 510), any linker described herein (e.g., those listed in TABLE 2), and any active and/or detectable agent described herein.

Generally, a synthetic method for producing a peptide-drug conjugate as described herein can comprise attaching a drug molecule to a linker, followed by the attachment of a peptide to the drug-linker conjugate. Solvents, reaction conditions, and/or additional reagents used during synthesis may be selected such that yield and purity allow scaling the production of a peptide-drug conjugate (PDC) for preclinical and/or clinical studies while retaining the biological activity of the peptide and/or the active or detectable agent used in the PDC.

Peptide-drug conjugates (PDC) of the present disclosure can be synthesized using various synthetic strategies. Examples of such synthetic strategies are shown in EXAMPLE 5-EXAMPLE 19, and EXAMPLE 29. PDC's of the present disclosure can be synthesized using various chemical reactions and/or chemical transformations. In some embodiments, a PDC is synthesized using hydrolysis, ester bond formation(s), NHS ester formation(s), amide formation(s), peptide conjugation(s), a carbamate formation(s), mesylate formation(s), sulfur alkylation(s), reductive amination(s), deprotection(s), or any combination thereof. A peptide conjugation reaction can include but is not limited to an amide bond formation, a carbamate formation, a carbonate formation, an ester bond formation, or a combination thereof. Synthetic approaches for producing PDCs can comprise use of one or more protecting groups (e.g., Boc, Fmoc, MOM, etc), and, as such, can include one or more protection and/or deprotection steps. In some cases, a PDC synthesis comprises the formation of activated carboxylic acids such as a conversion of a carboxylic acid into an activated ester such as an NHS ester.

In some cases, the synthesis of a PDC comprises a radiolabeling step. Radiolabeling can comprise attaching one or more radionuclides to a PDC, e.g., the linker, and/or the peptide. The radionuclide can be ¹⁴C. Radiolabeling of peptides with ¹⁴C can comprise reductive amination. Radiolabeling of PDCs with other radionuclides as described herein can comprise use of different synthetic strategies and/or use of chelator moieties. In some embodiments, a peptide and/or a peptide-drug conjugate is radiolabeled. A peptide and/or a peptide-drug conjugate can be radiolabeled with various radionuclides suitable for determining pharmacokinetic and/or pharmacodynamic (PD) parameters such as plasma half-life, organ and/or tissue uptake and/or retention, target engagement, etc.

In some embodiments, a peptide conjugate comprising a 2,5-dimethyladipic acid (DMA) linker and dexamethasone (also referred to herein as “Dex”) or des-ciclesonide (also referred to herein as “dCIC”) that can be synthesized using any one or more of hydrolysis, ester bond formation, NHS ester formation, and peptide conjugation. For example, a peptide conjugate comprising a peptide having the amino acid sequence set forth in SEQ ID NO: 105, a (carbamate) trans-1-aminomethyl-cyclohexyl-4-carboxylic linker and dexamethasone can be synthesized using any one or more of carbamate formation, NHS ester formation, peptide conjugation, or any combination thereof.

In some embodiments, a synthesis of a PDC of the present disclosure comprises any one or more of mesylate formation, sulfur alkylation, NHS ester formation, peptide conjugation, Boc deprotection, reductive amination, or any combination thereof. For example, a peptide-drug conjugate comprising a cysteine linker and triamcinolone acetonide (TAA) can be synthesized using mesylate formation, sulfur alkylation, NHS ester formation, peptide conjugation, Boc deprotection, reductive amination, or any combination thereof.

In some embodiments, a PDC comprising any one of the peptides which amino acid sequence is set forth in any one of SEQ ID NO: 1-SEQ ID NO: 510, a drug (e.g., glucocorticoid such as dexamethasone or des-ciclesonide), and any one of a glutaric acid linker, 3,3-tetramethylene-glutaric linker, a trans-1,2-cyclohexyl linker, a 1,3-cyclohexyl linker, a terephthalic acid linker, a 2,3-dimethyl-succinic acid linker, a succinic acid linker, a adipic acid linker, a trans-1,4-cyclohexyl linker can be produced using any one or more of ester bond formation, NHS ester formation, peptide conjugation, or any combination thereof. In some embodiments, a peptide-drug conjugate of the present disclosure comprises a peptide of the present disclosure linked to a glucocorticoid via a linker. In some cases, the peptide comprises an amino acid sequence set forth in any one of SEQ ID NO: 103, SEQ ID NO: 105, SEQ ID NO: 184, or SEQ ID NO: 509. In some cases, the glucocorticoid is dexamethasone or des-Ciclesonide. In some cases, the linker is any linker listed in TABLE 2 having compound number 1-22. The PCD comprising the peptide having the amino acid sequence set forth in SEQ ID NO: 105 (peptide(SEQ ID NO: 105)) linked to dexamethasone (i.e., Dex) via a cysteine linker (Cys), peptide(SEQ ID NO: 105)-Cys-Dex (“peptide(SEQ ID NO: 105)-Cys” is disclosed as SEQ ID NO: 512), can be synthesized using any one or more of mesylate formation, sulfur alkylation, NHS ester formation, peptide conjugation, Boc deprotection, reductive amination (see e.g., EXAMPLE 8). As another example, the PDC peptide(SEQ ID NO: 105)-DMA-dCIC (also referred to as peptide(SEQ ID NO: 105)-DMA-dCIC) can be synthesized using the following three synthetic steps of ester bond formation, sulfo-NHS ester formation, and peptide conjugation as described herein.

Pharmacokinetic, Pharmacodynamic, and/or Functional Testing.

In some embodiments, the present disclosure provides methods for determining a therapeutic effect of a peptide conjugate in a subject (e.g., for dose finding, dose testing, and/or dose escalation studies). Such methods include size, diameter, and/or weight measurements of various organs and/or tissues. For example, the effects of a peptide conjugate on arthritis can be determined by measuring ankle and/or joint diameters and weights (e.g., measurements of reduced ankle inflammation and/or ankle diameter compared to a control cohort can show functionality or effect of a peptide or peptide conjugate). Such methods also include determining uptake and/or retention of peptide or peptide conjugate in such organs and/or tissues, e.g., using in vivo imaging or visualization methods (e.g., autoradiography, nuclear imaging, etc) and/or ex vivo tissue staining (e.g., haemotoxylin and eosin (H&E) staining and/or staining with anti-drug (e.g., anti-dexamethasone) and/or anti-peptide (e.g., anti-peptide(SEQ ID NO: 105) antibodies) in conjunction with microscopy (e.g., immunohistochemistry). Organs and tissues that can be measured to determine therapeutic functionality or effect and/or side effects can include the knee, intervertebral disc (IVD), joints, ankles, blood, muscle, kidney, liver, spleen, bone, thymus, and bone marrow.

In some embodiments, the present disclosure provides methods for determining an efficacious dose range of an active agent, peptide or peptide conjugate as described herein (e.g., for dose finding, dose testing, and/or dose escalation studies). Such methods include measuring certain parameters that may be indicative of therapeutic functionality or effect (e.g., reduction in joint or ankle inflammation and/or swelling, pain reduction, increase in mobility and/or appetite, overall health, reduction in inflammatory markers such as cytokines such as IL-6) after administration of different doses of the active agent, peptide, or peptide conjugate to a subject. Additional parameters measured can include those indicative of systemic and/or prolonged exposure to an active agent. Such parameters can include blood cell viability and/or concentration (e.g., total white blood cell (WBC) count, lymphocyte count, etc.), organ weights (e.g., those of the knee, IVD, joints, ankles, blood, muscle, kidney, liver, spleen, bone, thymus, and/or bone marrow) and/or overall body weights, and other observable parameters such as mobility, loss of appetite, or the swelling of joints and ankles.

In some embodiments, the present disclosure provides methods for determining one or more side effects of an active agent, either when administered alone or as a peptide conjugate (e.g., for dose finding, dose testing, and/or dose escalation studies). Such side effects can be due to systemic exposure to an active agent (e.g., a glucocorticoid). The present disclosure provides markers that can be used to assess systemic exposure to an active agent (e.g., a glucocorticoid) and methods for determining such markers. The methods can include measuring blood cell viability and/or concentration (e.g., total white blood cell (WBC) count, lymphocyte count, etc.), organ weights (e.g., those of the knee, IVD, joints, ankles, blood, muscle, kidney, liver, spleen, bone, thymus, and/or bone marrow) and/or overall body weights, and other observable parameters such as mobility, loss of appetite, or the swelling of joints and ankles. Additional markers that can be measured to determine systemic exposure to an active agent can include enzyme levels, such as alanine aminotransferase (ALT) and/or aspartate aminotransferase (AST) levels, or levels of hormones or other signaling molecules such as chemokines (e.g., cytokines and interleukins (e.g., IL-1, IL-2, IL-6, IL-10, etc.).

In some embodiments, the side effect can be any one of body weight loss, immunosuppression, skin thinning, purpura, Cushingoid appearance, cataract or glaucoma in an eye, osteoporosis or bone fractures, hypothalamic-pituitary-adrenal (HPA) axis suppression, hyperglycemia and diabetes, increased incidence of serious cardiovascular events, dyslipidemia, myopathy, gastritis, gastrointestinal ulcers and bleeding, psychiatric disturbance, increased blood glucose, decreased serum cortisol or corticosterone, atrophy of adrenal gland, thymus, or spleen, reduction in circulating lymphocytes, decreased cellularity of bone marrow, muscular atrophy, decreased muscle function, pain, muscular pain, arthritic pain, joint pain, joint deformity, decreased mobility, decreased range of motion in a joint, decreased flexibility, decreased strength, decreased balance, impaired glucose tolerance, loss of appetite, decreased bone metabolism, impaired immunity, nephrotic syndrome, fatigability, fungal infection, viral infection, bacterial infection, GI perforation, behavioral and mood disturbances, secondary adrenocortical insufficiency, water retention, cataracts, glaucoma, elevated blood pressure, osteoporosis, suppression of growth in children, increased insulin requirements, weight gain, nausea, Cushing's syndrome, malfunctions of the musculoskeletal, gastrointestinal, dermatologic, neurologic, endocrine, ophthalmic, metabolic, or cardiovascular systems, or any combination thereof.

In some embodiments, the present disclosure provides a method for determining immunogenicity of a peptide or peptide conjugate. Such methods can include determining binding alleles of a peptide or peptide conjugate, e.g., MHC class II binding alleles. Such methods include in silico screening methods using, for example, computer programs and/or neural networks, such as the neural network program NetMHCII version 2.2. This can enable evaluation of all possible 15mer peptides in each sequence against each major MHC allele group. In some cases, a peptide or peptide conjugate has two or fewer strong binding alleles. In some cases, a peptide or peptide conjugate has one or fewer strong binding alleles. In some cases, a peptide or peptide conjugate has no strong binding alleles when tested using the methods described herein. Other methods for assessing immunogenicity can include assessing ex vivo T cell activation, or in vivo immunogenicity such as antibody generation after dosing to any species.

In some embodiments, the present disclosure provides a method for determining binding of a peptide or peptide conjugate to certain receptors, enzymes, or other proteins. Such receptors can be ion channels. Ion channel binding can be determined in vivo, ex vivo, and/or in silico. Ion channels to be tested for peptide binding can include KCNQ1, HCN4, Kᵥ1.5, Kᵢᵣ2.1, hERG, Caᵥ1.2, Kᵥ4.3, and/or Naᵥ1.5. In some cases, a peptide or peptide conjugate shows binding to less than 2 of the above described ion channels. In some cases, a peptide or peptide conjugate shows binding to less than 1 of the above described ion channels. In some cases, a peptide or peptide conjugate shows binding to none of the above described ion channels.

### EXAMPLES

The following examples are included to further describe some embodiments of the present disclosure, and should not be used to limit the scope of the disclosure.

### Example 1

### Manufacture of Peptides Using Recombinant Expression

This example provides a method for generating cystine-dense peptides. Peptides derived from cystine-dense peptides were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

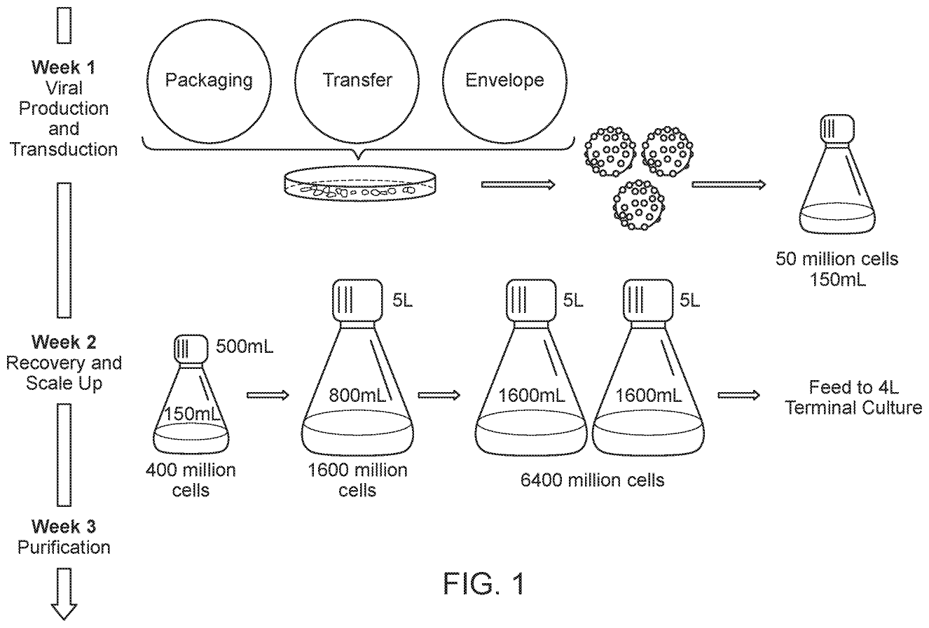

Generally, the peptide sequence was reverse-translated into DNA, synthesized, and cloned in-frame with siderocalin using standard molecular biology techniques. (M. R. Green, Joseph Sambrook. Molecular Cloning. 2012 Cold Spring Harbor Press.). The resulting construct was packaged into a lentivirus, transfected into HEK293 cells, expanded, isolated by immobilized metal affinity chromatography (IMAC), cleaved with tobacco etch virus protease, and purified to homogeneity by reverse-phase chromatography. Following purification, each peptide was lyophilized and stored frozen. FIG. 1 illustrates a general method of manufacturing a peptide of the disclosure.

Peptides can be made using various cell lines such as HEK293 or CHO—S cells. Peptides can be expressed by themselves or with an upstream leader such as siderocalin fused to the N-terminus of the peptide by a cleavable link. Various enzyme cleavable or chemically cleavage links can be used. Peptides (or peptide fusions) can be expressed by use of various leader peptides. Various tags such as His tags or FLAG tags may also be used, or other known methods.

As an example, described herein is a process that expressed the peptide as a C-terminal fusion to a larger protein (siderocalin) which directs the fusion protein through the mammalian secretory pathway to ensure proper formation of the disulfide bond structure of the cystine-dense peptides (the N-terminus of the peptide is fused to the C-terminus of siderocalin). After expression, the siderocalin was cleaved from the peptide by an optimized TEV enzyme. Co-expression of a protease or the use of chemical cleavage are possible alternatives. In addition, the fusion proteins were purified via a Ni-NTA capture of the His tag encoded upstream of siderocalin, and then, following TEV cleavage, the peptide was purified by RP-FPLC. Different purification techniques can be used as well. Alternatively the peptides can be expressed with a leader peptide but no additional larger fusion protein.

Expression of the two peptides having the amino acid sequences set forth in SEQ ID NO: 105 and SEQ ID NO: 184 were evaluated in CHO—S cells by transient transfection. Each peptide was expressed as a siderocalin fusion or as the peptide alone. The peptide did not contain any predicted glycosylation sites or other post-translational modifications. The fusion used the same leader peptide used in the process and the peptide alone used a leader peptide predicted in silico to cleave immediately upstream of the desired mature peptide. 25 mL cultures were maintained at 32° C. for 2 weeks. Good viability was maintained throughout culture. A significant band migrating at the expected position of each fusion protein was evident by SDS-PAGE at 7 days and 13 days of culture, though a host protein appears present at lower levels in a similar migration position throughout culture. No significant expression of the peptide alone was detected in the cell supernatant, indicating that the peptide was potentially not secreted, was degraded, or was not expressed at significant levels. The fusion proteins were captured by Ni-NTA chromatography and subjected to TEV cleavage, producing the expected new band on SDS-PAGE. RP-HPLC analysis of this product, both reduced and non-reduced, showed a major peak with the expected migration behavior. MS analysis of this product also showed the expected m/z that matched that seen for the peptide produced by conventional expression.

The data show that recombinant expression described herein produces functional peptides. The cartilage-homing peptides described herein were successfully produced by recombinant CHO expression with comparable biochemical properties to those produced with other production methods described herein (see e.g., SPPS in EXAMPLE 2 below) described herein.

Alternatively, chemical synthesis can be used to incorporate unnatural amino acids with functional groups (e.g., alkynes) into a peptide of the disclosure, such as those described below in EXAMPLE 2. This functional group is used as a functional handle as described herein.

### Example 2

### Manufacture of Peptides Using Solid-Phase Synthesis

This example provides a method for synthesizing and manufacturing cystine-dense peptides via solid-phase peptide synthesis (SPPS).

Peptides derived from cystine-dense peptides were synthesized using Fmoc solid-phase SPPS. Subsequent to SPPS, the peptides were cleaved from the resin followed by removal of protecting groups. The multiple disulfide bonds of each peptide were then formed by a single oxidative folding step in solution. The folded peptides were purified by reversed-phase chromatography and isolated as lyophilized TFA salts. Identity of each peptide synthesized having the amino acid sequences set forth in SEQ ID NO: 105, SEQ ID NO: 103, or SEQ ID NO: 184 was verified by ESI-MS and purities by RP-HPLC were >95%.

The synthetically produced peptides were then analyzed side-by-side with peptides produced in the recombinant expression system as described above in EXAMPLE 1. LC-MS analysis showed that the synthetic peptides displayed the same retention time and observed mass as the recombinant peptides. In addition, synthetic (SPPS) peptide having the amino acid sequence set forth in SEQ ID NO: 105 displayed similar resistance to chemical reduction displayed by the recombinant form, as evidenced by very little formation of a new peak in LC-MS after incubation in 50 mM DTT for 60 minutes at room temperature. Moreover, SDS-PAGE analysis also showed that the synthetic peptides showed comparable migration patterns, reduced and non-reduced, to the recombinant peptides.

This data demonstrates that recombinantly expressed and synthetically produced peptides as disclosed herein have similar properties, indicating that both methodologies produce functional peptides that can be used as carriers for active agents. Thus, the synthetic approaches described herein enable production of the herein described peptides.

### Example 3

### Radiolabeling of Peptides

This example describes radiolabeling of cystine-dense peptides. Several cystine-dense peptides (some sequences derived from spiders and scorpions) were radiolabeled by reductive amination on ¹⁴C formaldehyde and sodium cyanoborohydride with standard techniques. See J Biol Chem. 254(11):4359-65 (1979). The sequences were engineered to have the amino acids, “G” and “S” at the N terminus. See Methods in Enzymology V91:1983 p. 570 and Journal of Biological Chemistry 254(11):1979 p. 4359. An excess of formaldehyde was used to ensure complete methylation (dimethylation of every free amine). The labeled peptides were isolated via solid-phase extraction on Strata-X columns (Phenomenex 8B-S100-AAK), rinsed with water with 5% methanol, and recovered in methanol with 2% formic acid. Solvent was subsequently removed in a blowdown evaporator with gentle heat and a stream of nitrogen gas.

### Example 4

### Synthetic Routes of Glucocorticoid-Peptide Conjugates

The following conjugates comprising SEQ ID NO: 105 or SEQ ID NO: 509 were made with the protocols described below in EXAMPLES 5-19.

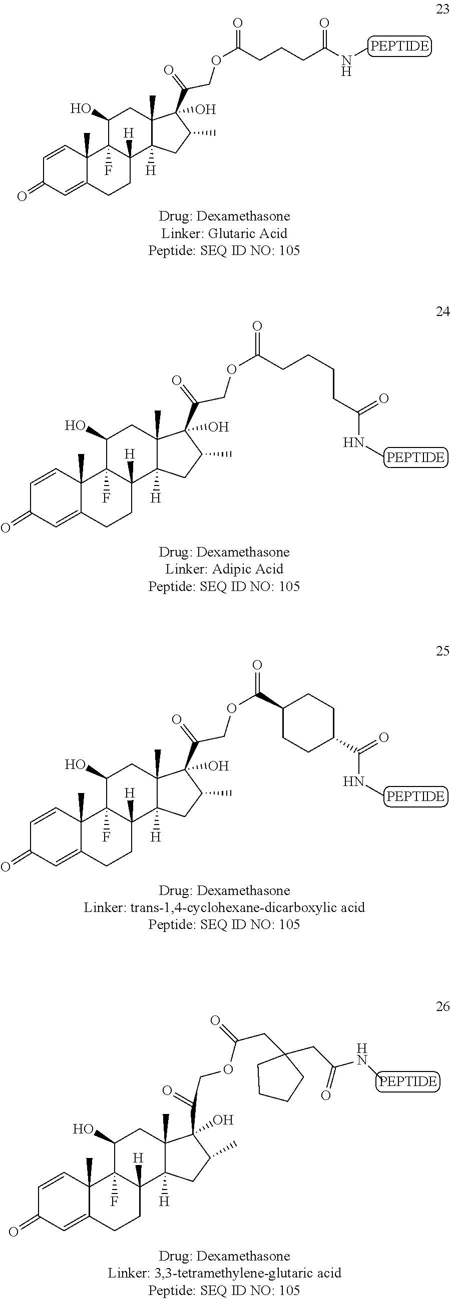

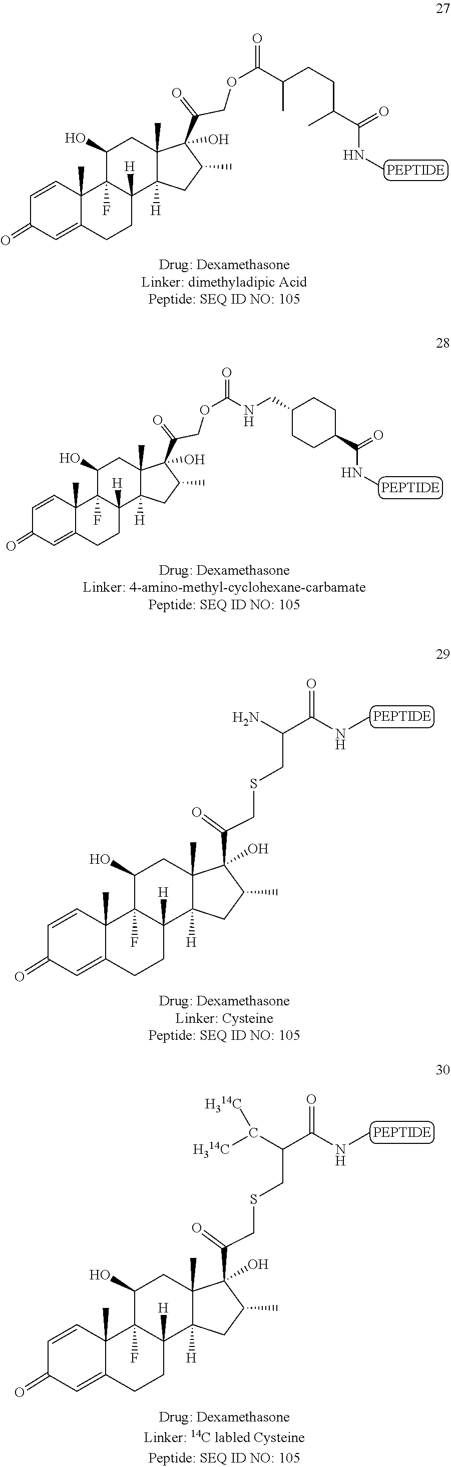

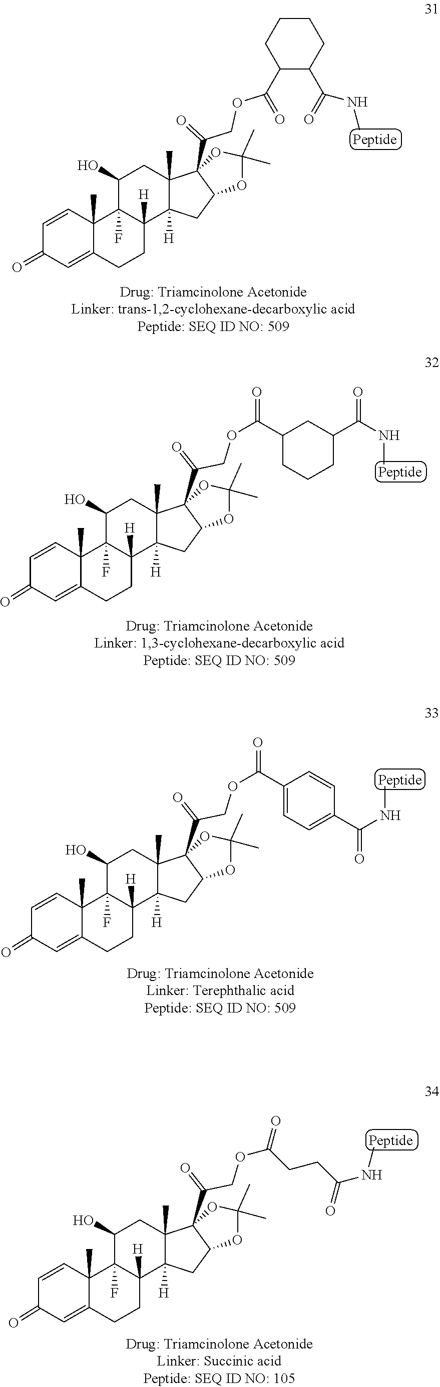

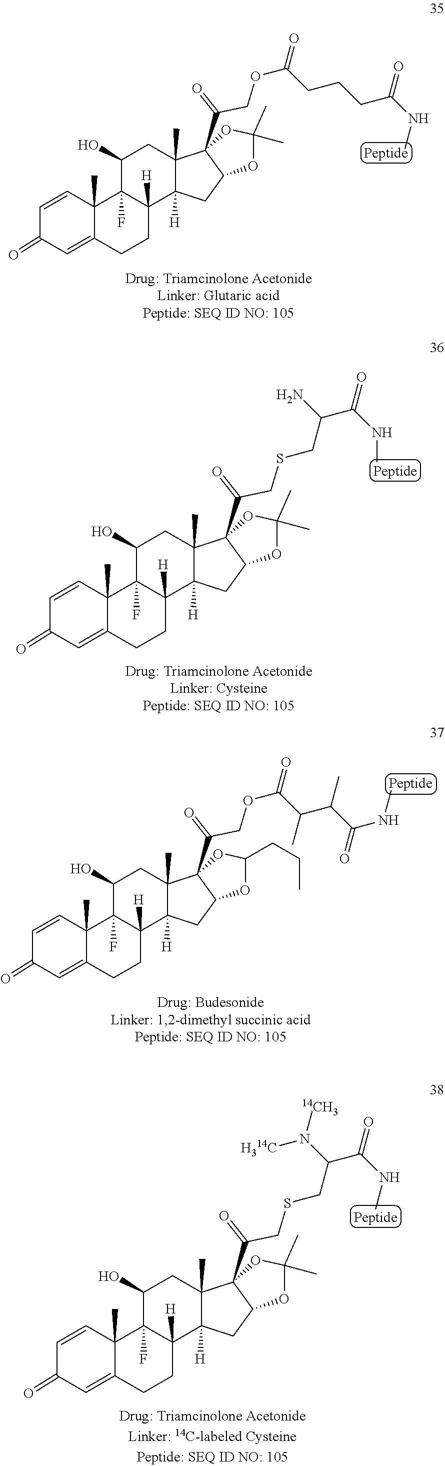

Alternatives to the peptides used in this EXAMPLE or another peptide disclosed herein, such as SEQ ID NO: 105, SEQ ID NO: 509, SEQ ID NO: 103, or SEQ ID NO: 184, are used as the peptide for the conjugation. Alternatives to dexamethasone, budesonide, triamcinolone acetonide, des-ciclesonide or other glucocorticoids disclosed herein are used as an active agent for the conjugation.

### Example 5

### Synthesis of Peptide(SEQ ID NO: 105)-DMA-Dex (27) Conjugate

This example demonstrates the synthesis of peptide(SEQ ID NO: 105)-[dimethyladipic acid]-Dex (27) as shown in the exemplified scheme below.

Materials: Caesium carbonate (Cs₂CO₃); Dichloromethane (DCM); Dimethylsulfoxide (DMSO); 1-Ethyl-3-(3-dimethylaminopropyl)carbodiimide hydrochloride (EDC); N-hydroxysuccinimide (HOSu); pyridine; N-methylmorpholine (NMM); 4-dimethylaminopyridine (DMAP); Dimethyl 2,5-dimethyl adipate; N,N′-dimethylformamide (DMF); phosphate-buffered saline (PBS); Trifluoroacetic acid (TFA), Tetrahydrofuran (THF); Lithioum hydroxide (LiOH); etc. were from Sigma-Aldrich. Dexamethasone (Dex) and triamcinolone acetonide (TAA) were purchased from MedChemExpress. Budesonide (Bud) was purchased from TRC Canada.

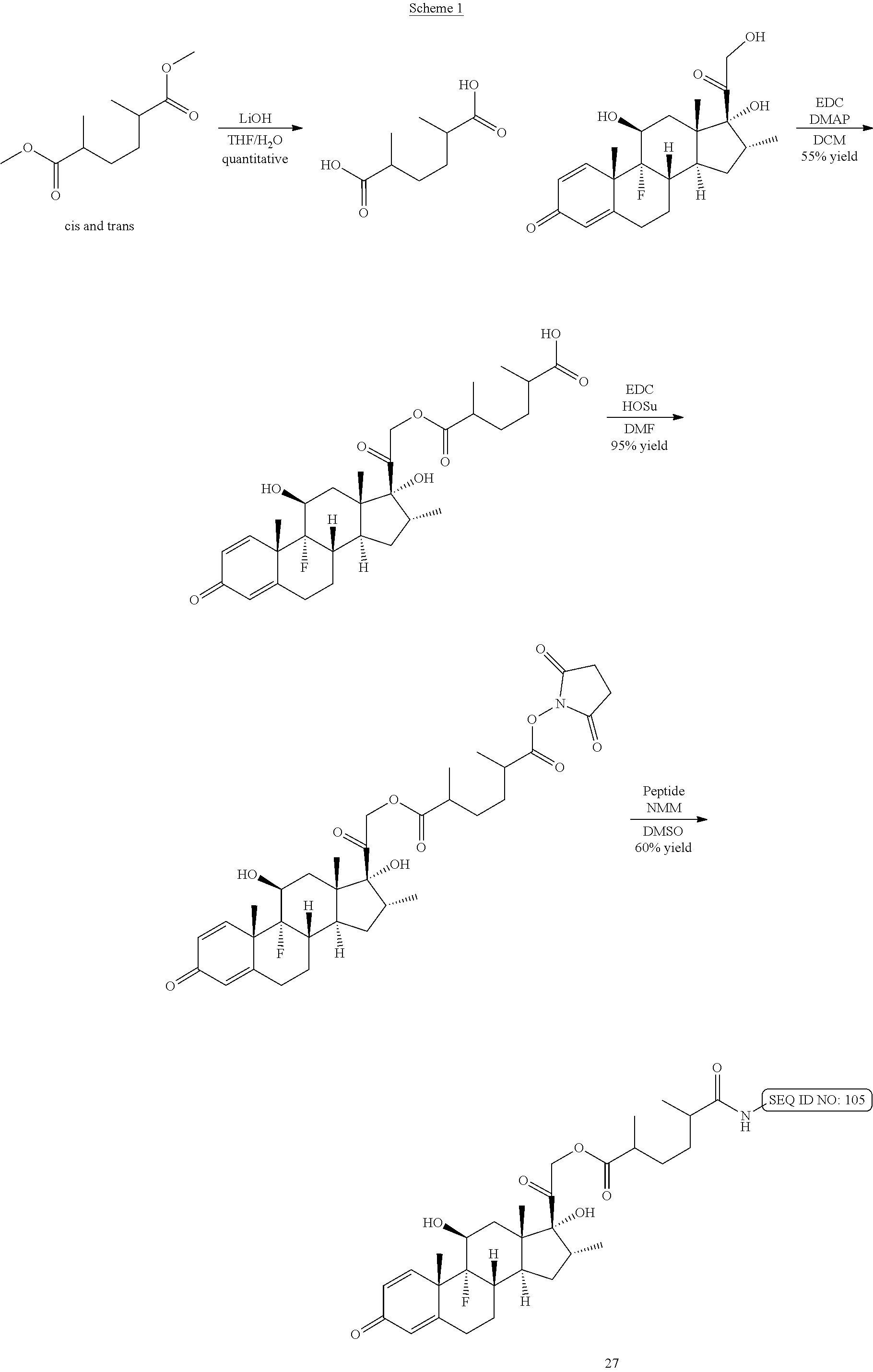

This example demonstrates a peptide conjugate comprising a 2,5-dimethyladipic acid (DMA) linker and dexamethasone that was synthesized using the following general, non-limiting steps:

i) Hydrolysis

ii) Ester bond formation

iii) NHS ester formation

iv) Peptide conjugation

These steps are illustrated in Scheme 1 and described in further detail below.

i) Hydrolysis—Synthesis of 2,5-Dimethyl Adipic Acid
Lithium hydroxide (236 mg, 9.89 mmol) in 2 mL water was added to Dimethyl 2,5-dimethyl adipate (1 g, 4.94 mmol) in 2 mL THF solution. The reaction mixture was stirred at room temperature for 2 days. Separated THF and H₂O layers, washed H₂O part with EtOAc (3×2 mL). 12M HCl solution was added dropwise to the water part until white precipitate came out of solution. The liquid was removed and then the solid was dried to give 0.878 g white solid quantitatively. The crude product was used for the next step without further purification.

ii) Ester Bond Formation-Synthesis of Dex-2,5-Dimethyl Adipic Acid
Dexamethasone (104 mg, 0.265 mmol), 2,5-dimethyl adipic acid (138 mg, 0.795 mmol), 1-Ethyl-3-(3-dimethylaminopropyl)carbodiimide hydrochloride (152 mg, 0.795 mmol) and 4-dimethylaminopyridine (97 mg, 0.795 mmol) were dissolved in 3 mL anhydrous DCM. The reaction mixture was stirred at room temperature for approximately 20 hours. The solvent was removed under reduced pressure. The residue was purified by HPLC to give 80 mg white powder with 55% yield. LC-MS showed greater than 95% purity with mass 531.3 (M−17).

iii) NHS Ester Formation-Synthesis of Dex-2,5-Dimethyl Adipic Acid NHS Ester
Dex-2,5-dimethyl-adipic acid (30 mg, 0.055 mmol), N-(3-dimethylaminopropyl)-N′-ethylcarbodiimide hydrochloride (31.5 mg, 0.165 mmol) and N-hydroxysuccinimide (19 mg, 0.165 mmol) were dissolved in 1 mL anhydrous DMF. The reaction mixture was stirred at room temperature for approximately 20 hours. The reaction mixture was purified by HPLC to give 33.6 mg white powder with 95% yield. LC-MS showed greater than 98% purity with mass 668.2 (M+23).

iv) Peptide Conjugation-Synthesis of Peptide(SEQ ID NO: 105)-DMA-Dex (27) Conjugate
To the stirred mixture of the peptide having amino acid sequence SEQ ID NO: 105 (40 mg, 0.0093 mmol) and N-methylmorpholine (10.23 μL, 0.014 mmol) in 1 mL anhydrous DMSO, was added Dexamethasone 2,5-dimethyl-adipic acid-NETS ester (9.1 mg, 99% purity, 0.093 mmol). The reaction mixture was stirred at room temperature for 2 days. The reaction mixture was purified by HPLC to give white powder (combined 2 reactions, total 90 mg of the peptide was used, 60.6 mg product was obtained with 60% yield). LC-MS showed 96% purity with MS: 1209 (M/4), 1611.3 (M/3).

Alternatives to the peptides used in this EXAMPLE or another peptide disclosed herein, such as SEQ ID NO: 103 or SEQ ID NO: 184 or SEQ ID NO: 509, are used as the peptide for the conjugation. Alternatives to dexamethasone, budesonide, triamcinolone acetonide, or other glucocorticoid disclosed herein are used as an active agent for the conjugation.

### Example 6

### Synthesis of Peptide(SEQ ID NO: 105)-[cyclohexyl-(N-4-aminomethyl-trans-1-carbamoyl)]-Dex (28)

This example demonstrates the synthesis of peptide(SEQ ID NO: 105)-[cyclohexyl-(N-4-aminomethyl-trans-1-carbamoyl)]-Dex (28) as shown in the exemplified scheme below.

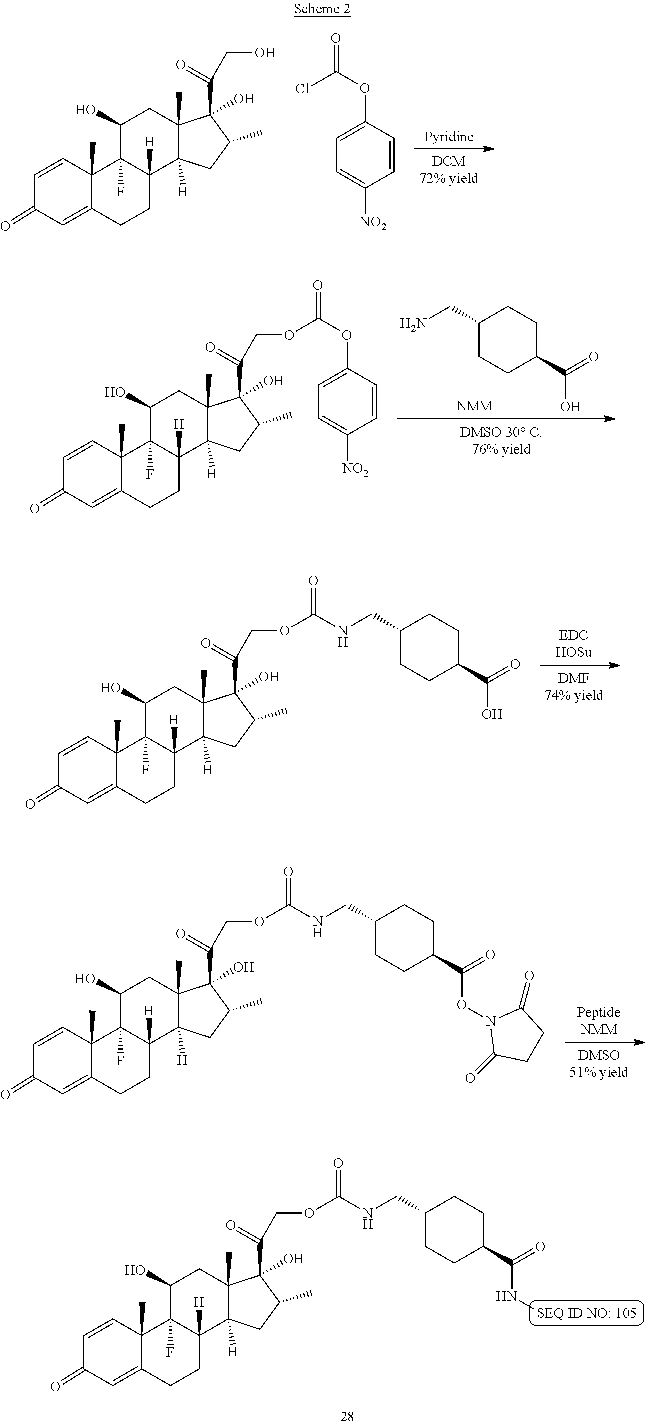

This example describes the synthesis of a peptide conjugate incorporating (carbamate) trans-1-aminomethyl-cyclohexyl-4-carboxylic linker and dexamethasone was synthesized using the following general, non-limiting steps:

i) Dex-COPNP formation

ii) Carbamate formation

iii) NHS ester formation

iv) Peptide conjugation

These steps are illustrated in Scheme 2, and described in further detail below.

### i) Dex-COPNP Formation-Synthesis of p-Nitrophenyl Dex-Formate

Dexamethasone (311 mg, 0.79 mmol) was dissolved in anhydrous DCM 8 mL (suspension) in a septum-sealed reaction vial. To the stirred suspension, pyridine (191 μL, 2.37 mmol) was added. Then 4-Nitrophenyl chloroformate (208 mg, 1.03 mmol) was added via syringe to the reaction mixture. The reaction mixture became clear and was stirred at room temperature for approximately 4 hours. The sealed reaction was poured into 25 mL iced water and the layers were isolated. The organic layer was washed with 1 mL 0.1 M HCl (aq), followed by saturated NaHCO₃ and then brine. Finally, the solution was dried over Na₂SO₄. The mixture was filtered to get the filtrate and the solvent was removed under reduced pressure to give the crude product (p-nitrophenyl Dex-formate). The crude mixture was purified by HPLC to give 315 mg white solid with 72% yield. LC-MS showed 95% purity and mass 558.2 (M+1).

### ii) Carbamate Formation-Synthesis of Dex-carbamate-trans-1-aminomethyl-cyclohexyl-4-carboxylic Acid

Dex-COPNP (229 mg, 0.41 mmol) and trans-4-(aminomethyl) cyclohexane carboxylic acid (193 mg, 1.23 mmol) in anhydrous DMSO 5 mL (not a clear solution) was added N-methylmorpholine (271 μL, 2.46 mmol). The reaction mixture was stirred at 30° C. for approximately 20 hours. The reaction mixture was purified by HPLC to give 180.1 mg white powder with 76% yield. LC-MS showed 95% purity with mass 576.3 (M+1).

### iii) NHS Ester Formation-Synthesis of Dex-carbamate-trans-1-aminomethyl-cyclohexyl-4-carboxylic NHS Ester

To the mixture of Dex-carbamate-trans-1-aminomethyl-cyclohexyl-4-carboxylic acid (26.3 mg, 0.046 mmol), 1-Ethyl-3-(3-dimethylaminopropyl)carbodiimide (26 mg, 0.14 mmol) and N-Hydroxysuccinimide (26 mg, 0.23 mmol) were added 1 mL DMF. The reaction mixture was stirred at room temperature for approximately 20 hours. The reaction mixture was purified by HPLC to give 22.8 mg white powder with 74% yield. LC-MS showed greater than 95% purity with mass 673.3 (M+1).

iv) Peptide Conjugation-Synthesis of Peptide(SEQ ID NO: 105)-[cyclohexyl-(N-4-aminomethyl-trans-1-carbamoyl)]-Dex (28)

To the stirred mixture of the peptide having amino acid sequence SEQ ID NO: 105 (40 mg, 0.0093 mmol) and N-methylmorpholine (10.23 μL, 0.093 mmol) in 1 mL DMSO, was added Dex-carbamate-trans-1-aminomethyl-cyclohexyl-4-carboxylic NHS ester (12.5 mg, 99% purity, 0.0186 mmol). The reaction mixture was stirred at room temperature for 2 days. The reaction mixture was purified by HPLC to give 23 mg white powder with 51% yield. LC-MS showed 99% purity with mass 1215.8 (M/4) and 1620.3 (M/3).

Alternatives to the peptides used in this EXAMPLE or another peptide disclosed herein, such as SEQ ID NO: 103 or SEQ ID NO: 184 or SEQ ID NO: 509, are used as the peptide for the conjugation. Alternatives to dexamethasone, budesonide, triamcinolone acetonide, or other glucocorticoid disclosed herein are used as an active agent for the conjugation.

### Example 7

### Synthesis of Peptide(SEQ ID NO: 105)-Cys-TAA (36) (“peptide(SEQ ID NO: 105)-Cys” is Disclosed as SEQ ID NO: 512) and Peptide(SEQ ID NO: 105)-¹⁴C-Cys-TAA (38) (“peptide(SEQ ID NO: 105)-¹⁴C-Cys” is disclosed as SEQ ID NO: 512)

This example demonstrates the synthesis of peptide(SEQ ID NO: 105)-Cys-TAA (36) (“peptide(SEQ ID NO: 105)-Cys” is disclosed as SEQ ID NO: 512) and peptide(SEQ ID NO: 105)-¹⁴C-Cys-TAA (38) (“peptide(SEQ ID NO: 105)-¹⁴C-Cys” is disclosed as SEQ ID NO: 512) as shown in the exemplified scheme below.

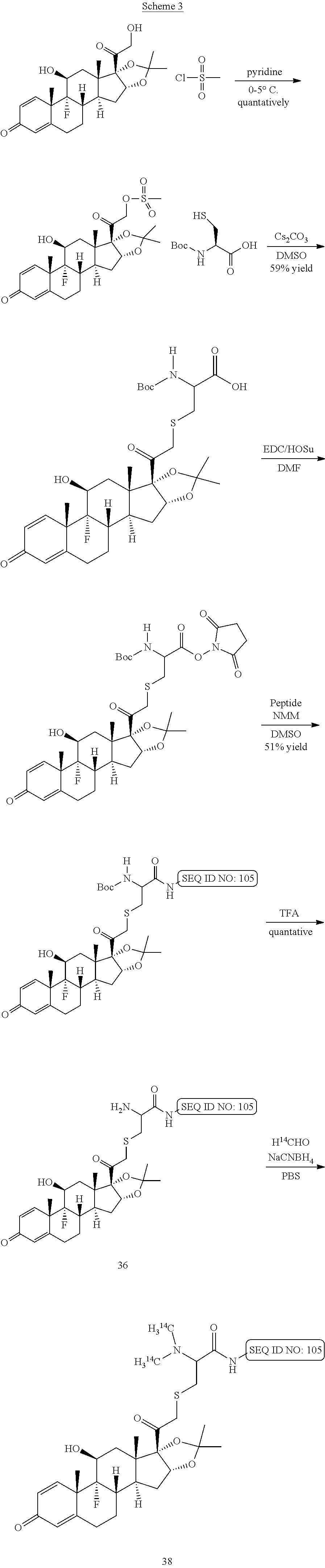

This example demonstrates a peptide conjugate comprising a cysteine linker and triamcinolone acetonide that was synthesized using the following general, non-limiting steps:
i) Mesylate formation
ii) Sulfur alkylation
iii) NHS ester formation
iv) Peptide conjugation
v) Boc Deprotection
vi) Reductive amination

These steps are illustrated in Scheme 3 and described in further detail below.

i) Mesylate Formation-Synthesis of TAA Mesylate

Triamcinolone acetonide (453.1 mg, 1.04 mmol) was dissolved in 5 mL anhydrous pyridine (not completely dissolved at the beginning, suspension). The reaction vial was sealed with a septum in an ice bath. Then methane sulfonyl chloride (116 μL, 2.08 mmol) was added dropwise to the suspension of triamcinolone acetonide through a syringe. The reaction mixture was stirred at 0° C. for half hour. Work up: the reaction mixture was poured into 25 mL iced water and the product was extracted with EtOAc. The organic part was washed by 1 mL 0.1 M HCl, saturated NaHCO₃ and brine, then dried over Na₂SO₄. The mixture was filtered to get the filtrate and the solvent was removed under reduced pressure to give the product quantatively.

ii) Sulfur Alkylation-Synthesis of TAA-Boc-Cysteine

To the stirred mixture of Boc-L-Cysteine (873 mg, 4.14 mmol) and Cs₂CO₃ (1610 mg, 4.94 mmol) in 5 mL anhydrous DMSO, were added triamcinolone acetonide mesylate (273.1 mg, 0.53 mmol) in 2 mL anhydrous DMSO. The reaction mixture was stirred at room temperature for approximately 20 hours. The reaction was filtered to remove the solid; the resulting solution was purified by HPLC to obtain 200 mg white powder with 59% yield. LC-MS showed greater than 96% purity with mass 620.2 (M−17).

iii) NHS Ester Formation-Synthesis of TAA-Boc-Cysteine-NHS Ester

To the stirred solution of triamcinolone acetonide-Boc-Cysteine (46 mg, 0.072 mmol) in 1 mL anhydrous DMF, were added 1-Ethyl-3-(3-dimethylaminopropyl)carbodiimide (27.5 mg, 0.144 mmol) and N-hydroxysuccinimide (16.6 mg, 0.144 mmol). The reaction mixture was stirred at room temperature for 16 hours. The reaction mixture was purified by HPLC to give 41.4 mg white powder with 65% purity (33% starting acid).

iv) Peptide Conjugation-Synthesis of Peptide(SEQ ID NO: 105)-Boc-Cys-TAA (“Peptide(SEQ ID NO: 105)-Boc-Cys” is Disclosed as SEQ ID NO: 512)

To the stirred mixture of the peptide having amino acid sequence SEQ ID NO: 105 (10 mg, 0.0023 mmol) and N-methylmorpholine (506 μL in 0.5 mL anhydrous DMSO; 100× dilution; 0.046 mmol), was added triamcinolone acetonide-Boc-Cysteine-NHS ester (5.1 mg, 50% purity, 0.0035 mmol). The reaction mixture was stirred at room temperature for overnight. The reaction mixture was purified by HPLC to get 5.8 mg white powder with 51% yield. LC-MS showed 98% purity with mass 1231.1 (M/4) and 1641.2 (M/3).

v) Boc Deprotection-Synthesis of Peptide(SEQ ID NO: 105)-Cys-TAA (36) (“Peptide(SEQ ID NO: 105)-Cys” is Disclosed as SEQ ID NO: 512)

To the white powder of triamcinolone acetonide-Boc-Cysteine-Peptide (12 mg, 0.00244 mmol) in 30 mL clear vial, was added 600 μL TFA (99% purity). After 5 min LC-MS analysis revealed the reaction was complete. 2 mL of acetonitrile and water (1:1 ratio) was added, then frozen at −78° C. followed by lyophilization to obtain 12 mg of white powder. LC-MS showed greater than 96% purity with mass 1206.3 (M/4), 1608.7 (M/3).

vii) Reductive Amination-Synthesis of ¹⁴C Labeled Conjugate (38)

To the solution of peptide(SEQ ID NO:105)-Cysteine-triamcinolone acetonide (5 mg, 0.00148 mmol) in 5 mL water, was added 470 μL 10×PBS and 25 μL ¹⁴C labeled formaldehyde (8% aq. solution) in a radioactive fume hood. NaCNBH₄ (aq) solution (4.6 mg in 1 mL) was added. The reaction mixture was vortexed and left for approximately 20 hours. The reaction mixture was passed over a Strata-X column previously activated with 3 mL methanol and equilibrated with 3 mL water. The adsorbed conjugate was washed with water (3 mL) and eluted with 4 mL of 2% formic acid in methanol. The methanol/formic acid was removed under a stream of nitrogen to give 4.7 mg product.

### Example 8

### Synthesis of Peptide(SEQ ID NO: 105)-Cys-Dex (29) (“peptide(SEQ ID NO: 105)-Cys” is Disclosed as SEQ ID NO: 512) and Peptide(SEQ ID NO: 105)-¹⁴Cys-Dex (30) (“Peptide(SEQ ID NO: 105)-¹⁴Cys” is Disclosed as SEQ ID NO: 512)

This example demonstrates the synthesis of peptide(SEQ ID NO: 105)-Cys-Dex (29) and peptide(SEQ ID NO: 105)-¹⁴Cys-Dex (30) (“peptide(SEQ ID NO: 105)-¹⁴Cys” is disclosed as SEQ ID NO: 512) as shown in the exemplified scheme below.

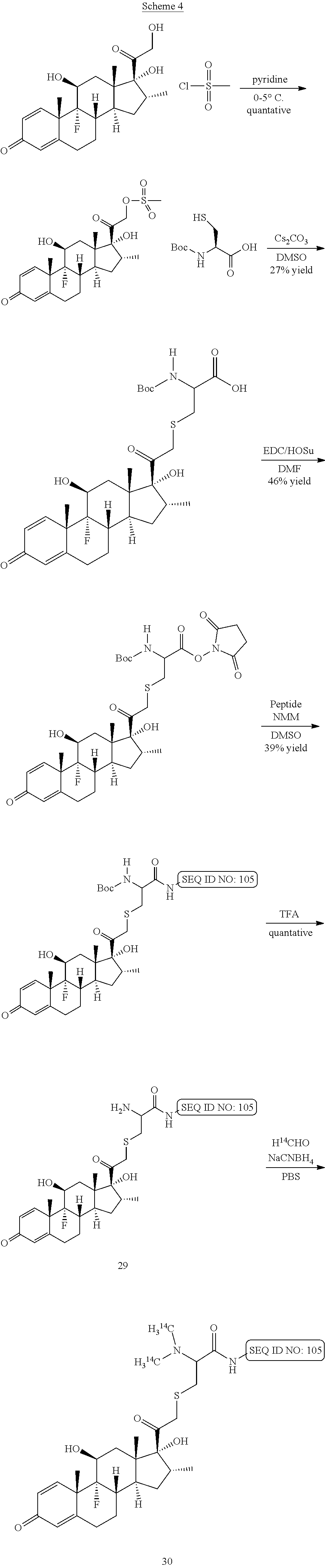

This example demonstrates a peptide conjugate comprising a cysteine linker and dexamesasone that was synthesized using the following general, non-limiting steps:
i) Mesylate formation
ii) Sulfur alkylation
iii) NHS ester formation
iv) Peptide conjugation
v) Boc Deprotection
vi) Reductive amination

These steps are illustrated in Scheme 4 and described in further detail below.

i) Mesylate Formation-Synthesis of Dex Mesylate

Procedure: Dexamethasone (455.6 mg, 1.16 mmol) was dissolved in 5 mL anhydrous pyridine. The reaction vial was sealed with a septum in an ice bath. Methane sulfonyl chloride (180 μL, 2.32 mmol) was added dropwise to the solution of dexamethasone through a syringe. The reaction mixture was stirred at 0° C. for half hour. Work up: the reaction mixture was poured into 25 mL iced water, the product was extracted with EtOAc. The organic part was washed by 1 mL 0.1 M HCl, saturated NaHCO₃ and brine, then dried over Na₂SO₄ The mixture was filtered to get the filtrate and the solvent was removed under reduced pressure to give the product quantitatively. LC-MS showed greater than 90% purity with mass 471.2 (M+1).

ii) Sulfur Alkylation-Synthesis of Dex-Boc-Cysteine

To the stirred mixture of Boc-L-Cysteine (278 mg, 1.32 mmol) and Cs₂CO₃ (443 mg, 1.36 mmol) in 2 mL anhydrous DMF, were added dexmethasone mesylate (58.5 mg, 0.124 mmol). The reaction mixture was stirred at room temperature for one hour. The solid was removed by filtration and the solution purified by HPLC to give 20 mg white powder with 27% yield. LC-MS showed 99% purity with MS 578.3 (M−17).

iii) NHS Ester Formation-Synthesis of Dex-Boc-Cysteine-NHS Ester

To the stirred mixture of Dexmethasone-Boc-Cysteine (97.3 mg, 0.16 mmol) in 1 mL anhydrous DMF, were added 1-Ethyl-3-(3-dimethylaminopropyl)carbodiimide (93.4 mg, 0.49 mmol) and N-Hydroxysuccinimide (147 mg, 1.28 mmol). The reaction mixture was stirred at room temperature for 2 hours. The reaction mixture was purified by HPLC to get 51 mg white powder with 46% yield. LC-MS showed 82% purity (15% starting acid) with MS 715 (M+23).

iv) Peptide Conjugation-Synthesis of Peptide(SEQ ID NO: 105)-Cys-Dex (“Peptide(SEQ ID NO: 105)-Cys” is Disclosed as SEQ ID NO: 512)

To the stirred mixture of the peptide having amino acid sequence SEQ ID NO: 105 (25 mg, 0.00582 mmol) and N-methylmorpholine (640 μL, 100× dilution, 0.0582 mmol) in 0.5 mL DMSO, was added dexamethasone-Boc-Cysteine-NHS ester (7.4 mg, 82% purity, 0.00872 mmol) in 0.5 mL DMSO. The reaction mixture was stirred at room temperature for overnight. The reaction mixture was purified by HPLC to give 11 mg white powder with 39% yield. LC-MS showed 96% purity. MS, 1221.0 (M/4), 1627.1 (M/3).

v) Boc Deprotection-Synthesis of Peptide(SEQ ID NO: 105)-Cys-Dex (29) (“Peptide(SEQ ID NO: 105)-Cys” is Disclosed as SEQ ID NO: 512)

To the white powder of dexamethasone-Boc-Cysteine-Peptide(SEQ ID NO: 105) (73.5 mg, 0.015 mmol) (“Cysteine-Peptide(SEQ ID NO: 105)” is disclosed as SEQ ID NO: 512) in 30 mL clear glass vial, was added 500 μL TFA (99% purity). The reaction finished by LC-MS. Acetonitrile and water (1:1 ratio) 4 mL were added, frozen at −78° C. then dried in Lyophilizer to get 71.6 mg white powder. LC-MS showed 96% purity with MS, 1195.5 (M/4).

vi) Reductive Amination-Synthesis of ¹⁴C Labeled Conjugate (30) (“Peptide(SEQ ID NO: 105)-Cys” is Disclosed as SEQ ID NO: 512)

To the solution of peptide(SEQ ID NO: 105)-Cysteine-Dexamethasone (36 mg, 0.00742 mmol) (“peptide(SEQ ID NO: 105)-Cys” is disclosed as SEQ ID NO: 512) in 16 mL water, was added 2.35 mL PBS (10×) and 0.15 mL 8% ¹⁴C labeled formaldehyde (8% aq. solution) in a radioactive fume hood. NaCNBH₄ (aq) solution (18 mg in 3 mL) was added. The reaction mixture was vortexed and left for approximately 20 hours. The reaction mixture was passed over a Strata-X column previously activated with 3 mL methanol and equilibrated with 3 mL water. The adsorbed conjugate was washed with water (3 mL) and eluted with 4 mL of 2% formic acid in methanol. The methanol/formic acid was removed under a stream of nitrogen to give 20.3 mg product.

### Example 9

### Synthesis of Peptide(SEQ ID NO: 105)-Glutaric Acid-Dex (23)

This example demonstrates the synthesis of peptide(SEQ ID NO: 105)-glutaric acid-Dex (23) as shown in the exemplified scheme below.

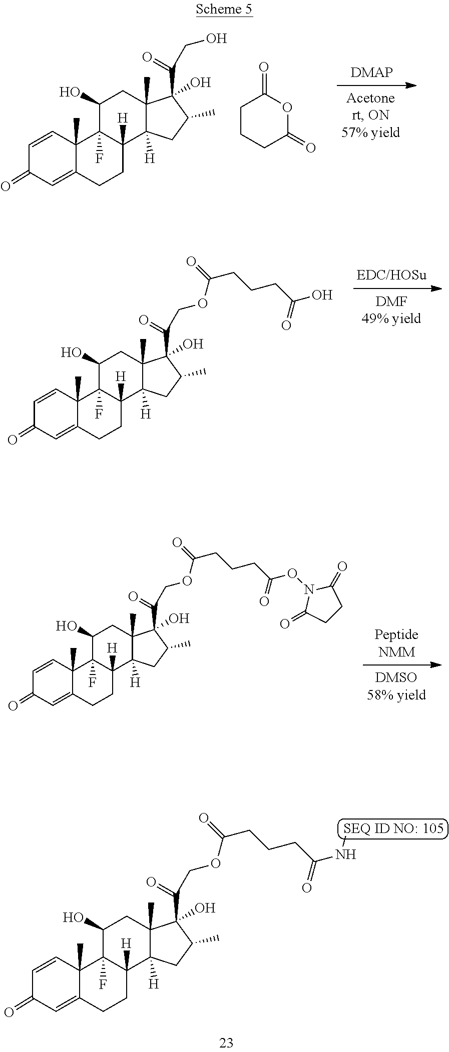

This example demonstrates a peptide conjugate comprising a glutaric linker and dexamethasone that was synthesized using the following general, non-limiting steps:
i) Ester bond formation
ii) NHS ester formation
iii) Peptide conjugation

These steps are illustrated in Scheme 5 and described in further detail below.

i) Ester Bond Formation-Synthesis of Dex-Gutaric Acid

Dexamethasone (1074.4 mg, 2.73 mmol), Glutaric anhydride (393.1 mg, 3.28 mmol) and DMAP (400.7 mg, 3.28 mmol) were dissolved in anhydrous acetone 25 mL (not completely dissolved at the beginning, become clear after 1 h). The reaction mixture was stirred at room temperature for approximately 20 hours. The solvent was removed under reduced pressure. The residue was purified by HPLC to give 787 mg white powder with 57% yield. LC-MS showed greater than 99% purity with mass 489.1 (M−17).

ii) NHS Ester Formation-Synthesis of Dex-Glutaric NHS Ester

To the stirred mixture of Dexmethasone-glutaric acid (68 mg, 0.134 mmol) in 1 mL DMF, were added 1-Ethyl-3-(3-dimethylaminopropyl)carbodiimide (77 mg, 0.403 mmol) and N-Hydroxysuccinimide (77 mg, 0.67 mmol). The reaction mixture was stirred at room temperature for approximately 20 hours. The reaction mixture was purified by HPLC to give 40 mg white powder with 49% yield. LC-MS showed greater than 98% purity with mass 584.2 (M−19).

iii) Peptide Conjugation-Synthesis Peptide(SEQ ID NO: 105)-Glutaric Acid-Dex (23)

To the stirred mixture of the peptide having amino acid sequence SEQ ID NO: 105 (20 mg, 0.0046 mmol) and N-methylmorpholine (522 μL, 100× dilution, 0.046 mmol) in 1 mL DMSO, was added Dexamethasone-glutaric acid-NETS ester (4.2 mg, 0.0070 mmol). The reaction mixture was stirred at room temperature for approximately 20 hours. The reaction mixture was purified by HPLC to get 13 mg white powder with 58% yield. LC-MS showed 99% purity with MS: 1198.4 (M/4).

Alternatives to the peptides used in this EXAMPLE or another peptide disclosed herein, such as SEQ ID NO: 103 or SEQ ID NO: 184 or SEQ ID NO: 509, are used as the peptide for the conjugation. Alternatives to dexamethasone, budesonide, triamcinolone acetonide, or other glucocorticoid disclosed herein are used as an active agent for the conjugation.

### Example 10

### Synthesis of Peptide(SEQ ID NO: 105)-3,3,-Tetramethylene-Glutaric Acid-Dex (26)

This example demonstrates the synthesis of peptide(SEQ ID NO: 105)-3,3,-tetramethylene-glutaric acid-Dex (26) as shown in the exemplified scheme below.

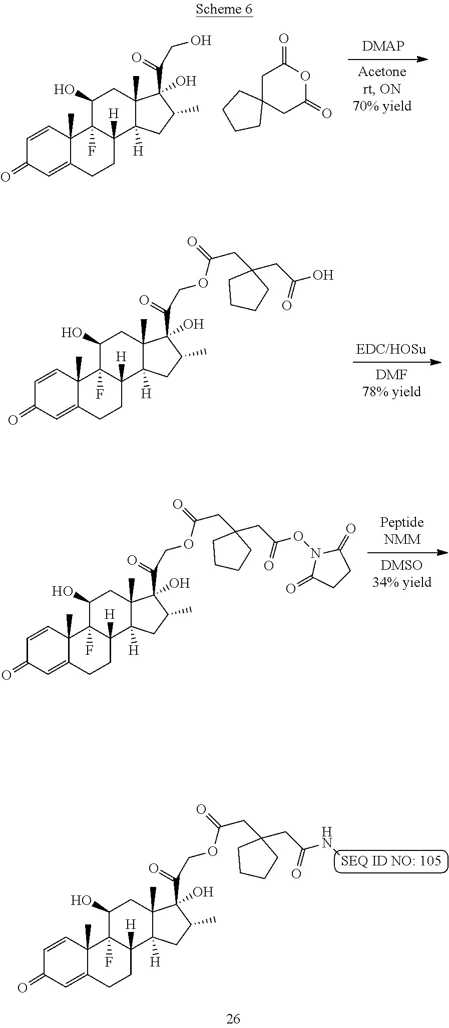

This example demonstrates a peptide conjugate comprising a 3,3-tetramethylene-glutaric linker and dexamethasone that was synthesized using the following general, non-limiting steps:
i) Ester bond formation
ii) NHS ester formation
iii) Peptide conjugation

These steps are illustrated in Scheme 6 and described in further detail below.

i) Ester Bond Formation-Synthesis of Dex-3,3-Tetramethylene-Gutaric Acid

Dexamethasone (90.5 mg, 0.23 mmol), 3,3-tetramethylene-glutaric anhydride (79 mg, 0.46 mmol) and DMAP (33.7 mg, 0.28 mmol) were dissolved in anhydrous acetone 5 mL (not completely dissolved at the beginning, become clear after 5 minute). The reaction mixture was stirred at room temperature for approximately 20 hours. The solvent was removed under reduced pressure. The residue was purified by HPLC to give 90.4 mg white powder with 70% yield. LC-MS showed greater than 95% purity with mass 543.3 (M−17).

ii) NHS Ester Formation-Synthesis of Dex-3,3-Tetramethylene-Glutaric NHS Ester

To the stirred mixture of Dexmethasone-3,3-tetramethylene-glutaric acid (90 mg, 0.16 mmol), 1-Ethyl-3-(3-dimethylaminopropyl)carbodiimide (51 mg, 0.26 mmol) and N-Hydroxysuccinimide (51 mg, 0.45 mmol) was added 1 mL DMF. The reaction mixture was stirred at room temperature for approximately 20 hours. The reaction mixture was purified by HPLC to give 82 mg white powder with 78% yield. LC-MS showed greater than 95% purity with mass 638.2 (M−19).

iii) Peptide Conjugation-Synthesis of Peptide(SEQ ID NO: 105)-3,3,-Tetramethylene-Glutaric Acid-Dex (26)

To the stirred mixture of the peptide having amino acid sequence SEQ ID NO: 105 (10 mg, 0.00232 mmol) and N-methylmorpholine (261 μL, 100× dilution, 0.0232) in 1 mL DMSO, was added Dexamethasone-3,3-tetramethylene-glutarate-NHS ester (3.2 mg, 95% purity, 0.00464 mmol). The reaction mixture was stirred at room temperature for approximately 20 hours.

LC-MS showed ⅓ peptide and ⅔ product by TIC signal. N-methylmorpholine (54 μL, 100× dilution, 0.0048) and Dexamethasone-3,3-tetramethylene-glutarate-NHS ester (3.2 mg, 95% purity, 0.00464 mmol) was added. The reaction mixture was stirred at room temperature for approximately 24 hours (another overnight).

The reaction mixture was purified by HPLC to get 3.8 mg white powder with 34% yield. LC-MS showed 96% purity with MS: 1212.0 (M/4).

### Example 11

### Synthesis of Peptide(SEQ ID NO: 105)-Glutaric Acid-TAA (35)

This example demonstrates the synthesis of peptide(SEQ ID NO: 105)-glutaric acid-TAA (35) as shown in the exemplified scheme below.

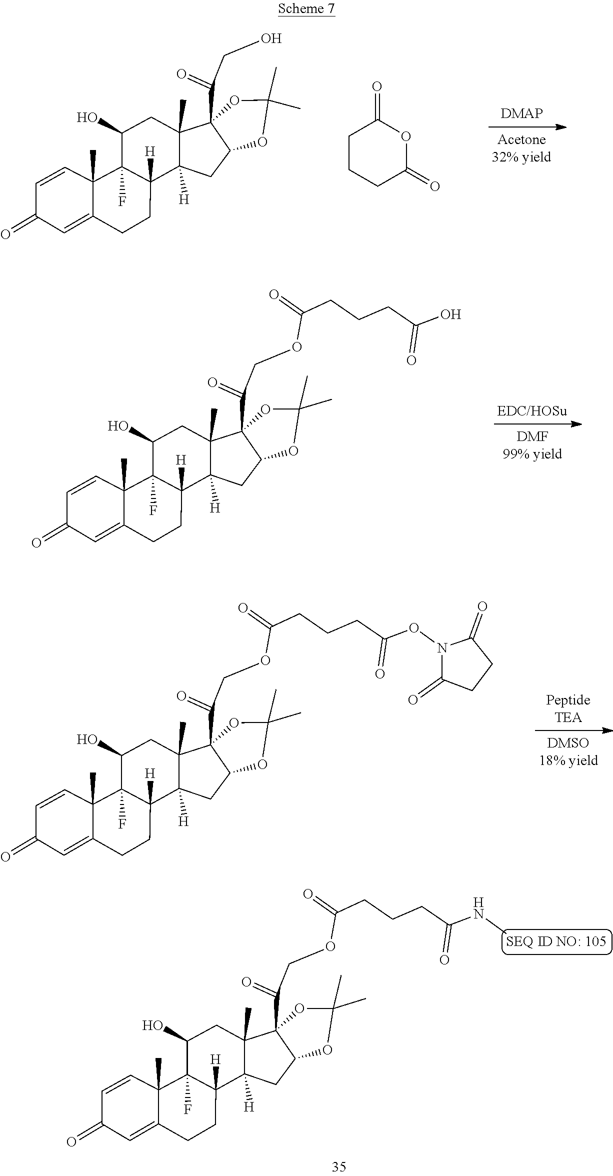

This example demonstrates a peptide conjugate comprising a glutaric linker and triamcinolone acetonide that was synthesized using the following general, non-limiting steps:
i) Ester bond formation
ii) NHS ester formation
iii) Peptide conjugation

These steps are illustrated in Scheme 7 and described in further detail below.

i) Ester Bond Formation-Synthesis of TAA-Glutaric Acid

Triamcinolone acetonide (120.7 mg, 0.28 mmol), glutaric anhydride (37.7 mg, 0.33 mmol) and DMAP (38.2 mg, 0.31 mmol) were dissolved in anhydrous acetone 4 mL (not completely dissolved at the beginning, become clear after 2 hours). The reaction mixture was stirred at room temperature for approximately 20 hours. LC-MS showed 10% triamcinolone acetonide starting material, 66% product and 23% dimer TAA-glutaric acid-TAA.

Glutaric anhydride (10 mg, 0.09 mmol) and DMAP (9 mg, 0.07 mol) were added. The reaction mixture was stirred at room temperature for approximately 20 hours. The solvent was removed under reduced pressure. The residue was purified by HPLC to give 50 mg white powder with 32% yield. LC-MS showed greater than 95% purity with mass 531.2 (M−17).

ii) NHS Ester Formation-Synthesis of TAA-Glutaric NHS Ester

To the stirred mixture of triamcinolone acetonide-glutaric acid (50 mg, 0.091 mmol) in 1 mL DMF, were added 1-Ethyl-3-(3-dimethylaminopropyl)carbodiimide (26 mg, 0.137 mmol) and N-Hydroxysuccinimide (16 mg, 0.137 mmol). The reaction mixture was stirred at room temperature for approximately 20 hours. LC-MS showed half starting material and half product.

1-Ethyl-3-(3-dimethylaminopropyl)carbodiimide (38 mg, 0.20 mmol) and N-Hydroxysuccinimide (34 mg, 0.30 mmol) were added. The reaction mixture was purified by HPLC to give 52 mg white powder with 99% yield. LC-MS showed greater than 98% purity with mass 668.2 (M+23).

iii) Peptide Conjugation-Synthesis of Peptide(SEQ ID NO: 105)-Glutaric Acid-TAA (35)

To the stirred mixture of the peptide having amino acid sequence SEQ ID NO: 105 (5 mg, 0.00116 mmol) and triamcinolone acetonide-glutaric acid-NETS ester (0.76 mg, 0.00116 mmol) in 0.5 mL DMSO, was added triethylamine (0.94 mg, 129 μL, 100× dilution in DMSO, 0.00928 mmol). The reaction mixture was stirred at room temperature for approximately 48 hours (2 days). The reaction mixture was purified by HPLC to get 1 mg white powder with 18% yield. LC-MS showed greater than 90% purity with MS: 1208.9 (M/4).

### Example 12

### Synthesis of Peptide(SEQ ID NO: 509)-trans-1,2-cyclohexyl-TAA (31)

This example demonstrates the synthesis of peptide(SEQ ID NO: 509)-trans-1,2-cyclohexyl-TAA (31) as shown in the exemplified scheme below.

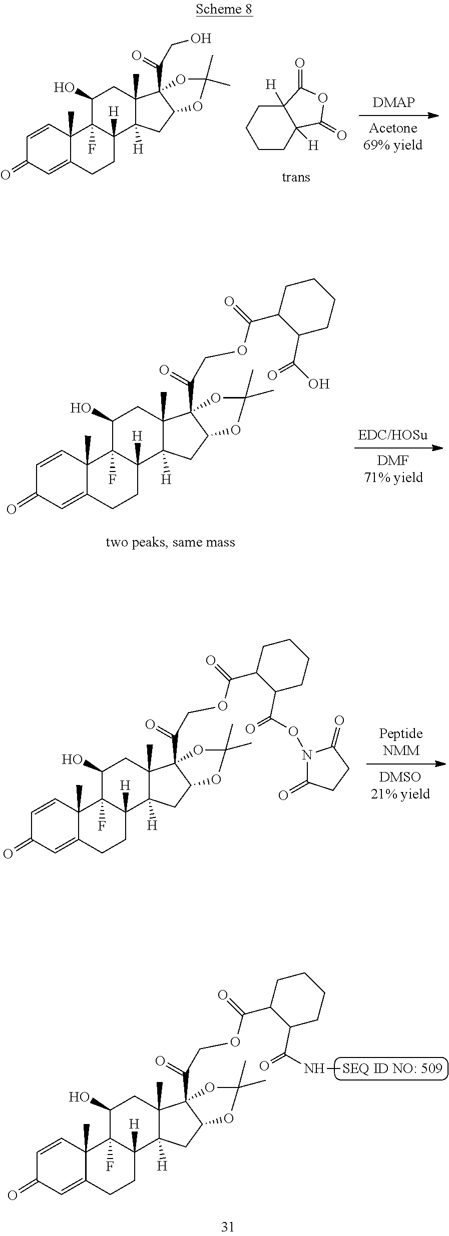

This example demonstrates a peptide conjugate comprising a trans-1,2-cyclohexyl linker and triamcinolone acetonide that was synthesized using the following general, non-limiting steps:
i) Ester bond formation
ii) NHS ester formation
iii) Peptide conjugation

These steps are illustrated in Scheme 8 and described in further detail below.

i) Ester Bond Formation-Synthesis of TAA-trans-1,2-cyclohexane-carboxylic Acid

Triamcinolone acetonide (65 mg, 0.15 mmol), trans-1,2-cyclohexane-dicarboxylic anhydride (30 mg, 0.20 mmol) and DMAP (24 mg, 0.20 mmol) were dissolved in anhydrous acetone 3 mL (not completely dissolved at the beginning, become clear after 2 hours). The reaction mixture was stirred at room temperature for approximately 20 hours. The solvent was removed under reduced pressure. The residue was purified by HPLC to give 60.4 mg white powder with 69% yield (two peaks with the same mass). LC-MS showed greater than 90% purity with mass 571.3 (M−17).

ii) NHS Ester Formation-Synthesis of TAA-trans-1,2-cyclohexane-carboxylic NHS Ester

To the stirred mixture of triamcinolone acetonide-trans-1,2-cyclohexane-carboxylic acid (30.4 mg, 0.051 mmol) in 1 mL DMF, were added 1-Ethyl-3-(3-dimethylaminopropyl)carbodiimide (19.5 mg, 0.102 mmol) and N-Hydroxysuccinimide (11.7 mg, 0.102 mmol). The reaction mixture was stirred at room temperature for approximately 20 hours. The reaction mixture was purified by HPLC to give 24.7 mg white powder with 71% yield. LC-MS showed greater than 98% purity (two peaks) with mass 708.3 (M+23).

iii) Peptide Conjugation-Synthesis of Peptide(SEQ ID NO: 509)-trans-1,2-cyclohexyl-TAA (31)

To the stirred mixture of the peptide having amino acid sequence SEQ ID NO: 509 (5 mg, 0.00114 mmol) and N-methylmorpholine (125 μL, 100× dilution, 0.0114) in 1 mL DMSO, was added triamcinolone acetonide-trans-1,2-cyclohexane-carboxylic-NHS ester (1.56 mg, 0.00228 mmol). The reaction mixture was stirred at room temperature for approximately 48 hours (2 days). The reaction mixture was purified by HPLC to get 1.2 mg white powder with 21% yield. LC-MS showed greater than 98% purity with MS: 1238.8 (M/4) and 1651.6 (M/3).

### Example 13

### Synthesis of Peptide(SEQ ID NO: 509)-1,3-cyclohexyl-TAA (32)

This example demonstrates the synthesis of peptide(SEQ ID NO: 509)-1,3-cyclohexyl-TAA (32) as shown in the exemplified scheme below.

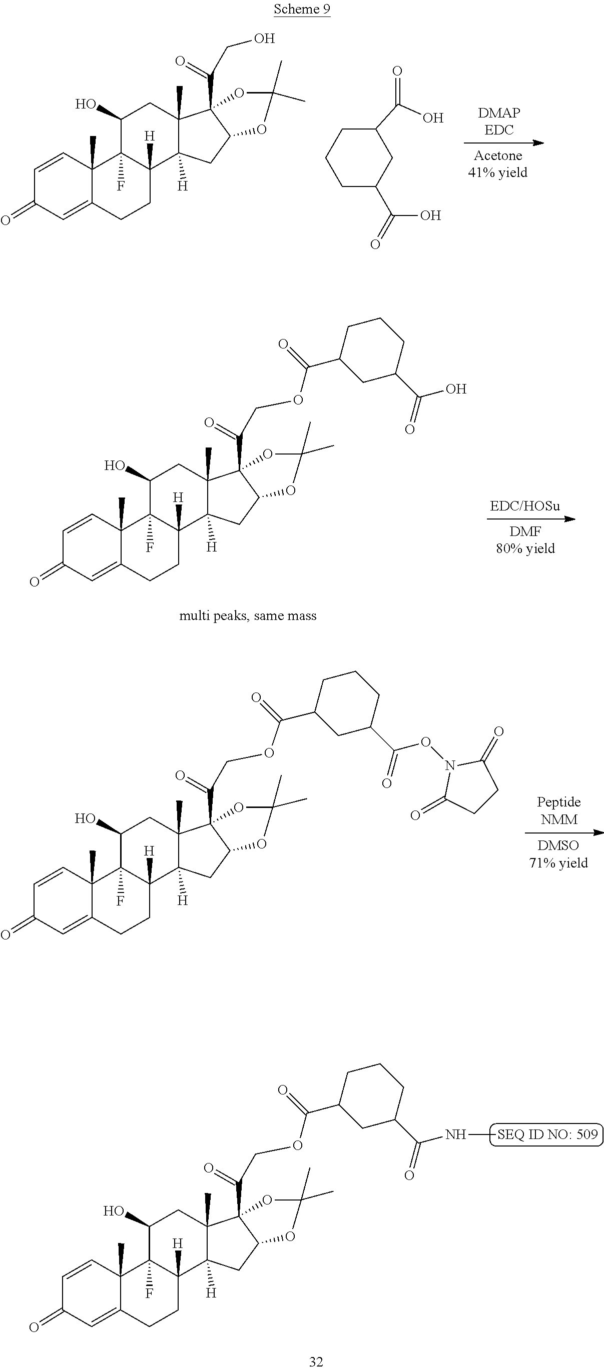

This example demonstrates a peptide conjugate comprising a 1,3-cyclohexyl linker and triamcinolone acetonide that was synthesized using the following general, non-limiting steps:
i) Ester bond formation
ii) NHS ester formation
iii) Peptide conjugation

These steps are illustrated in Scheme 9 and described in further detail below.

i) Ester Bond Formation-Synthesis of TAA-1,3-cyclohexane-carboxylic Acid

Triamcinolone acetonide (56 mg, 0.13 mmol), 1,3-cyclohexane-dicarboxylic acid (29 mg, 0.17 mmol) and EDC (32 mg, 0.17 mmol) and DMAP (21 mg, 0.17 mmol) were dissolved in anhydrous acetone 4 mL. The reaction mixture was stirred at room temperature for approximately 20 hours. LC-MS showed ⅓ triamcinolone acetonide starting material. 1,3-cyclohexane-dicarboxylic acid (20 mg, 0.12 mmol) and EDC (19 mg, 0.10 mmol) and DMAP (17 mg, 0.14 mmol) were added again. The reaction mixture was stirred at room temperature for approximately 24 hours.

The solvent was removed under reduced pressure. The residue was purified by HPLC to give 31 mg white powder with 41% yield (multi peaks with the same mass). LC-MS showed greater than 90% purity with mass 571.2 (M−17).

ii) NHS Ester Formation-Synthesis of TAA-1,3-cyclohexane-carboxylic NHS Ester

To the stirred mixture of triamcinolone acetonide-1,3-cyclohexane-carboxylic acid (24 mg, 0.041 mmol) in 1 mL DMF, were added 1-Ethyl-3-(3-dimethylaminopropyl)carbodiimide (15 mg, 0.082 mmol) and N-Hydroxysuccinimide (9.5 mg, 0.082 mmol). The reaction mixture was stirred at room temperature for approximately 20 hours. The reaction mixture was purified by HPLC to give 22.3 mg white powder with 80% yield. LC-MS showed greater than 98% purity (multi peaks) with mass 708.2 (M+23).

iii) Peptide Conjugation-Synthesis of Peptide(SEQ ID NO: 509)-1,3-cyclohexyl-TAA (32)

To the stirred mixture of the peptide having amino acid sequence SEQ ID NO: 509 (5 mg, 0.00116 mmol) and N-methylmorpholine (125 μL, 100× dilution, 0.0114) in 1 mL DMSO, was added triamcinolone acetonide-1,3-cyclohexane-carboxylic-NHS ester (1.56 mg, 0.00228 mmol). The reaction mixture was stirred at room temperature for approximately 48 hours (2 days). The reaction mixture was purified by HPLC to get 4 mg white powder with 71% yield. LC-MS showed greater than 98% purity with MS: 1239.1 (M/4) and 1651.8 (M/3).

### Example 14

### Synthesis of Peptide(SEQ ID NO: 509)-terephthalic Acid-TAA (33)

This demonstrates the synthesis of peptide(SEQ ID NO: 509)-terephthalic acid-TAA (33) as shown in the exemplified scheme below.

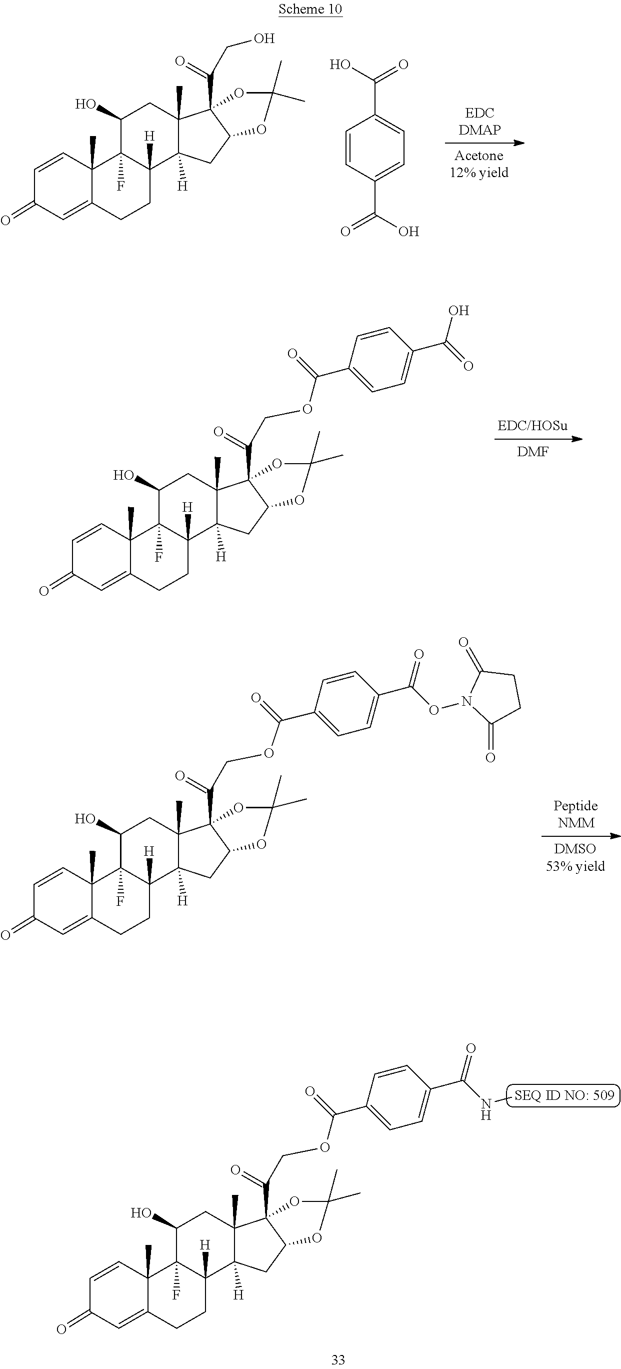

This example demonstrates a peptide conjugate comprising a terephthalic linker and triamcinolone acetonide that was synthesized using the following general, non-limiting steps:
i) Ester bond formation
ii) NHS ester formation
iii) Peptide conjugation

These steps are illustrated in Scheme 10 and described in further detail below.

i) Ester Bond Formation-Synthesis of TAA-Terephthalic Acid

Triamcinolone acetonide (55.2 mg, 0.13 mmol), terephthalic acid (27.4 mg, 0.17 mmol), EDC (31.6 mg, 0.17 mmol) and DMAP (20.2 mg, 0.17 mmol) were dissolved in anhydrous acetone 4 mL. The reaction mixture was stirred at room temperature for approximately 20 hours. LC-MS showed major triamcinolone acetonide starting material. Terephthalic acid (42 mg, 0.25 mmol), EDC (49 mg, 0.26 mmol) and DMAP (31 mg, 0.25 mmol) dissolved in 1 mL DMF and 1 mL DMSO was further added. The reaction mixture was stirred at room temperature for approximately 24 hours.

The solvent was removed under reduced pressure. The residue was purified by HPLC to give 8.5 mg white powder with 12% yield. LC-MS showed greater than 90% purity with mass 583.2 (M+1).

ii) NHS Ester Formation-Synthesis of TAA-Terephthalic NHS Ester

To the stirred mixture of triamcinolone acetonide-terephthalic acid (8.5 mg, 0.015 mmol) in 1 mL DMF, were added 1-Ethyl-3-(3-dimethylaminopropyl)carbodiimide (5.6 mg, 0.029 mmol) and N-Hydroxysuccinimide (3.4 mg, 0.029 mmol). The reaction mixture was stirred at room temperature for approximately 48 hours (2 days). The reaction mixture was purified by HPLC to give 1.3 mg white powder with 27% purity by LC-MS and 0.4 mg with 70% purity. Total 0.63 mg product with 6% yield. LC-MS showed mass 680.2 (M+1).

iii) Peptide Conjugation-Synthesis of Peptide(SEQ ID NO: 509)-Terephthalic Acid-TAA (33)

To the stirred mixture of the peptide having amino acid sequence SEQ ID NO: 509 (3 mg, 0.00069 mmol) and N-methylmorpholine (226 μL, 100× dilution, 0.021) in 1 mL DMSO, was added triamcinolone acetonide-terephthalic-NHS ester (0.63 mg, 0.0093 mmol). The reaction mixture was stirred at room temperature for approximately 20 hours. The reaction mixture was purified by HPLC to get 1.8 mg white powder with 53% yield. LC-MS showed greater than 98% purity with MS: 1237.3 (M/4) and 1649.3 (M/3).

### Example 15

### Synthesis of Peptide(SEQ ID NO: 105)-2,3-dimethyl-succinic Acid-BUD (37)

This demonstrates the synthesis of peptide(SEQ ID NO: 105)-2,3-dimethyl-succinic acid-BUD (37) as shown in the exemplified scheme below.

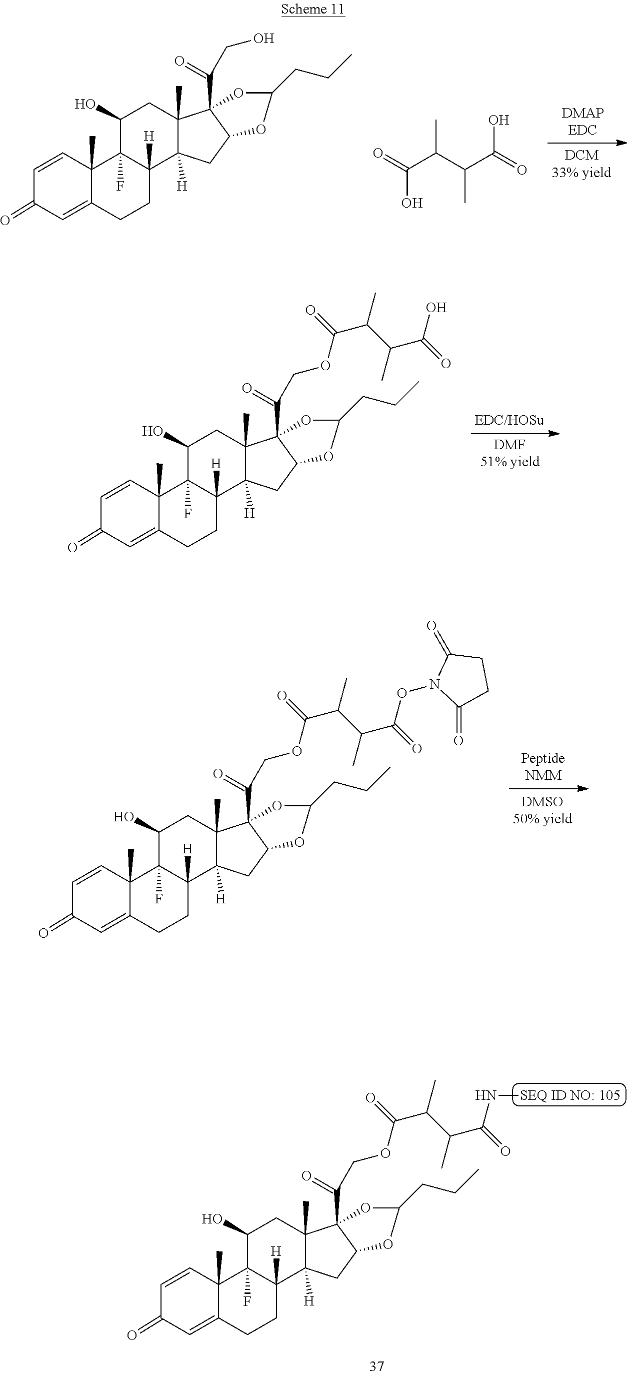

This example demonstrates a peptide conjugate comprising a 2,3-dimethyl-succinic linker and budesonide that was synthesized using the following general, non-limiting steps:
i) Ester bond formation
ii) NHS ester formation
iii) Peptide conjugation

These steps are illustrated in Scheme 11 and described in further detail below.

i) Ester Bond Formation-Synthesis of BUD-2,3-dimethyl-succinic Acid

Budesonide (46.5 mg, 0.11 mmol), 2,3-dimethyl-succinic acid (20.2 mg, 0.14 mmol), EDC (25 mg, 0.13 mmol) and DMAP (16 mg, 0.13 mmol) were dissolved in anhydrous dichloromethane 2 mL. The reaction mixture was stirred at room temperature for approximately 20 hours. The solvent was removed under reduced pressure. The residue was purified by HPLC to give 19.7 mg white powder with 33% yield. LC-MS showed greater than 95% purity with mass 559.3 (M+1).

ii) NHS Ester Formation-Synthesis of BUD-2,3-dimethyl-succinic NHS Ester

To the stirred mixture of budesonide-2,3-dimethyl-succinic acid (32 mg, 0.057 mmol) in 1 mL DMF, were added 1-Ethyl-3-(3-dimethylaminopropyl)carbodiimide (27.4 mg, 0.14 mmol) and N-Hydroxysuccinimide (16 mg, 0.137 mmol). The reaction mixture was stirred at room temperature for approximately 5 hours. The reaction mixture was purified by HPLC to give 18.9 mg white powder with 51% yield. LC-MS showed greater than 95% purity with mass 678.1 (M+23).

iii) Peptide Conjugation-Synthesis of Peptide(SEQ ID NO: 105)-2,3-dimethyl-succinic Acid-BUD (37)

To the stirred mixture of the peptide having amino acid sequence SEQ ID NO: 105 (5 mg, 0.00116 mmol) and N-methylmorpholine (131 μL, 100× dilution, 0.023) in 1 mL DMSO, was added budesonide-2,3-dimethyl-succinic NHS ester (1.6 mg, 95% purity 0.0023 mmol). The reaction mixture was stirred at room temperature for approximately 20 hours. LC-MS showed 80% unreacted peptide and 20% product by TIC signal. N-methylmorpholine (131 μL, 100× dilution, 0.023) and budesonide-2,3-dimethyl-succinic NHS ester (2 mg, 95% purity, 0.0029 mmol) was further added. The reaction mixture was stirred at room temperature for a further 24 hours. The reaction mixture was purified by HPLC to get 1.8 mg white powder with 50% yield. LC-MS showed greater than 90% purity with MS: 1211.8 (M/4).

### Example 16

### Synthesis of Peptide(SEQ ID NO: 105)-Succinic Acid-Dex (39)

This demonstrates the synthesis of peptide(SEQ ID NO: 105)-succinic acid-Dex (39) as shown in the exemplified scheme below.

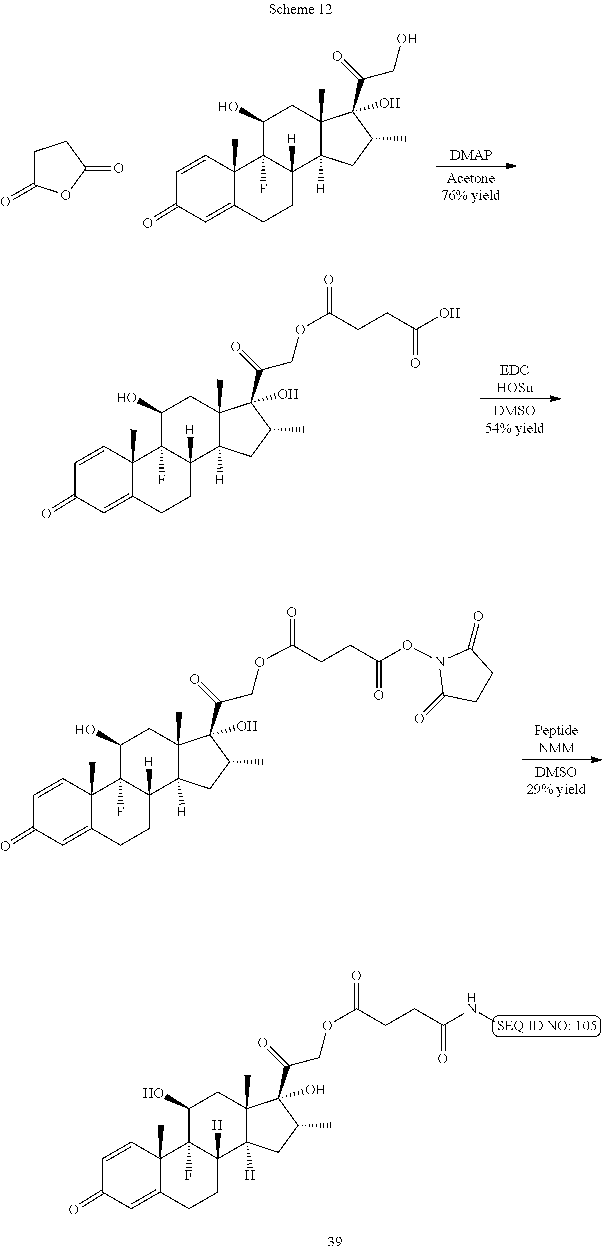

This example demonstrates a peptide conjugate comprising a succinic acid linker and dexamethasone was synthesized using the following general, non-limiting steps:
i) Ester bond formation
ii) NHS ester formation
iii) Peptide conjugation

These steps are illustrated in Scheme 12 and described in further detail below.

i) Ester Bond Formation-Synthesis of Dex-Succinic Acid

Dexamethasone (50 mg, 0.127 mmol), succinic anhydride (15 mg, 0.153 mmol) and DMAP (19 mg, 0.153 mmol) were dissolved in 2 mL anhydrous acetone. The reaction mixture was stirred at room temperature for approximately 96 hours (4 days). The solvent was removed under reduced pressure. The residue was purified by HPLC to give 48 mg white powder with 76% yield. LC-MS showed greater than 98% purity with mass 473.1 (M−19).

ii) NHS Ester Formation-Synthesis of Dex-Succinic NHS Ester

Dex-succinic acid (48 mg, 0.0975 mmol), N-(3-dimethylaminopropyl)-N′-ethylcarbodiimide hydrochloride (60 mg, 0.312 mmol) and N-hydroxysuccinimide (36 mg, 0.312 mmol) were dissolved in 2 mL anhydrous DMSO. The reaction mixture was stirred at room temperature for 2 days (48 hrs) The reaction mixture was purified by HPLC to give 31.1 mg white powder with 54% yield. LC-MS showed greater than 98% purity with mass 590.2.

iii) Peptide Conjugation-Synthesis of Peptide(SEQ ID NO: 105)-Succinic Acid-Dex (39)

To the stirred mixture of the peptide having amino acid sequence SEQ ID NO: 105 (10 mg, 0.00233 mmol) and N-methylmorpholine (312 μL, 100× dilution, 0.0284 mmol) in 1 mL anhydrous DMSO, was added Dex-succinic NHS ester (4.5 mg, 99% purity, 0.00769 mmol). The reaction mixture was stirred at room temperature for approximately 20 hours. The reaction mixture was purified by HPLC (10 to 50 in 20 min) to give 3.2 mg white powder with 29% yield. LC-MS showed greater than 98% purity with MS: 1195.0 (M/4), 1592.2 (M/3).

### Example 17

### Synthesis of Peptide(SEQ ID NO: 105)-Adipic Acid-Dex (24)

This demonstrates the synthesis of peptide(SEQ ID NO: 105)-adipic acid-Dex (24) as shown in the exemplified scheme below.

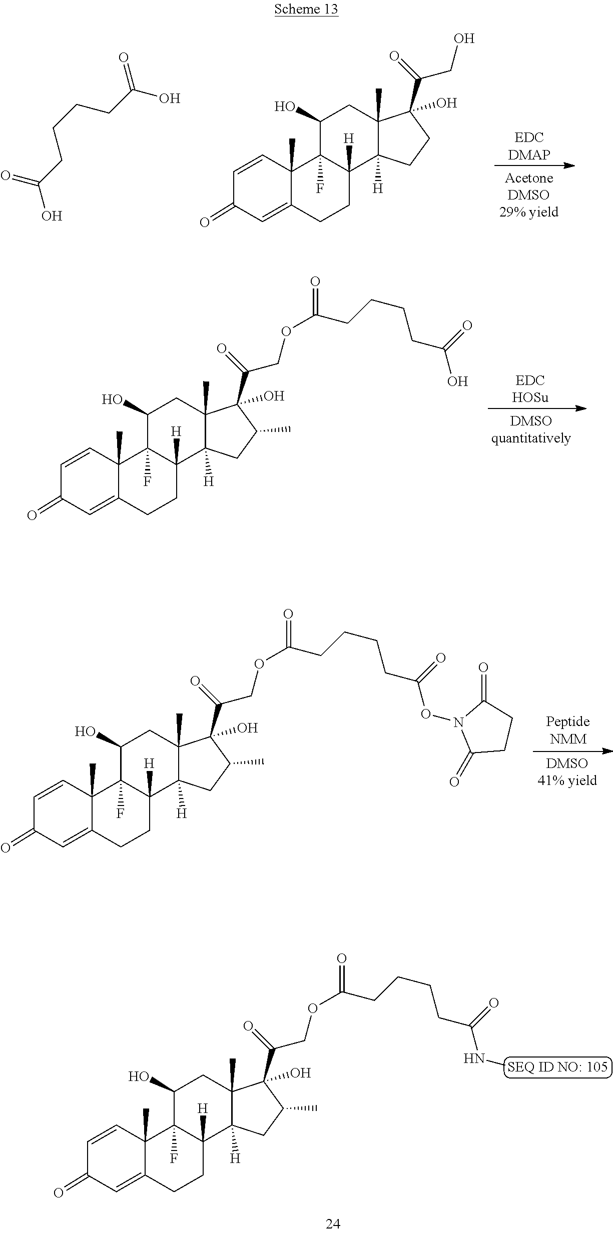

This example demonstrates a peptide conjugate comprising an adipic acid linker and dexamethasone was synthesized using the following general, non-limiting steps:
i) Ester bond formation
ii) NHS ester formation
iii) Peptide conjugation

These steps are illustrated in Scheme 13 and described in further detail below.

i) Ester Bond Formation-Synthesis of Dex-Adipic Acid

Dexamethasone (250 mg, 0.637 mmol), adipic acid (112 mg, 0.764 mmol), EDC (147 mg, 0.764 mmol) and DMAP (93 mg, 0.764 mmol) were dissolved in 9 mL anhydrous acetone and 1 mL anhydrous DMSO. The reaction mixture was stirred at room temperature for approximately 20 hours. Acetone was removed under reduced pressure. The residue was purified by HPLC to give 98 mg white powder with 29% yield. LC-MS showed greater than 98% purity with mass 503.1 (M−18).

ii) NHS Ester Formation-Synthesis of Dex-Adipic NHS Ester

Dex-adipic-carboxylic acid (98 mg, 0.188 mmol), N-(3-dimethylaminopropyl)-N′-ethylcarbodiimide hydrochloride (86 mg, 0.450 mmol) and N-hydroxysuccinimide (52 mg, 0.450 mmol) were dissolved in 2 mL anhydrous DMSO. The reaction mixture was stirred at room temperature for 2 days (48 hrs). The reaction mixture was purified by HPLC to give 117 mg white powder quantitatively. LC-MS showed greater than 98% purity with mass 640.2 (M−22).

iii) Peptide Conjugation-Synthesis of Peptide(SEQ ID NO: 105)-Adipic Acid-Dex (24)

To the stirred mixture of the peptide having amino acid sequence SEQ ID NO: 105 (10 mg, 0.00233 mmol) and N-methylmorpholine (100× dilution, 314 μL, 0.0286 mmol) in 1 mL anhydrous DMSO, was added Dex-adipic NHS ester (2.6 mg, 99% purity, 0.00421 mmol). The reaction mixture was stirred at room temperature for 2 days (48 hrs). The reaction mixture was purified by HPLC (10 to 50 in 20 min) to give 4.6 mg white powder with 41% yield. LC-MS showed greater than 98% purity with MS: 1202.2 (M/4), 1601.7 (M/3).

### Example 18

### Synthesis of Peptide(SEQ ID NO: 105)-trans-1,4-cyclohexyl-Dex (25)

This demonstrates the synthesis of peptide(SEQ ID NO: 105)-trans-1,4-cyclohexyl-Dex (25) as shown in the exemplified scheme below.

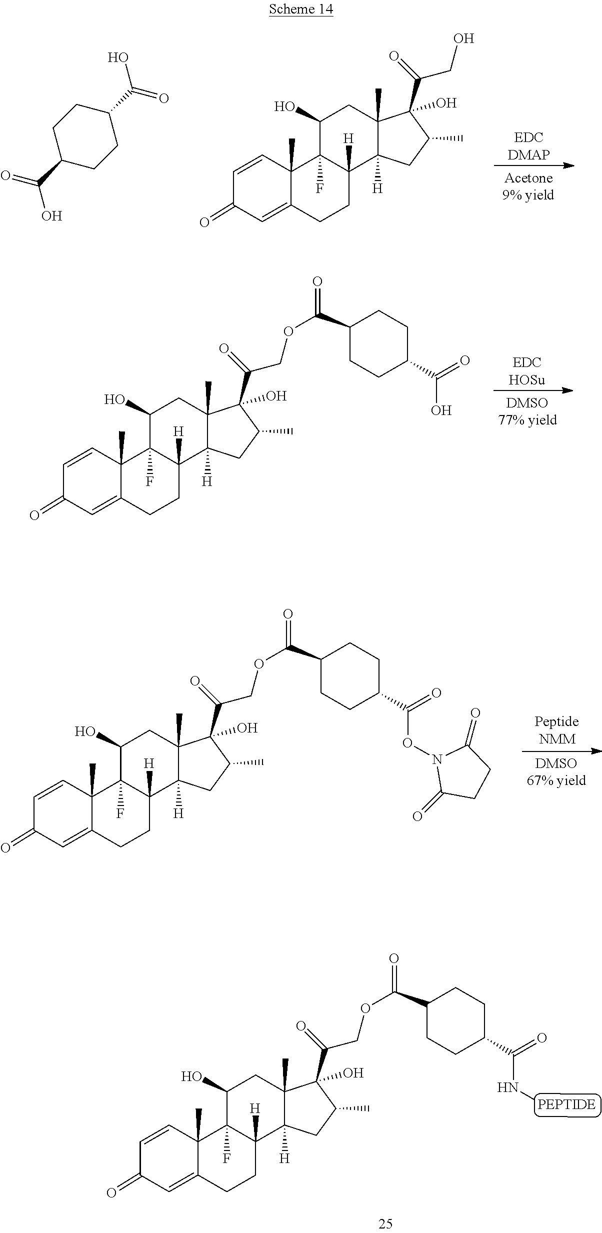

This example demonstrates a peptide conjugate comprising a trans-1,4-cyclohexyl linker and dexamethasone that was synthesized using the following general, non-limiting steps:
i) Ester bond formation
ii) NHS ester formation
iii) Peptide conjugation

These steps are illustrated in Scheme 14 and described in further detail below.

i) Ester Bond Formation-Synthesis of Dex-trans-1,4-cyclohexyl-carboxylic Acid

Dexamethasone (500 mg, 1.27 mmol), trans-1,4-Cyclohexanedicarboxylic acid (241 mg, 1.40 mmol), EDC (269 mg, 1.40 mmol) and DMAP (171 mg, 1.40 mmol) were dissolved in 35 mL anhydrous acetone. The reaction mixture was stirred at room temperature for 4 days (96 hrs). The solvent was removed under reduced pressure. The residue was purified by HPLC to give 63 mg white powder with 9% yield. LC-MS showed greater than 98% purity with mass 527.2 (M−19).

ii) Ii) NHS Ester Formation-Synthesis of Dex-trans-1,4-cyclohexyl NHS Ester

Dex-trans-1,4-cyclohexyl-carboxylic acid (63 mg, 0.115 mmol), N-(3-dimethylaminopropyl)-N′-ethylcarbodiimide hydrochloride (26.6 mg, 0.139 mmol) and N-hydroxysuccinimide (16 mg, 0.139 mmol) were dissolved in 1 mL anhydrous DMSO. The reaction mixture was stirred at room temperature for approximately 5 hours. The reaction mixture was purified by HPLC to give 57.1 mg white powder with 77% yield. LC-MS showed greater than 98% purity with mass 644.2.

iii) Peptide Conjugation-Synthesis of Peptide(SEQ ID NO: 105)-trans-1,4-cyclohexyl-Dex (25)

To the stirred mixture of the peptide having amino acid sequence SEQ ID NO: 105 (20 mg, 0.00465 mmol) and N-methylmorpholine (4.0 μL, 0.00363 mmol) in 1.5 mL anhydrous DMSO, was added Dex-trans-1,4-cyclohexyl NHS ester (7.8 mg, 99% purity, 0.0121 mmol). The reaction mixture was stirred at room temperature for 2 days (48 hrs). The reaction mixture was purified by HPLC (10 to 50 in 20 min) to give 15.0 mg white powder with 67% yield. LC-MS showed greater than 98% purity with MS: 1208.4 (M/4), 1610.8 (M/3).

Alternatives to the peptides used in this EXAMPLE or another peptide disclosed herein, such as SEQ ID NO: 103 or SEQ ID NO: 184 or SEQ ID NO: 509, are used as the peptide for the conjugation. Alternatives to dexamethasone, budesonide, triamcinolone acetonide, or other glucocorticoid disclosed herein are used as an active agent for the conjugation.

### Example 19

### Synthesis of Additional Conjugates

This example shows non-limiting examples of additional conjugates incorporating additional ester linkers with cyclic groups that are conjugated to any peptide or drug of this disclosure.

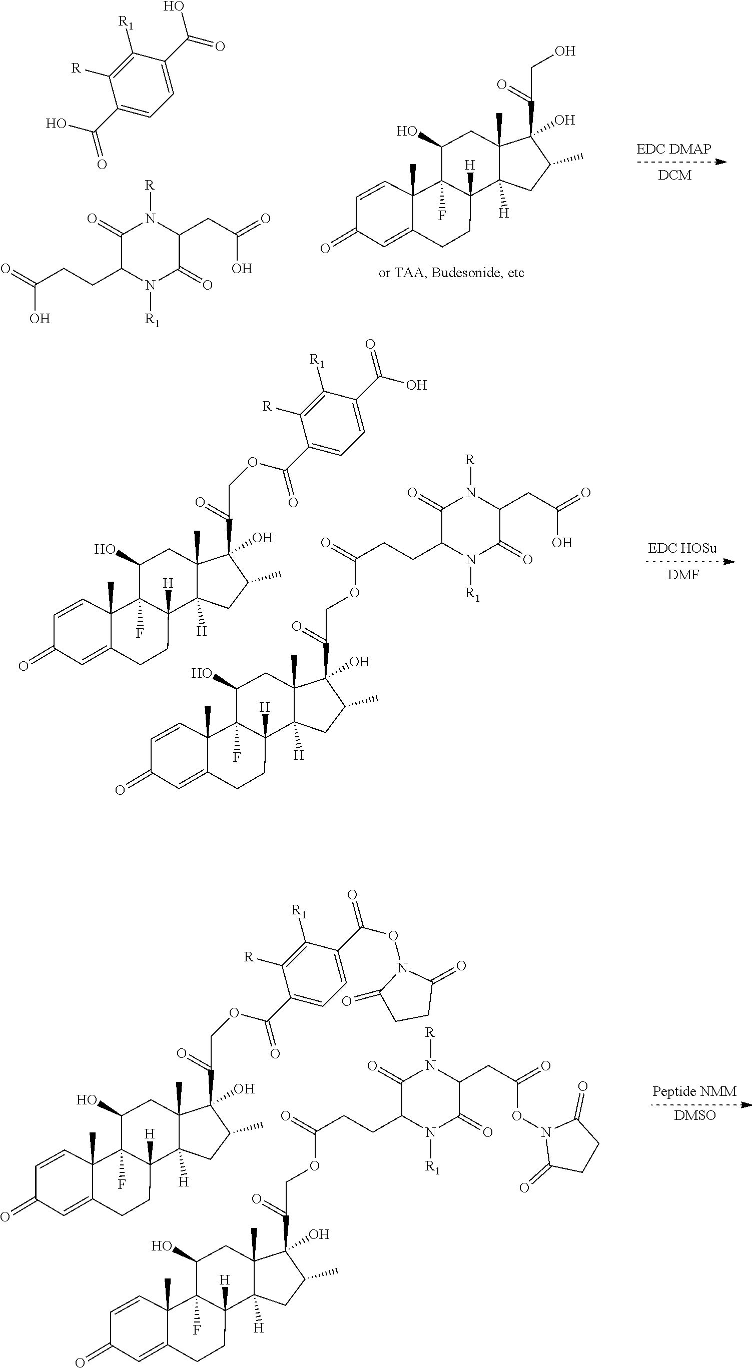

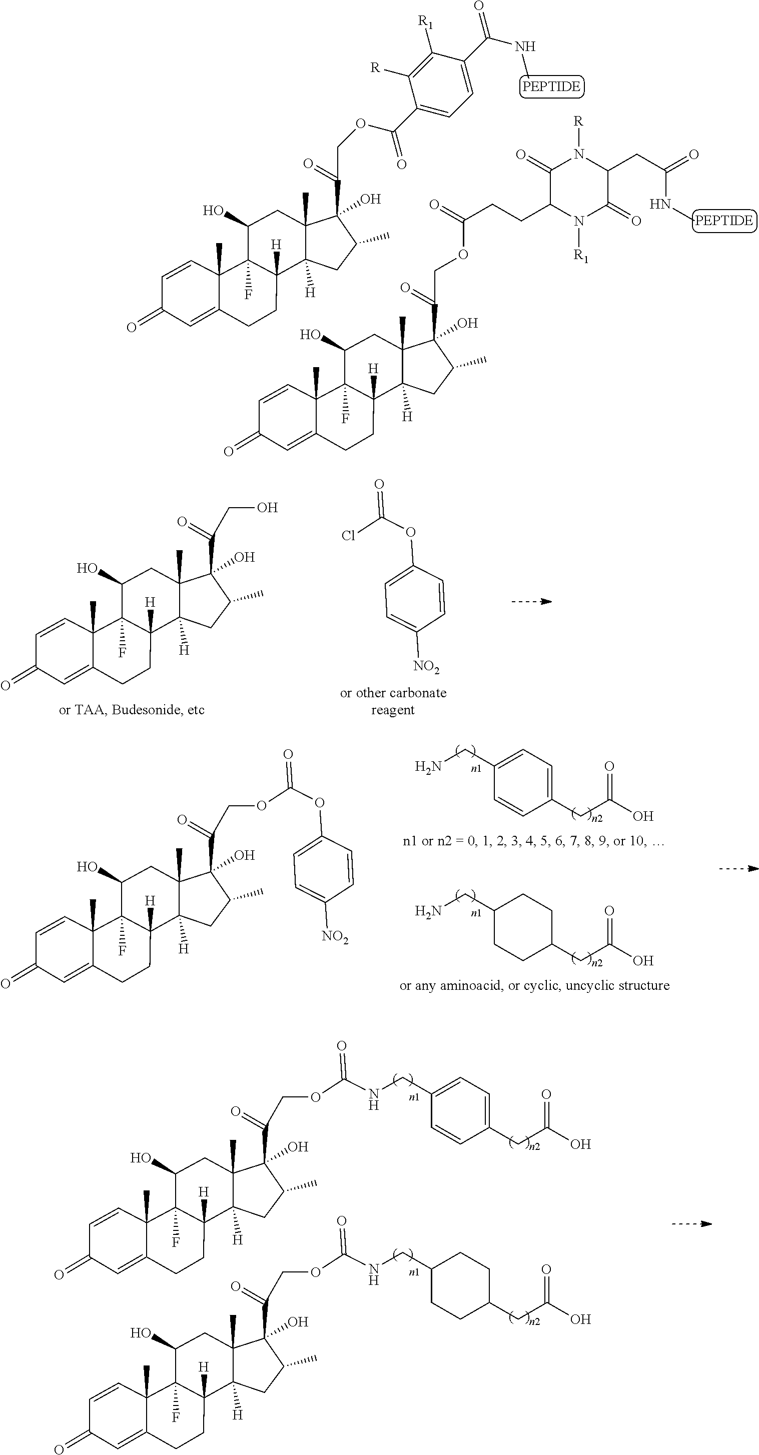

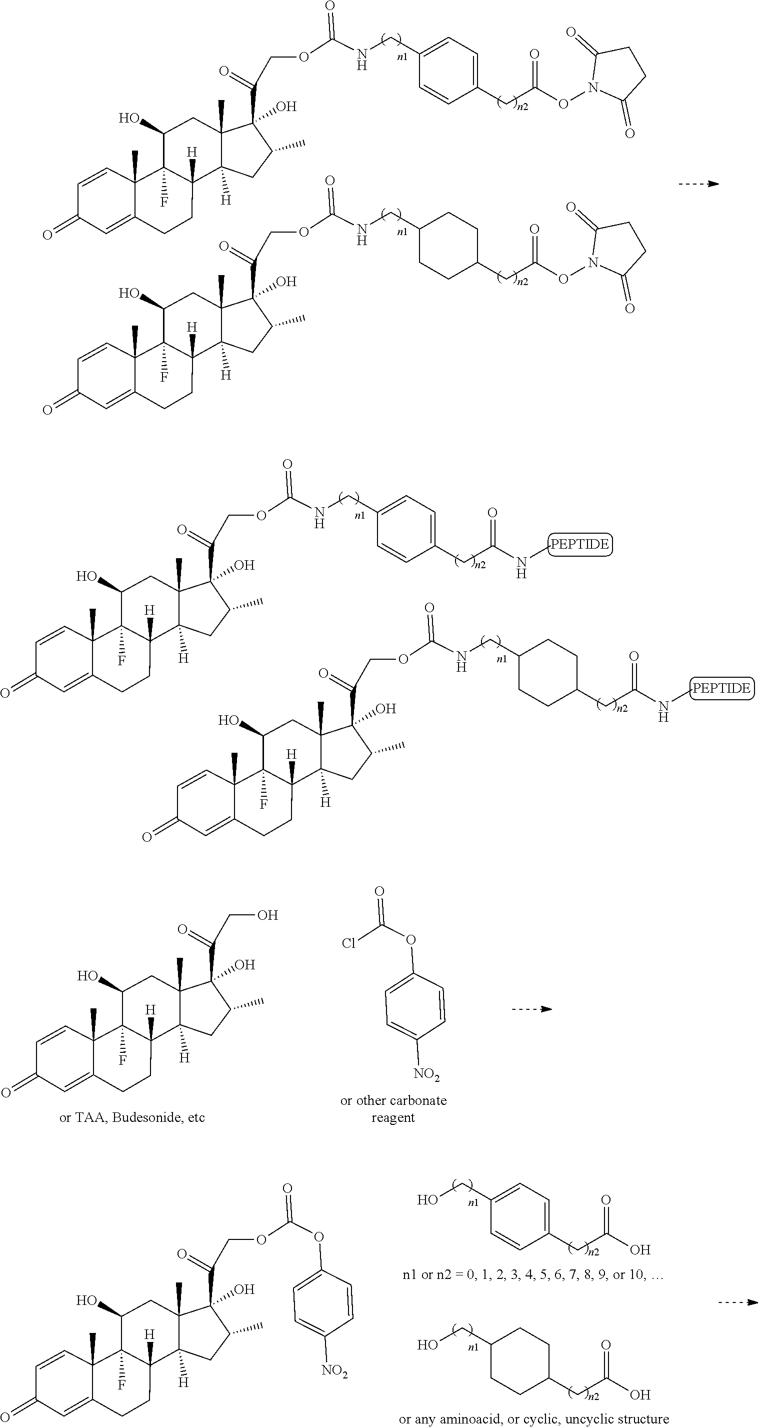

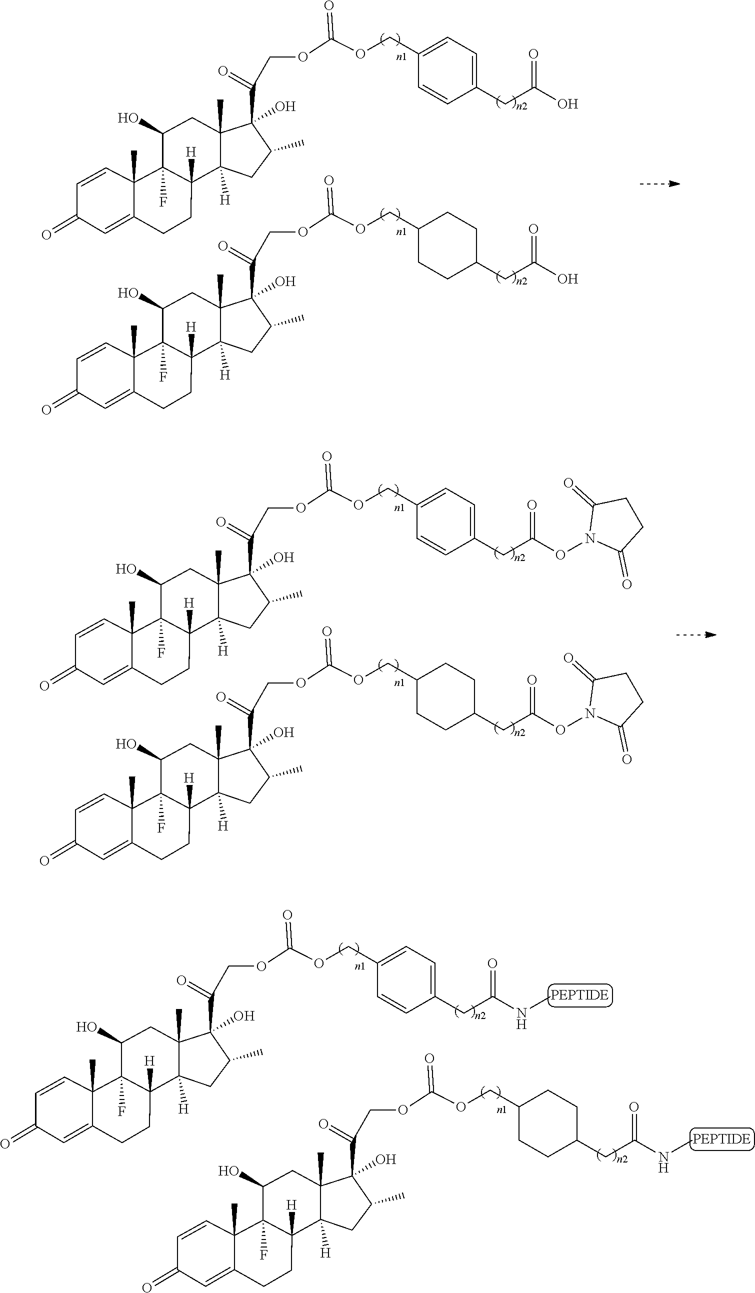

Alternatives to the peptides used in this EXAMPLE or another peptide disclosed herein, such as SEQ ID NO: 105, SEQ ID NO: 509, SEQ ID NO: 103, or SEQ ID NO: 184, are used as the peptide for the conjugation. Alternatives to dexamethasone, budesonide, triamcinolone acetonide, des-ciclesonide or other glucocorticoid disclosed herein are used as an active agent for the conjugation.

### Example 20

### Hydrolysis Assay

This example demonstrates a hydrolysis assay to determine the hydrolysis half-lives and/or in vitro stabilities of peptide-drug conjugates (PDC) of the present disclosure in PBS, rat plasma, and human plasma.

### General Procedure

Lyophilized, purified peptide-drug conjugates were reconstituted in DMSO following production to generate stock solution at 20 mg/mL. Each conjugate stock was brought to 0.25 mg/mL in each hydrolysis condition (1×DPBS, human plasma and rat plasma) in triplicate and rocked at 37° C.

To quantitate free dexamethasone (Dex), 100 μl of the hydrolyzed conjugate solution was transferred into 1 mL acetonitrile at 4° C. at which time all plasma proteins and intact conjugates precipitated. This was performed repeatedly to generate a series of time points (0 hr, 0.5 hr, 1 hr, 2 hr, 4 hr, 6 hr, 8 hr, 24 hr, 32 hr). The corticosteroid triamcinolone acetonide (25 μl, 0.1 mg/mL in 45:55 ACN:Citrate 10 mM pH 5.5) was supplemented into the acetonitrile solution as an internal standard. The acetonitrile solution was spun at 17,000 g for 5 minutes, and the supernatant removed and added to a 96 well block. The pellet was resuspended in 500 μl acetonitrile and re-pelleted as before. The supernatant was removed and added to the same well in the block.

Samples were prepared for analysis by drying under a nitrogen stream followed by reconstitution in 110 μl 45:55 ACN:Citrate 10 mM pH 5.5. To ensure optimal recovery the block was shaken via plate shaker at 7,000 rpm for 5 minutes during the reconstitution step. Prior to LC/MS the samples were transferred to a 96 well plate and centrifuged at 6,500 g for 15 minutes. Analysis was performed on an Agilent 1260 Infinity series HPLC with inline 6120 single quad MS using a gradient of 0.1% TFA in acetonitrile (solvent B), and 0.1% TFA (aq) (solvent A) gradient (shown below) on an InfinityLab Poroshell SB-C18 column. Quantification of free dexamethasone was achieved through integration of dexamethasone and triamcinolone acetonide peaks of the TIC. Acetonitrile (ACN): P.N. HB98134.

LC/MS System: Agilent 1260 Infinity series HPLC with inline 6120 single quad MS.

Column: InfinityLab Porochell 120 SB-C18 P.N. 687975-902.

Gradient:

| Time | A [%] | B [%] |
|---|---|---|
| 0 | 75 | 25 |
| 1.25 | 75 | 25 |
| 11.25 | 35 | 65 |
| 12.50 | 5 | 95 |
| 13.75 | 5 | 95 |
| 15.00 | 75 | 25 |
| 16.25 | 75 | 25 |

### Mass Spectrometry Settings:

| Scan Mode | Mass Range | Fragmentor | Threshold | Gain | Step Size | Speed (μ/sec) |
|---|---|---|---|---|---|---|
| Positive | 250-2000 | 150 | 150 | 1.0 | 0.1 | 1733 |

### Hydrolysis Results

The conjugates were diluted in PBS, rat plasma, or human plasma and incubated at 37 degrees. Samples were taken at the designated time-points, TAA was added as an internal standard, and then Dex/TAA were extracted using acetonitrile. The samples were dried down, reconstituted, and analyzed by LC/MS. Data were normalized using Dex AUC/TAA AUC. Percent hydrolysis was calculated using the average ratio for Dex AUC/TAA AUC at 32 hours as the value for maximal drug release. Hydrolysis measurements are shown in FIG. 2A-FIG. 2E and the table below. This method measures the rate of release of free unmodified dexamethasone. It may be released by chemical hydrolysis or by enzymatic hydrolysis such as by plasma esterases or by other means. Possible presence of other molecules that may be active, such as other protease or degradation products, were not assessed.

The results show variable release rates between the conjugates and in different fluids and that conjugates with faster or slower release rates can be used depending on the desired pharmacokinetic profile for an intended outcome of an active agent or imaging agent.

TABLE 4
Half-life (hours) as measured in the hydrolysis experiments (Dex: Dexamethasone)

| | | Peptide(SEQ | | Peptide(SEQ ID | |
|---|---|---|---|---|---|
| | | ID NO: | | NO: 105)- | |
| | Peptide(SEQ | 105)-trans- | | [cyclohexyl-(N- | |
| | ID NO: | 1,4- | | 4-aminomethyl- | Peptide(SEQ |
| | 105)-glutaric | cyclohexyl- | Peptide(SEQ ID | trans-1- | ID NO: 105)- |
| | acid-Dex | Dex | NO: 105)- | carbamoyl)]- | adipic acid- |
| | (23) | (25) | DMA-Dex (27) | Dex(28) | Dex (24) |
| Human | 1.7 | 21.3 | 22.4 | Dex | — |
| Plasma | | | | undetectable ≤32 | |
| | | | | hr | |
| Rat | 1.9 | 3.4 | 9.9 | Dex | 2.8 |
| Plasma | | | | undetectable ≤32 | |
| | | | | hr | |
| PBS | 6 | Dex | Dex | Dex | — |
| | | undetectable ≤32 | undetectable ≤32 | undetectable ≤32 | |
| | | hr | hr | hr | |

### Example 21

### In Vivo Testing

This example demonstrates in vivo testing and comparison of ¹⁴C-Cys-Dexamethasone (or ¹⁴C-cys-Dex) and peptide(SEQ ID NO: 105)-¹⁴C-Cys-Dexamethasone (or peptide(SEQ ID NO: 105)-¹⁴C-Cys-Dex) (“peptide(SEQ ID NO: 105)-¹⁴C-Cys” is disclosed as SEQ ID NO: 512).

### Protocols

### Collagen Induced Arthritis (CIA) Model

Arthritis was induced in 9-week-old female Lewis rats (Envigo or Charles River Laboratories) by intradermal injection while anesthetized of 400 ug bovine type II collagen (Chondrex Inc, Redmond Wash.) in 2 adjacent 200 ul (1 mg/ml) doses on day 0. Collagen is prepared for injection by dissolving at 2 mg/ml in 0.01N glacial acetic acid in sterile water and rocking at 4° C. overnight then emulsifying in Freund's incomplete adjuvant (IFA, Sigma Aldrich). To emulsify, equal volumes of collagen solution and IFA are drawn up into separate syringes, which are joined by a 3-way stopcock. While on ice, the collagen and IFA are mixed by pressing between the two syringes rapidly for 10 minutes. Quality of the emulsification is tested after 10 minutes of mixing by dropping a small amount of mixture in water. A properly emulsified solution will remain as a discrete droplet and not disperse in the water. On day 7, rats are challenged with a second intradermal injection of 100 ug of collagen in 100 ul, 1 mg/ml, collagen in IFA solution prepared fresh. Body weight and ankle diameter measurements are recorded daily from day 7 through the end of the study. Ankle diameter was measure by a single researcher using a Fowler Digitrix 2 micrometer. Three measurements of each ankle were taken of the lateral dimension at the tarsus of lightly anesthetized rats. The reported ankle diameter measurement is the mean of the three measurements.

### Rat CIA Biodistribution Study

To quantify accumulation of CDP (cystine-dense peptide) and CDP-steroid conjugates in arthritic joints, arthritis was induced in female rats as described. ¹⁴C-cys-Dexamethasone and peptide(SEQ ID NO: 105)-¹⁴C-Cys-Dex (30) (“peptide(SEQ ID NO: 105)-¹⁴C-Cys” is disclosed as SEQ ID NO: 512) (the radiolabeled conjugate prepared in EXAMPLE 8) were administered on day 12 after initially collagen inoculation. ¹⁴C-peptide(SEQ ID NO: 105) (radiolabeled according to EXAMPLE 3) was administered on day 14 to a second cohort of rats when they demonstrated edema in the ankle equal to that observed on day 12 for the first cohort. 50 uCi, 400 nmol of each compound was given intravenously (IV) through the tail vein and allowed to circulate 1 hour, 3 hours, or 24 hours. Rats were euthanized by CO₂ asphyxiation, shaved, and frozen in dry ice chilled hexane. QWBA was performed and quantified as described.

### CIA Response Study 1

Arthritis was induced as described in four cohorts of 9-week-old female Lewis rats. Rats were randomly placed in treatment groups, balancing compound and dose groups across cohorts. The peptide-dexamethasone conjugates or free dexamethasone were administered at 510 nmol/kg to 2-4 rats each (all doses described are based on the mass of dexamethasone that is administered; the dose does not include the mass of the peptide or linker). Rats were dosed by tail vein injection on days 11 and 12 and euthanized on day 13 by CO₂ asphyxiation. Immediately after cessation of respiratory movement, blood was collected by cardiac puncture, synovial fluid was collected from both knees, one hind limb was fixed in formalin and the alternate ankle was frozen on dry ice. Thymus, spleen, liver, and kidney weights were recorded.

### Biodistribution in Mice

Peptide(SEQ ID NO: 105)-¹⁴C-Cys-TAA (38, the radiolabeled conjugate prepared in EXAMPLE 7) (“peptide(SEQ ID NO: 105)-¹⁴C-Cys” is disclosed as SEQ ID NO: 512) or ¹⁴C-Cys-TAA was resuspended in 5% DMSO in water. The amount of radioactivity per μ1 of solution was measured using liquid scintillation counting (LSC). Each compound was administered intravenously to mice at a dose of 12.4 μCi (100 nmol) and allowed to circulate for the designated time intervals. The mice were then humanely euthanized and carcasses were frozen in dry ice chilled hexane. QWBA was performed and quantified as described.

### QWBA

Frozen carcasses were allowed to off gas hexane overnight at −20° and then embedded in chilled 2% carboxymethylcellulose (Sigma Aldrich) and sectioned sagittally at a thickness of 40 μm on an H/I Bright 8250 Cryostat (Hacker Instruments). Radioactive control guides were drilled into each block to verify even cutting depth along the length of the section. The radioactive guide consisted of ¹⁴C-Glycine (American Radiolabeled Chemicals, “ARC”) at 0.5 uCi/ml in 0.5% BSA (Sigma Aldrich) in PBS. Sections were collected onto 4-inch wide tape (Scotch 821, ULINE) at 2-4 depths to sample approximately 30 tissues with particular focus on the knees and spine. Collected sections were freeze dried in the cryostat for 48-72 hours then mounted on sturdy paper and covered with a single protective layer of cellophane (Reynolds Food Service Film). Mounted sections were exposed to phosphor imager plates (Raytest) along with a radioactive standard Curve (1465-PL, ARC) for 7-days, scanned on a Raytest CR-35 at “25 μm sensitive” setting.

### QWBA Data Analysis

Quantification of the radioactivity detected in WBA samples was evaluated by measuring the pixel density/mm² (“SI”: Signal Intensity/Area−background) using AIDA Image Analyzer v 5.1 Whole Body Autoradiography Professional software (Raytest). Regions of interest (ROIs) were drawn manually around the entire visible tissue, comparing the sectioned sample to the autoradiogram. For quantification, all visible knee or IVD (intervertebral disc) tissues for one rodent across all collected sections were measured and averaged together to generate one knee or IVD value per animal. Typically were sampled 1-2 knee values (from femoral and tibial cartilage) per 1-2 sections per animal and 3-10 IVD per 1-2 sections per animal. To collect quantified values for other tissues, ROIs were drawn around blood in the heart ventricle, spinotrapezius muscle, the liver, or the kidney (including cortex and medulla) in all sections in which the tissue was observed and then averaged to yield one value per tissue per animal.

Data were presented as nmol of compound per gram of tissue. The commercial standard strips co-exposed with each WBA sample set were calibrated so that a known amount of radioactive counts per minute (CPM) per gram of tissue, determined by liquid scintillation counting (LSC), corresponded to the measured WBA SI at 11 standard strip concentration data points. The regression line of the log 10 transformed WBA signal intensity plotted against the log 10 transformed CPM/g gave a linear range of 1×10⁵ to 7.5×10⁷ WBA SI, corresponding to 6.3 nCi/g to 3.3 μCi/g. The log 10 concentration of radioactivity in each tissue (CPM/g) was interpolated from the standard curve, converted to linear values, and translated from CPM/g to uCi/g by interpolation against an LSC CPM to μCi standard curve. Uptake (nmol of compound/g of tissue) was determined by dividing the μCi/g tissue by the μCi/nmol specific activity of the peptide.

### Results

### Stable Peptide-TAA Conjugate in Biodistribution Studies in Mice

TABLE 5
Study Design

| | | Peptide | Time | | # |
|---|---|---|---|---|---|
| Group | Peptide | dose | point (hr) | Route | mice |
| 1 | Peptide(SEQ ID | 100 nmol | 3 | IV | 6 |
| | NO: 105)-¹⁴C-Cys-TAA | | | | |
| | (38) (“peptide(SEQ | | | | |
| | ID NO: 105)-¹⁴C- | | | | |
| | Cys” is disclosed | | | | |
| | as SEQ ID NO: 512) | | | | |
| 1 | Peptide(SEQ ID | 100 nmol | 24 | IV | 6 |
| | NO: 105)-¹⁴C-Cys-TAA | | | | |
| | (38) (“peptide(SEQ | | | | |
| | ID NO: 105)-¹⁴C- | | | | |
| | Cys” is disclosed | | | | |
| | as SEQ ID NO: 512) | | | | |
| 3 | ¹⁴C-Cys-TAA | 100 nmol | 3 | IV | 6 |
| 3 | ¹⁴C-Cys-TAA | 100 nmol | 24 | IV | 6 |

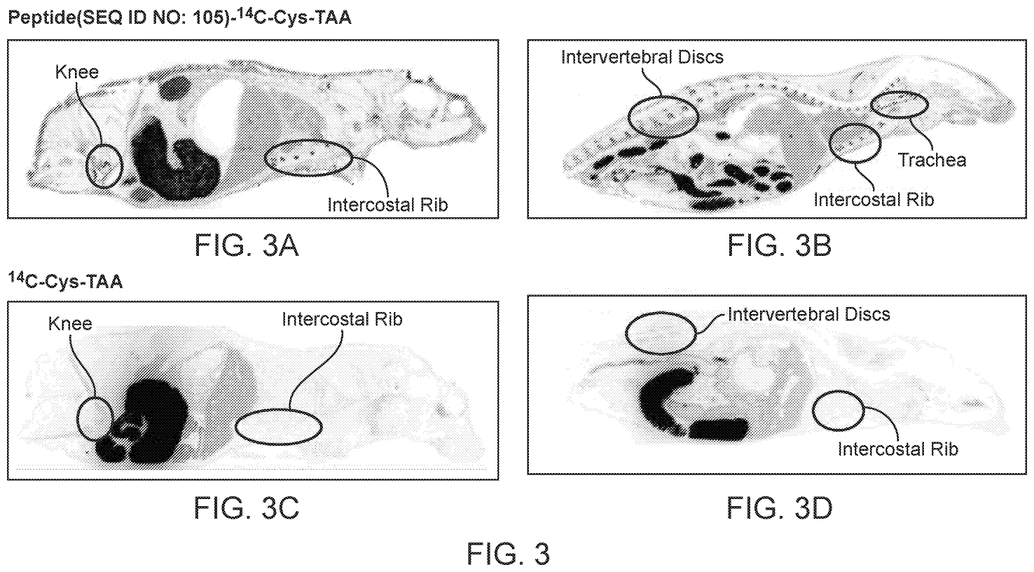

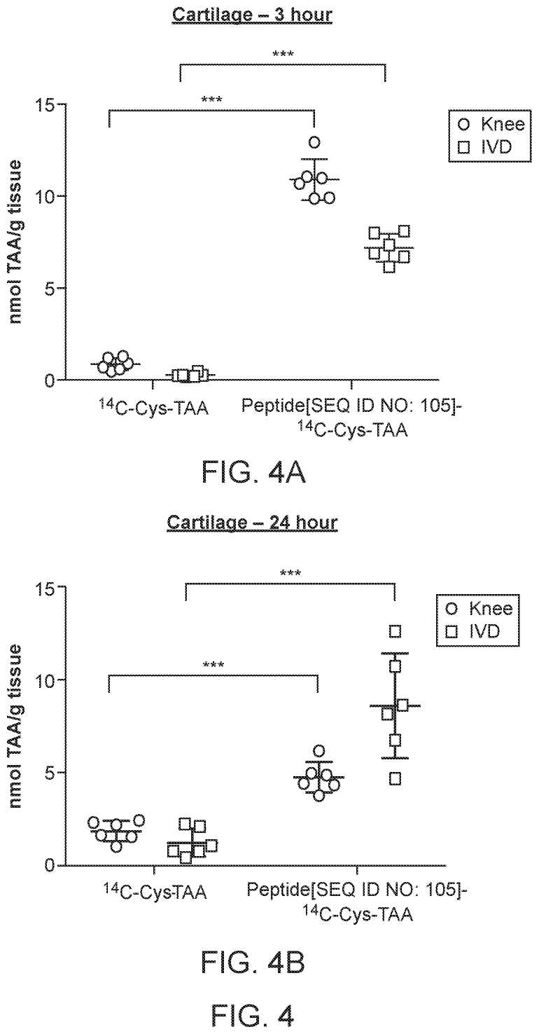

The peptide having the amino acid sequence set forth in SEQ ID NO: 105 was conjugated to TAA using a stable linker comprised of a cysteine residue. The cysteine linker was radiolabeled using reductive methylation. The biodistribution of the peptide-drug conjugate peptide(SEQ ID NO: 105)-Cys-TAA conjugate (“peptide(SEQ ID NO: 105)-Cys” is disclosed as SEQ ID NO: 512) was then assessed in mice using whole body autoradiography. The cysteine linker was also conjugated to TAA and radiolabeled so that the biodistribution of TAA could be assessed in similar manner. Results are shown in FIG. 3 and FIG. 4. Peptide(SEQ ID NO: 105)-¹⁴C-Cys-TAA (“peptide(SEQ ID NO: 105)-¹⁴C-Cys” is disclosed as SEQ ID NO: 512) signal was visually apparent in cartilage of the knee, costal cartilages, intervertebral discs (IVDs), and trachea in these sections at both 3 hours and 24 hours (FIG. 3A and FIG. 3B for the radiolabeled peptide and FIG. 3C and FIG. 3D for the radiolabeled drug as control, respectively). There was no observable signal for ¹⁴C-Cys-TAA in cartilage but there was signal apparent in the bone marrow of the vertebrae and long bones (FIG. 3C and FIG. 3D). Quantitation of the signal demonstrated approximately 12-fold higher levels of peptide(SEQ ID NO: 105)-¹⁴C-Cys-TAA (“peptide(SEQ ID NO: 105)-¹⁴C-Cys” is disclosed as SEQ ID NO: 512) in cartilage as compared to ¹⁴C-Cys-TAA. This indicates that conjugating the active agent TAA to the peptide can target TAA to the cartilage of mice, whereas administration of TAA without the peptide results in minimal targeting of delivery to the joints (see FIG. 4A for the 3 h and FIG. 4B for the 24 h time point).

Stable Peptide-Dex Conjugate in Biodistribution Studies in Rats with Arthritis

TABLE 6
Study Design of Biodistribution in Collagen
Induced Arthritis (CIA) Rat Model.

| Group | Treatment | Dose | Time point | # of rats |
|---|---|---|---|---|
| 1 | ¹⁴C-Cys- | 400 nmol | 1 hr, 3 hr, 24 hr | 2 each |
| | Dexamethasone | | | (6 total) |
| 2 | Peptide(SEQ | 400 nmol | 1 hr, 3 hr, 24 hr | 2 each |
| | ID NO: 105)- | | | (6 total) |
| | ¹⁴C-Cys-Dex | | | |
| | (30) | | | |
| | (“peptide(SEQ | | | |
| | ID NO: 105)- | | | |
| | ¹⁴C-Cys” is | | | |
| | disclosed as | | | |
| | SEQ ID NO: | | | |
| | 512) | | | |
| 3 | ¹⁴C-Peptide(SEQ | 400 nmol | 1 hr, 3 hr, 24 hr | 2 each |
| | ID NO: 105) | | | (6 total) |

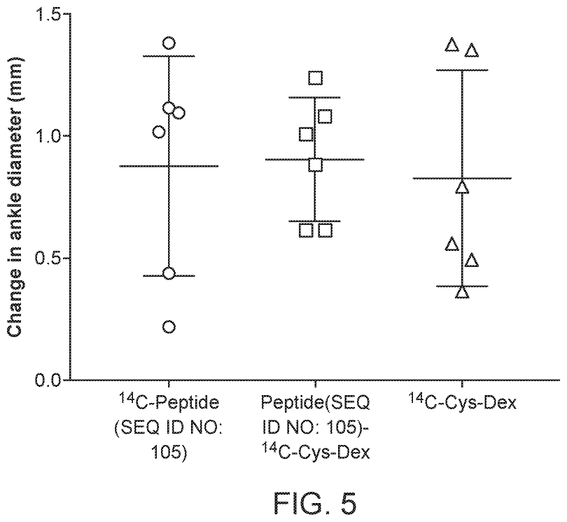

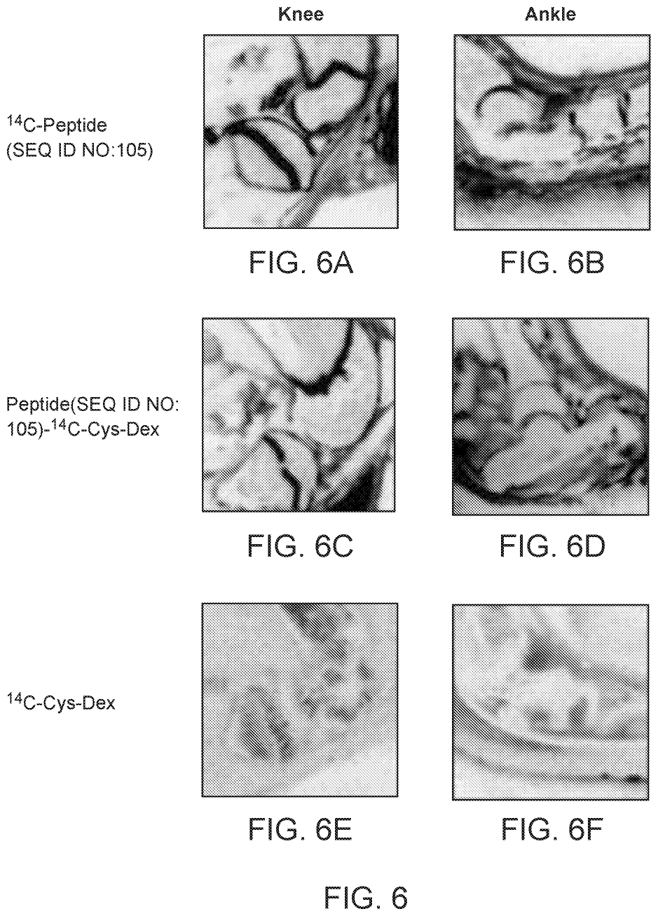

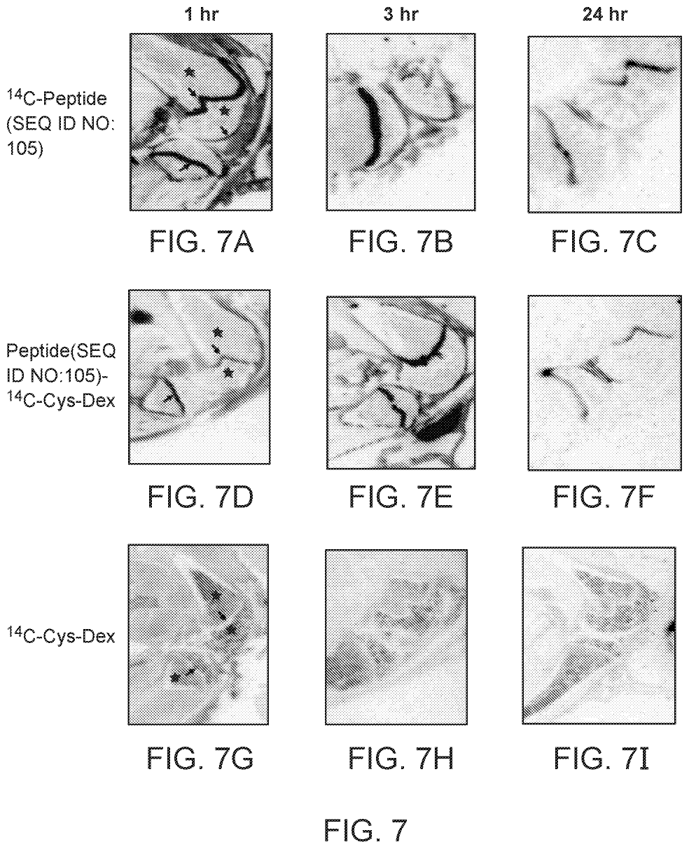

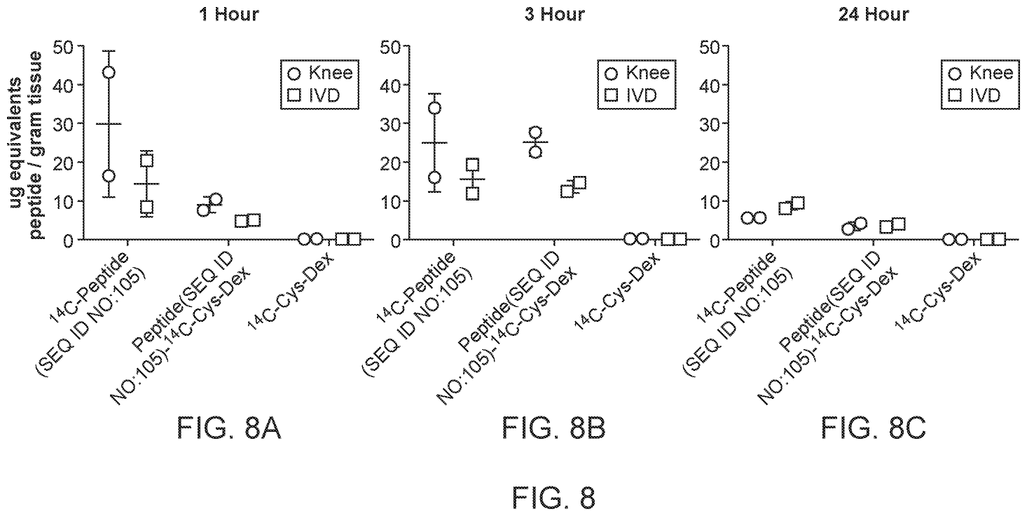

Peptide(SEQ ID NO: 105) was conjugated to dexamethasone using a stable linker comprised of a cysteine residue. The cysteine linker was radiolabeled using reductive methylation. The biodistribution of the peptide(SEQ ID NO: 105)-¹⁴C-Cys-Dex conjugate (“peptide(SEQ ID NO: 105)-¹⁴C-Cys” is disclosed as SEQ ID NO: 512) was then assessed in rats with arthritis using whole body autoradiography. The cysteine linker was also conjugated to dexamethasone and radiolabeled so that the biodistribution of dexamethasone could be assessed in similar manner. The biodistribution of the peptide(SEQ ID NO: 105) in arthritic rats was evaluated in the same experiment. The peptide was radiolabeled on the N-terminus using reductive methylation. FIG. 5 shows the increase in ankle diameter (in mm) in all groups of the study, confirming that arthritis was induced. From these data and FIG. 6, signal can be detected in cartilage of rats that received ¹⁴C-Peptide(SEQ ID NO: 105, FIG. 6A and FIG. 6B) or peptide(SEQ ID NO: 105)-¹⁴C-Cys-Dex (“peptide(SEQ ID NO: 105)-¹⁴C-Cys” is disclosed as SEQ ID NO: 512) (FIG. 6C and FIG. 6D) at all time-points, including the knee, shoulder, hip, ankle, costal cartilage, and intervertebral discs. There was no visually detectable signal in cartilage in rats that received ¹⁴C-Cys Dex (FIG. 6E and FIG. 6F). In rats treated with ¹⁴C-Cys-Dex, the signal that was apparent in the joints was localized in the bone marrow, and the cartilage was largely devoid of signal. QWBA results are shown in FIG. 7 to FIG. 8. This indicates that conjugating the active agent dexamethasone to the peptide can target dexamethasone to the cartilage of rats with arthritis, whereas administration of dexamethasone without the peptide results in minimal targeting of delivery to the joints.

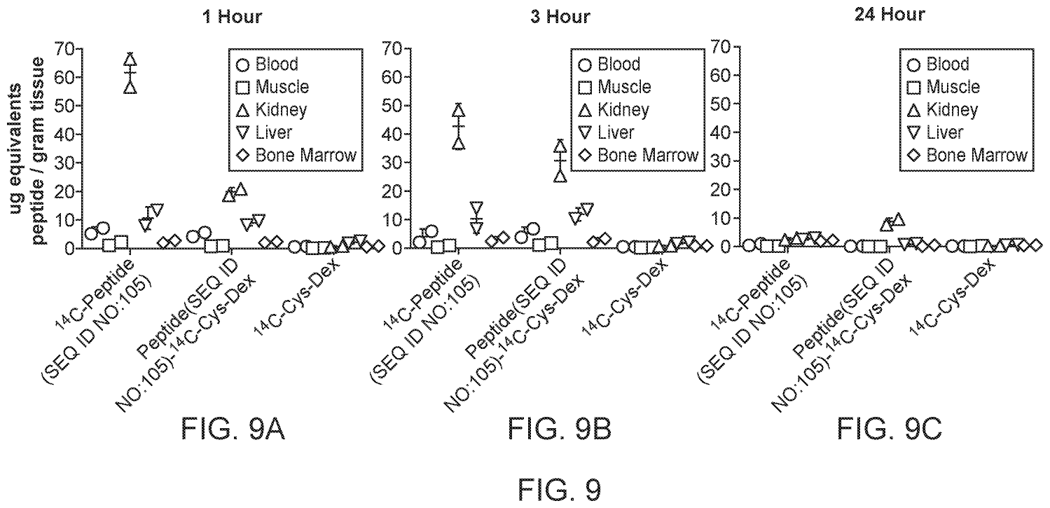

In an additional experiment, the knee as the primary tissue for evaluation of cartilage accumulation was examined, both by visual comparison (FIG. 7) and quantitation (FIG. 8). The IVDs were used as an additional cartilaginous tissue that is readily quantifiable in all sections (FIG. 8). The conjugates ¹⁴C-peptide(SEQ ID NO: 105) and peptide(SEQ ID NO: 105)-¹⁴C-Cys-Dex (“peptide(SEQ ID NO: 105)-¹⁴C-Cys” is disclosed as SEQ ID NO: 512) were detected in cartilage at all three time-points (1 hr (FIG. 8A), 3 hrs (FIG. 8B), and 24 hrs (FIG. 8C); however, there was minimal signal for ¹⁴C-Cys-Dex in cartilage at any time-point, though it is visible in the bone marrow. At 3 hrs, the mean concentration for both ¹⁴C-peptide(SEQ ID NO: 105) and peptide(SEQ ID NO: 105)-¹⁴C-Cys-Dex (“peptide(SEQ ID NO: 105)-¹⁴C-Cys” is disclosed as SEQ ID NO: 512) was approximately 25 μg equivalents peptide/g of tissue (5-6 μM peptide(SEQ ID NO: 105) or peptide(SEQ ID NO: 105)-Cys-Dex (“peptide(SEQ ID NO: 105)-Cys” is disclosed as SEQ ID NO: 512)) in cartilage of the knee and approximately 14-16 μg equivalents peptide/g of tissue (2-3 μM ¹⁴C-peptide(SEQ ID NO: 105) or peptide(SEQ ID NO: 105)-¹⁴C-Cys-Dex (“peptide(SEQ ID NO: 105)-¹⁴C-Cys” is disclosed as SEQ ID NO: 512), respectively, in cartilage of the IVD. There was minimal accumulation of ¹⁴C-peptide(SEQ ID NO: 105) and peptide(SEQ ID NO: 105)-¹⁴C-Cys-Dex (“peptide(SEQ ID NO: 105)-¹⁴C-Cys” is disclosed as SEQ ID NO: 512) in most other tissues of the body (FIG. 9), except liver and kidney, which are likely sites of metabolism and excretion. ¹⁴C-Cys-Dex was not detected at significant levels in any tissue at the time-points tested (FIG. 9A-FIG. 9C).

The data confirms that both peptide(SEQ ID NO: 105) and the conjugate peptide(SEQ ID NO: 105)-Cys-Dex (“peptide(SEQ ID NO: 105)-Cys” is disclosed as SEQ ID NO: 512) accumulate at high levels and persist in the cartilage of diseased joints, whereas Dex alone is not detected in these joints at any tested time point, demonstrating that the peptides of the present disclosure are capable of efficient delivery of drug molecules to cartilage tissue.

Peptide-Dex Conjugates with Labile Linkers in Functionality Studies

CIA Response study design. Dosing (Dex, mg/kg) in TABLE 7 below is displayed based on amount of dexamethasone that was dosed to the animals (excluding the weight of peptide or linker).

TABLE 7
Study Design

| Treatment (Days 11 and | | | |
|---|---|---|---|
| 12, euthanize Day 13) | Dose (Dex, mg/kg)* | Doses | Rats |
| Vehicle | — | 2 | 5 |
| Dexamethasone sodium | 0.2 | 2 | 4 |
| phosphate “Dex” | | | |
| Peptide(SEQ ID NO: 105)- | 0.2 | 2 | 4 |
| glutaric acid-Dex (23) | | | |
| (Prepared per EXAMPLE 9) | | | |
| “Glutaric acid” | | | |
| Peptide(SEQ ID NO: 105)- | 0.2 | 2 | 4 |
| trans-1,4-cylcohexyl-Dex (25) | | | |
| (Prepared per EXAMPLE 18) | | | |
| “1,4-cyclohexyl” | | | |
| Peptide(SEQ ID NO: 105)- | 0.2 | 2 | 4 |
| DMA-Dex (27) | | | |
| (Prepared per EXAMPLE 5) | | | |
| “dimethyl adipic acid” | | | |
| Peptide(SEQ ID NO: 105)- | 0.2 | 2 | 4 |
| [cyclohexyl-(N-4- | | | |
| aminomethyl-trans-1-carbamoyl)]- | | | |
| Dex (28) | | | |
| (Prepared per EXAMPLE 6) | | | |
| “carbamate” | | | |

The aim of the study was to test the 4 conjugates with various linkers at two different doses in vivo in CIA rats to assess the following: ability to deliver sufficient dexamethasone to the joint to show functionality and potential therapeutic effect in the disease model.

Data Collection: Live Phase: Body weight, Ankle diameter.

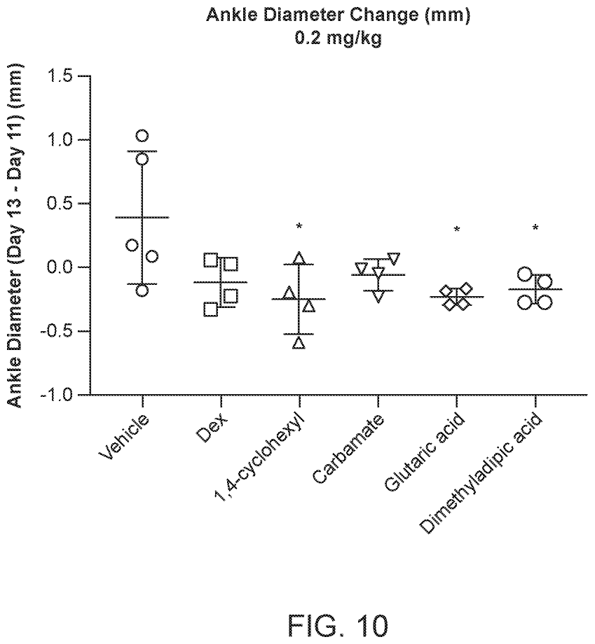

Euthanasia: Thymus, Spleen, Liver, Kidney weight, Blood (CBC, Chemistry, Frozen plasma), and synovial fluid were collected from both knees, one hindlimb fixed in formalin, alternate ankle frozen on dry ice. Functionality and potential therapeutic effect in the joint; effect of the conjugates on disease progression in animal models. FIG. 10 shows ankle diameter measurements in millimeters and changes during the treatment.

Animals treated with vehicle showed increasing ankle diameter from days 11-13, indicative of the inflammation and swelling of arthritis in the joint. Animals treated with 0.2 mg/kg of any of all 4 conjugates showed reduced ankle diameter measurements relative to vehicle. This indicates that all 4 of the conjugates were able to deliver sufficient dexamethasone to reduce disease symptoms in the joint.

### Comparison of Joint Retention Time Among Peptides

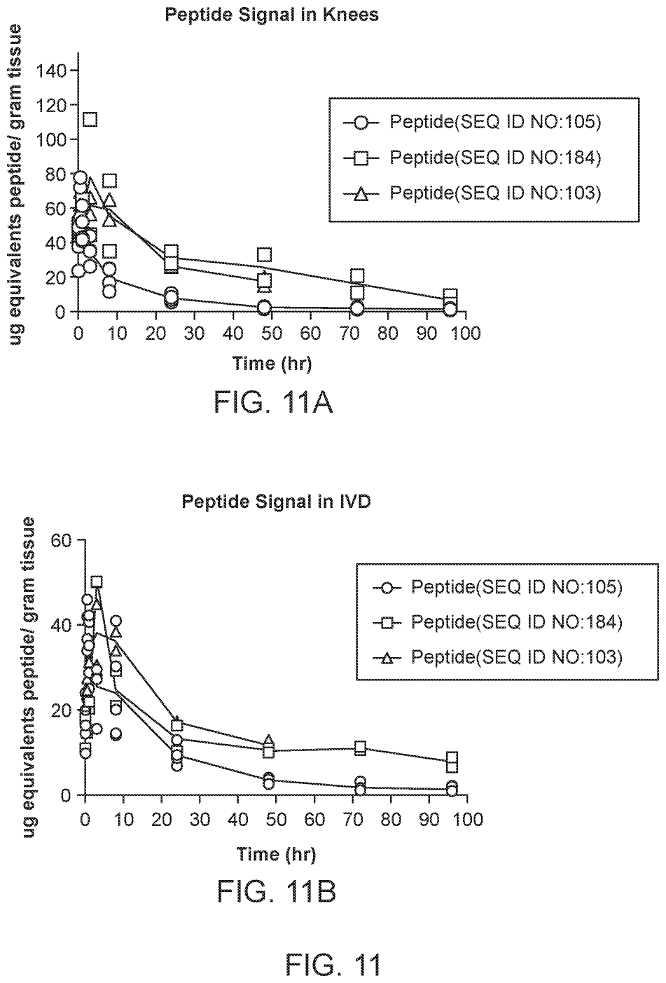

Peptide(SEQ ID NO: 105) was dosed at 57-60 nmol. Peptide(SEQ ID NO: 103) was dosed at 130 nmol. Peptide(SEQ ID NO: 184) was dosed at 150 nmol. Longer joint retention was observed with the latter two peptides with the dosing regimens tested. It should be noted that detection of retention is based on an assay that detects radiolabeling of the N-terminus of each of the tested conjugates, thus decrease in signal over time may be due not only to peptide diffusion back out of the cartilage but also aminopeptidase-mediated removal of N-terminal amino acid, general peptide cleavage, or other mechanisms. Results as measured by QWBA methods described herein are shown in Tables 6 and 7 below and FIG. 11.

TABLE 8
Comparison of Peptide Signal Retention in Knee (FIG. 11A)
Peptide Signal Retention in Knee

| Hours | SEQ ID NO: 105 | SEQ ID NO: 184 | SEQ ID NO: 103 |
|---|---|---|---|
| 0.08 | 91.00 | 96.94 | 79.20 |
| 0.5 | 102.49 | 69.85 | 110.57 |
| 1 | 100.00 | 100.00 | 100.00 |
| 3 | 72.64 | 158.43 | 103.00 |
| 8 | 42.42 | 111.32 | 98.94 |
| 24 | 22.61 | 57.81 | 44.60 |
| 48 | 9.73 | 50.44 | 29.30 |
| 72 | 8.76 | 28.30 | |
| 96 | 5.05 | 11.84 | |

TABLE 9
Comparison of Peptide Signal Retention in IVD (FIG. 11B)
Peptide Signal Retention in IVD

| Hours | SEQ ID NO: 105 | SEQ ID NO: 184 | SEQ ID NO: 103 |
|---|---|---|---|
| 0.08 | 44.42 | 53.02 | 59.15 |
| 0.5 | 101.41 | 49.70 | 76.62 |
| 1 | 100.00 | 100.00 | 100.00 |
| 3 | 106.39 | 238.11 | 113.18 |
| 8 | 81.45 | 119.97 | 107.52 |
| 24 | 36.89 | 64.00 | 50.75 |
| 48 | 19.96 | 49.99 | 34.86 |
| 72 | 13.43 | 52.20 | |
| 96 | 9.17 | 36.54 | |

### Example 22

### Measurements of Systemic Markers for Glucocorticoid Exposure

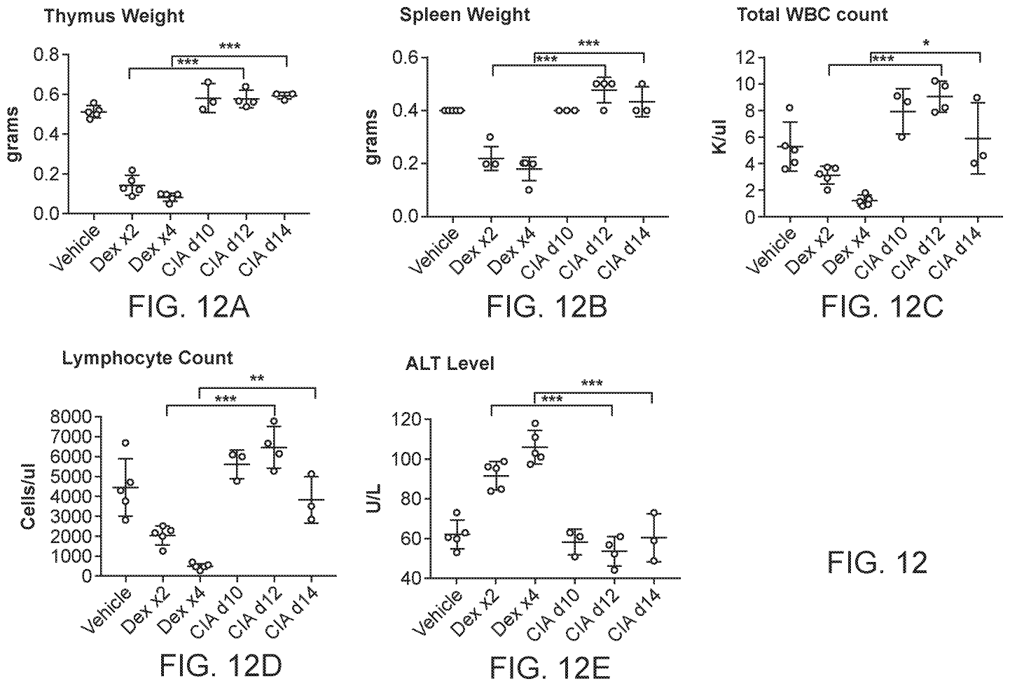

This example illustrates the measurements of systemic markers for glucocorticoid exposure in treated normal rats and for comparison in untreated CIA rats at various stages of disease progression (FIG. 12).

In order to evaluate markers of systemic dexamethasone exposure in normal rats, time points were selected to simulate drug administration for studies (e.g., dosing on days 10 and 11, euthanize on day 12 or dosing on days 11-14 and euthanize on day 14). To establish the baseline response to systemic dexamethasone, normal female Lewis rats were dosed with vehicle or 1 mg/kg/day dexamethasone sodium phosphate (abbreviated as “DexSP” and shown in FIG. 12 as “Dex”) for 2 days or 4 days. Rats were euthanized 24 hours after the second dose, or 5 hours after the fourth dose. Body weight was measured daily. At euthanasia, liver, spleen, thymus, and kidney weights were recorded and blood was collected for CBC and serum chemistry analysis. To establish the CIA model, female Lewis rats were inoculated with an intradermal injection of bovine collagen II on day 0 and a challenge dose was administered on day 7. Ankle and knee joint diameters were measured daily. Rats were euthanized on day 10, day 12 and day 14 at which time liver, spleen, thymus, and kidney weights were recorded and blood was collected for complete blood count (CBC)/blood chemistry analysis. Ankle swelling was visually apparent and the rats had alterations in gait that were consistent with disease induction; however, measurements of joint swelling were inconclusive due to high variability.

FIG. 12A and FIG. 12B show that thymus and spleen weights, respectively, were found to be robust markers of systemic exposure to dexamethasone. The weight of both organs was significantly decreased in both dexamethasone treatment groups as compared to vehicle-treated normal rats and CIA rats at all time points tested. Body weight declined with dexamethasone treatment as compared to vehicle and CIA rats, but the effect on body mass was not separated from changes in feeding.

FIG. 12C and FIG. 12D show that total white blood cell (WBC) count and absolute lymphocyte count, respectively, were additional measures that can be used to assess systemic dexamethasone exposure. These parameters both decreased in response to dexamethasone dosed for 2 days and 4 days as compared to vehicle and CIA rats at various time-points. There were no significant changes in red blood cell (RBC) count, hematocrit, neutrophil, monocyte or eosinophil counts. Nucleated RBCs were detected in four of the five rats that received 4 doses of dexamethasone.

The blood chemistry screen demonstrated a significant change in ALT (alanine aminotransferase) enzyme in response to dexamethasone treatment as compared to vehicle treatment and CIA at all time points (FIG. 12E). It is notable that there were no significant changes in other liver enzymes in response to dexamethasone. This may indicate an effect of the drug at the transcriptional level to increase ALT for gluconeogenesis, rather than a toxic effect on hepatocytes which would likely lead to increased levels of both ALT and AST (aspartate aminotransferase).

This data demonstrates that spleen and thymus weight as well as lymphocyte count, total WBC count, and ALT/AST level may be useful markers to analyze when evaluating effects of systemic exposure to glucocorticoids, which may be particularly useful in methods utilizing the herein described peptide-drug conjugates for prevention and/or treatment of disease.

### Example 23

### In Vivo Dose Ranging Study

This example illustrates a dose-ranging study that was conducted in CIA rats to identify doses of each peptide-drug conjugate that reduced inflammation in the joint and to measure the effect on systemic markers of Dex exposure (e.g., total body weight, thymus weight, spleen weight, etc.). Total white blood cell (WBC) count, total lymphocyte count, and alanine aminotransferase (ALT) levels were also analyzed since these were identified as markers of systemic exposure to Dex in our preliminary studies in normal rats.

CIA was induced in female Lewis rats (as described above, see EXAMPLE 21). Body weight and ankle joint diameters were measured on day 0 and then daily from day 7 until euthanasia. Rats were randomly assigned to treatment groups, balancing compound and dose groups across the cohorts, and were all treated with a bolus of Dex or Dex equivalents on day 11 and 12 as follows: peptide-drug conjugates or free Dex was administered intravenously at 0.2, 0.5, or 1 mg/kg Dex equivalents on both days 11 and 12, and rats were euthanized on day 13 (all dosing is described based on the mass of Dex that was dosed and does not include the mass of linker or peptide). At euthanasia, synovial fluid was collected from both knees, one hind limb was fixed in formalin and the alternate ankle was frozen on dry ice. Thymus, spleen, liver, and kidney weights were recorded. Blood samples were submitted for CBC/Chemistry analysis, and the remaining plasma was frozen. Some rats that were dosed with 1 mg/kg of peptide(SEQ ID NO:105)-glutaric acid-Dex (denoted as “Glutaric Acid”), peptide(SEQ ID NO:105)-DMA-Dex (denoted as “Dimethyladipic acid”), and peptide(SEQ ID NO: 105)-[cyclohexyl-(N-4-aminomethyl-trans-1-carbamoyl)]-Dex (denoted as “Carbamate”) died overnight for unknown causes and some others showed lethargy and prolonged recovery from anesthesia, and as such the remaining treatment groups were redistributed at 0.2 and 0.5 mg/kg dose levels, with random reassignments and balancing compound and dose across cohorts.

The final animal numbers were as follows: peptide-drug conjugate 1 mg/kg (n=2 (1,4-cyclohexyl), n=1 (carbamate), n=2 (glutaric acid), and n=1 (dimethyladipic acid)); peptide-drug conjugate 0.5 mg/kg (n=3); peptide-drug conjugate 0.2 mg/kg (n=4); Dex 1 mg/kg (n=2); Dex 0.5 mg/kg (n=2); Dex 0.2 mg/kg (n=4); vehicle (n=5) (followed through euthanasia on day 13).

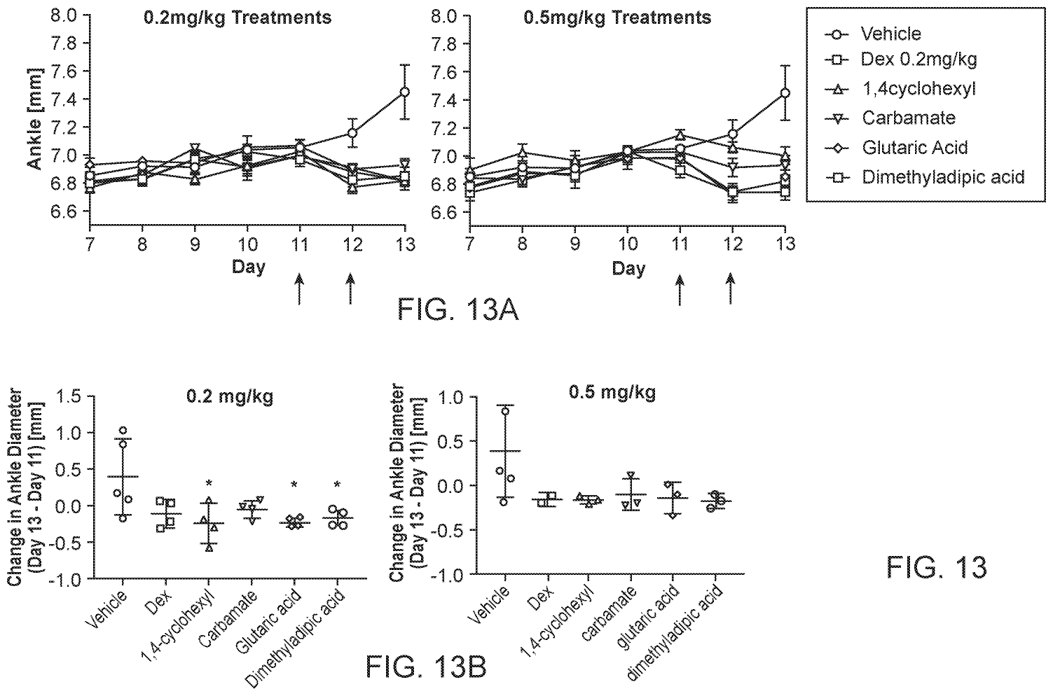

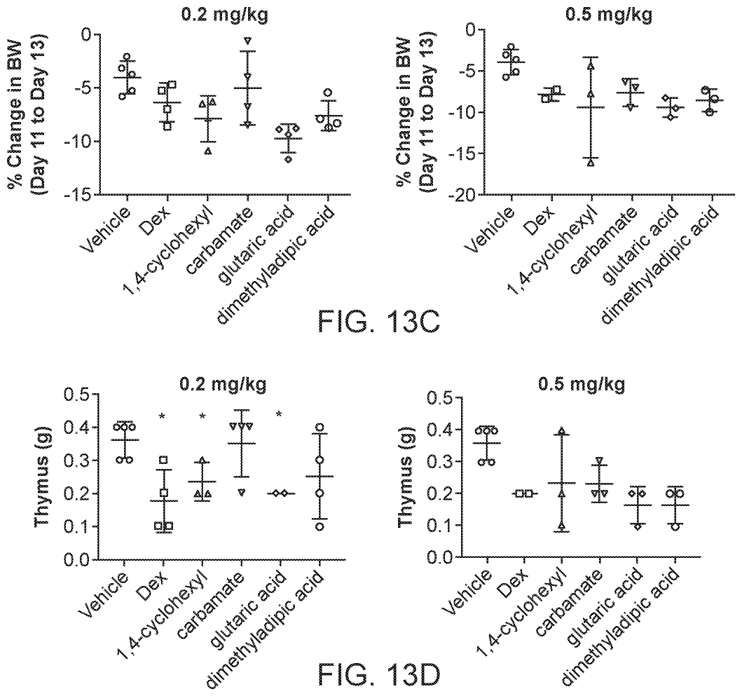

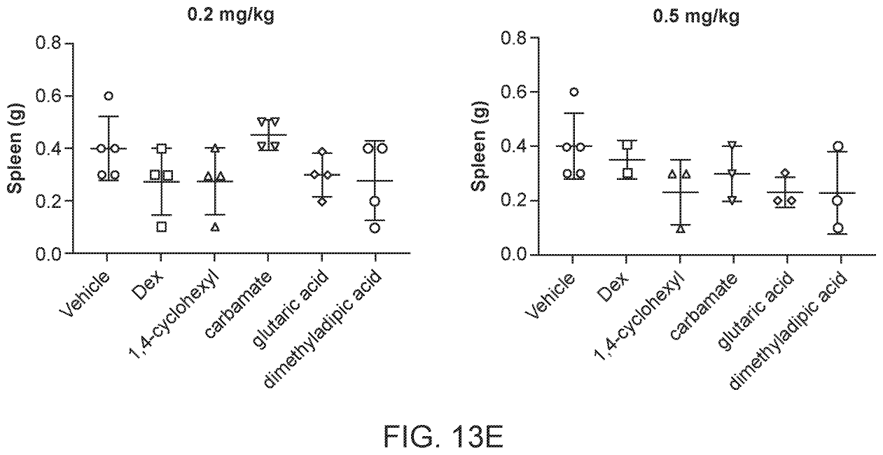

This data (FIG. 13) demonstrates that all four peptide-drug conjugates tested in this study (peptide(SEQ ID NO:105)-glutaric acid-Dex, peptide(SEQ ID NO:105)-trans-1,4-cyclohexyl-Dex, peptide(SEQ ID NO:105)-DMA-Dex, and peptide(SEQ ID NO: 105)-[cyclohexyl-(N-4-aminomethyl-trans-1-carbamoyl)]-Dex) caused a reduction in ankle swelling over time as compared to vehicle at both the 0.2 mg/kg and 0.5 mg/kg dose levels (FIG. 13A). Comparison of the change in median ankle diameter between day 11 and day 13 demonstrated a statistically significant reduction in ankle diameter as compared to vehicle for three of the four peptide-drug conjugates tested: peptide(SEQ ID NO:105)-glutaric acid-Dex, peptide(SEQ ID NO:105)-trans-1,4-cyclohexyl-Dex, and peptide(SEQ ID NO:105)-DMA-Dex at the 0.2 mg/kg dose (FIG. 13B). The change in ankle diameter did not reach statistical significance for free Dex or the remaining peptide-drug conjugate tested: peptide(SEQ ID NO: 105)-[cyclohexyl-(N-4-aminomethyl-trans-1-carbamoyl)]-Dex at the 0.2 mg/kg dose. There was no statistically significant difference between Dex and any of the four peptide-drug conjugates peptide(SEQ ID NO:105)-glutaric acid-Dex, peptide(SEQ ID NO:105)-trans-1,4-cyclohexyl-Dex, peptide(SEQ ID NO:105)-DMA-Dex, and peptide(SEQ ID NO: 105)-[cyclohexyl-(N-4-aminomethyl-trans-1-carbamoyl)]-Dex at this 0.2 mg/kg dose. Statistical analysis on the change in ankle diameter was not performed on the 0.5 mg/kg dose group due to the small animal numbers. Moreover, the peptide(SEQ ID NO:105)-glutaric acid-Dex, peptide(SEQ ID NO:105)-trans-1,4-cyclohexyl-Dex, peptide(SEQ ID NO:105)-DMA-Dex and free Dex groups all showed a reduction in total body weight (FIG. 13C), thymus weight (FIG. 13D), and spleen weight (FIG. 13E) at both the 0.2 mg/kg and 0.5 mg/kg dose. The reduction in thymus weight was statistically significant for peptide(SEQ ID NO:105)-glutaric acid-Dex, peptide(SEQ ID NO:105)-trans-1,4-cyclohexyl-Dex, and free Dex as compared to vehicle in the 0.2 mg/kg dose groups. The reduction in body weight and spleen weight although trending in this manner did not reach statistical significance as compared to vehicle likely due to the small cohorts of animals tested. Overall, animals treated with 0.2 mg/kg of peptide(SEQ ID NO: 105)-[cyclohexyl-(N-4-aminomethyl-trans-1-carbamoyl)]-Dex appeared to retain total body weight and tissue weights better compared to any of the other PDC candidates or Dex alone. (FIG. 13C-FIG. 13E).

In summary, this initial dosing study demonstrated reduction of ankle inflammation by all 4 four PDCs tested (peptide(SEQ ID NO:105)-glutaric acid-Dex, peptide(SEQ ID NO:105)-trans-1,4-cyclohexyl-Dex, peptide(SEQ ID NO:105)-DMA-Dex, and peptide(SEQ ID NO: 105)-[cyclohexyl-(N-4-aminomethyl-trans-1-carbamoyl)]-Dex) in a rat model of CIA. The carbamate linker, while stable in in vitro testing, presumably released active Dex sufficient to affect disease. This data demonstrates that the herein described peptide-drug conjugates are capable of providing therapeutically relevant drug concentration to tissues of interest (e.g., cartilage in joints).

### Example 24

### Pharmacokinetic Comparison of Peptide-Drug Conjugates to Drug Alone

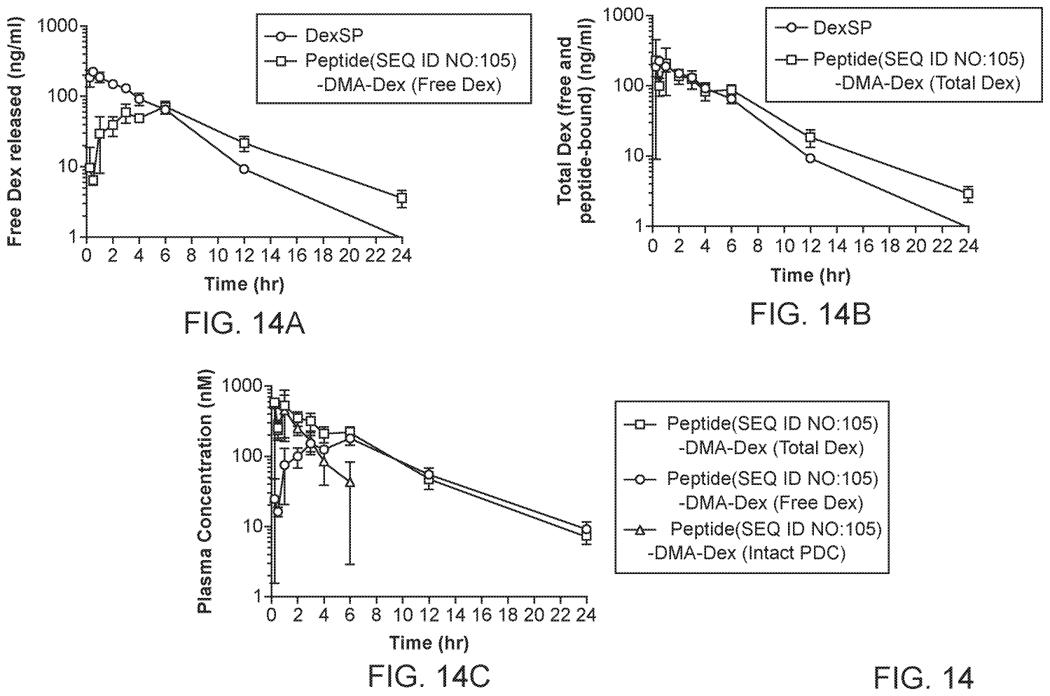

This example demonstrates a comparative study assessing the pharmacokinetic (PK) profiles of the peptide-drug conjugate comprising the peptide which amino acid sequence is set forth in SEQ ID NO: 105 conjugated to Dex via the dimethyladipic acid (DMA) linker, and Dex alone (FIG. 14).

This PK study was conducted in non-diseased 10-week old female Lewis rats with the primary objective of measuring Dex concentration in plasma following intravenous administration of peptide(SEQ ID NO: 105)-DMA-Dex and comparing the profile to that of DexSP (Dexamethasone sodium phosphate, the water soluble IV formulation of Dex). Dexamethasone alone has low water solubility. DexSP is more water soluble and was used for parenteral dosing of dexamethasone. In vivo, DexSP is rapidly hydrolyzed to the active molecule dexamethasone. A secondary objective was to measure free Dex levels in knee joints.

Peptide(SEQ ID NO: 105)-DMA-Dex or DexSP was administered at a dose of 0.2 mg/kg (referring to mass of dosed Dex only, not including mass of peptide or linkers) and the concentration of free Dex was quantified in plasma and knee joints at nine time-points (15 min, 30 min, 1 h, 2 h, 3 h, 4 h, 6 h, 12 h, 24 h) with four rats per time-point (FIG. 14). All samples were collected at euthanasia. Free Dex was extracted from plasma samples and quantified by LC/MS. The assay had a lower limit of quantitation (LLOQ) of 0.4 ng/ml and an upper limit of quantitation (ULOQ) of 400 ng/ml. To measure the total Dex concentration (free Dex released in vivo+Dex associated with intact peptide-drug conjugate) in plasma of rats treated with Peptide(SEQ ID NO: 105)-DMA-Dex, a separate plasma sample was subjected to forced hydrolysis followed by extraction of free Dex. Dexamethasone extraction was achieved by acetonitrile precipitation from plasma samples. The forced hydrolysis protocol involved addition of 10 U porcine esterase to the plasma and incubation for 22 h at 37° C. Total white blood cell counts, absolute lymphocyte counts, and thymus and spleen weights were measured to assess systemic markers of Dex exposure over the time-course.

In order to quantify the concentration of free Dex in knee joints, knee joints were harvested, the joint capsule was opened, and the knee joints were then shaken in ethyl acetate for 12 h at 4° C. The extract was centrifuged and the supernatant was dried under nitrogen in a 96 well plate. The samples were then reconstituted and analyzed by LC/MS using the same protocol developed for quantifying free Dex in plasma. Analysis of free Dex levels in plasma following administration of peptide(SEQ ID NO: 105)-DMA-Dex and DexSP demonstrated a reduced exposure to Dex when equivalent doses were administered in the form of the peptide conjugated to Dex versus administered as Dex alone. With administration of DexSP, the Cₘₐₓ of 224 ng/ml was reached at 30 minutes and steadily decreased over time with a half-life of 2.99 h (FIG. 14A), TABLE 14). With peptide(SEQ ID NO: 105)-DMA-Dex, the Cₘₐₓ for free Dex was delayed to 6 h and was only 71 ng/mL, consistent with the DMA linker hydrolyzing slowly over time in plasma. The free Dex levels then decreased with time, more slowly than with DexSP, but the elimination half-life could not be determined with the number of time points available. Total exposure (AUC) was reduced with (SEQ ID NO: 105)-DMA-Dex versus DexSP (700 vs 1060 hr*ng/ml) (FIG. 14A, TABLE 14).

TABLE 14
PK Parameters Following a Single IV Dose of Peptide(SEQ
ID NO: 105)-DMA-Dex or DexSP to Female Lewis Rats

| | Tₘₐₓ | Cₘₐₓ | AUC₀₋ₜ | MRT | t_{1/2} | CL | V_{SS} |
|---|---|---|---|---|---|---|---|
| Treatment | (hr) | (ng/mL) | (hr * ng/mL) | (hr) | (hr) | (mL/hr/Kg) | (mL/Kg) |
| Dex (no hydrolysis; | 0.5 | 224 | 1060 | 3.90 | 2.99 | 187 | 746 |
| free Dex) | | | | | | | |
| (SEQ ID NO: 105)- | 6.0 | 70.9 | 700 | 7.34 | NE | NE | NE |
| DMA-Dex (no | | | | | | | |
| hydrolysis; free Dex) | | | | | | | |
| (SEQ ID NO: 105)- | 0.25 | 229 | 1240 | 4.73 | 3.93 | 160 | 804 |
| DMA-Dex (with | | | | | | | |
| hydrolysis; total Dex) | | | | | | | |

Forced hydrolysis analysis was performed on peptide(SEQ ID NO: 105)-DMA-Dex plasma samples in order to measure total Dex present in the plasma (free Dex that was released by hydrolysis in vivo+Dex present in intact peptide-drug conjugate). The Cₘₐₓ, Tₘₐₓ, and AUC are very similar for total Dex following treatment with peptide(SEQ ID NO: 105)-DMA-Dex versus DexSP, indicating that an equivalent dose of Dex was administered and that a similar level of Dex is available in the system for delivery to the joint with both treatments. The t_{1/2} was slightly longer for total Dex in the peptide group compared to the Dex dose group (3.93 vs 2.99 h, respectively) (FIG. 14B, TABLE 14).

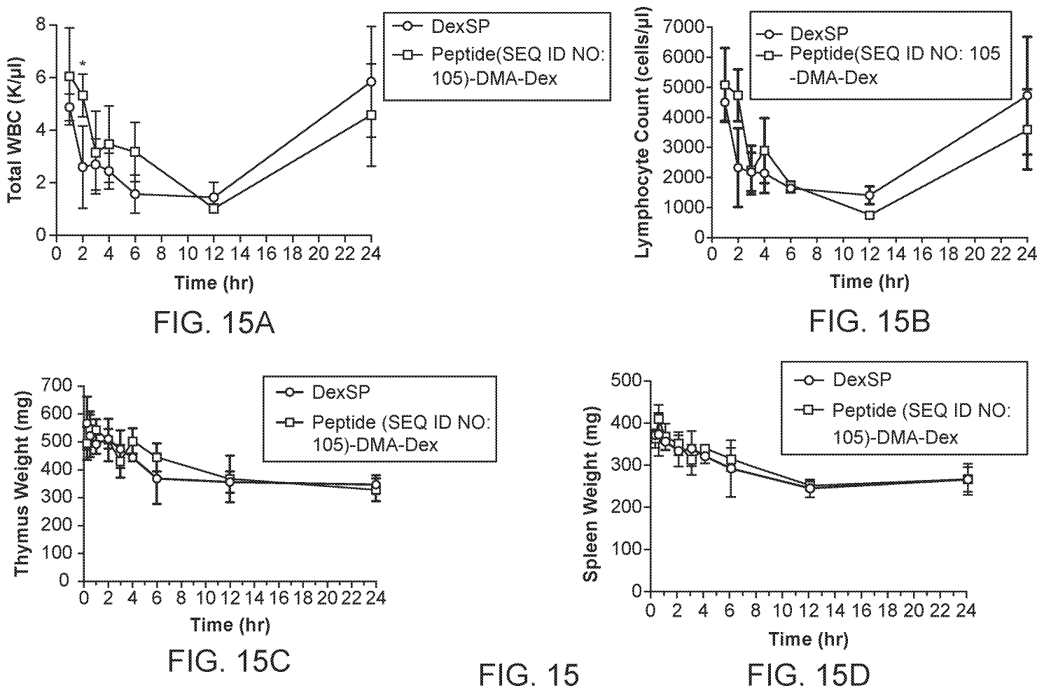

An indirect estimate of intact peptide-drug conjugate concentration at each time-point can be made by subtracting the free Dex concentration (from direct extraction) from the total Dex concentration (from forced hydrolysis). This analysis demonstrated a peak concentration of intact peptide-Dex conjugate that was similar to that of total Dex immediately after dosing but declined at a faster rate than that of DexSP (FIG. 14C). The intact peptide-drug conjugate was not detectable after 6 h and the apparent t_{1/2} of the intact peptide-drug conjugate was approximately 1.5 h. Systemic exposure to Dex was assessed by multiple parameters: total white blood cell (WBC) count; absolute lymphocyte count; thymus weight; and spleen weight (FIG. 15). Interestingly, despite the significantly lower levels of free Dex in plasma of animals treated with peptide, the measures of systemic exposure to Dex had some similarities between animals in both treatment groups (FIG. 15). The CBC data may show a delay of WBC (FIG. 15A) and lymphocyte (FIG. 15B) response to the peptide(SEQ ID NO: 105)-DMA-Dex versus DexSP, though the only time-points with a statistically significant difference between Dex and peptide(SEQ ID NO: 105)-DMA-Dex are at 2 h, with cell counts being higher in the peptide(SEQ ID NO: 105)-DMA-Dex group as compared to DexSP, and at 12 h, when lymphocyte counts are lower in the peptide(SEQ ID NO: 105)-DMA-Dex group as compared to DexSP (FIG. 15A and FIG. 15B). Thymus weight (FIG. 15C) and spleen (FIG. 15D) weight were not significantly different between DexSP and peptide(SEQ ID NO: 105)-DMA-Dex at any time-point and these organs remained reduced in size at 24 h when plasma WBC counts had returned to normal (FIG. 15C and FIG. 15D).

The data, including the LC/MS data, demonstrate a substantial difference in plasma free Dex levels in the peptide(SEQ ID NO: 105)-DMA-Dex-treated group as compared to DexSP over the first six hours, but the CBC and tissue weight data have similarities in both treatment groups, suggesting that the effect of Dex on lymphocyte redistribution may be saturable at very low concentrations of free Dex in the circulation.

### Example 25

### In Vivo Pharmacodynamic Response of Peptide-Drug Conjugates in CIA Rats

This example demonstrates a study evaluating the effects of the peptide-drug conjugates in the rat CIA model. In one part of this study, the peptide-carbamate and peptide-DMA conjugates were tested at a single dose level. In the second study, the peptide-DMA conjugate was tested at a range of doses. All doses are stated as Dex equivalents and therefore do not incorporate the mass of the peptide for the peptide or the phosphate group for Dex SP.

### Peptide(SEQ ID NO: 105)-Carbamate-Dex and Peptide(SEQ ID NO: 105)-DMA-Dex Comparison Study

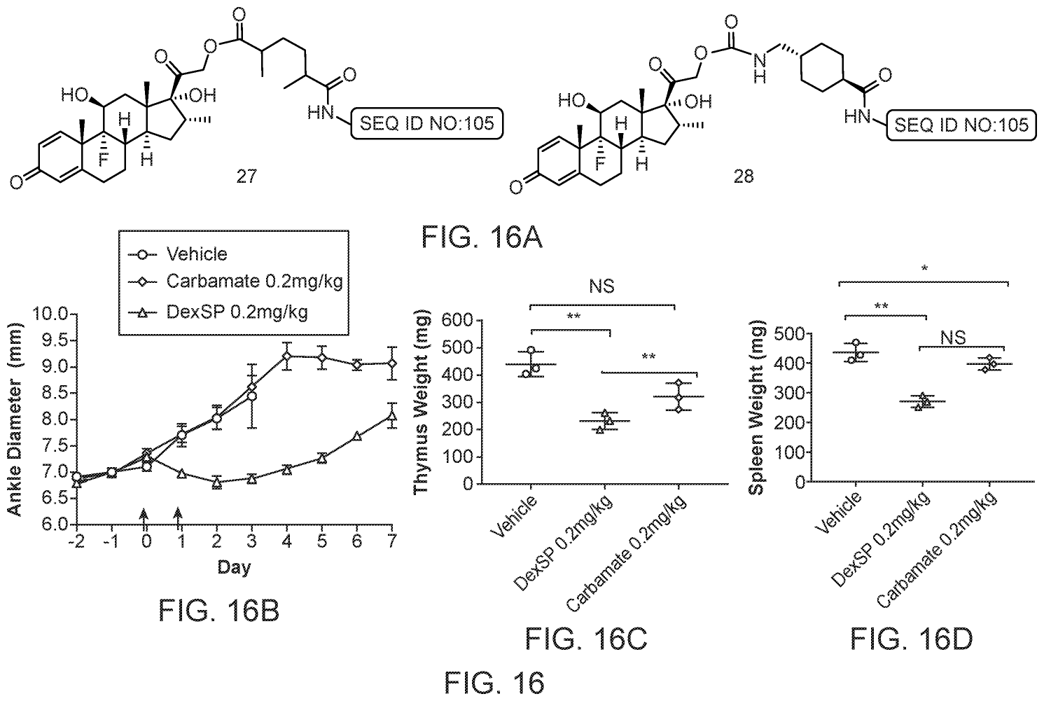

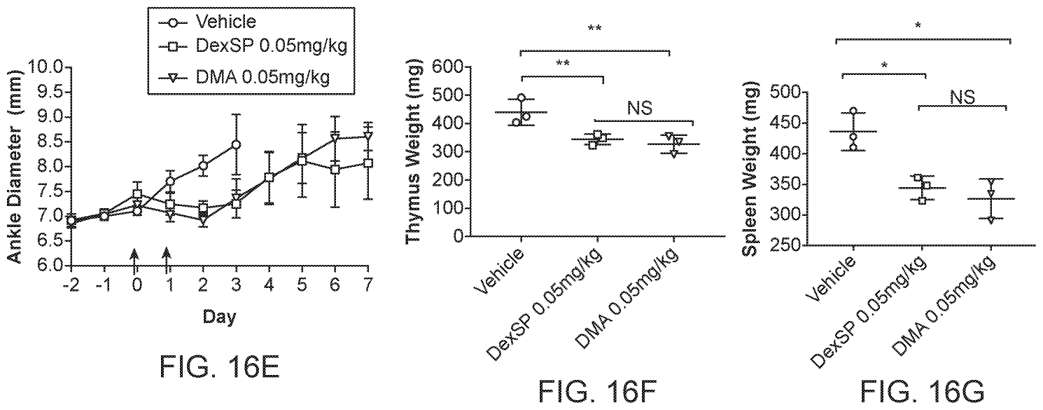

Based on the preliminary in vitro and in vivo studies, the conjugates peptide(SEQ ID NO: 105)-DMA-Dex (27) and peptide(SEQ ID NO: 105)-carbamate-Dex (28) (structures shown in FIG. 16A) were further investigated in vivo in CIA rats (FIG. 16).

A study was conducted in CIA rats to address the following: 1) functionality and duration of effect for peptide(SEQ ID NO: 105)-carbamate-Dex at 0.2 mg/kg as compared to Dex alone at the same dose; 2) functionality and duration of effect for peptide(SEQ ID NO: 105)-DMA-Dex at 0.05 mg/kg as compared to Dex alone at the same dose; 3) systemic markers of Dex exposure in rats treated with each PDC and Dex at their respective doses (FIG. 16B-FIG. 16G).

CIA was induced as described above in EXAMPLE 21. Rats were enrolled in the study following onset of arthritis in at least one ankle and randomly assigned into one of five treatment groups (vehicle; peptide-carbamate 0.2 mg/kg; Dex 0.2 mg/kg; peptide-DMA conjugate 0.05 mg/kg; Dex 0.05 mg/kg) with 5 rats per group. Treatment was administered on study day 0 and study day 1 and rats were euthanized on study day 7. The primary read-out in this cohort was change in ankle diameter during and after treatment, as a measure of functionality and duration of effect. Once enrollment was complete for the functionality study, remaining rats were randomly assigned to the same five treatment groups with 3 rats per group to assess systemic markers of Dex exposure. Twelve rats in this cohort developed arthritis while three had not developed disease at enrollment. Treatment was administered on study day 0 and day 1 and rats were euthanized 24 h after the last dose. The primary read-out for this cohort was measures of systemic exposure to Dex which included thymus weight, spleen weight, total white blood cell counts, lymphocyte counts, and change in body weight.

Peptide-carbamate-Dex at 0.2 mg/kg demonstrated no significant effect on ankle inflammation in CIA rats as compared to vehicle treatment. Dexamethasone at an equivalent dose was highly effective in reducing ankle diameter to baseline levels (FIG. 16B). Peptide(SEQ ID NO: 105)-carbamate-Dex caused a statistically significant reduction in thymus weight relative to vehicle but had no significant effect on spleen weight; whereas Dex at a similar dose had a significant reduction in both thymus and spleen weights (FIG. 16C-FIG. 16D). Peptide(SEQ ID NO: 105)-DMA-Dex caused a significant reduction in ankle diameter as compared to vehicle. The magnitude and duration of effect was indistinguishable between peptide-DMA-conjugate and Dex administered at the same dose. (FIG. 16E). Examination of systemic markers of Dex exposure demonstrated that peptide(SEQ ID NO: 105)-DMA-Dex and Dex caused similar significant reductions of thymus and spleen weight at the 0.05 mg/g dose as compared to vehicle (FIG. 16F and FIG. 16G). There were less significant differences in total WBC counts, lymphocyte counts, or change in body weight between treatment groups.

This data demonstrates that peptide-drug conjugates (e.g., peptide-DMA-Dex conjugates) of the present disclosure provide dose-dependent reduction of joint inflammation in the CIA model.

### Peptide(SEQ ID NO: 105)-DMA-Dex Dose Ranging Study

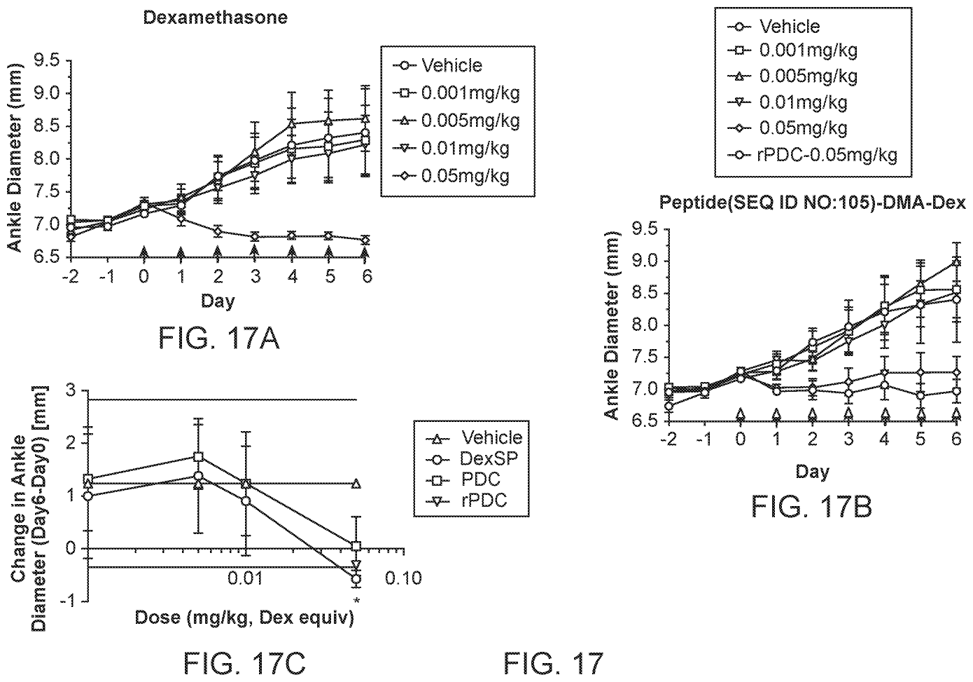

A dose-ranging study was conducted in CIA rats to titrate the effects of the peptide-drug conjugate peptide(SEQ ID NO: 105)-DMA-Dex on arthritic joints and on systemic Dex exposure, with comparison to DexSP (FIG. 17).

CIA was induced in female Lewis rats as described above in EXAMPLE 21. Ankle diameter measurements were recorded daily starting on induction day 9 and continuing for the duration of the study. Rats were enrolled following onset of arthritis in at least one ankle and randomly assigned to one of ten treatment groups (vehicle; DexSP 0.001 mg/kg; DexSP 0.005 mg/kg; DexSP 0.01 mg/kg; DexSP 0.05 mg/kg; peptide(SEQ ID NO: 105)-DMA-Dex 0.001 mg/kg; peptide(SEQ ID NO: 105)-DMA-Dex 0.005 mg/kg; peptide(SEQ ID NO: 105)-DMA-Dex 0.01 mg/kg; peptide(SEQ ID NO: 105)-DMA-Dex 0.05 mg/kg; recombinantly produced peptide(SEQ ID NO: 105)-DMA-Dex 0.05 mg/kg). This study used the synthetically produced peptide having the amino acid sequence set forth in SEQ ID NO: 105 and as described in EXAMPLE 2, as well as the recombinantly produced peptide-DMA-Dex conjugate (denoted as “rPDC”) refers to the PDC which was recombinantly produced and was included as a control to confirm that the synthetically and recombinantly produced peptides show comparable in vivo behavior. Each treatment was administered on the day of enrollment (study day 0) and repeated daily for 7 days (study day 6). Rats were euthanized 3 h after the final dose and blood and organs were collected for testing.

Peptide(SEQ ID NO: 105)-DMA-Dex and DexSP were both found to be effective at reducing ankle inflammation at the highest dose of 0.05 mg/kg but were indistinguishable from vehicle at the lower doses (FIG. 17A-FIG. 17C). Comparing the ankle diameter on study day 6 versus day 0, only DexSP 0.05 mg/kg was found to be statistically different than vehicle. DexSP was found to be statistically different from synthetic peptide-DMA conjugate (p=0.0452) but not the recombinant version recombinantly produced peptide-DMA conjugate (denoted as “rPDC”, p=0.1538) at 0.05 mg/kg. There was no significant difference between synthetic peptide and recombinant peptide at this dose.

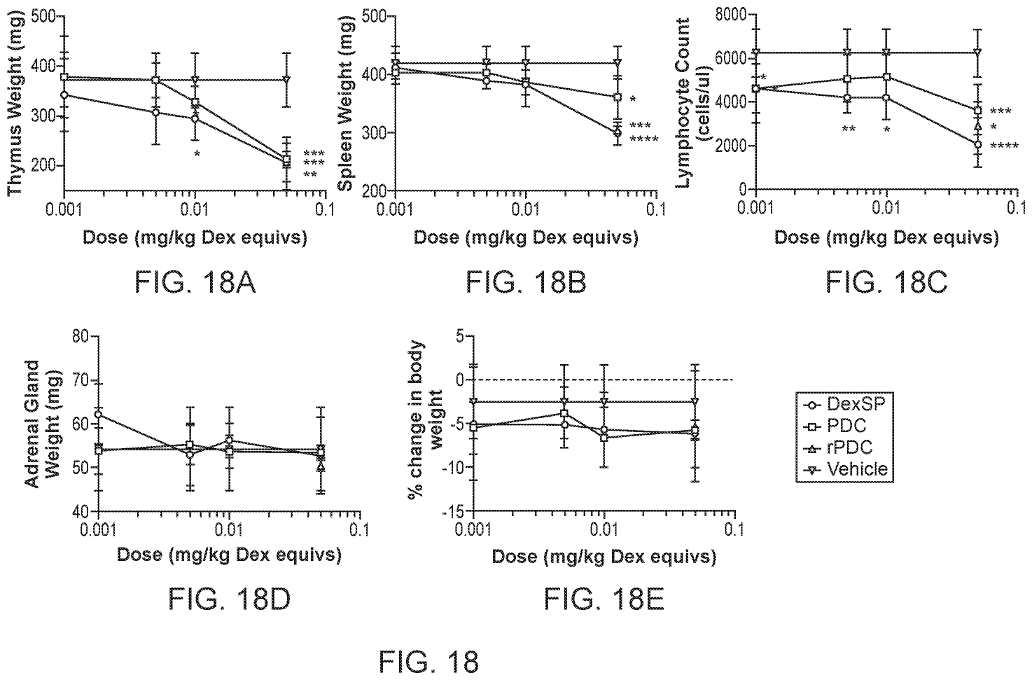

Systemic exposure to Dex was assessed by measuring thymus, spleen, adrenal gland weights, and change in body weight and changes in lymphocyte counts between treatment groups (FIG. 18). Peptide(SEQ ID NO: 105)-DMA-Dex (PDC) and Dex affected a number of these parameters but there were differences in their dose response profiles. At the highest dose of 0.05 mg/kg, thymus weight (FIG. 18A), spleen weight (FIG. 18B), and plasma lymphocyte counts (FIG. 18C) were found to be significantly reduced as compared to vehicle for Dex SP, peptide(SEQ ID NO: 105)-DMA-Dex, and recombinantly produced peptide(SEQ ID NO: 105)-DMA-Dex (denoted as “rPDC”) (FIG. 18A-FIG. 18C). However, spleen weight and plasma lymphocyte counts were significantly higher in the PDC treated group as compared to DexSP at this dose level (FIG. 18B and FIG. 18C), indicating reduced systemic Dex exposure in animals receiving the peptide-Dex conjugates vs. Dex alone. There was less significant difference between peptide(SEQ ID NO: 105)-DMA-Dex and DexSP with regard to thymus weight (FIG. 18A). There was also less significant difference in adrenal gland weight for any treatment as compared to vehicle (FIG. 18D).

The general health status of the rats was also monitored. Overall, the rats tolerated the treatments well with no significant changes in total body weight (FIG. 18E) and only mild, if any, alteration in serum chemistry parameters were observed. In sum, delivery of Dex using the herein disclosed peptide-DMA-Dex conjugates has demonstrated reduced and delayed systemic exposure to Dex while reproducibly reducing ankle swelling in an arthritis model. The pharmacokinetic profile of peptide-DMA-drug in plasma demonstrates that the DMA linker is functioning to reduce systemic exposure to free Dex as compared to administration of DexSP. Thus, the herein described peptide-drug conjugates provide therapeutic and potential safety benefit compared to administration of a drug alone.

### Example 26

### Biodistribution and Pharmacokinetics of Peptides In Vivo

This example illustrates biodistribution and pharmacokinetics in vivo of the peptides comprising the amino acid sequences set forth in SEQ ID NO: 105, SEQ ID NO: 103, and SEQ ID NO: 184, respectively.

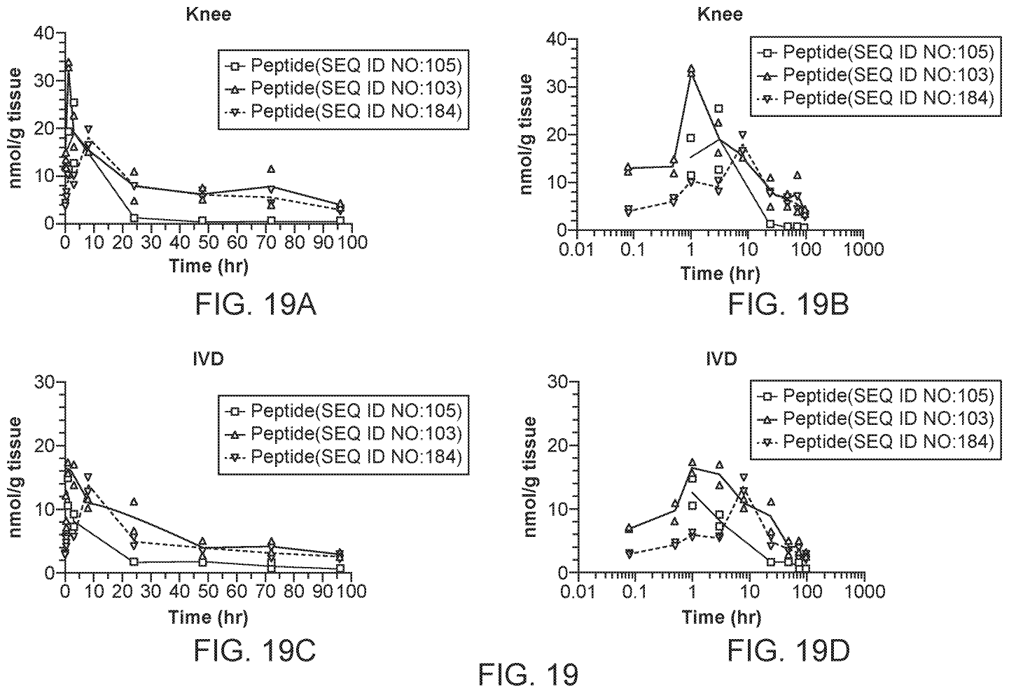

To that end, the biodistribution pharmacokinetics of ¹⁴C-labeled peptides ¹⁴C-peptide(SEQ ID NO: 105), ¹⁴C-peptide(SEQ ID NO: 103), and ¹⁴C-peptide(SEQ ID NO: 184) to the knee (FIG. 19A and FIG. 19B) and intraverterbral discs (IVD, FIG. 19C and FIG. 19D) was assessed in mice (FIG. 19). Tissue levels were quantified by whole body autoradiography of peptides that had been radiolabeled by reductive methylation of the N-terminus. ¹⁴C-peptide(SEQ ID NO: 105) and ¹⁴C-peptide(SEQ ID NO: 103) both peaked in knee and IVD around 1-3 hours whereas ¹⁴C-peptide(SEQ ID NO: 184) peaked later at 8 hours. After 24 hours, all peptides had a slow decreasing plateau in both tissues, with peptide(SEQ ID NO: 103) and peptide(SEQ ID NO: 184) detected at substantially higher levels than peptide(SEQ ID NO: 105). All peptides still had observable signal at the last time point of 96 hours. The results show sustained levels of all three peptides in the knee and IVD through 96 hours, but higher sustained signals in cartilage from peptide(SEQ ID NO: 103) and peptide(SEQ ID NO: 184) than peptide(SEQ ID NO: 105), and a later tₘₐₓ for peptide(SEQ ID NO: 184) than for peptide(SEQ ID NO: 105) and peptide(SEQ ID NO: 103). As the peptide levels are based on the radiolabel attached to the N-terminus of the peptide, the decrease in signal with time may be due to aminopeptidase activity on the N-terminus of the peptides (which would not be relevant on PDC conjugates, where the drug linker is attached to the N-terminus), due to peptide diffusion back out of the cartilage, or due to general peptide backbone cleavage. For example, higher sustained levels of peptide in the cartilage may be desirable if drug cleavage has a slower pharmacokinetics, and if the peptide-drug conjugate is not saturating peptide binding sites in the cartilage when using repeat dosing. As another example, an earlier tₘₐₓ in cartilage may be desirable to minimize premature cleavage of a conjugated drug in serum.

This data demonstrates that the peptides peptide(SEQ ID NO: 105), peptide(SEQ ID NO: 103), and peptide(SEQ ID NO: 103) can biodistribute to knee and IVD after systemic administration and be retained for days. Thus, the herein described peptides can be used in peptide-drug conjugates to both increase drug levels at a target site (e.g., cartilage in knee, ankles, etc.) and decrease systemic levels of drug during therapeutic intervention.

### Example 27

### Dexamethasone Delivery to Cartilage in Knee and Ankle Joints Using Peptide-Dex Conjugates

This example illustrates dexamethasone delivery to cartilage in knee and ankle joints using a peptide-Dex conjugate comprising the peptides comprising the amino acid sequence set forth in SEQ ID NO: 105 linked to Dex via a cysteine linker, peptide(SEQ ID NO: 105)-Cys-Dex (“peptide(SEQ ID NO: 105)-Cys” is disclosed as SEQ ID NO: 512).

Quantitative whole body autoradiography (QWBA) has been used in previous EXAMPLES to demonstrate that a peptide-Dex conjugate with a stable linker (e.g., peptide(SEQ ID NO: 105)-Cys-Dex (“peptide(SEQ ID NO: 105)-Cys” is disclosed as SEQ ID NO: 512)) distributes to cartilage of joints throughout the body in a similar manner to peptide(SEQ ID NO: 105) and that Dex conjugated to the stable linker without peptide (Cys-Dex) has minimal accumulation in cartilage (see e.g., EXAMPLE 21). Since QWBA relies upon detection of the radiolabel rather than the peptide or the drug directly, an orthogonal assay was developed to confirm these results. Immunohistochemistry methods were developed to detect peptide(SEQ ID NO: 105) and dexamethasone in cartilage of joints using a generated rabbit polyclonal anti-peptide(SEQ ID NO: 105) antibody and a commercially available anti-Dex antibody (Abcam, ab35000). Standard immunization methods were used to generate the anti-peptide(SEQ ID NO: 105) polyclonal antibody, which increases the immunogenicity of the dosed material and are not representative of potential immunogenicity of the unmodified peptide or therapeutic candidate molecules. To produce the immunogen for peptide(SEQ ID NO: 105) antibody generation, peptide(SEQ ID NO: 105) was conjugated to keyhole limpet hemocyanin (KLH) using ImjectTMmcKLH. Peptide(SEQ ID NO: 105)-KLH was administered as part of a pool of other KLH-conjugated peptides emulsified with Freund's complete adjuvant and administered to New Zealand White Rabbits following a standard schedule. The polyclonal antibody was purified from the serum using a HiTrap® mAb Select cartridge (GE Healthcare). Western blot was used to confirm antibody binding to the peptide.

To provide tissues for immunohistochemistry, one of four treatments (peptide(SEQ ID NO: 105)-Cys-Dex (n=3) (“peptide(SEQ ID NO: 105)-Cys” is disclosed as SEQ ID NO: 512); Cys-Dex (n=3); peptide(SEQ ID NO: 105) (n=2); or vehicle (n=2)) was administered intravenously to C57/Bl6 mice at a dose of 100 nmol and allowed to circulate for three hours prior to euthanasia. Hindlimbs were collected, fixed in formalin, decalcified and sectioned prior to staining with either anti-peptide(SEQ ID NO: 105) antibody, anti-Dex antibody, toluidine blue, or H&E.

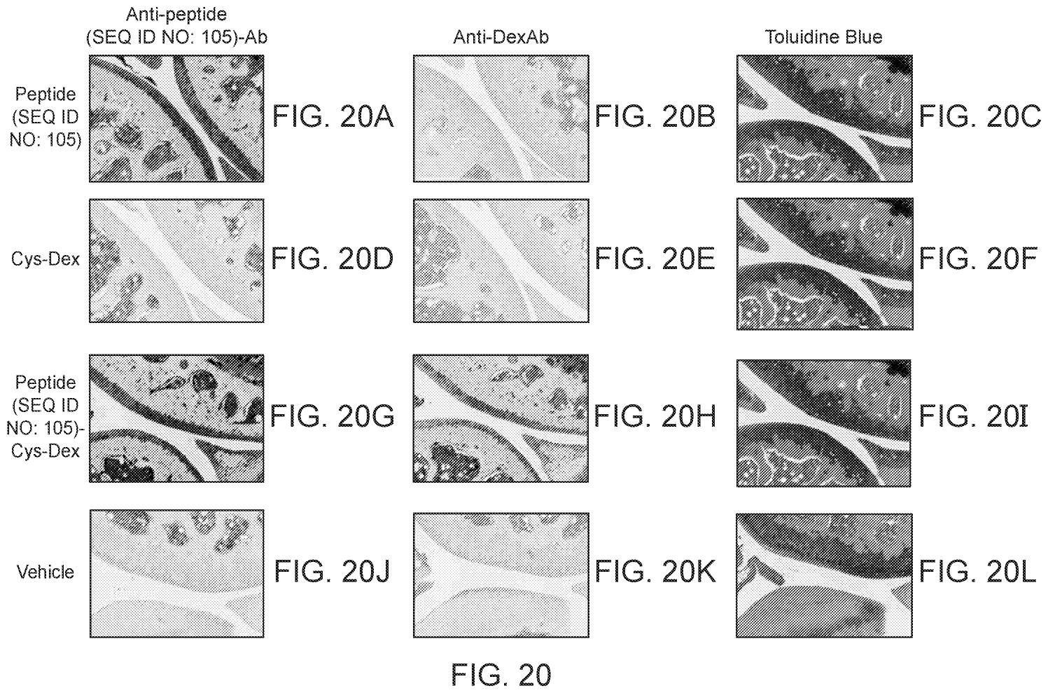

Immunostaining with anti-peptide(SEQ ID NO: 105) antibody demonstrated strong positive staining in articular cartilage and growth plates of knee joint sections from mice treated with either peptide(SEQ ID NO: 105) (FIG. 20A) or peptide(SEQ ID NO: 105)-Cys-Dex (“peptide(SEQ ID NO: 105)-Cys” is disclosed as SEQ ID NO: 512) (FIG. 20G). However, there was minimal staining in the cartilage of mice treated with Cys-Dex (FIG. 20D) or vehicle (FIG. 20J). IHC with anti-Dex antibody demonstrated strong positive staining in articular and growth plate cartilage of mice treated with peptide(SEQ ID NO: 105)-Cys-Dex (“peptide(SEQ ID NO: 105)-Cys” is disclosed as SEQ ID NO: 512) (FIG. 20H), but minimal cartilage staining in any other treatment group (e.g., FIG. 20B, FIG. 20E, FIG. 20K). Toluidine blue staining, which stains the proteoglycans in cartilage, demonstrated that there is similar proteoglycan content in knee sections from all treatment groups. A similar pattern of staining was observed in ankle sections. In both knee and ankle sections, there is strong staining in articular cartilage and growth plate cartilage by IHC staining of anti-Dex after administration of pep-cys-Dex. The staining is diffuse throughout the extracellular matrix and extends to the lacunae surrounding chondrocytes. There is some signal in bone marrow, tendons, and intracellular in osteocytes and chondrocytes. In a knee section from a mouse treated with Cys-Dex, there is no signal in the extracellular matrix of cartilage, but there is signal in bone marrow, tendons, osteocytes and chondrocytes. The distribution of staining is similar between knee sections from a Cys-Dex treated mouse and an untreated mouse. This provides confirmation that the peptide(SEQ ID NO: 105)-Cys-Dex conjugate (“peptide(SEQ ID NO: 105)-Cys” is disclosed as SEQ ID NO: 512) is delivering both peptide(SEQ ID NO: 105) and Dex to the cartilage in joints, whereas administration of Cys-Dex resulted in minimal levels of Dex in the cartilage.

Staining with anti-peptide(SEQ ID NO: 105) and anti-Dex antibodies was not restricted to cartilage. Anti-peptide(SEQ ID NO: 105) antibody staining resulted in prominent bone marrow signal in all treatment groups; however, the signal appeared stronger in sections treated with peptide(SEQ ID NO: 105) or peptide(SEQ ID NO: 105)-Cys-Dex (“peptide(SEQ ID NO: 105)-Cys” is disclosed as SEQ ID NO: 512) as compared to Dex or vehicle treatment. Anti-Dex antibody staining demonstrated weak to moderate bone marrow signal in sections from animals treated with peptide(SEQ ID NO: 105), Dex, or vehicle, but stronger staining in sections from animals treated with peptide(SEQ ID NO: 105)-Cys-Dex (“peptide(SEQ ID NO: 105)-Cys” is disclosed as SEQ ID NO: 512). Generally, peptide(SEQ ID NO: 105)-Cys-Dex (“peptide(SEQ ID NO: 105)-Cys” is disclosed as SEQ ID NO: 512) treated sections stained with both antibodies and peptide(SEQ ID NO: 105) treated sections stained with anti-peptide(SEQ ID NO: 105)-Ab appeared to have higher signal in tendons/ligaments, synovium, periosteum and osteocytes as compared to the other treatment groups.

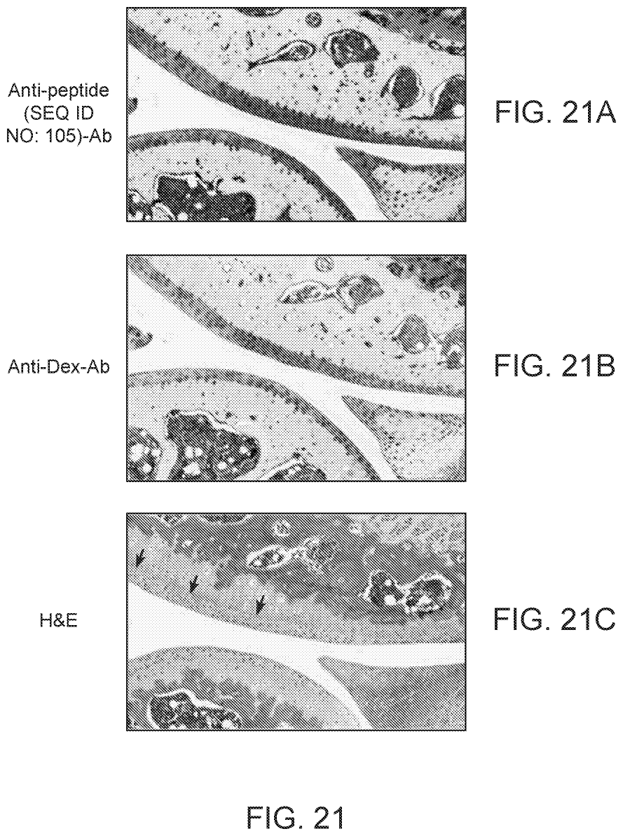

The distribution of staining with anti-peptide(SEQ ID NO: 105) and anti-Dex antibodies is very similar in the cartilage of peptide(SEQ ID NO: 105)-Cys-Dex (“peptide(SEQ ID NO: 105)-Cys” is disclosed as SEQ ID NO: 512) treated animals (FIG. 21). The conjugate is present throughout the extracellular matrix. Interestingly, the chondrocytes also stain positively with both antibodies, suggesting that the conjugate may be accumulating in or on the surface of these cells. Staining with anti-peptide(SEQ ID NO: 105) antibody (FIG. 21A) in the peptide(SEQ ID NO: 105)-Cys-Dex (“peptide(SEQ ID NO: 105)-Cys” is disclosed as SEQ ID NO: 512) treated animals, and staining with anti-Dex antibody (FIG. 21B) in the peptide(SEQ ID NO: 105)-Cys-Dex (“peptide(SEQ ID NO: 105)-Cys” is disclosed as SEQ ID NO: 512) treated animals extends only part-way through the depth of the cartilage and there appears to be a sharp boundary to the staining (FIG. 20 and FIG. 21). This boundary coincides closely with the location of the “tide-mark” of the cartilage that is visible in the H&E section (FIG. 21C). The tide mark demarcates the transition between non-calcified cartilage and calcified cartilage.

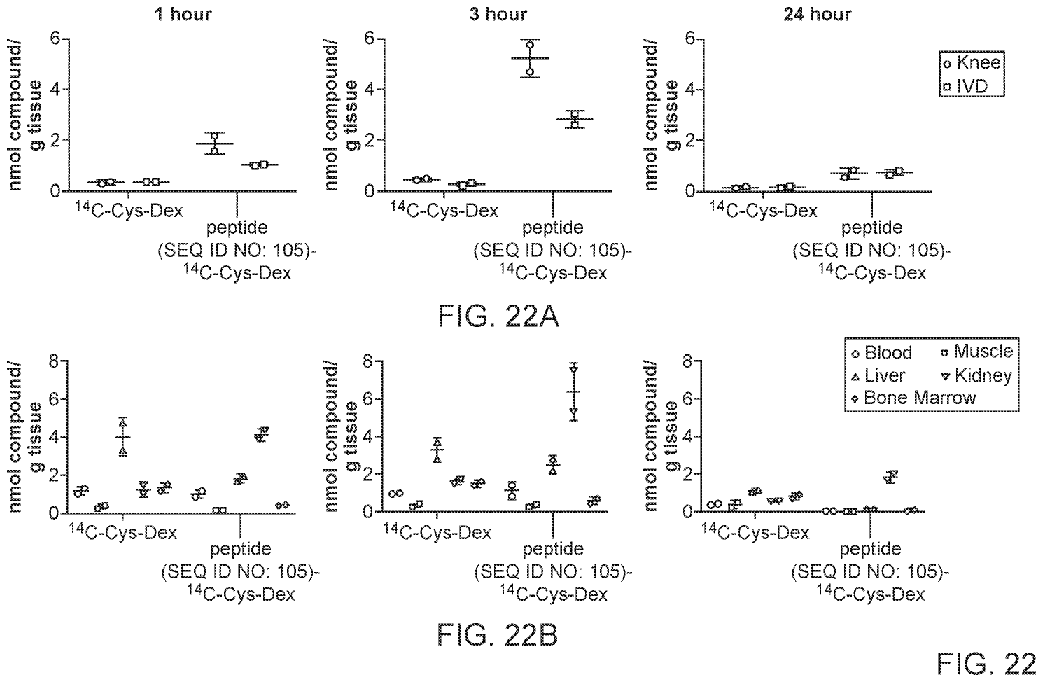

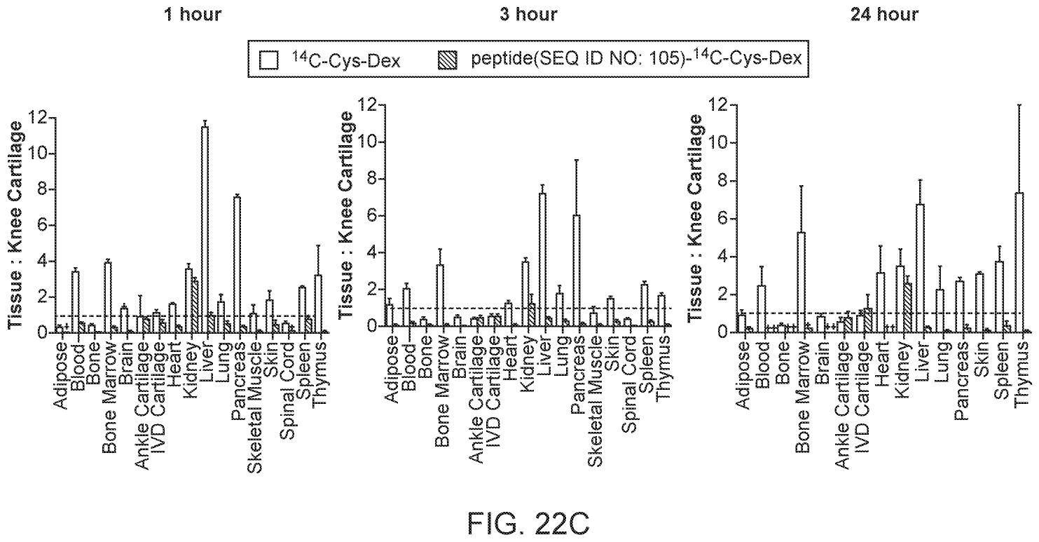

QWBA data in CIA rats was further analyzed to compare accumulation of peptide(SEQ ID NO: 105)-Cys-Dex (“peptide(SEQ ID NO: 105)-Cys” is disclosed as SEQ ID NO: 512) and Cys-Dex in cartilage and other tissues on a molar basis (nmol compound/g of tissue, FIG. 22A-FIG. 22C). In order to determine extent to which cartilage accumulation may impact the therapeutic window of Dex, we also calculated the ratio of Dex concentration in other tissues to the concentration in cartilage of the knee (FIG. 22C). This analysis demonstrated that non-targeted Dex accumulates in a wide array of different tissues in the body at concentrations that significantly exceed its concentration in the knee. On the other hand, when the concentration of peptide(SEQ ID NO: 105)-Cys-Dex (“peptide(SEQ ID NO: 105)-Cys” is disclosed as SEQ ID NO: 512) in tissues is compared to its concentration in knee cartilage, relatively few tissues are found to have equivalent or higher concentration than the knee. The tissues with highest peptide-drug conjugate accumulation include knee and other cartilaginous sites like IVDs and ankle, as well as tissues of metabolism and excretion such as liver and kidney (FIG. 22C). However, levels of Dex delivered to liver and many other non-target tissues such as bone marrow, spleen, thymus, blood, and pancreas are greatly reduced compared to cartilage levels when delivered by the peptide(SEQ ID NO: 105)-Cys-Dex (“peptide(SEQ ID NO: 105)-Cys” is disclosed as SEQ ID NO: 512) versus delivery as non-targeted Dex. This demonstrates potential for a substantially increased therapeutic window of targeting drug delivery via peptide(SEQ ID NO: 105)-Cys-Dex (“peptide(SEQ ID NO: 105)-Cys” is disclosed as SEQ ID NO: 512) to the desired cartilaginous tissues.

Overall, these data confirm, using an orthogonal method to QWBA, that conjugation of Dex to the peptides described herein (e.g., peptide(SEQ ID NO: 105)) using a stable linker results in delivery of both Dex and peptide(SEQ ID NO: 105) to cartilage, and that systemic administration of Dex alone does not result in any significant accumulation of the drug in knee and ankle joints. Thus, the herein disclosed peptides can be used to deliver a drug to target tissues to which the drug itself has no or only limited access to provide therapeutically effective concentrations.

### Example 28

### Development of Additional Linkers for Use in Peptide-Drug Conjugates

This example illustrates the development of additional linkers that can be used in combination with the herein described methods and compositions. The linkers described in this example can be used with any peptide described herein. Cleavable linkers for drug (e.g., steroid) delivery were developed based on the localization and kinetics of peptide accumulation in cartilage. Some peptides described herein predominantly localize extracellularly in the cartilage and thus, ester, carbonate, and carbamate linkers were chosen for their potential to hydrolyze chemically and via esterases and to release the steroid in unmodified form. The structures and hydrolysis rates of some peptide-Dex conjugates synthesized in the course of this study are presented below in TABLE 15.

Hydrolysis times for the peptide-drug conjugates shown in TABLE 15 below were determined as follows: peptide drug conjugates (PDCs) were incubated in PBS, rat plasma, or human plasma at 37° C. Samples were removed at regular intervals, processed by solvent extraction, and analyzed by LC/MS to quantitate free Dex. Each assay consisted of at least 9 time-points (ranging from 2 min to 32 h) with 3 replicate samples per time-point. Peptide(SEQ ID NO: 105)-DMA-Dex was monitored for up to 56 h in human plasma. The half-life was calculated based upon the rate of release in the time-frame monitored, however, some conjugates may not have hydrolyzed completely by the end of the assay.

TABLE 15
Chemical Structure and Hydrolysis Half-life of Peptide-Dex Conjugates
Synthesized Comprising Various Linkers.

| Peptide (SEQ ID | | |
|---|---|---|
| NO)-linker-drug | | |
| conjugate | | Hydrolysis |
| (Compound number) | Chemical structure | half-life |
| Peptide (SEQ ID NO: 105)-succinic acid-Dex (39) | 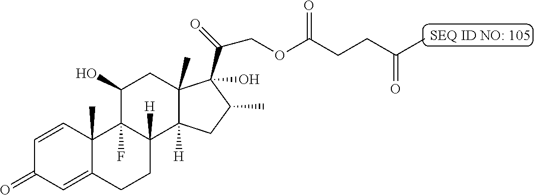 | Minutes in PBS |
| Peptide (SEQ ID NO: 105)- tetramethylene glutaric acid-Dex (26) | 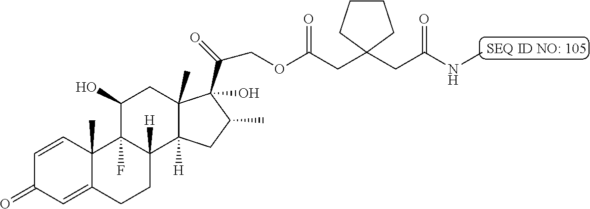 | 1.1 h in rat plasma |
| Peptide (SEQ ID NO: 105)-glutaric acid-Dex (23) | 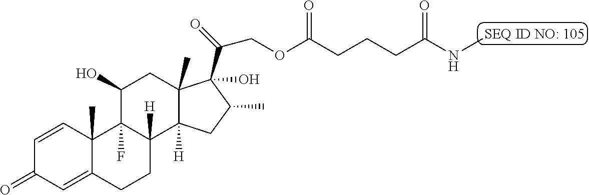 | 6 h in PBS 1.9 h in rat plasma 1.7 h in human plasma |
| Peptide (SEQ ID NO: 105)-adipic acid-Dex (24) | 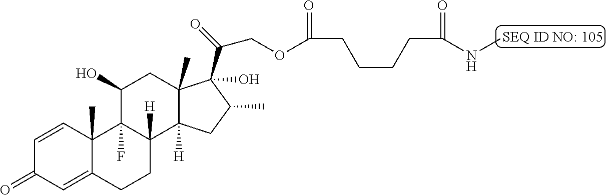 | 2.6 h in rat plasma |
| Peptide (SEQ ID NO: 105)-trans-1,4- cyclohexane- dicarboxylic acid- Dex (25) | 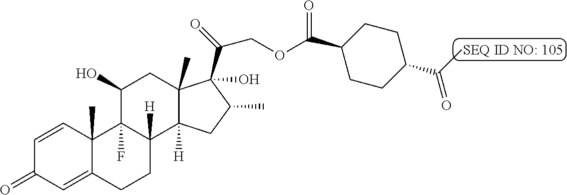 | No Dex release ≤32 h in PBS 3.4 h in rat plasma 21.3 h in human plasma |
| Peptide (SEQ ID NO: 105)- dimethyladipic Acid-Dex (27) | 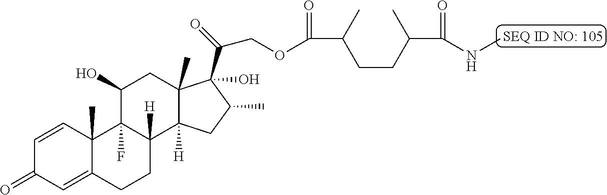 | No Dex release ≤32 h in PBS 9.9. h in rat plasma 22.4 h in human plasma |
| Peptide (SEQ ID NO: 105)-trans-1- aminomethylamine- cyclohexane-4- carboxylic Acid- Dex (28) | 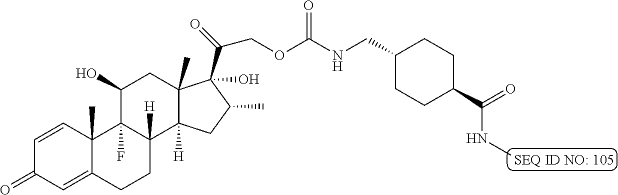 | No Dex release ≤32 h in PBS No Dex release ≤32 h in rat plasma No Dex release ≤32 h in human plasma |
| Peptide (SEQ ID NO: 105)-1,4-trans- cyclohexyl- carbamate-Dex (40) | 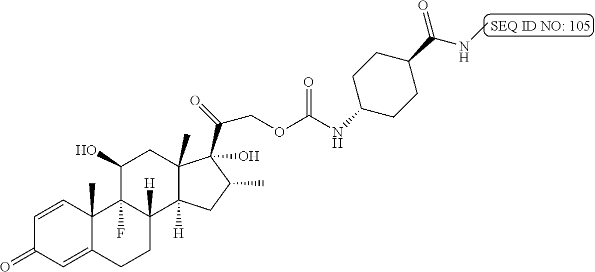 | Negligible Dex release ≤2 h in rat plasma |
| Peptide (SEQ ID NO: 105)-(direct)- carbamate-Dex (41) | 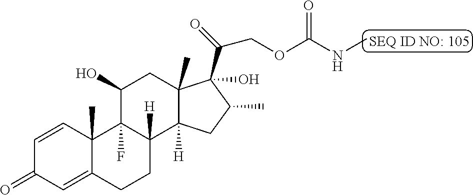 | No Dex release ≤32 h in rat plasma |
| Peptide (SEQ ID NO: 105)-beta- alanine-carbamate- Dex (42) (“Peptide (SEQ ID NO: 105)-beta- alanine” is disclosed as SEQ ID NO: 513) | 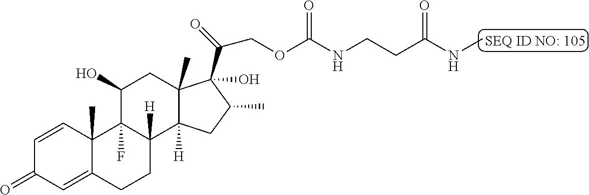 | No Dex release ≤32 h in rat plasma |
| Peptide (SEQ ID NO: 105)-1,4- benzoic acid- Dex (43) | 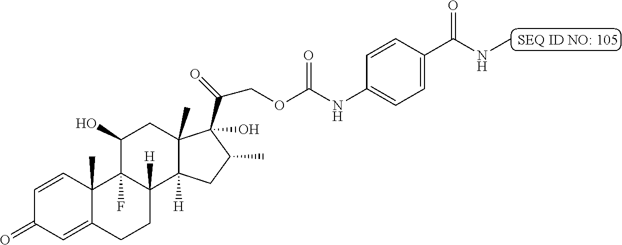 | No Dex release ≤32 h in rat plasma |
| Peptide (SEQ ID NO: 105)-1,4-acetic- phenylcarbonate- Dex (44) | 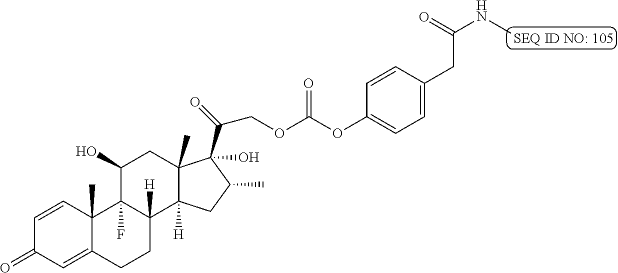 | Minutes in rat plasma |
| Peptide (SEQ ID NO: 105)-1,4- benzoic- methylcarbonate- Dex (45) | 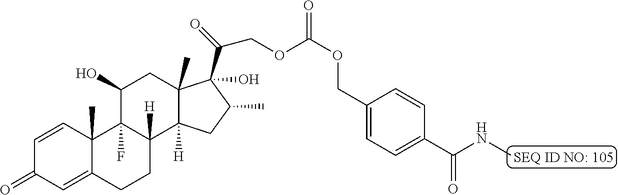 | Minutes in rat plasma |

The effects of various parameters such as the length of the carbon chain of the linker and the steric hindrance on the hydrolysis rate was evaluated. It was found that, in general, increasing the length of the aliphatic chain and increasing steric hindrance around the ester bond can result in slower release of dexamethasone (Dex) from the conjugates (TABLE 15). As expected, hydrolysis rates were generally fastest in rat plasma, followed by human plasma, and slowest in PBS, presumably due to decreasing levels of enzymes such as serum carboxylesterases present. Carbonate and carbamate linkers were also investigated to increase chemical diversity and to broaden the range of hydrolysis rates that are available in the linker inventory. It was found that carbonate linkers hydrolyzed very rapidly in vitro, often reaching complete hydrolysis in less than one hour, while the carbamate linkers were quite stable and did not release Dex within the 32 hour observation period in the tested fluids

This data shows that peptide drug conjugates synthesized and described herein (e.g., those shown above in TABLE 15) can provide a wide range of lability to chemical and enzymatic hydrolysis for optimal drug delivery (e.g., Dex, des-ciclesonide, or other drugs) to target tissue(s) (e.g., joints) while minimizing systemic exposure to the drug (e.g., a glucocorticoid such as Dex). Without being bound by any theory, it was assumed that the chemical and enzymatic environment of an inflamed joint can provide altered local cleavage rates from those measured in plasma. The cleavage rate can also vary in various fluids such as synovial fluid, plasma, and urine. The difference in cleavage rates in rat versus human plasma, may be due to varying levels of esterases.

### Example 29

### Synthesis and In Vitro Hydrolysis of Peptide-Des-Ciclesonide Conjugates

This example demonstrates the synthesis and hydrolysis properties of peptide-des-ciclesonide (peptide-dCIC) conjugates (FIG. 23).

### Synthesis

The conjugate peptide(SEQ ID NO: 105)-DMA-dCIC (46) was synthesized using the following three synthetic steps of ester bond formation, sulfo-NHS ester formation, and peptide conjugation to yield SEQ ID NO: 105-DMA-dCIC as described in Scheme 15 below and FIG. 23A.

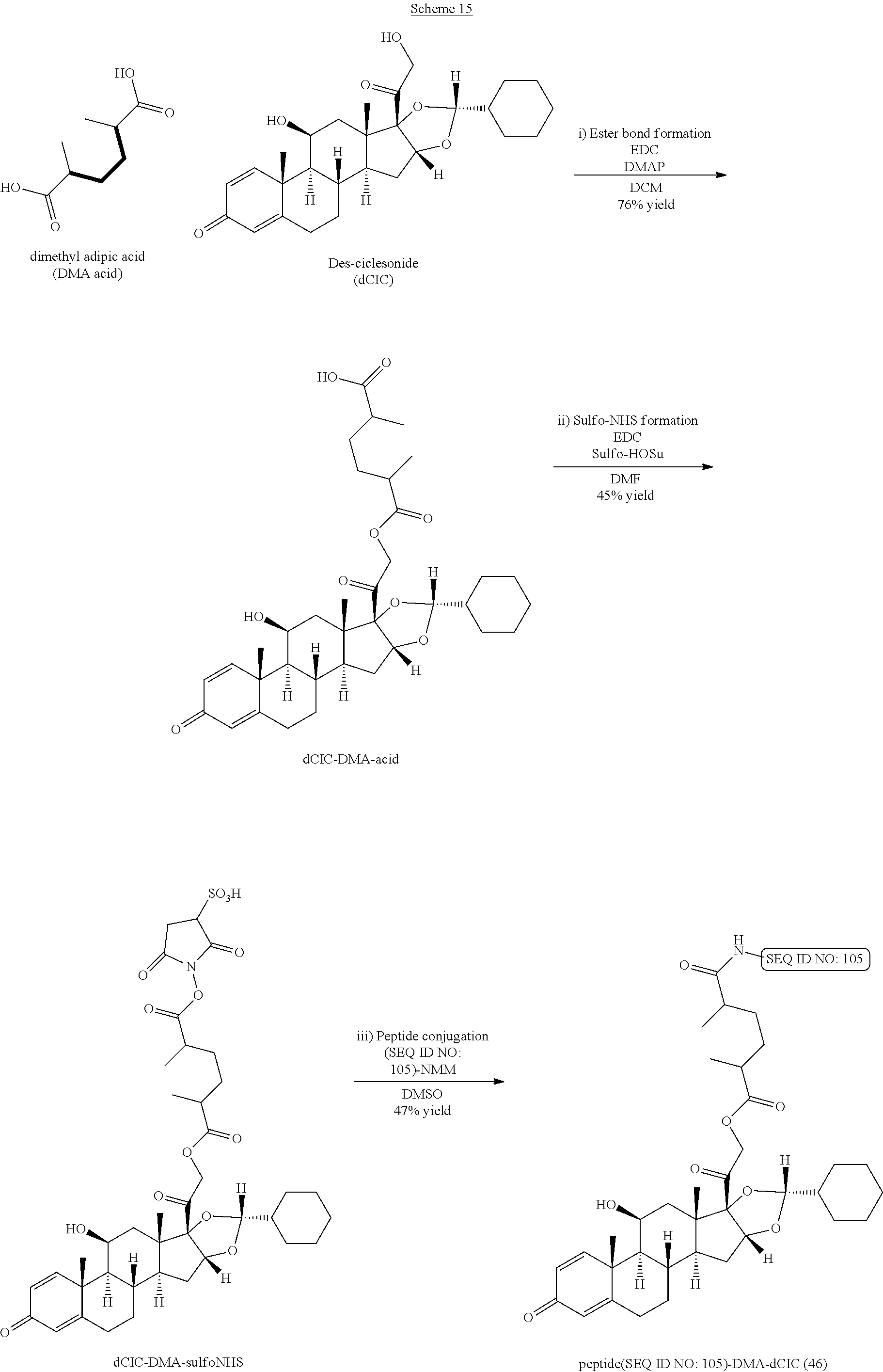

### Step 1: Ester Bond Formation-Synthesis of dCIC-2,5-Dimethyl Adipic Acid

Des-ciclesonide (128.8 mg, 0.27 mmol), 2,5-dimethyl adipic acid (235 mg, 1.35 mmol), 1-Ethyl-3-(3-dimethylaminopropyl)carbodiimide hydrochloride (518 mg, 2.7 mmol) and 4-dimethylaminopyridine (330 mg, 2.7 mmol) were dissolved in 10 mL anhydrous dichloromethane (DCM). The reaction mixture was stirred at room temperature for approximately 20 hours. The solvent was removed under reduced pressure. The residue was purified by HPLC to give 129 mg white powder with 76% yield. LC-MS showed greater than 97% purity with mass 609.3 (M−17).

### Step 2: Sulfo-NHS Ester Formation-Synthesis of dCIC-2,5-Dimethyl Adipic NHS Ester

dCIC-2,5-dimethyl-adipic acid (86.4 mg, 0.14 mmol), N-(3-dimethylaminopropyl)-N′-ethylcarbodiimide hydrochloride (80.1 mg, 0.42 mmol) and N-hydroxysulfosuccinimide sodium salt (152 mg, 0.7 mmol) were dissolved in 3 mL anhydrous DMSO. The reaction mixture was stirred at room temperature for approximately 1 hour. LCMS showed 65% product, 7% EDC urea by-product and 25% starting material. N-hydroxysulfosuccinimide sodium salt (90 mg) and N-(3-dimethylaminopropyl)-N′-ethylcarbodiimide hydrochloride (51 mg) were added. The reaction mixture was stirred at room temperature for approximately 2 hours. LCMS showed 88% product, 7% EDC urea by-product and 3% starting material. The reaction mixture was purified by HPLC to give 54.5 mg white powder with 45% yield. LC-MS showed greater than 93% purity with mass 804.3 (M+1).

### Step 3: Peptide Conjugation-Synthesis of (SEQ ID NO: 105)-DMA-dCIC (46)

To the stirred mixture of the peptide having the amino acid sequence set forth in SEQ ID NO: 105 (50 mg, 0.012 mmol) and N-methylmorpholine (13.2 μL, 0.12 mmol) in 1 mL anhydrous DMSO, was added Des-ciclesonide-2,5-dimethyl-adipic-sulfo-NHS ester (22 mg, 90% purity, 0.024 mmol). The reaction mixture was stirred at room temperature for 2 days. The reaction mixture was purified by HPLC to give the product peptide(SEQ ID NO: 105)-DMA-dCIC as a white powder (combined 2 reactions, total 100 mg of the peptide was used, 83.4 mg product was obtained with 47% yield). LC-MS showed 99% purity with MS: 1228.4 (M/4), 1637.6 (M/3).

These steps were also successfully performed using the peptides having the amino acid sequences set forth in SEQ ID NO: 103, SEQ ID NO: 184 and SEQ ID NO: 509, and in any combination with the glucocorticoids dexamethasone, budesonide, and triamcinolone acetonide.

In addition to the conjugate peptide(SEQ ID NO: 105)-DMA-dCIC, a peptide(SEQ ID NO: 105)-Cys-dCIC (47) conjugate (“peptide(SEQ ID NO: 105)-Cys” is disclosed as SEQ ID NO: 512) was prepared, wherein the cysteine linker was further modified to comprise ¹⁴C-containing methyl groups furnishing the conjugate peptide(SEQ ID NO: 105)-¹⁴C-Cys-dCIC (48) (“peptide(SEQ ID NO: 105)-Cys” is disclosed as SEQ ID NO: 512). The peptides used included, but were not limited to those with amino acid sequences set forth in SEQ ID NO: 103, SEQ ID NO: 105, SEQ ID NO: 184 and SEQ ID NO: 509.

An exemplary synthetic scheme for obtaining ¹⁴C-labeled peptide-drug conjugates is shown below (Scheme 16):

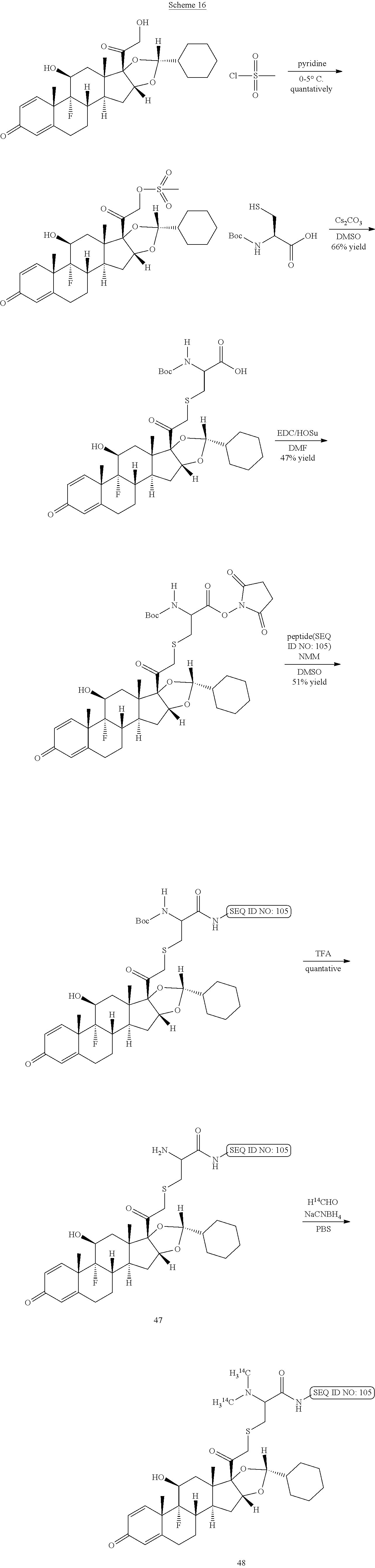

The synthetic route shown above can include the following steps: mesylate formation (1), sulfur alkylation (2), NHS formation (3), peptide conjugates (4), Boc-group deprotection (5), and reductive amination (6).

### Step 1: Mesylate Formation-Synthesis of dCIC Mesylate

Des-ciclesonide (83.8 mg, 0.18 mmol) was dissolved in 1 mL anhydrous pyridine (not completely dissolved at the beginning, suspension). The reaction vial was sealed with a septum in an ice bath. Then methane sulfonyl chloride (28 μL, 0.36 mmol) was added dropwise to the suspension of Des-ciclesonide through a syringe. The reaction mixture was stirred at 0° C. for half hour. The work up of the reaction mixture was performed as follows: the reaction mixture was poured into 25 mL iced water and the product was extracted with EtOAc. The organic part was washed by 1 mL 0.1 M HCl, saturated NaHCO₃ and brine, then dried over Na₂SO₄. The mixture was filtered to get the filtrate and the solvent was removed under reduced pressure to give the product quantitatively.

### Step 2: Sulfur Alkylation-Synthesis of dCIC-Boc-Cysteine

To the stirred mixture of Boc-L-Cysteine (304 mg, 1.44 mmol) and Cs₂CO₃ (469 mg, 1.44 mmol) in 1 mL anhydrous DMSO, were added des-ciclesonide mesylate (98 mg, 0.18 mmol) in 1 mL anhydrous DMSO. The reaction mixture was stirred at room temperature for approximately 1.5 hours. The reaction was filtered to remove the solid; the resulting solution was purified by HPLC to obtain 80 mg white powder with 66% yield. LC-MS showed greater than 90% purity with mass 656.3 (M−17).

### Step 3: NHS Ester Formation-Synthesis of dCIC-Boc-Cysteine-NHS Ester

To the stirred solution of des-ciclesonide-Boc-Cysteine (18.8 mg, 0.028 mmol) in 2 mL anhydrous DMF, were added 1-Ethyl-3-(3-dimethylaminopropyl)carbodiimide (44 mg, 0.228 mmol) and N-hydroxysuccinimide (44 mg, 0.38 mmol). The reaction mixture was stirred at room temperature for approximately 20 hours. The reaction mixture was purified by HPLC to give 10.1 mg white powder with 47% yield. The purity is 92% with mass 671.3 (M−100).

### Step 4: Peptide Conjugation Synthesis

To the stirred mixture of the peptide having amino acid sequence SEQ ID NO: 105 (20 mg, 0.0047 mmol) and N-methylmorpholine (5.2 μL in 0.52 mL anhydrous DMSO; 100× dilution; 0.047 mmol), was added des-ciclesonide-Boc-Cysteine-NHS ester (7.2 mg, 0.0093 mmol). The reaction mixture was stirred at room temperature for overnight. The reaction mixture was purified by HPLC to get 14.8 mg white powder with 62% yield. LC-MS showed 97% purity with mass 1240.2 (M/4) and 1653.3 (M/3).

### Step 5: Boc-Deprotection Synthesis

To the white powder of des-ciclesonide-Boc-Cysteine-Peptide (10 mg, 0.0020 mmol) in 30 mL clear vial, was added 100 μL TFA (99% purity). After 5 min LC-MS analysis revealed the reaction was complete. 2 mL of acetonitrile and water (1:1 ratio) was added, then frozen at −78° C. followed by lyophilization to obtain 12 mg of white powder. LC-MS showed greater than 97% purity with mass 1215.2 (M/4), 1620.0 (M/3).

### Step 6: Reductive Amination-Synthesis of C14 Labeled Conjugate

To the solution of peptide(SEQ ID NO:105)-Cys-dCIC (10 mg, 0.0020 mmol) in 5 mL water, was added 470 μL 10×PBS and 25 μL ¹⁴C labeled formaldehyde (8% aq. solution) in a radioactive fume hood. NaCNBH₄ (aq) solution (4.6 mg in 1 mL) was added. The reaction mixture was vortexed and left for approximately 20 hours. The reaction mixture was passed over a Strata-X column previously activated with 3 mL methanol and equilibrated with 3 mL water. The adsorbed conjugate was washed with water (3 mL) and eluted with 4 mL of 2% formic acid in methanol. The methanol/formic acid mixture was removed under a stream of nitrogen to give 6.99 mg product.

This data demonstrates a robust, reproducible and versatile synthetic method for obtaining peptide-drug conjugates (e.g., peptide-linker-glucocorticoid conjugates) as described herein that can be used in therapeutic and/or diagnostic applications.

The following peptide-des-ciclesonide conjugates were prepared according to the synthetic procedures described in this and EXAMPLE 5-EXAMPLE 19 of this disclosure.

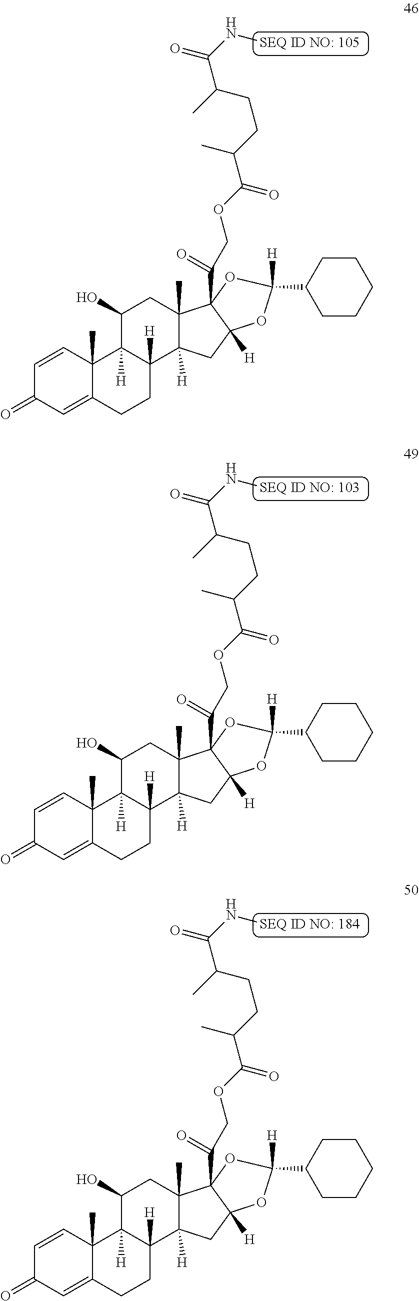

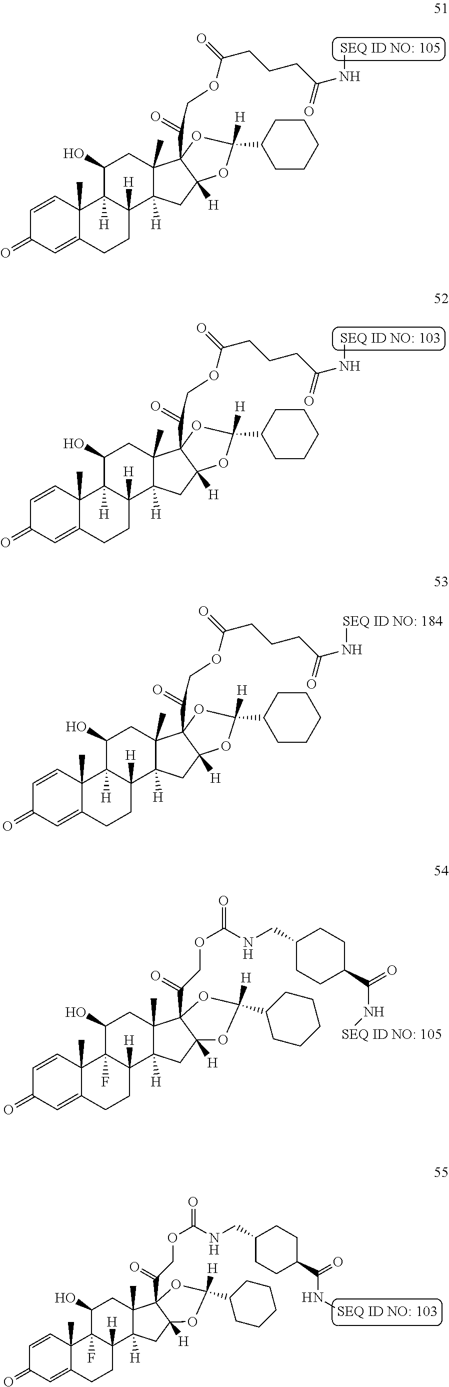

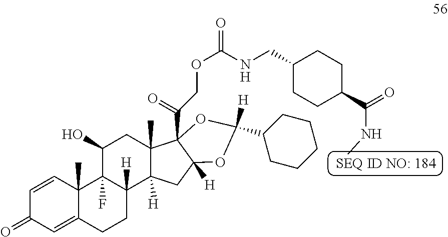

### Example 30

### Development of Additional Disease Models

This example demonstrates development of an additional disease model to assess function and therapeutic effect of the herein described peptides and peptide-drug conjugates.

To that end, local versus systemic administration of DexSP in an acute intra-articular (IA) IL-1-induced IL-6 challenge rat model was assessed. For that study, 30 ng of IL-1b was injected IA in the knee of Male Sprague-Dawley rats, in addition to injection of 30 or 3 μg of DexSP dosed either IA or IV. After 4 h, IL-6 levels in synovial fluid of the same knee were measured by Luminex and normalized to protein content of the knee lavage. Dex administered IA (co-injected with IL-1b) caused no decrease in IL-6 secretion, whereas Dex administered IV did cause some decrease in IL-6 secretion in the synovial fluid (Hulme, et al (2017).

The results show that the systemic immune suppression effects of Dex can be very significant. This model could also be used to test suitability of des-ciclesonide peptide-drug conjugates (PDCs) for testing in this model as well.

### Example 31

### In Vivo Pharmacodynamic Studies of Peptide-dCIC Conjugates

This example illustrates the in vivo pharmacodynamic (PD) evaluation of peptide-dCIC conjugates in vivo.

In this example, an IL-1β Challenge rat model was used for the evaluation of peptides-drug conjugates. In this model, IL-1β is injected intra-articularly (i.e., IA) into a single knee joint to induce an inflammatory state that can be quantified by measuring IL-6 levels in joint lavage fluid (EXAMPLE 30). The primary goal of using this model was to induce a localized inflammatory state in the joint, as opposed to the systemic inflammation induced in other models, as a way to differentiate localized delivery of dCIC to the joint from systemic administration of the steroid. An additional advantage with this model is that it can be run rapidly and allows for a faster decision-making process. CBC counts were used to assess any reductions in various white blood cell levels, which are established markers of systemic exposure to glucocorticoids.

This study was designed to test whether dCIC delivered by a peptide would enable local suppression of inflammation in the joint while reducing systemic exposure to dCIC compared to that obtained when dosing therapeutic levels of dCIC alone. To enable systemic delivery of dCIC (unconjugated), it was formulated in 40% propylene glycol and then heated in order to solubilize the molecule. Peptide-dCIC-conjugate was soluble in PBS and did not require propylene glycol or heat. The peptide-drug conjugates of the disclosure can have a water solubility that allows conjugates to be formulated for systemic delivery, wherein at least some of the drugs used in the peptide-drug conjugates may not have sufficient water solubility for systemic administration when not conjugated to peptide. The conjugates comprising those drugs can home, target, migrate to, accumulate in, bind to, be retained by, or be redirected to a cartilage of a subject.

### Initial Dose-Ranging Study

IL-6 Measurements in Knee Lavage Fluid.

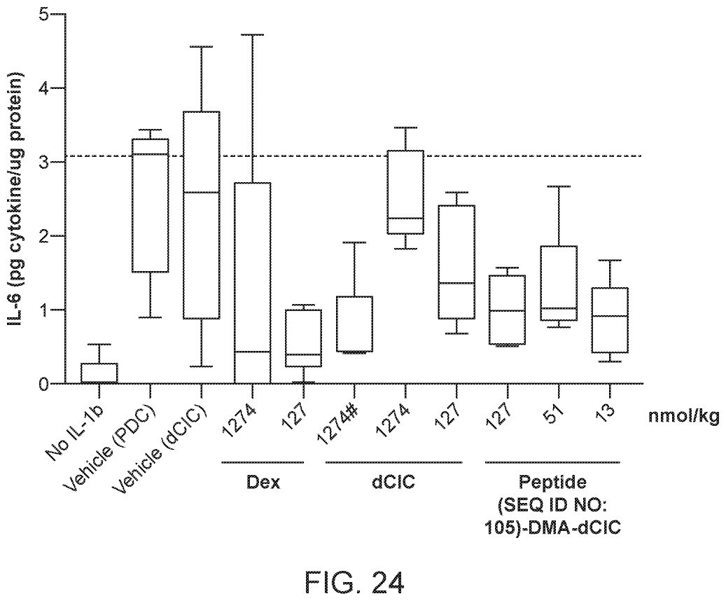

A dose-ranging study was conducted in an IL-1β challenge model to identify a dose of both peptide-dCIC conjugate (peptide(SEQ ID NO: 105)-DMA-dCIC) and dCIC that would reduce IL-6 secretion levels in the joint and to understand the effects of each treatment on systemic markers of steroid exposure in the model. Peptide(SEQ ID NO: 105)-DMA-dCIC was tested at 0.1-10 fold the effective dose level for peptide-Dex conjugate in the rat CIA model. On study day 0, male Sprague-Dawley rats (minimum 250 g) were weighed and randomized by body weight into one of thirteen treatment groups (FIG. 24).

IL-6 was measured in knee lavage fluid using a Luminex assay and levels were normalized to total protein in the lavage fluid (FIG. 24). Assessment of the PD inflammatory marker IL-6 in the knee joints of rats challenged with IL-1β was performed. Terminal synovial fluid was collected from all animals and analyzed by Luminex 200 for IL-6 cytokine concentrations using EMD Milliplex MAP Rat Cytokine/Chemokine Magnetic Panel (RECYTMAG-65K). IL-6 levels were normalized to total protein levels (measured as IL-6 ([pg]) to total protein amp) measured by Bradford assay (Pierce Coomassie Plus Assay Kit (ThermoFisher Scientific, 23236). All treatments were administered intravenously via the tail vein (1.67 ml/kg), except for one group that received intra-articular injection of dCIC at one dose (denoted as “1274 #”). The treatment groups included the following test articles as shown in FIG. 24: (i) no IL-1β negative control (denoted “No IL-1b”); (ii) vehicle only control for the peptide-drug conjugate (denoted as “vehicle (PDC)”, which was 5% DMSO in PBS); (iii) vehicle only control for dCIC (40% propylene glycol, 5% DMSO in PBS, denoted as “vehicle (dCIC)”); (iv) 2 dose levels of Dexamethasone sodium phosphate, i.e, DexSP (denoted as “Dex”) at 1274 nmol/kg and 127 nmol/kg (denoted over “Dex” as “1274” and “127” respectively); (v) 3 dose levels of dCIC, 1274 nmol/kg IA, 1274 nmol/kg IV, 127 nmol/kg IV (denoted over “dCIC” as “1274 #”, “1274” and “127” respectively); and (vi) 3 dose levels of peptide(SEQ ID NO: 105)-DMA-dCIC (127 nmol/kg, 51 nmol/kg, and 13 nmol/kg) (denoted over “Peptide(SEQ ID NO: 105)-DMA-dCIC” as “127”, “51” and “13” respectively), each of (i)-(vi) with n=5 animals per dose group. Doses are listed as nmol/kg The masses of these doses are given below in TABLE 16. Two hours and forty-five minutes (i.e., 2.75 hrs) after administration of the test agents, animals were gently heated and blood was drawn from the tail vein for CBC analysis. Animals were then anesthetized with inhaled isoflurane and injected with 30 ng of IL-1β into the right knee (except for no IL-1β group). Four hours after administration of IL-1β (seven hours after administration of test agent) the animals were euthanized. Blood was drawn for a second CBC analysis and the right knee was lavaged for detection of IL-6 in the lavage fluid using a Luminex assay. Thymus and spleen were harvested and their organ weights determined. Graphs are displayed as box and whiskers plots with median, 25^{th} and 75^{th} percentile, and max and min displayed, with vehicle median shown as a dashed line.

TABLE 16
Molar and Mass Doses of Compounds.

| Dose level (nmol/kg) | mg/kg of dCIC or Dex | mg/kg of peptide |
|---|---|---|
| 1274 | 0.6 | 6 |
| 510 | 0.2 | 2 |
| 127 | 0.06 | 0.6 |
| 51 | 0.02 | 0.2 |
| 13 | 0.006 | 0.06 |

All doses (TABLE 16 above) were well-tolerated except at the highest dose levels of peptide-dCIC. All animals in the highest dose group (1274 nmol/kg IV) died within 30 minutes of dosing and three out of five animals in the second highest dose group (510 nmol/kg) died within 30-90 minutes of dosing. The other two animals in that the second highest dose group (510 nmol/kg) group survived until euthanasia. All other animals in the study survived until euthanasia. Gross examination did not reveal the cause of death.

Animals that had no injection of IL-1β showed negligible IL-6 concentrations in the knee, while both vehicle control groups had substantially higher median IL-6 concentrations (FIG. 24). The positive control with Dex only demonstrated a substantial reduction in IL-6 levels compared to vehicle at both dose levels (although variability in the cytokine levels was observed, particularly in the high-dose Dex group). Together, these data suggest that the IL-1β injection is functioning to induce inflammation that can be measured by an increase in IL-6 levels in the joint in vehicle-treated animals. Moreover, IL-6 levels can be reduced by treatment with a high potency steroid such as Dex. Treatment with the peptide-drug conjugate peptide(SEQ ID NO: 105)-DMA-dCIC resulted in a clear reduction in IL-6 concentration following the administration of all 3 doses that were analyzed (i.e., 127 nmol/kg, 51 nmol/kg, and 13 nmol/kg) (FIG. 24). The drug mass of dCIC in these dose levels was 0.006-0.06 mg/kg. No clear dose response was observed in this study, suggesting that the peptide-drug conjugate peptide(SEQ ID NO: 105)-DMA-dCIC may be effective at even lower doses. Administration of dCIC as a single agent at a dose of 1274 nmol/kg IA or 127 nmol/kg via IV administration also resulted in a decrease in IL-6 concentrations relative to vehicle.

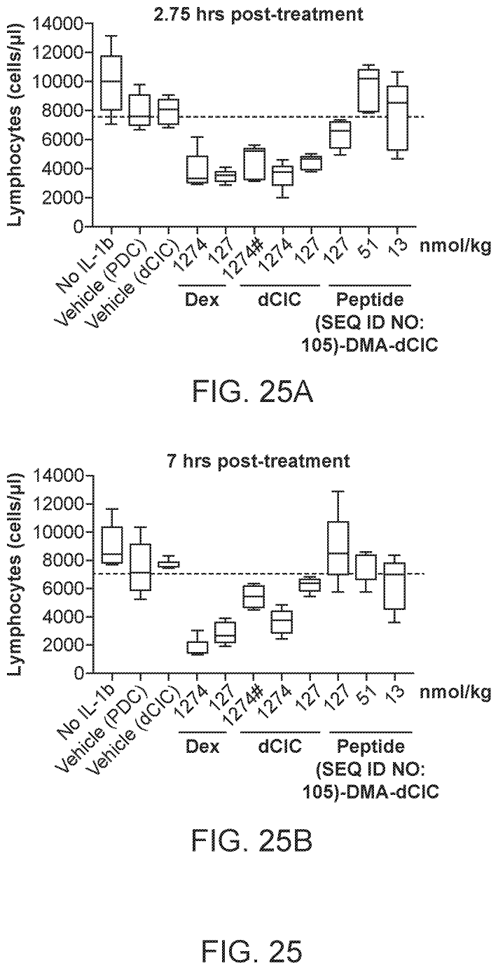

Measurements of Lymphocyte Counts (FIG. 25).

In the same animals for which IL-6 secretion was measured, CBC data (e.g., lymphocyte counts) were also analyzed (FIG. 25) at an early time-point, 2.75 hrs post administration of the test article (FIG. 25A), and at euthanasia 7 hrs post administration (FIG. 25B). All treatments were administered as described above for the measurement of IL-6 secretion.

Lymphocyte counts measured at the early time-point, 2.75 hrs post administration, provided insights into the extent of exposure to systemic steroids with each treatment (FIG. 25A). Both vehicle control groups showed lymphocyte counts that were similar, although slightly reduced, compared to animals that received no IL-1β. The positive control, Dex, resulted in a substantial reduction in lymphocyte counts as compared to vehicle control at both doses tested. Similarly, administration of dCIC either IA or IV, also resulted in a reduction in lymphocyte counts as compared to vehicle. Administration of the peptide-drug conjugate peptide(SEQ ID NO: 105)-DMA-dCIC appeared to have less effect on lymphocyte counts as compared to Dex and dCIC alone when administered at an equimolar dose of 127 nmol/kg, and appeared to be similar to vehicle control when administered at lower doses of 51 nmol/kg and 13 nmol/kg. Thus, doses of the peptide-drug conjugate peptide(SEQ ID NO: 105)-DMA-dCIC that were found to cause a reduction in PD marker(s) of inflammation in the joint, had only a minimal effect on systemic markers of steroid exposure were observed, suggesting that the peptide-drug conjugate peptide(SEQ ID NO: 105)-DMA-dCIC is capable of reducing inflammation in the joint without causing significant systemic exposure to the drug.

In addition, CBC data (e.g., lymphocyte counts) collected at euthanasia provided additional insights into the extent of systemic steroid exposure with each treatment. Lymphocyte counts appeared to be lower at 7 hours as compared to 2.75 hours in animals treated with both dose levels of Dex (FIG. 25B). However, lymphocyte counts in animals that received dCIC either IA or IV appeared to be either stable or starting to recover at 7 hrs as compared to 2.75 hours. Animals treated with all three doses of the peptide-drug conjugate peptide(SEQ ID NO: 105)-DMA-dCIC had lymphocyte counts similar to those observed for vehicle at the 7 hour time-point (FIG. 25B). Hence, the data obtained at euthanesia confirmed the finding that the peptide-drug conjugate peptide(SEQ ID NO: 105)-DMA-dCIC has minimal impact on systemic markers of steroid exposure while reducing inflammation in cartilage. Moreover, the data highlighted some of the pharmacokinetic (PK) differences observed between dCIC and DexSP: a faster clearance may be responsible for the shorter duration and lower impact of dCIC on lymphocyte counts as compared to Dex, confirming the use of dCIC in peptide-drug conjugates of the present disclosure. Comparison of spleen and thymus weights at euthanasia supported that observation, showing that treatments with the peptide-drug conjugate peptide(SEQ ID NO: 105)-DMA-dCIC or dCIC alone had minimal effect on the weight of both organs, but that DexSP treatment resulted in a reduction in the weight of both organs.

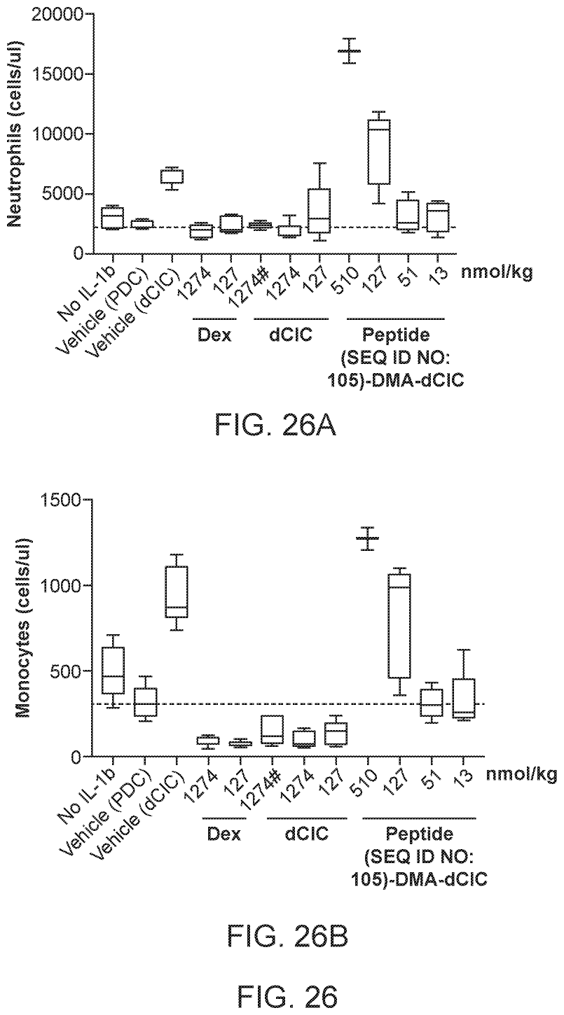

Measurements of Neutrophil Counts (FIG. 26).

All treatments and test articles were administered as described above, except for an additional treatment group that received 510 nmol/kg of the peptide-drug conjugate peptide(SEQ ID NO: 105)-DMA-dCIC.

Neutrophil and monocyte counts from CBC were also analyzed. There was a dose-dependent increase in neutrophils (FIG. 26A) and monocytes (FIG. 26B) following treatment with peptide(SEQ ID NO: 105)-DMA-dCIC at higher doses, as well as in animals treated with vehicle (dCIC) (FIG. 26). There was a dose dependent decrease in monocytes in animals treated with Dex or dCIC at all dose levels tested. The groups experiencing toxicity were eliminated from further analysis. The cause of the toxicity at high doses in rats is unknown, but the CBC counts and all observations at the lower effective doses of peptide-dCIC conjugate (13-51 nmol/kg) were normal.

This data demonstrate that the herein disclosed peptide-drug conjugates are capable of reducing inflammation in cartilage while reducing systemic drug exposure when compared to administration of drug alone.

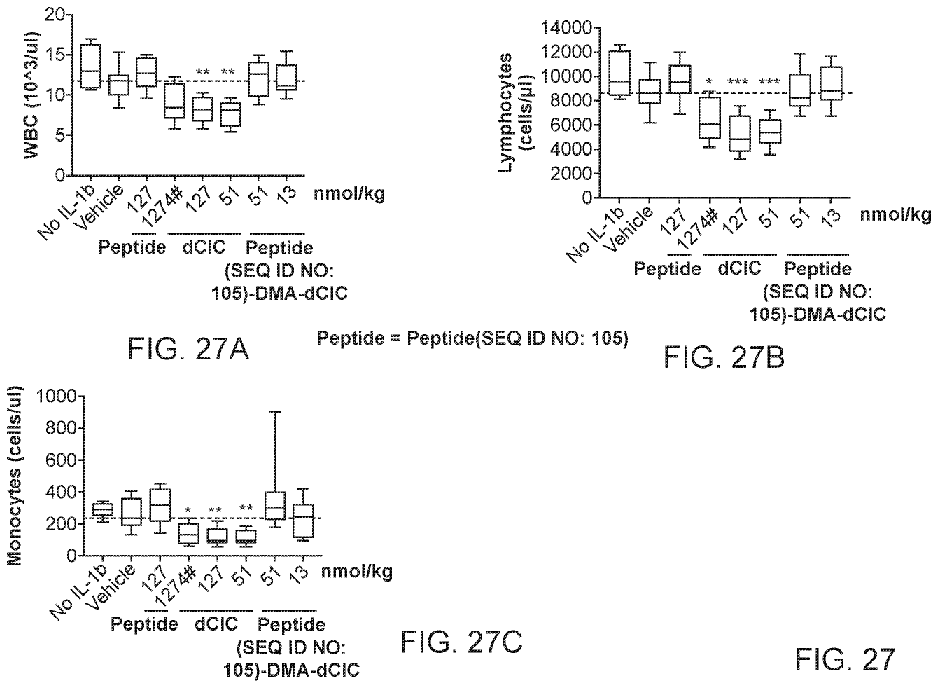

### Follow-Up Study Measurements of WBC, Lymphocyte and Monocyte Counts (FIG. 27)

This study was conducted in an identical manner to the dose-ranging study described further above in this EXAMPLE with regard to timing of test agent administration, collection of blood for CBC, injection of IL-1β, and lavage of the knee following euthanasia. In this study, the blood collected at euthanasia was submitted for both CBC and serum chemistry, and thymus and spleen weights were not measured because there was no evidence of treatment effect for dCIC and peptide-dCIC on these organs in the dose-ranging study. Assessment of biomarkers of steroid exposure in blood of rats as part of a PD study as shown in FIG. 27: # denotes dose administered by IA injection, all other treatments were administered IV. The treatment groups included the following test articles: (i) no IL-1β negative control (denoted “No IL-1b”); (ii); vehicle only control for the peptide-drug conjugate (denoted as “vehicle”), which was (5% DMSO in PBS); (iii) the peptide-only control peptide(SEQ ID NO: 105) at 127 nmol/kg (denoted over “Peptide” as “127”); (iv) 3 dose levels of dCIC, 1274 nmol/kg IA, 127 nmol/kg IV, 51 nmol/kg IV (denoted over “dCIC” as “1274 #”, “127” and “51” respectively); (v) and 2 dose levels of peptide(SEQ ID NO: 105)-DMA-dCIC (51 nmol/kg, 13 nmol/kg) (denoted over “Peptide(SEQ ID NO: 105)-DMA-dCIC” as “51” and “13” respectively), each of (i)-(v) with n=10 animals per dose group, except for no Il-1β control and peptide-only control arms which had n=6 animals per dose group. Doses are listed as nmol/kg. Statistical analysis was conducted using Wilcoxon rank-sum test with correction for multiple comparisons using Holm's method. Measurement of IL-6 levels in the joint lavage fluid was conducted in a similar manner to the dose-ranging study; however analysis of the IL-6 levels in joints of vehicle-treated animals suggested that the IL-1β-mediated induction of IL-6 was suboptimal in this study. In the vehicle control group 40% of animals had negligible levels of IL-6 in the joint and 30% had IL-6 concentrations that were below the level expected for successful IL-1β-mediated induction, suggesting technical issues with model IL-6 induction in this study.

CBC data measured at 2.75 hours confirmed a lack of effect on blood count markers by administration of peptide(SEQ ID NO: 105)-DMA-dCIC, whereas dCIC administration significantly reduced WBC, lymphocytes, and monocytes. The 2.75 hour CBC measurements are valid because blood was collected prior to the IL-1β injection and is analogous to running the study in normal animals. Total WBC counts (FIG. 27A), lymphocyte counts (FIG. 27B) and monocyte counts (FIG. 27C) were analyzed as PD biomarkers that are expected to decrease in response to systemic steroid exposure (FIG. 27). The No IL-1β and vehicle control groups are within the normal range for all 3 parameters. Peptide(SEQ ID NO: 105) alone and peptide(SEQ ID NO: 105)-DMA-dCIC at both doses tested are similar to the control groups for all 3 parameters. However, dCIC administered IA and IV caused a substantial reduction in WBC counts (FIG. 27A), lymphocyte counts (FIG. 27B), and monocyte counts (FIG. 27C). This was found to be statistically significant compared to vehicle for all dCIC treatments across the 3 CBC parameters, except the IA injection of dCIC was not statistically significant with regard to total WBC counts. CBC and serum chemistry analyses conducted following euthanasia demonstrated no meaningful differences between treatment groups.

Overall, this study confirms the hematology findings from the dose-ranging study described above in this EXAMPLE and demonstrates that conjugation of dCIC to a peptide as described herein (e.g., peptide(SEQ ID NO: 105)) significantly reduces systemic exposure to the steroid. In addition, dCIC alone is not soluble in water and required heating in 40% propylene glycol to solubilize, whereas delivery of dCIC by conjugation to peptide SEQ ID NO: 105 allowed solubilization and delivery in aqueous buffer. Demonstrating solubility of the peptide-drug conjugates of the disclosure shows that such PDCs can be formulated for systemic delivery of drugs (including that otherwise are not water soluble, such as dCIC) that can home, target, migrate to, accumulate in, bind to, be retained by, or are directed to a cartilage of a subject. Thus, taken together these studies confirm that systemic dosing of a peptide-drug conjugate (e.g., peptide(SEQ ID NO: 105)-DMA-dCIC) can significantly reduce systemic exposure and PD changes that are associated with steroid dosing (e.g., Dex or dCIC) alone, and at the same time show reduced inflammation biomarkers in joints.

### Example 32

### Dose Finding Study for Peptide(SEQ ID NO: 105)-DMA-dCIC in CIA Rats

This example illustrates an exemplary dose finding study for peptide(SEQ ID NO: 105)-DMA-dCIC (44) in CIA rats.

This study is performed to identify a dose of the conjugate peptide(SEQ ID NO: 105)-DMA-dCIC that reduces ankle swelling in the CIA model of rheumatoid arthritis in rats. A secondary goal of this study is to evaluate the biomarkers of systemic steroid exposure after treatment.

For this study, rats receive 5 doses of the peptide-drug conjugate peptide(SEQ ID NO: 105)-DMA-dCIC, that is synthesized as described above in EXAMPLE 29, into the tail vein. Tissues are collected 3 hours after the final dose. TABLE 17 shows the general study design.

TABLE 17
Study Design

| Group | Treatment | Dose nmol/kg | Schedule | # of rats |
|---|---|---|---|---|
| 1 | Vehicle | n/a | n/a | 5 |
| 2 | (SEQ ID NO: 105)- | 450 | Qdx5, iv | 5 |
| | DMA-dCIC | | | |
| 3 | (SEQ ID NO: 105)- | 300 | Qdx5, iv | 5 |
| | DMA-dCIC | | | |
| 4 | (SEQ ID NO: 105)- | 125 | Qdx5, iv | 5 |
| | DMA-dCIC | | | |
| 5 | (SEQ ID NO: 105)- | 75 | Qdx5, iv | 5 |
| | DMA-dCIC | | | |

The procedure for this study is as follows: on day 0, arthritis is initiated by injecting 2×200 ug bovine type II collagen in IFA, ID. On day 7, the development of arthritis is boosted with 1×100 ug bovine type II collagen in IFA, ID. On day 10 ankle diameters are started to be measured. Between days 13-18, rats are enrolled in the study when at least 1 ankle reaches a diameter of 7.25 mm. Between days 13-22, rats are dosed with SEQ ID NO: 105-DMA-dCIC daily for 5 days via IV. Between days 17-22, rats are euthanized 3 hours after the final dose. The body weight, serum and plasma, hind limbs, thymus, and spleen are collected and analyzed.

### Example 33

### Peptide-Drug-Detectable Agent Conjugates

This example describes the labeling of a peptide-drug conjugate with a detectable agent (e.g., a fluorescent dye, PET ligand) that can be used as a therapeutic and/or diagnostic (e.g., theranostic) agent.

A peptide of this disclosure (e.g., a peptide having the amino acid sequence set forth in any one of SEQ ID NO: 1-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The peptide can comprise at least one amino acid in a D or L configuration.

The synthesized peptide-drug conjugate is subsequently attached (e.g., via the C-terminus of the peptide) to a detectable agent using any of the synthetic strategies described in any one of EXAMPLE 5-EXAMPLE 19, or EXAMPLE 29 to produce a peptide-drug-detectable agent conjugate. The detectable agent is a fluorescent dye, wherein the fluorescent dye is Cy5.5 or an Alexa fluorophore, such as Alexa488 or Alexa647.

The peptide-drug-detectable agent conjugate is administered to a subject. Administration can be oral administration, intravenous administration, subcutaneous, intramuscular or direct injection into a joint of a patient and targeted to cartilage. The subject can be a human or a non-human animal. After administration, the peptide-drug-detectable agent conjugate homes to, targets, and/or is retained in cartilage (e.g., in a joint or an ankle). The pharmacokinetics (e.g., biodistribution) and pharmacodynamics of the conjugate is visualized either ex vivo and/or in vivo. The plasma half-life, target engagement, tissue uptake and/or tissue retention of the peptide-drug-detectable agent conjugate is determined in vivo.

The visualization of the peptide-drug-detectable agent conjugate in vivo is used in diagnosis of arthritis, cartilage damage, or any other cartilage disorder, and further provides information about the extent of drug delivery in vivo and potential dosing regimens.

### Example 34

### Treatment of Osteoarthritis

This example describes a method for treating osteoarthritis using peptide-active agent (or peptide-drug) conjugates of the present disclosure. This method is used as a treatment for acute and/or chronic symptoms associated with osteoarthritis.

A peptide of this disclosure (e.g., a peptide having the amino acid sequence set forth in any one of SEQ ID NO: 1-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The peptide can comprise at least one amino acid in a D or L configuration.

This study uses anti-inflammatory drugs, such as triamcinolone acetonide, budesonide, dexamethasone, des-ciclesonide or a disease-modifying osteoarthritic drug (DMOAD), e.g., an ADAMTS4/5 inhibitor, cathepsin K inhibitor, Wnt antagonist, or MMP-13 inhibitor.

The resulting conjugate is administered in a pharmaceutical composition subcutaneously, intravenously, intramuscularly, or orally, or is injected directly into a joint of a subject and targeted to cartilage. The subject can be a human or a non-human animal. The formulation can be modified physically or chemically to increase the time of exposure in the cartilage. The peptide-drug conjugate accumulates in cartilage. The arthritis condition in the patient is subsequently improved, indicated by reduced pain levels and/or increased mobility.

### Example 35

### Treatment of a Cartilage Injury

This example describes a method for treating a cartilage injury using a peptide-drug conjugate of the present disclosure.

A peptide of this disclosure (e.g., a peptide having the amino acid sequence set forth in any one of SEQ ID NO: 1-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to triamcinolone acetonide, budesonide, des-ciclesonide and dexamethasone. The peptide can comprise at least one amino acid in a D or L configuration.

The resulting peptide-drug conjugate is administered in a pharmaceutical composition subcutaneously, intravenously, intramuscularly or orally, or is injected directly into a joint of a subject and targeted to cartilage. The subject can be a human or a non-human animal. The formulation can be modified physically or chemically to increase the time of exposure in the cartilage. The peptide-drug conjugate accumulates in cartilage. The cartilage injury condition in the subject is subsequently improved, indicated by reduced pain levels and/or increased mobility.

### Example 36

### Treatment of Rheumatoid Arthritis

This example describes a method for treating rheumatoid arthritis with a conjugate disclosed herein. This method is used as a treatment for acute and/or chronic symptoms associated with rheumatoid arthritis.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to anti-inflammatory agents, such as triamcinolone acetonide, budesonide, dexamethasone, des-ciclesonide or a disease-modifying antirheumatic drug (DMARD), such as methotrexate or tofacitinib. The peptide can comprise at least one amino acid in a D or L configuration. The peptide can comprise at least one amino acid in a D or L configuration.

The resulting peptide-drug conjugate is administered in a pharmaceutical composition subcutaneously, intravenously, or orally, intramuscularly or is injected directly into a joint of a subject and targeted to cartilage. The subject can be a human or a non-human animal. The formulation can be modified physically or chemically to increase the time of exposure in the cartilage. The peptide-drug conjugate accumulates in cartilage. The rheumatoid arthritis in the subject is subsequently improved, indicated by reduced pain levels and/or increased mobility.

### Example 37

### Treatment of Gout

This example describes a method for treating gout using conjugates of the present disclosure. This method is used as a treatment for acute and/or chronic symptoms associated with gout.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to nonsteroidal anti-inflammatory drugs, colchicine, a steroid, or uricase. The peptide can comprise at least one amino acid in a D or L configuration.

The resulting peptide-drug conjugate is administered in a pharmaceutical composition subcutaneously, intravenously, intramuscularly or orally, or is injected directly into a joint of a subject and targeted to cartilage affected by gout. The subject can be a human or a non-human animal. The formulation can be modified physically or chemically to increase the time of exposure in the cartilage. The peptide-drug conjugate accumulates at the target site, e.g., in cartilage. The gout condition of the subject is subsequently improved, indicated by reduced pain levels and/or increased mobility.

### Example 38

### Treatment or Management of Pain

This example describes a method for treating or managing pain associated with a cartilage injury or disorder with a conjugate disclosed herein.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to an anti-inflammatory agent such as NSAID (nonsteroidal anti-inflammatory drug), glucocorticoid, or PGE receptor antagonist, a Trk inhibitor, TRPV1 antagonist, or CGRP inhibitor, or other small molecule with analgesic activity in the joint. The peptide can comprise at least one amino acid in a D or L configuration.

The resulting peptide-drug conjugate is administered in a pharmaceutical composition subcutaneously, intravenously, intramuscularly or orally, or is injected directly into a tissue and/or organ, or directly into a joint. The subject can be a human or a non-human animal. The formulation can be modified physically or chemically to increase the time of exposure in the cartilage. The peptide-drug conjugate accumulates at the target site, e.g., in cartilage, joint, ankle, and targets to the cartilage affected by pain. The condition of the subject is subsequently improved, indicated by reduced pain levels, increased mobility, and/or increased appetite.

### Example 39

### Treatment of Ankylosing Spondylitis

This example describes a method for treating ankylosing spondylitis using conjugates of the present disclosure. This method is used as a treatment for acute and/or chronic symptoms associated with ankylosing spondylitis.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to an anti-inflammatory compound, such as triamcinolone acetonide, dexamethasone, des-ciclesonide or budesonide. The peptide can comprise at least one amino acid in a D or L configuration.

The resulting conjugate is administered in a pharmaceutical composition subcutaneously, intravenously, intramuscularly, or orally, or is injected directly into a joint of a subject and targeted to cartilage. The subject can be a human or a non-human animal. The formulation can be modified physically or chemically to increase the time of exposure in the cartilage. The peptide-drug conjugate accumulates at the target site, e.g., in cartilage, joint, ankle, etc. The ankylosing spondylitis condition of the subject is subsequently improved, indicated by reduced pain levels and/or increased mobility.

### Example 40

### Treatment of Psoriatic Arthritis

This example describes a method for treating psoriatic arthritis using conjugates of the present disclosure. This method is used as a treatment for acute and/or chronic symptoms associated with psoriatic arthritis.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to an anti-inflammatory compound, such as triamcinolone acetonide, dexamethasone, des-ciclesonide, or budesonide. The peptide can comprise at least one amino acid in a D or L configuration.

The resulting conjugate is administered in a pharmaceutical composition subcutaneously, intravenously, intramuscularly, or orally, or is injected directly into a joint of a subject and targeted to cartilage. The subject can be a human or a non-human animal. The formulation can be modified physically or chemically to increase the time of exposure in the cartilage. The peptide-drug conjugate accumulates at the target site, e.g., in cartilage, joint, ankle, etc. The psoriatic arthritis condition of the subject is subsequently improved, indicated by reduced pain levels and/or increased mobility.

### Example 41

### Treatment of Lupus Conditions

A peptide of this disclosure (e.g., a peptide having the amino acid sequence set forth in any one of SEQ ID NO: 21-SEQ ID NO: 247 or SEQ ID NO: 282-SEQ ID NO: 510 is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to an anti-inflammatory compound, such as triamcinolone acetonide, dexamethasone, desciclesonide, or budesonide. The peptide can comprise at least one amino acid in a D or L configuration.

The resulting conjugate is administered in a pharmaceutical composition subcutaneously, intravenously, intramuscularly, or orally, or is injected directly into a joint of a subject and targeted to cartilage. The subject can be a human or a non-human animal. The formulation can be modified physically or chemically to increase the time of exposure in the cartilage. The peptide-drug conjugate accumulates at the target site, e.g., in cartilage, joint, ankle, etc. The lupus condition of the subject is subsequently improved, indicated by reduced pain levels and/or increased mobility.

### Example 42

### Treatment Using Des-Ciclesonide Conjugates

This example demonstrates the use of des-Ciclesonide (i.e., dCIC) Conjugates in the prevention and treatment of disease.

Ciclesonide is a prodrug that is metabolized in vivo to the active metabolite des-ciclesonide. By conjugating des-ciclesonide to a peptide via an ester linker, dicarboxylic acid linker or other labile linker described herein, upon hydrolysis, the drug that is released from the conjugate is the active derivative des-ciclesonide, just as after systemic administration of ciclesonide alone.

Des-ciclesonide is readily complexed, conjugated, or fused to any peptide disclosed herein via a dicarboxylic acid linker or other labile linker described herein. The peptide in the resulting drug-peptide conjugate can be any peptide with the amino acid sequence selected from SEQ ID NO: 21-SEQ ID NO: 247 or SEQ ID NO: 282-SEQ ID NO: 510. The peptide can be a peptide having the amino acid sequence set forth in SEQ ID NO: 105, SEQ ID NO: 103, or SEQ ID NO: 184. Such peptide-drug conjugates are produced using either a cleavable or stable linker as described herein (e.g, those shown in TABLE 2), and using any of the synthetic methods described herein, e.g., those shown in EXAMPLE 5-EXAMPLE 19, or EXAMPLE 29.

The linkers used in the PDCs of this example can include any linker moiety 1-22 shown in TABLE 2, isomer, stereoisomer, or derivative thereof. The dicarboxylic acid linker is a linear dicarboxylic acid, such as succinic acid, or a related cyclic anhydride, such as succinic anhydride. Reactions with anhydrides can proceed under various conditions. For example, the reaction of des-ciclesonide with five molar equivalents of glutaric anhydride is carried out in anhydrous pyridine at room temperature. Reactions with dicarboxylic acids can be conducted using carbodiimide coupling methods. For example, des-ciclesonide is reacted with one molar equivalent dimethyl succinic acid, one molar equivalent 1-ethyl-3-(3-dimethylaminopropyl)carbodiimide (or another carbodiimide), and 0.2 molar equivalents of 4-dimethylamino pyridine. Additional conjugates used in this example include those that comprise a DMA linker or a trans-1-aminomethyl-cyclohexyl-4-carboxylic (i.e., carbamate) linker. Conjugates comprising a DMA linker are synthesized using strategies as described herein, including those for analogous compounds, including e.g., hydrolysis and synthesis of 2,5-dimethyl adipic acid using, e.g., lithium hydroxide as described herein, and ester bond formation-synthesis e.g., as described in EXAMPLE 5. Conjugates comprising a carbamate linker are synthesized as described herein for similar conjugates, e.g., as described in in EXAMPLE 6.

The peptide-des-ciclesonide conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Optionally, the subject has osteoarthritis, rheumatoid arthritis, ankylosing spondylitis, lupus arthritis, systemic lupus erythematosus, or lupus nephritis. Upon administration of the peptide-des-ciclesonide conjugates, the cartilage and/or kidney inflammation is alleviated.

### Example 43

### Peptide Homing with Therapeutic Agents

This example describes certain exemplary conjugates comprising therapeutic agents that are conjugated to a cystine-dense peptide.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to paclitaxel, dexamethasone, budesonide, des-ciclesonide, or triamcinolone acetonide using techniques described above. One or more drugs are conjugated per peptide, or an average of less than one drug is conjugated per peptide.

Coupling of these drugs to a peptide of any of SEQ ID NO: 21-SEQ ID NO: 247 or SEQ ID NO: 282-SEQ ID NO: 510 targets the drug to the cartilage of the subject. One or more drug-peptide conjugates are administered to a human or animal. The peptide can comprise at least one amino acid in a D or L configuration.

### Example 44

### Conjugate Binding to Cartilage Explants

This example illustrates a peptide of a conjugate of this disclosure binding to human and animal cartilage explants in culture. A peptide is selected from any one of the peptides of SEQ ID NO: 21-SEQ ID NO: 247 or SEQ ID NO: 282-SEQ ID NO: 510. The peptide can comprise at least one amino acid in a D or L configuration.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to paclitaxel, dexamethasone, budesonide, des-ciclesonide, or triamcinolone acetonide.

Such a peptide conjugate is then incubated with cartilage explants derived from humans or animals. The conjugates are found to bind to cartilage explants. Binding is confirmed using various methods that include but are not limited to liquid scintillation counting, confocal microscopy, immunohistochemistry, HPLC, or LC/MS.

### Example 45

### Conjugate Localization in Chondrocytes

This example illustrates binding of conjugates of this disclosure to chondrocytes within cartilage in animals. Whole animal sagittal slices are prepared that result in thin frozen sections being available for staining and imaging. At the end of the dosing period, animals are euthanized and cartilage is removed for use in staining and imaging procedures. One or more of the following cartilage components are identified in thin frozen sections or live cartilage explants using standard staining techniques: collagen fibrils, glycosaminoglycans, or chondrocytes. A peptide conjugate of this disclosure (e.g., those described and synthesized as shown in EXAMPLE 5-EXAMPLE 19, or EXAMPLE 29 above) is found to localize to chondrocytes in cartilage. Localization is visualized and confirmed by microscopy. The peptide can comprise at least one amino acid in a D or L configuration.

### Example 46

### Conjugate Localization in Cartilage Extracellular Matrix

This example illustrates localization of peptides of conjugates of this disclosure in cartilage extracellular matrix. Peptides of conjugates of this disclosure are bound to extracellular matrix within cartilage in animals. Thin frozen sections or live cartilage explants are acquired, stained, and visualized as described in EXAMPLE 21. A peptide conjugate of this disclosure (e.g., those described and synthesized as shown in EXAMPLE 5-EXAMPLE 19, or EXAMPLE 29 above, e.g., compounds 23-56) is found to localize to the extracellular matrix in cartilage. Localization is visualized and confirmed by microscopy. The peptide can comprise at least one amino acid in a D or L configuration.

### Example 47

### Conjugate Homing to an Arthritic Joint

This example illustrates conjugate homing to cartilage in humans or animals with arthritis.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-22). The peptide-drug conjugate is synthesized as described in any one of EXAMPLE 5-EXAMPLE 19, or EXAMPLE 29, e.g., compounds 23-56. The drug used in this study is a therapeutic compound, such as those described herein, including, but not limited to paclitaxel, dexamethasone, budesonide, des-ciclesonide, or triamcinolone acetonide.

The peptide can comprise at least one amino acid in a D or L configuration. The conjugate is administered to a human or animal subcutaneously, intravenously, or orally, or is injected directly into a joint. The conjugate homes to cartilage (e.g., cartilage of ankle or joint) and is retained in the cartilage suitable for eliciting a therapeutic effect.

### Example 48

### Conjugate Homing to Cartilage in Non-Human Animals

This example illustrates a conjugate of this disclosure homing to cartilage in non-human animals. Non-human animals include but are not limited to guinea pigs, rabbits, dog, cats, horses, non-human primates, rats, mice, and other non-human animals.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-20). The peptide-drug conjugate is synthesized as described in any one of EXAMPLE 5-EXAMPLE 19, or EXAMPLE 29. The drug used in this study is a therapeutic compound, such as those described herein, including, but not limited to paclitaxel, dexamethasone, budesonide, des-ciclesonide, or triamcinolone acetonide.

The peptide can comprise at least one amino acid in a D or L configuration. The conjugate is administered to a non-human animal subcutaneously, intravenously, or orally, or is injected directly into a joint. Biodistribution is assessed by LC/MS, autoradiography, positron emission tomography (PET), or fluorescence imaging. A conjugate is homed to cartilage in non-human animals.

### Example 49

### Conjugates with Cleavable Linkers

This example describes preparation of conjugates having cleavable linkers.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively.

The purified peptide is then conjugated to an anti-arthritic agent such as an anti-inflammatory agent (e.g., dexamethasone, des-ciclesonide, etc.) via a cleavable linker, such as an ester bond using standard 1-ethyl-3-(3-dimethylaminopropyl)carbodiimide (EDC) or other suitable bioconjugation chemistries (e.g., dicylcohexylcarbodiimide (DCC) based chemistry or thionyl chloride or phosphorous chloride-based bioconjugation). The linker is cleaved by esterases, MMP, cathepsin B, a protease, or thrombin. The peptide can comprise at least one amino acid in a D or L configuration.

The resulting conjugates are administered to a human or animal subcutaneously, intravenously, orally, or injected directly into a joint to treat disease. The peptide can be cleaved from the anti-arthritic/anti-inflammatory agent by digestion by a cleaving agent.

### Example 50

### Intra-Articular Administration of Conjugates

This example illustrates intra-articular administration of conjugates of this disclosure.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The purified peptide is then linked to a drug (e.g., an anti-arthritic agent such as a glucocorticoid) via a linker (e.g., any linker described in TABLE 2, compounds 1-22). The peptide-drug conjugate is synthesized as described in any one of EXAMPLE 5-EXAMPLE 19, or EXAMPLE 29. The drug used in this study is a therapeutic compound, such as those described herein, including, but not limited to paclitaxel, dexamethasone, budesonide, des-ciclesonide or triamcinolone acetonide. The peptide can comprise at least one amino acid in a D or L configuration.

The conjugate is administered to a subject in need thereof via intra-articular administration. The cartilage is penetrated by the conjugate due to the small size of the conjugate, and due to binding of cartilage components by the conjugate. The conjugate is bound to cartilage and the residence time in the cartilage is longer due to this binding. Optionally, the injected material is aggregated, is crystallized, or complexes are formed, further extending the depot effect and contributing to longer residence time.

### Example 51

### Treatment for Systemic Lupus Erythematosus

This example illustrates treatment of systemic lupus erythematosus, including forms of the disease known as lupus nephritis and/or lupus arthritis using peptide-drug conjugates of this disclosure. A peptide of the present disclosure is recombinantly expressed or chemically synthesized and conjugated or fused to a therapeutic compound, such as a glucocorticoid including an exemplary drug, such as dexamethasone, budesonide, des-ciclesonide, or triamcinolone acetonide. The peptide in the drug-peptide conjugate can be a peptide which amino acid sequence is set forth in any one of SEQ ID NO: 105, SEQ ID NO: 103, or SEQ ID NO: 184. The peptide can be any peptide with the amino acid sequence selected from SEQ ID NO: 21-SEQ ID NO: 247 or SEQ ID NO: 282-SEQ ID NO: 510. Such peptide-drug conjugates are synthesized using either a cleavable or stable linker as described herein.

The peptide-drug conjugate is administered in a pharmaceutical composition to a subject as a therapeutic for lupus. The peptide is selected from any one of the peptides of SEQ ID NO: 21-SEQ ID NO: 247 or SEQ ID NO: 282-SEQ ID NO: 510. One or more peptides or peptide conjugates of the present disclosure are administered to a subject. A subject can be a human or an animal. The pharmaceutical composition is administered subcutaneously, intravenously, orally, or injected directly. The peptides or peptide conjugates target kidney affected by lupus nephritis and/or cartilage affected by lupus arthritis. The lupus condition of the subject is slowed, mitigated or improved.

### Example 52

### Functionalization of Peptide-Drug Conjugates Using Non-Terminal Alkenes or Alkynes

This example demonstrates the functionalization of peptide-drug conjugates (PDCs) of the present disclosure using non-terminal alkenes and/or alkynes.

A peptide of this disclosure (e.g., a peptide having the amino acid sequence set forth in any one of SEQ ID NO: 21-SEQ ID NO: 247 or SEQ ID NO: 282-SEQ ID NO: 510) is expressed recombinantly or chemically synthesized as described herein, e.g., in EXAMPLE 1 and EXAMPLE 2, respectively. The peptide-drug conjugate is synthesized as described in any one of EXAMPLE 5-EXAMPLE 19, or EXAMPLE 29. The drug used in this study is a therapeutic compound, such as those described herein, including, but not limited to paclitaxel, dexamethasone, budesonide, des-ciclesonide or triamcinolone acetonide. The peptide can comprise at least one amino acid in a D or L configuration.

### Functionalization of a PDC Via Hydrohalogenation Followed by Nucleophilic Substitution

Functionalization of a PDC includes the addition of a molecule such as an active agent (e.g., a glucocorticoid), a detectable agent (e.g., a fluorescent dye), an agent that alters the half-life of the PDC, an agent that alters the depot effect of the PDC, an agent that alters the uptake and/or retention of the PDC in cartilage, or a combination thereof.

The purified peptide is linked to the molecule via a linker. Such linker comprises an alkene functionality having the following structure:

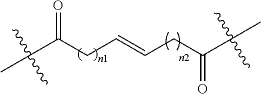

A molecule is attached to the PDC via hydrohalogenation of an alkene functional group of the linker of the PDC using hydrogen bromide. The PDC comprising the alkene is reacted with aqueous hydrogen bromide to form a mono-brominated PDC product, which is subsequently purified and isolated. The PDC comprising the bromide substituent is then reacted with a molecule (e.g., an active agent (e.g., a glucocorticoid), a detectable agent (e.g., a fluorescent dye), an agent that modulates the half-life of the PDC, an agent that the depot effect of the PDC, etc.) that comprises a nucleophilic functional group and that replaces the bromide substituent on the PDC in a nucleophilic substitution reaction, thereby attaching the molecule to the PDC. The functionalized PDC is then purified and characterized.

### Functionalization of a PDC Via 1,3-Dipolar Cycloaddition

Functionalization of a PDC includes the addition of a molecule such as an active agent (e.g., a glucocorticoid), a detectable agent (e.g., a fluorescent dye), an agent that alters the half-life of the PDC, an agent that alters the depot effect of the PDC, an agent that alters the uptake and/or retention of the PDC in cartilage, or a combination thereof.

The purified peptide is linked to the molecule via a linker. Such linker comprises an alkyne functionality having the following structure:

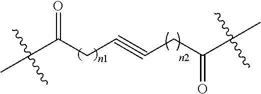

A molecule is attached to the PDC via 1,3-dipolar cycloaddition of an alkyne functional group of a linker of the PDC and a molecule containing a 1,3-dipole.

The PDC comprising the alkyne functional group is reacted with a molecule containing a 1,3-dipole to form a substituted 5 membered ring, thereby attaching the molecule to the PDC to form a functionalized PDC. The functionalized PDC is then purified and characterized.

The functionalized PDC is administered to a subject in need thereof via intra-articular, systemic, or oral administration and accumulates in cartilage tissue.

The results demonstrate that a functionalized PDC can be synthesized in good chemical yields and without impairing the targeting, homing, binding, or accumulation properties of the peptide and/or the PDC. These functionalized PDCs can be used increase a therapeutic function, track the PDC in vivo or in vitro, alter the plasma half-life of the PDC, alter the depot effect, and/or alter the hydrolysis properties of the PDC.

While preferred embodiments of the present disclosure have been shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A conjugate, wherein the conjugate comprises:
an anti-arthritic agent;
a cystine-dense peptide, wherein upon administration to a subject the cystine-dense peptide homes, targets, migrates to, accumulates in, binds to, is retained by, or is directed to a cartilage of the subject; and
a linker, wherein the linker comprises a cyclic carboxylic acid, a cyclic dicarboxylic acid, an aromatic dicarboxylic acid, or an amino acid, and wherein the linker conjugates the anti-arthritic agent and the cystine-dense peptide via an ester bond, a carbamate bond, a carbonate bond, or an amide bond.

2. The conjugate of claim 1, wherein the anti-arthritic agent is an anti-inflammatory agent.

3. The conjugate of claim 2, wherein the anti-inflammatory agent is a glucocorticoid or an NSAID.

4. The conjugate of claim 3, wherein the anti-inflammatory agent is the glucocorticoid that is dexamethasone, budesonide, triamcinolone, triamcinolone acetonide, beclomethasone, betamethasone, butixicort, cortisol (hydrocortisone), clobetasol, estriol, diflorasone, diflucortolone, difluprednate, des-ciclesonide, desisobutyryl-ciclesonide, hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof.

5. The conjugate of claim 4, wherein the glucocorticoid is dexamethasone.

6. The conjugate of claim 4, wherein the glucocorticoid is des-ciclesonide.

7. The conjugate of claim 4, wherein the glucocorticoid is budesonide.

8. The conjugate of claim 4, wherein the glucocorticoid is triamcinolone acetonide.

9. The conjugate of any one of claims 1-8, wherein the cyclic carboxylic acid, the cyclic dicarboxylic acid, or the aromatic dicarboxylic acid is monocyclic, bicyclic, tricyclic, or any combination thereof.

10. The conjugate of any one of claims 1-9, wherein the cyclic carboxylic acid, the cyclic dicarboxylic acid, or the aromatic dicarboxylic acid comprises a 4, 5, 6, 7, or 8 membered ring, or a combination thereof.

11. The conjugate of any one of claims 1-10, wherein the linker comprises the cyclic carboxylic acid.

12. The conjugate of claim 11, wherein the cyclic carboxylic acid comprises 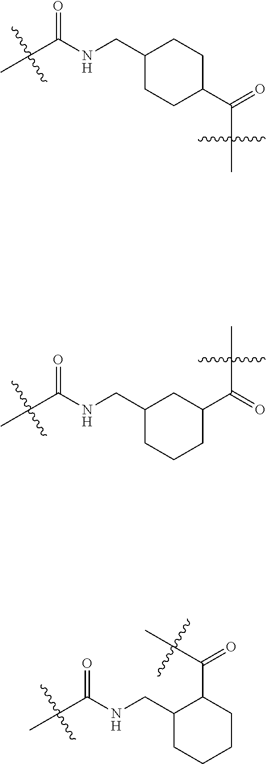 [SMILES: *C(=O)NCC1CCC(C(*)=O)CC1 ; *C(=O)NCC1CCCC(C(*)=O)C1 ; *C(=O)NCC1CCCCC1C(*)=O]
or a substituted analog or a stereoisomer thereof.

13. The conjugate of claim 11, wherein the cyclic carboxylic acid comprises 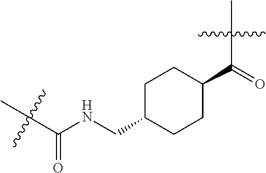 [SMILES: *C(=O)NC[C@H]1CC[C@H](C(*)=O)CC1]
or a substituted analog or a stereoisomer thereof.

14. The conjugate of any one of claims 1-10, wherein the linker comprises the cyclic dicarboxylic acid.

15. The conjugate of claim 14, wherein the cyclic dicarboxylic acid comprises one of the following groups: 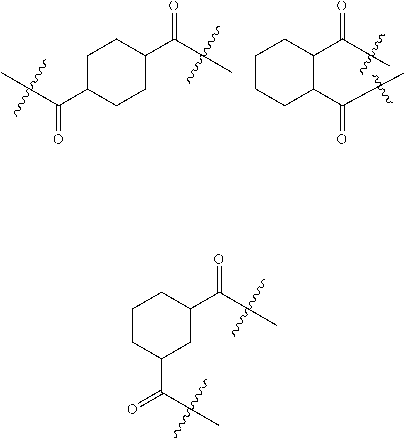 [SMILES: *C(=O)C1CCC(C(*)=O)CC1 ; *C(=O)C1CCCCC1C(*)=O ; *C(=O)C1CCCC(C(*)=O)C1]
or a substituted analog or a stereoisomer thereof.

16. The conjugate of claim 14, wherein the cyclic dicarboxylic acid comprises one of the following groups:

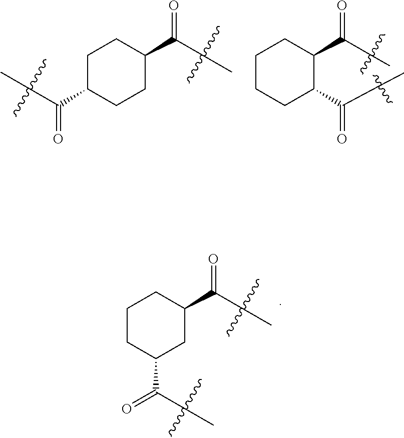 [SMILES: *C(=O)[C@H]1CC[C@H](C(*)=O)CC1 ; *C(=O)[C@@H]1CCCC[C@H]1C(*)=O ; *C(=O)[C@@H]1CCC[C@@H](C(*)=O)C1]

17. The conjugate of claim 14, wherein the cyclic dicarboxylic acid comprises 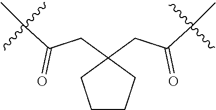 [SMILES: *C(=O)CC1(CC(*)=O)CCCC1]
or a substituted analog or a stereoisomer thereof.

18. The conjugate of any one of claims 1-10, wherein the linker comprises the aromatic dicarboxylic acid.

19. The conjugate of claim 18, wherein the aromatic dicarboxylic acid comprises 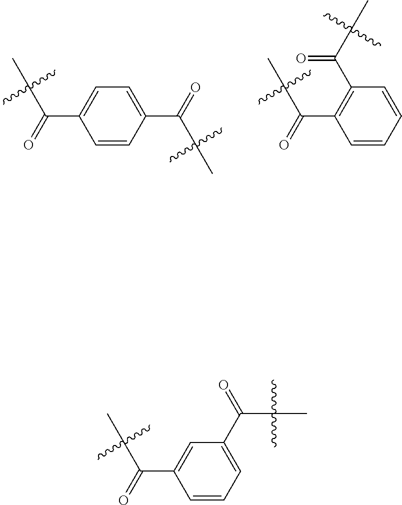 [SMILES: *C(=O)c1ccc(C(*)=O)cc1 ; *C(=O)c1ccccc1C(*)=O ; *C(=O)c1cccc(C(*)=O)c1]
or a substituted analog thereof.

20. The conjugate of any one of claims 1-10, wherein the linker comprises the amino acid.

21. The conjugate of claim 20, wherein the amino acid comprises 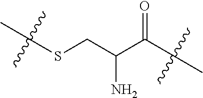 [SMILES: *SCC(N)C(*)=O]
or a substituted analog or a stereoisomer thereof.

22. The conjugate of any one of claims 1-10, wherein the linker comprises at least one of compound 2-17 listed in TABLE 2.

23. A conjugate, wherein the conjugate comprises:
a glucocorticoid, wherein the glucocorticoid is not budesonide or dexamethasone;
a cystine-dense peptide, wherein upon administration to a subject the cystine-dense peptide homes, targets, migrates to, accumulates in, binds to, is retained by, or is directed to a cartilage of the subject; and
a linker, wherein the linker comprises a linear dicarboxylic acid, and wherein the linker conjugates the glucocorticoid and the cystine-dense peptide via an ester bond, a carbamate bond, or an amide bond.

24. The conjugate of claim 23, wherein the glucocorticoid is triamcinolone acetonide, triamcinolone, beclomethasone, betamethasone, butixicort, cortisol (hydrocortisone), clobetasol, estriol, diflorasone, diflucortolone, difluprednate, des-ciclesonide, desisobutyryl-ciclesonide hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof.

25. A conjugate, wherein the conjugate comprises:
a glucocorticoid, wherein the glucocorticoid is triamcinolone acetonide, triamcinolone, beclomethasone, betamethasone, budesonide, butixicort, cortisol (hydrocortisone), clobetasol, estriol, diflorasone, diflucortolone, difluprednate, des-ciclesonide, desisobutyryl-ciclesonide, hydrocortine, cortisone, deoxycorticosterone, fluticasone, fluticasone furoate, fluticasone propionate, fluocinonide, fludrocortisone, flunisolide, fluorometholone, hexestrol, methimazole, methylprednisolone, mometasone, mometasone furoate, 17-monopropionate, paramethasone, prednisone, prednisolone, or a pharmaceutically acceptable salt thereof;
a cystine-dense peptide, wherein upon administration to a subject the cystine-dense peptide homes, targets, migrates to, accumulates in, binds to, is retained by, or is directed to a cartilage of the subject; and
a linker, wherein the linker comprises a linear dicarboxylic acid, and wherein the linker conjugates the glucocorticoid and the cystine-dense peptide via an ester bond, a carbamate bond, a carbonate bond, or an amide bond.

26. The conjugate of claim 23 or 25, wherein the glucocorticoid is triamcinolone acetonide.

27. The conjugate of any one of claims 23-26, wherein the linear dicarboxylic acid comprises one of the following groups: 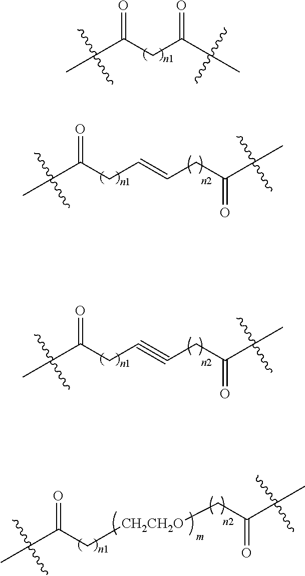
or a substituted analog or a stereoisomer thereof,
wherein each n1, n2, and m is independently a value from 1 to 10, and wherein m is a value from 0 to 10.

28. The conjugate of any one of claims 23-26, wherein the linear dicarboxylic acid comprises one of the following groups: 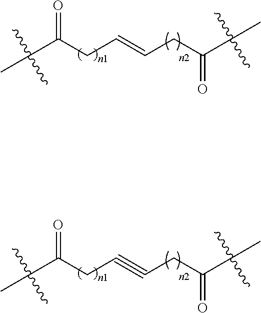
or a substituted analog or a stereoisomer thereof, wherein each n1 and n2 is independently a value from 1 to 10.

29. The conjugate of claim 28, wherein the linear dicarboxylic acid is functionalized using a multiple bond of the linear dicarboxylic acid.

30. The conjugate of claim 29, wherein the functionalization comprises attaching at least one molecule to the linear dicarboxylic acid.

31. The conjugate of any one of claims 29-30, wherein the functionalization via the multiple bond of the linear dicarboxylic acid comprises one or more of an addition reaction, a substitution reaction, a cycloaddition, or any combination thereof.

32. The conjugate of claim 31, wherein the addition reaction is a nucleophilic or an electrophilic addition reaction.

33. The conjugate of claim 32, wherein the addition reaction comprises the use of hydrogen bromide.

34. The conjugate of any one of claims 29-33, wherein the functionalization further comprises a nucleophilic substitution reaction.

35. The conjugate of any one of claims 29-34, wherein the nucleophilic substitution reaction occurs after the addition reaction.

36. The conjugate of claim 31, wherein the cycloaddition is a 1,3-dipolar cycloaddition.

37. The conjugate of any one of claims 29-36, wherein the at least one molecule is an active agent or a detectable agent.

38. The conjugate of any one of claims 29-37, wherein the at least one molecule alters (i) the uptake of the conjugate in a cartilage; (ii) the retention of the conjugate in a cartilage; (iii) the hydrolysis rate of the conjugate, or any combination thereof.

39. The conjugate of any one of claims 23-26, wherein the linear dicarboxylic acid is one of the following groups: 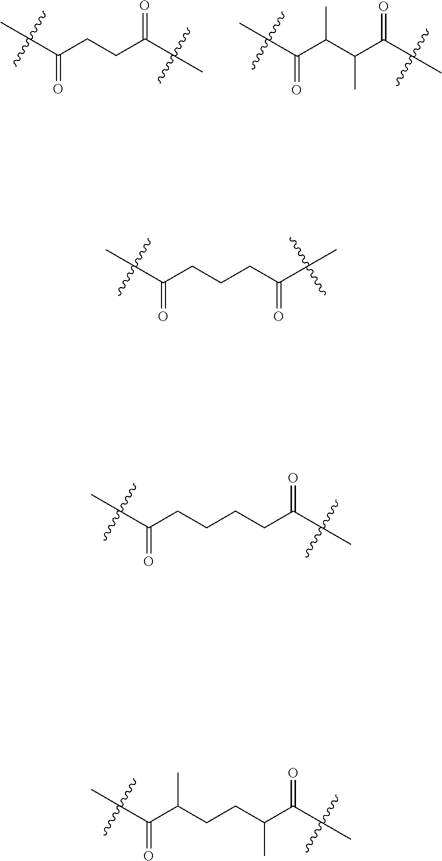
or a substituted analog or a stereoisomer thereof.

40. The conjugate of any one of claims 23-26, wherein the linker comprises at least one of compounds 18-22 listed in TABLE 2.

41. The conjugate of any one of claims 1-40, wherein the linker is stable.

42. The conjugate of any one of claims 1-40, wherein the linker is cleavable.

43. The conjugate of any one of claim 1-40, or 42, wherein the linker is cleavable by hydrolysis, an enzyme, a pH change, a reduction, a self-immolation, radiation, or a chemical reaction.

44. The conjugate of any one of claims 42-43, wherein less than 50% of the conjugates are cleaved within 24 hours, 32 hours, 56 hours, or 100 hours, at 20° C. to 37° C. or to 40° C. in a phosphate buffered saline, human plasma, or rat plasma as measured by LC/MS.

45. The conjugate of any one of claims 42-43, wherein less than 50% of the conjugates are cleaved within 10 hours, 30 hours, or 60 hours, at 20° C. to 37° C. or to 40° C. in a human plasma or rat plasma as measured by LC/MS.

46. The conjugate of claim 44 or 45, wherein the linker of the conjugate comprises a carbamate bond.

47. The conjugate of claim 46, wherein less than 50% of the conjugates are cleaved after 32 hours at 20° C. to 40° C. in a phosphate buffered saline, human plasma, or rat plasma as measured by LC/MS.

48. The conjugate of claim 46, wherein less than 50% of the conjugates are cleaved after 32 hours at 20° C. to 40° C. in a human plasma as measured by LC/MS.

49. The conjugate of claim 46, wherein less than 50% of the conjugates are cleaved after 32 hours at 20° C. to 40° C. in a rat plasma as measured by LC/MS.

50. The conjugate of any one of claims 47-49, wherein the linker comprises one of the following groups: 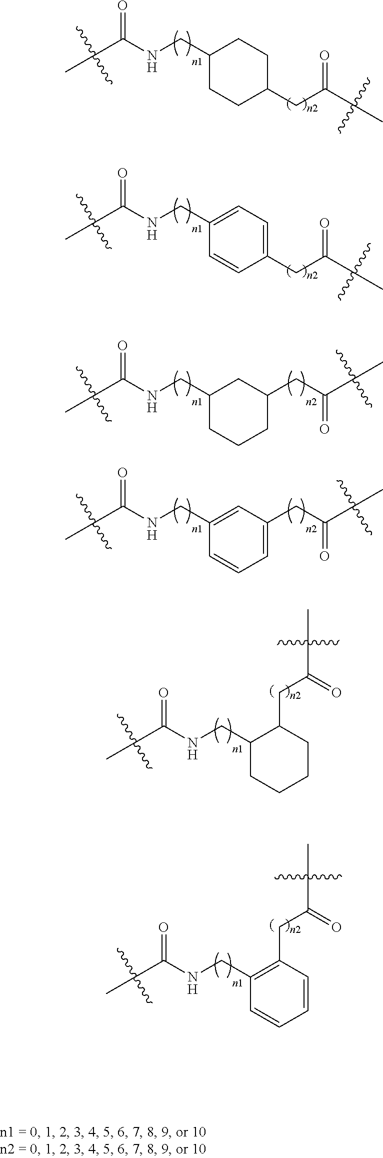
or a substituted analog or a stereoisomer thereof.

51. The conjugate of any one of claims 47-49, wherein the linker comprises one of the following groups: 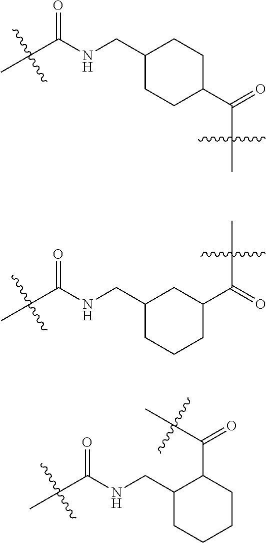 [SMILES: *C(=O)NCC1CCC(C(*)=O)CC1 ; *C(=O)NCC1CCCC(C(*)=O)C1 ; *C(=O)NCC1CCCCC1C(*)=O]
or a substituted analog or a stereoisomer thereof.

52. The conjugate of any one of claims 47-49, wherein the linker comprises: 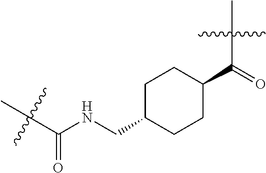 [SMILES: *C(=O)NC[C@H]1CC[C@H](C(*)=O)CC1]
or a substituted analog or a stereoisomer thereof.

53. The conjugate of any one of claims 1-45, wherein the linker comprises one of the following groups: 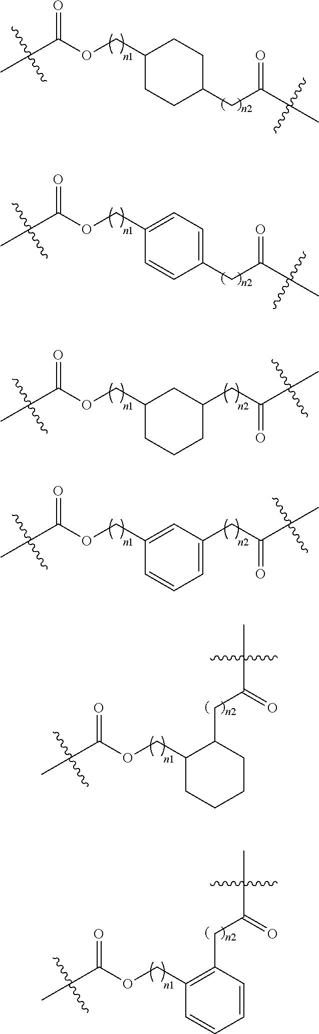
or a substituted analog or a stereoisomer thereof, and wherein n1 and n2 are each independently 0, 1, 2, 3, 4, 5, 6, 7, 8, 9, or 10.

54. The conjugate of claim 42 or 43, wherein 50%-100% of the conjugates are cleaved within 10-30 hours or 10-40 hours at 20° C. to 37° C. or to 40° C. in a human plasma as measured by LC/MS.

55. The conjugate of claim 42 or 43, wherein at least 50% of the conjugates are cleaved within 0.5 to 100 hours, 1-50 hours, 1-20 hours, or 2-10 hours, at 20° C. to 37° C. or to 40° C. in a phosphate buffered saline, human plasma, or rat plasma as measured by LC/MS.

56. The conjugate of claim 42 or 43, wherein the linker comprises 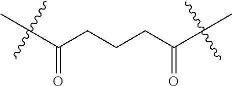 [SMILES: *C(=O)CCCC(*)=O]
or a substituted analog thereof,
and wherein 50%-100% of the conjugates are cleaved within 1-8 hours at 37° C. in a human plasma or rat plasma as measured by LC/MS.

57. The conjugate of claim 42 or 43, wherein the linker comprises 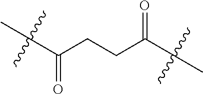 [SMILES: *C(=O)CCC(*)=O]
or a substituted analog thereof,
and wherein 50%-100% of the conjugates are cleaved within 1-8 hours at 37° C. in a phosphate buffered saline as measured by LC/MS.

58. The conjugate of claim 42 or 43, wherein the linker comprises 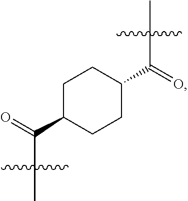 [SMILES: *C(=O)[C@H]1CC[C@H](C(*)=O)CC1]
or a substituted analog or a stereoisomer thereof, and wherein 50%-100% of the conjugates are cleaved within 1-8 hours at 37° C. in a rat plasma as measured by LC/MS.

59. The conjugate of claim 42 or 43, wherein the linker comprises 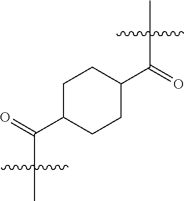 [SMILES: *C(=O)C1CCC(C(*)=O)CC1]
or a substituted analog or a stereoisomer thereof,
and wherein 25%-50% of the conjugates are cleaved within 1-8 hours at 37° C. in a human plasma as measured by LC/MS.

60. The conjugate of claim 42 or 43, wherein the linker comprises 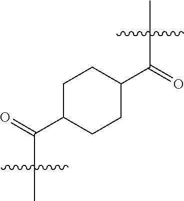 [SMILES: *C(=O)C1CCC(C(*)=O)CC1]
or a substituted analog or a stereoisomer thereof,
and wherein 50%-100% of the conjugates are cleaved within 10-30 hours at 37° C. in a human plasma as measured by LC/MS.

61. The conjugate of claim 42 or 43, wherein the linker comprises 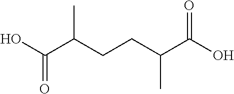
or a substituted analog or a stereoisomer thereof,
and wherein 5%-50% of the conjugates are cleaved within 1-8 hours at 37° C. in a rat plasma as measured by LC/MS.

62. The conjugate of claim 42 or 43, wherein the linker comprises 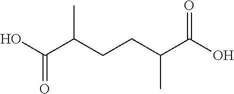
or a substituted analog or a stereoisomer thereof,
and wherein 2%-25% of the conjugates are cleaved within 1-8 hours at 37° C. in a human plasma as measured by LC/MS.

63. The conjugate of claim 42 or 43, wherein the linker comprises 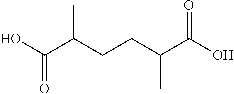
or a substituted analog or a stereoisomer thereof,
and wherein 50%-100% of the conjugates are cleaved within 10-40 hours at 37° C. in a human plasma as measured by LC/MS.

64. The conjugate of claim 42 or 43, wherein the linker comprises 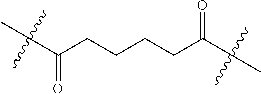 [SMILES: *C(=O)CCCCC(*)=O]
or a substituted analog thereof,
and wherein 75%-100% of the conjugates are cleaved within 1-8 hours at 37° C. in a rat plasma as measured by LC/MS.

65. The conjugate of claim 42 or 43, wherein the linker comprises 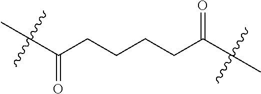 [SMILES: *C(=O)CCCCC(*)=O]
or a substituted analog thereof,
and wherein greater than 50% of the conjugates are cleaved by 10, 30, or 60 hours at 20° C. to 37° C. in rat plasma or a human plasma as measured by LC/MS.

66. The conjugate of any one of claims 1-43, wherein the linker comprises: 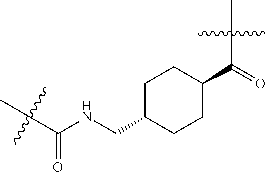 [SMILES: *C(=O)NC[C@H]1CC[C@H](C(*)=O)CC1]
or a substituted analog or a stereoisomer thereof,
and wherein less than 50% of the conjugates are cleaved within 1-8 hours at 37° C. in a phosphate buffered saline, human plasma, or rat plasma as measured by LC/MS.

67. The conjugate of any one of claims 1-43, wherein the linker comprises: 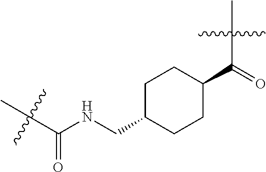 [SMILES: *C(=O)NC[C@H]1CC[C@H](C(*)=O)CC1]
or a substituted analog or a stereoisomer thereof,
and wherein less than 50% of the conjugates are cleaved within 8-32 hours at 37° C. in a phosphate buffered saline, human plasma, or rat plasma as measured by LC/MS.

68. The conjugate of any one of claims 42-67, wherein the conjugates are cleaved in vivo.

69. The conjugate of claim 68, wherein the conjugates are cleaved when administered to an animal.

70. The conjugate of claim 68, wherein the conjugates are cleaved when administered to a human.

71. The conjugate of any one of claims 43-70, wherein the conjugates are cleaved by hydrolysis.

72. The conjugate of any one of claims 43-70, wherein the conjugates are cleaved by a pH change, reduction, self-immolation, radiation or chemical reaction.

73. The conjugate of any one of claims 1-72, wherein the conjugate further comprises an amino acid sequence cleavable by enzymatic proteinase activity.

74. The conjugate of claim 73, wherein the conjugate comprises a cleavage site for a matrix metalloproteinase (MMP).

75. The conjugate of claim 74, wherein the MMP is MMP13.

76. The conjugate of claim 73, wherein the conjugate comprises a cleavage site for cathepsin.

77. The conjugate of claim 73, wherein the conjugate comprises a cathepsin cleavable linker.

78. The conjugate of claim 77, wherein the cathepsin cleavable linker is a valine-citrulline linker.

79. The conjugate of claim 76, wherein the cathepsin is cathepsin K.

80. The conjugate of claim 73, wherein the conjugate comprises a cleavage site for urokinase-type plasminogen activator.

81. The conjugate of claim 73, wherein the conjugate comprises a cleavage site for thrombin.

82. The conjugate of any one of claims 1-81, wherein the cystine-dense peptide comprises a disulfide through a disulfide knot.

83. The conjugate of any one of claims 1-73, wherein the cystine-dense peptide comprises a plurality of disulfide bridges formed between cysteine residues.

84. The conjugate of any one of claims 1-83, wherein the cystine-dense peptide comprises three or more disulfide bridges formed between cysteine residues, wherein one of the disulfide bridges passes through a loop formed by two other disulfide bridges.

85. The conjugate of any one of claims 1-84, wherein the cystine-dense peptide comprises 4 or more cysteine residues.

86. The conjugate of any one of claims 1-85, wherein the cystine-dense peptide comprises 6 or more basic residues and 2 or fewer acidic residues.

87. The conjugate of any one of claims 1-86, wherein the cystine-dense peptide comprises a 4-19 amino acid residue fragment containing at least 2 cysteine residues, and at least 2 positively charged amino acid residues.

88. The conjugate of any one of claims 1-86, wherein the cystine-dense peptide comprises a 20-70 amino acid residue fragment containing at least 2 cysteine residues, no more than 2 basic residues and at least 2 positively charged amino acid residues.

89. The conjugate of any one of claims 1-88, wherein the cystine-dense peptide comprises at least 3 positively charged amino acid residues.

90. The conjugate of claim 89, wherein the positively charged amino acid residues are selected from K, R, or a combination thereof.

91. The conjugate of any one of claims 1-90, wherein the cystine-dense peptide comprises an amino acid sequence that has at least 80%, at least 85%, at least 90%, at least 95%, at least 96%, at least 97%, at least 98%, or at least 99% sequence identity with the amino acid sequence of any one of SEQ ID NO: 21-SEQ ID NO: 247 or SEQ ID NO: 282-SEQ ID NO: 510, or a fragment thereof.

92. The conjugate of claim 91, wherein the cystine-dense peptide comprises an amino acid sequence of any one of SEQ ID NO: 1-SEQ ID NO: 20, SEQ ID NO: 248-SEQ ID NO: 267, or a fragment thereof.

93. The conjugate of any one of claims 1-90, wherein the cystine-dense peptide comprises the amino acid sequence set forth in SEQ ID NO: 103.

94. The conjugate of any one of claims 1-90, wherein the cystine-dense peptide comprises the amino acid sequence set forth in SEQ ID NO: 184.

95. The conjugate of any one of claims 1-90, wherein the cystine-dense peptide comprises the amino acid sequence set forth in SEQ ID NO: 105.

96. The conjugate of any one of claims 1-4, wherein the conjugate comprises any one of compounds 23, 26-31, 34, 36, 38, 40 43, 45-46, or 49-56.

97. The conjugate of claim 5 comprising any one of compounds 23, 26-28, or 40-46.

98. The conjugate of claim 6 comprising any one of compounds 45-46, or 49-56.

99. The conjugate of claim 8 comprising any one of compounds 29-31, 34, or 36.

100. The conjugate of any one of claims 21-23 comprising any one of compounds 44 or 49-56.

101. The conjugate of claim 24 comprising any one of compounds 32, 33, 35, 46, or 49-56.

102. The conjugate of any one of claims 1-90, wherein the cystine-dense peptide comprises the amino acid sequence set forth in any one of SEQ ID NO: 103, SEQ ID NO: 105, or SEQ ID NO: 184.

103. The conjugate of claim 102 comprising any one of compounds 46, or 49-56.

104. A pharmaceutical composition that comprises the conjugate of any one of claims 1-103 or a pharmaceutically acceptable salt thereof, and a pharmaceutically acceptable carrier.

105. The pharmaceutical composition of claim 104, wherein the pharmaceutical composition is formulated for inhalation, intranasal administration, oral administration, topical administration, intravenous administration, subcutaneous administration, intra-articular administration, intramuscular administration, intraperitoneal administration, intra joint administration, or any combination thereof.

106. The pharmaceutical composition of claim 104 or 105, wherein the pharmaceutical composition is in a single unit dose.

107. The pharmaceutical composition of any one of claims 104-106, wherein the pharmaceutical composition is a liquid.

108. The pharmaceutical composition of any one of claims 104-106, wherein the pharmaceutical composition is a solid dosage form.

109. The pharmaceutical composition of any one of claims 104-108, wherein the pharmaceutical composition is lyophilized.

110. A kit that comprises the conjugate of any one of claims 1-103 or the pharmaceutical composition of any one of claims 104-109 in a container and instructions for use thereof.

111. A method, comprising administering to a subject in need thereof the conjugate of any one of claims 1-103 or the pharmaceutical composition of any one of claims 104-109.

112. The method of claim 111, wherein the method provides the subject with reduction or prevention of an anti-arthritic agent-associated adverse effect, compared to that provided by a corresponding administration of the anti-arthritic agent alone.

113. The method of claim 111, wherein the method reduces occurrence of the adverse effect in the subject, compared to the administration of the anti-arthritic agent alone.

114. The method of claim 111, wherein the method reduces intensity of the adverse effect in the subject, compared to the administration of the anti-arthritic agent alone.

115. The method of any one of claims 111-114, wherein the method reduces the occurrence or intensity of the adverse effect by at least 10%-20%.

116. The method of claim 115, wherein the method reduces the occurrence or intensity of the adverse effect or both by at least 10%-50%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

117. The method of claim 115 or 116, wherein the reduction is measured at 1, 2, 3, 6, 9, 12, 18, or 24 months following the administration.

118. The method of claim 115 or 116, wherein the reduction is measured at 1, 2, 3, 4, 5, 6, 7, 8, 9, 10, 11, 12, 13, 14, or 15 days following the administration.

119. The method of claim 115 or 116, wherein the reduction is measured at 1, 2, 3, 4, 5, 6, 7, 8, 9, or 10 weeks following the administration.

120. The method of claim 115 or 116, wherein the reduction is measured at 1, 2, 3, 4, 5, 6, 7, 8, 9, 10, 11, or 12 months following the administration.

121. The method of any one of claims 111-120, wherein the adverse effect comprises: body weight loss, immunosuppression, skin thinning, purpura, Cushingoid appearance, cataract or glaucoma in an eye, osteoporosis or bone fractures, hypothalamic-pituitary-adrenal (HPA) axis suppression, hyperglycemia and diabetes, increased incidence of serious cardiovascular events, dyslipidemia, myopathy, gastritis, gastrointestinal ulcers and bleeding, psychiatric disturbance, increased blood glucose, decreased serum cortisol or corticosterone, atrophy of adrenal gland, thymus, or spleen, reduction in circulating lymphocytes, decreased cellularity of bone marrow, muscular atrophy, decreased muscle function, pain, muscular pain, arthritic pain, joint pain, joint deformity, decreased mobility, decreased range of motion in a joint, decreased flexibility, decreased strength, decreased balance, impaired glucose tolerance, loss of appetite, decreased bone metabolism, impaired immunity, nephrotic syndrome, fatigability, fungal infection, viral infection, bacterial infection, GI perforation, behavioral and mood disturbances, secondary adrenocortical insufficiency, water retention, cataracts, glaucoma, elevated blood pressure, osteoporosis, suppression of growth in children, increased insulin requirements, weight gain, nausea, Cushing's syndrome, malfunctions of the musculoskeletal, gastrointestinal, dermatologic, neurologic, endocrine, ophthalmic, metabolic, or cardiovascular systems, or any combination thereof.

122. The method of claim 120, wherein the adverse effect is the body weight loss.

123. The method of claim 122, wherein the method results in less than 5% reduction of a total body weight of the subject over 12 days following the administration, compared to the administration of the anti-arthritic agent alone.

124. The method of claim 122, wherein the administration of the conjugate results in less than 10% reduction of a total body weight of the subject over 13 days following the administration, compared to the administration of the anti-arthritic agent alone.

125. The method of claim 120, wherein the adverse effect comprises immunosuppression that is characterized by decreased function or numbers of neutrophils, lymphocytes, monocytes, macrophages, or any combination thereof.

126. The method of claim 120, wherein the adverse effect comprises immunosuppression that is characterized by T cell deficiency, humoral immune deficiency, neutropenia, or any combination thereof.

127. The method of any one of claims 111-126, wherein the method results in lower toxicity to the subject, compared to a corresponding administration of the anti-arthritic agent alone.

128. The method of any one of claims 111-127, wherein the conjugate is therapeutically effective at a lower dosage compared to the anti-arthritic agent alone.

129. The method of any one of claims 111-128, wherein the conjugate is therapeutically effective at a less dosing frequency compared to the anti-arthritic agent alone.

130. The method of any one of claims 111-129, wherein the conjugate is released within 15-60 minutes following the administration.

131. The method of claim 130, wherein the conjugate is released within 15-30 minutes following the administration.

132. The method of any one of claims 111-131, wherein the conjugate has a half-life greater than: 1, 3, 6, 12, 24, or 32 hours.

133. The method of any one of claims 111-132, wherein the conjugate accumulates in a target cartilage or joint within 1-3 hours.

134. The method of any one of claims 111-133, wherein the conjugate is cleaved at a target cartilage or joint after the administration.

135. The method of any one of claims 111-134, wherein the administration is by inhalation, intranasally, orally, topically, intravenously, subcutaneously, intra-articularly, intramuscularly administration, intraperitoneally, or any combination thereof.

136. The method of any one of claims 111-135, wherein the method treats or prevents a condition associated with a function of cartilage in the subject.

137. The method of claim 136, wherein the method provides the subject with increased amelioration of a condition associated with a function of cartilage compared to that provided by a corresponding administration of the anti-arthritic agent alone.

138. The method of claim 136 or 137, wherein the condition is an inflammation, a cancer, a degradation, a growth disturbance, a genetic disease, a tear, an infection, or an injury.

139. The method of claim 136 or 137, wherein the condition is a chondrodystrophy.

140. The method of claim 136 or 137, wherein the condition is a traumatic rupture or detachment.

141. The method of claim 136 or 137, wherein the condition is a costochondritis.

142. The method of claim 136 or 137, wherein the condition is a herniation.

143. The method of claim 136 or 137, wherein the condition is a polychondritis.

144. The method of claim 136 or 137, wherein the condition is a chordoma.

145. The method of claim 136 or 137, wherein the condition is a type of arthritis.

146. The method of claim 145, wherein the type of arthritis is rheumatoid arthritis.

147. The method of claim 145, wherein the type of arthritis is osteoarthritis.

148. The method of claim 145, wherein the type of arthritis is ankylosing spondylitis.

149. The method of claim 145, wherein the type of arthritis is psoriatic arthritis.

150. The method of claim 145, wherein the type of arthritis is gout.

151. The method of claim 136 or 137, wherein the condition is achondroplasia.

152. The method of claim 136 or 137, wherein the condition is benign chondroma or malignant chondrosarcoma.

153. The method of claim 136 or 137, wherein the condition is a lupus nephritis, lupus arthritis, or systemic lupus erythematosus.

154. The method of claim 136 or 137, wherein the condition is bursitis, tendinitis, gout, pseudogout, an arthropathy, or an infection.

155. The method of claim 136 or 137, wherein the condition is an injury, damaged tissue from an injury, or pain caused by an injury.

156. The method of claim 136 or 137, wherein the condition is a tear or damaged tissue from a tear.

157. The method of any one of claims 111-156, wherein the administration occurs 1, 2, 3, or 4 times yearly.

158. The method of any one of claims 111-156, wherein the administration occurs 1, 2, 3, 4, 5, 6, 7, or 8 times daily.

159. The method of any one of claims 111-156, wherein the administration occurs 1, 2, 3, 4, 5, 6, or 7 times weekly.

160. The method of any one of claims 111-156, wherein the administration occurs 1, 2, 3, 4, 5, 6, 7, 8, 9, or 10 times monthly.

161. The method of any one of claims 111-156, wherein the conjugate is administered at 0.2-20 mg/kg, 0.01-0.2 mg/kg, 0.0001-0.001 mg/kg, or 0.001-0.01 mg/kg per body weight of the subject.

162. The method of any one of claims 111-161, wherein the subject is a human.

163. A method of making the conjugate of any one of claims 1-103, the method comprising:
a) mixing the linker and the anti-arthritic agent to form an ester bond, a carbamate bond, or an amide bond; and
b) adding the cystine-dense peptide to form an ester bond, a carbamate bond, or an amide bond with the linker.

164. The method of claim 163, further comprising activating a conjugating site of the anti-arthritic agent before step a).

165. The method of claim 163 or 164, further comprising activating a functional group of the linker before step b).

166. A method of lowering a side effect in a patient undergoing treatment with an anti-arthritic agent, comprising administering to the patient the conjugate of any one of claims 1-103 or the pharmaceutical composition of any one of claims 104-109.

167. The method of claim 166, wherein the side effect comprises: body weight loss, immunosuppression, skin thinning, purpura, Cushingoid appearance, cataract or glaucoma in an eye, osteoporosis or bone fractures, hypothalamic-pituitary-adrenal (HPA) axis suppression, hyperglycemia and diabetes, increased incidence of serious cardiovascular events, dyslipidemia, myopathy, gastritis, gastrointestinal ulcers and bleeding, psychiatric disturbance, increased blood glucose, decreased serum cortisol or corticosterone, atrophy of adrenal gland, thymus, or spleen, reduction in circulating lymphocytes, decreased cellularity of bone marrow, muscular atrophy, decreased muscle function, pain, muscular pain, arthritic pain, joint pain, joint deformity, decreased mobility, decreased range of motion in a joint, decreased flexibility, decreased strength, decreased balance, impaired glucose tolerance, loss of appetite, decreased bone metabolism, impaired immunity, nephrotic syndrome, fatigability, fungal infection, viral infection, bacterial infection, GI perforation, behavioral and mood disturbances, secondary adrenocortical insufficiency, water retention, cataracts, glaucoma, elevated blood pressure, osteoporosis, suppression of growth in children, increased insulin requirements, weight gain, nausea, Cushing's syndrome, malfunctions of the musculoskeletal, gastrointestinal, dermatologic, neurologic, endocrine, ophthalmic, metabolic, or cardiovascular systems, or any combination thereof.
